(19) United States
(12) Patent Application Publication
(10) Pub. No.: US 2015/0216168 A1
(43) Pub. Date: Aug. 6, 2015

(54) USE OF SUBSTITUTED 2-AMIDOBENZIMIDAZOLES, 2-AMIDOBENZOXAZOLES AND 2-AMIDOBENZOTHIAZOLES OR SALTS THEREOF AS ACTIVE SUBSTANCES AGAINST ABIOTIC PLANT STRESS

(71) Applicant: BAYER CROPSCIENCE AG, Monhelm (DE)

(72) Inventors: Jens Frackenpohl, Frankfurt (DE); Ines Heinemann, Hofheim (DE); Thomas Müller, Frankfurt (DE); Jan Dittgen, Frankfurt (DE); Pascal Von Koskull-Döring, Frankfurt (DE); Dirk Schmutzler, Hattersheim (DE); Martin Jeffrey Hills, Idstein (DE)

(21) Appl. No.: 14/424,260

(22) PCT Filed: Sep. 3, 2013
(86) PCT No.: PCT/EP2013/068167

§ 371 Date: Feb. 26, 2015

(30) Foreign Application Priority Data
Sep. 5, 2012 (EP) 12183149.9

(51) Int. Cl. A01N 43/78; A01N 43/52; C07D 277/64; C07D 263/57; A01N 43/76; C07D 235/18; C07D 401/04

(52) U.S. Cl. CPC A01N 43/78; C07D 235/18; A01N 43/52; C07D 401/04; C07D 263/57; A01N 43/76; C07D 277/64

(57) ABSTRACT

The use of substituted 2-amidobenzimidazoles, 2-amidobenzoxazoles and 2-amidobenzothiazoles of the general formula (I) or salts thereof

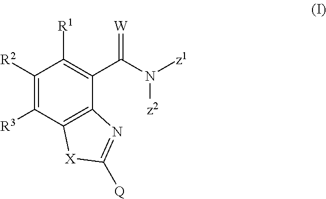

where the radicals in the general formula (I) correspond to the definitions given in the description,
for enhancing stress tolerance in plants to abiotic stress, for strengthening plant growth and/or for increasing plant yield, and to selected processes for preparing the compounds mentioned above.

The invention relates to the use of substituted 2-amidobenzimidazoles, 2-amidobenzoxazoles and 2-amidobenzothiazoles or their respective salts as active compounds for increasing stress tolerance in plants with respect to abiotic stress, in particular for increasing plant growth and/or for increasing plant yield.

It is known that certain substituted benzimidazoles can be used as pesticides (cf. WO94/11349) and that certain haloalkyl-substituted 2-amidobenzimidazoles can be used as active compounds against abiotic plant stress (cf. WO2011107504).

It is also known that substituted amidobenzimidazoles can be used as active pharmaceutical ingredients (cf. WO2000029384 and WO2000026192) and for cosmetic uses (cf. WO2001082877). WO97/04771 likewise describes the pharmaceutical use of predominantly aryl-substituted benzimidazoles, while WO2000032579 describes heterocyclyl-substituted benzimidazoles. The preparation of heterocyclyl-substituted benzimidazoles and their inhibiting action on enzymes from the family of the poly(ADP-ribose)polymerase is described, for example, in Org. Proc. Res Devel. 2007, 11, 693; J. Med. Chem. 2009, 52, 1619 and in J. Med. Chem. 2009, 52, 514, whereas J. Med. Chem. 2010, 53, 3142 lists preparation methods for providing specific aryl-substituted benzimidazoles.

WO2010083220, WO199524379 and US20090197863 describe substituted 2-amidobenzoxazoles as pharmaceutically active compounds and chemotherapeutics. The use of substituted 2-amidobenzoxazoles as antiviral active compounds for the treatment of hepatitis C is likewise known (WO2011047390). Moreover, the literature describes various 2-substituted benzoxazoles as 5-HT₃ receptor antagonists (cf. Bioorg. Med. Chem. Lett. 2010, 20, 6538). The preparation of certain substituted benzoxazoles and benzothiazoles and their cytostatic action is described in Bioorg. Med. Chem. Lett. 2006, 14, 6106. However, the amidobenzoxazoles and -thiazoles according to the invention have not been described as having been used for increasing the stress tolerance in plants with respect to abiotic stress, for enhancing plant growth and/or for increasing the plant yield.

It is also known that substituted 2-amidobenzothiazoles can be used as pharmaceutically active compounds (cf. WO2010083199).

It is known that plants react to natural stress conditions, for example cold, heat, drought, injury, pathogenic attack (viruses, bacteria, fungi, insects), etc., but also to herbicides, with specific or unspecific defense mechanisms [Pflanzenbiochemie, pp. 393-462, Spektrum Akademischer Verlag, Heidelberg, Berlin, Oxford, Hans W. Heldt, 1996; Biochemistry and Molecular Biology of Plants, pp. 1102-1203, American Society of Plant Physiologists, Rockville, Md., eds. Buchanan, Gruissem, Jones, 2000].

In plants, there is knowledge of numerous proteins, and the genes which code for them, which are involved in defense reactions to abiotic stress (for example cold, heat, drought, salt, flooding). Some of these form part of signal transduction chains (e.g. transcription factors, kinases, phosphatases) or cause a physiological response of the plant cell (e.g. ion transport, detoxification of reactive oxygen species). The signaling chain genes of the abiotic stress reaction include inter alia transcription factors of the DREB and CBF classes (Jaglo-Ottosen et al., 1998, Science 280: 104-106). Phosphatases of the ATPK and MP2C type are involved in the reaction to salt stress. In addition, in the event of salt stress, the biosynthesis of osmolytes such as proline or sucrose is frequently activated. This involves, for example, sucrose synthase and proline transporters (Hasegawa et al., 2000, Annu Rev Plant Physiol Plant Mol Biol 51: 463-499). The stress defense of the plants to cold and drought uses some of the same molecular mechanisms. There is a known accumulation of what are called late embryogenesis abundant proteins (LEA proteins), which include the dehydrins as an important class (Ingram and Bartels, 1996, Annu Rev Plant Physiol Plant Mol Biol 47: 277-403, Close, 1997, Physiol Plant 100: 291-296). These are chaperones which stabilize vesicles, proteins and membrane structures in stressed plants (Bray, 1993, Plant Physiol 103: 1035-1040). In addition, there is frequently induction of aldehyde dehydrogenases, which detoxicate the reactive oxygen species (ROS) which form in the event of oxidative stress (Kirch et al., 2005, Plant Mol Biol 57: 315-332).

Heat shock factors (HSF) and heat shock proteins (HSP) are activated in the event of heat stress and play a similar role here as chaperones to that of dehydrins in the event of cold and drought stress (Yu et al., 2005, Mol Cells 19: 328-333).

A number of plant-endogenous signaling substances involved in stress tolerance or pathogen defense are already known. Examples of these include salicylic acid, benzoic acid, jasmonic acid or ethylene [Biochemistry and Molecular Biology of Plants, pp. 850-929, American Society of Plant Physiologists, Rockville, Md., eds. Buchanan, Gruissem, Jones, 2000]. Some of these substances or the stable synthetic derivatives and derived structures thereof are also effective on external application to plants or in seed dressing, and activate defense reactions which cause elevated stress tolerance or pathogen tolerance of the plant [Sembdner, and Parthier, 1993, Ann. Rev. Plant Physiol. Plant Mol. Biol. 44: 569-589].

It is additionally known that chemical substances can increase tolerance of plants to abiotic stress. Such substances are applied either by seed dressing, by leaf spraying or by soil treatment. For instance, an increase in abiotic stress tolerance of crop plants by treatment with elicitors of systemic acquired resistance (SAR) or abscisic acid derivatives is described (Schading and Wei, WO-200028055, Abrams and Gusta, US-5201931, Churchill et al., 1998, Plant Growth Regul 25: 35-45) or azibenzolar-S-methyl. In the case of use of fungicides, especially from the group of the strobilurins or of the succinate dehydrogenase inhibitors, similar effects are also observed, and are frequently also accompanied by an enhanced yield (Draber et al., DE-3534948, Bartlett et al., 2002, Pest Manag Sci 60: 309). It is likewise known that the herbicide glyphosate in low dosage stimulates the growth of some plant species (Cedergreen, Env. Pollution 2008, 156, 1099).

In addition, effects of growth regulators on the stress tolerance of crop plants have been described (Morrison and Andrews, 1992, J Plant Growth Regul 11: 113-117, RD-259027). In the event of osmotic stress, a protective effect resulting from application of osmolytes, for example glycine betaine or the biochemical precursors thereof, for example choline derivatives, has been observed (Chen et al., 2000, Plant Cell Environ 23: 609-618, Bergmann et al., DE-4103253). The effect of antioxidants, for example naphthols and xanthines, to increase abiotic stress tolerance in plants has also already been described (Bergmann et al., DD-277832, Bergmann et al., DD-277835). The molecular causes of the antistress action of these substances are, however, largely unknown.

It is additionally known that the tolerance of plants to abiotic stress can be increased by a modification of the activity of endogenous poly-ADP-ribose polymerases (PARP) or poly-(ADP-ribose)glycohydrolases (PARG) (de Block et al., The Plant Journal, 2005, 41, 95; Levine et al., FEBS Lett. 1998, 440, 1; WO0004173; WO04090140).

It is thus known that plants possess several endogenous reaction mechanisms which can cause effective defense against a wide variety of harmful organisms and/or natural abiotic stress.

Since, however, the ecologic and economic demands on modern crop treatment compositions are increasing constantly, for example with respect to toxicity, selectivity, application rate, formation of residues and favorable manufacture, there is a constant need to develop novel crop treatment compositions which have advantages over those known, at least in some areas.

It was therefore an object of the present invention to provide further compounds which increase tolerance to abiotic stress in plants.

The present invention accordingly provides for the use of substituted 2-amidobenzimidazoles, 2-amidobenzoxazoles and 2-amidobenzothiazoles of the general formula (I) or salts thereof

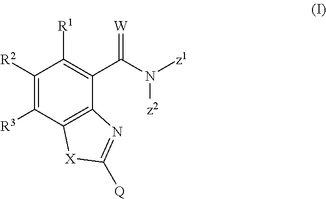

for increasing tolerance to abiotic stress in plants, where
R¹, R², R³ independently of one another represent hydrogen, halogen, alkyl, cycloalkyl, cycloalkenyl, halocycloalkyl, alkenyl, alkynyl, aryl, arylalkyl, arylalkenyl, heteroaryl, heteroarylalkyl, heterocyclyl, heterocyclylalkyl, alkoxyalkyl, alkylthio, haloalkylthio, haloalkyl, alkoxy, haloalkoxy, cycloalkoxy, cycloalkylalkoxy, aryloxy, heteroaryloxy, alkoxyalkoxy, alkynylalkoxy, alkenyloxy, bisalkylaminoalkoxy, tris[alkyl]silyl, bis[alkyl]arylsilyl, bis[alkyl]alkylsilyl, tris[alkyl]silylalkynyl, arylalkynyl, heteroarylalkynyl, alkylalkynyl, cycloalkylalkynyl, haloalkylalkynyl, heterocyclyl-N-alkoxy, nitro, cyano, amino, alkylamino, bisalkylamino, alkylcarbonylamino, cycloalkylcarbonylamino, arylcarbonylamino, alkoxycarbonylamino, heteroarylalkoxy, arylalkoxy, heterocyclylalkoxy, cycloalkylalkyl, haloalkenyl, haloalkynyl, heterocyclylalkynyl, halocycloalkoxy, haloalkynyloxy, arylthio, heteroarylthio, alkylsulfinyl, haloalkylsulfinyl, arylsulfinyl, heteroarylsulfinyl, alkylsulfonyl, haloalkylsulfonyl, arylsulfonyl, heteroarylsulfonyl, thiocyanato, isothiocyanato, cycloalkylamino, cycloalkyl(alkyl)amino, alkenylamino,
R¹ and R² with the atoms to which they are attached form a fully saturated, partially saturated or unsaturated 5- to 7-membered ring which is optionally interrupted by heteroatoms and optionally substituted further,
R² and R³ with the atoms to which they are attached form a fully saturated, partially saturated or unsaturated 5- to 7-membered ring which is optionally interrupted by heteroatoms and optionally substituted further,
W represents oxygen, sulfur,
X represents oxygen, sulfur, N—R⁴,
R⁴ represents hydrogen, alkyl, cycloalkyl, cycloalkylalkyl, cycloalkenyl, cyanoalkyl, alkenylalkyl, haloalkyl, alkynylalkyl, arylalkyl, heteroarylalkyl, heterocyclylalkyl, alkylcarbonyl, cycloalkylcarbonyl, arylcarbonyl, heteroarylcarbonyl, haloalkylcarbonyl, heterocyclylcarbonyl, arylalkylcarbonyl, alkoxycarbonyl, cycloalkylalkoxycarbonyl, cycloalkoxycarbonyl, alkoxycarbonylalkyl, alkylsulfonyl, cycloalkylsulfonyl, arylsulfonyl, heteroarylsulfonyl, alkoxycarbonylcarbonyl, arylalkoxycarbonylcarbonyl, alkylaminothiocarbonyl, alkylaminocarbonyl, cycloalkylaminocarbonyl, alkoxyalkyl, bis[alkyl]aminocarbonyl, aryl, heteroaryl, heterocyclyl, cycloalkoxycarbonylalkyl,
Q represents alkyl, alkenyl, cycloalkyl, cycloalkylalkyl, cycloalkenyl, aryl, arylalkyl, heteroarylalkyl, heterocyclylalkyl, heteroaryl, heterocyclyl, heterocyclylaryl, heterocyclylheteroaryl, heteroarylheteroaryl, heteroarylaryl, arylaryl, aryloxyaryl, arylalkenyl, heteroarylalkenyl, heterocyclylalkenyl, arylalkynyl, heteroarylalkynyl, heterocyclylalkynyl, cycloalkylalkynyl, alkylaminoalkyl, bisalkylaminoalkyl, hydroxyalkyl, alkoxyalkyl, tris[alkyl]silyloxyalkyl, bis[alkyl]arylsilyloxyalkyl, bis[alkyl]alkylsilyloxyalkyl, bisalkylaminoalkoxyalkyl, alkoxyalkoxyalkyl, aryloxyalkyl, heteroaryloxyalkyl, alkylthioalkyl, arylthioalkyl, heteroarylthioalkyl, alkoxycarbonyl-N-heterocyclyl, arylalkoxycarbonyl-N-heterocyclyl, alkyl-N-heterocyclyl, alkylsulfonyl-N-heterocyclyl, arylsulfonyl-N-heterocyclyl, heteroarylsulfonyl-N-heterocyclyl, cycloalkylsulfonyl-N-heterocyclyl, haloalkylsulfonyl-N-heterocyclyl, alkylcarbonyl-N-heterocyclyl, arylcarbonyl-N-heterocyclyl, heteroarylcarbonyl-N-heterocyclyl, cycloalkylcarbonyl-N-heterocyclyl, cycloalkyl-N-heterocyclyl, aryl-N-heterocyclyl, arylalkyl-N-heterocyclyl, bisalkylaminoalkyl-N-heterocyclyl, bisalkylaminosulfonyl-N-heterocyclyl, heteroaryloxyaryl, heteroaryloxyheteroaryl, aryloxyheteroaryl, alkylsulfinyl, alkylthio, alkylsulfonyl, cycloalkylsulfinyl, cycloalkylthio, cycloalkylsulfonyl, arylsulfinyl, arylthio, arylsulfonyl, amino, alkylamino, arylamino, arylalkylamino, cycloalkylamino, formyl, alkylcarbonyl, arylcarbonyl, iminoalkyl, alkyliminoalkyl, aryliminoalkyl, alkoxycarbonyl, cycloalkoxycarbonyl, cycloalkylalkoxycarbonyl, arylalkoxycarbonyl, arylalkylaminocarbonyl, aminocarbonyl, alkylaminocarbonyl, cycloalkylaminocarbonyl, bisalkylaminocarbonyl heterocyclyl-N-carbonyl, imino, alkylimino, arylimino, cycloalkylimino, cycloalkylalkylimino, hydroxyimino, alkoxyimino, cycloalkoxyimino, cycloalkylalkoxyimino, heteroaryloxyimino, aryloxyimino, arylalkoxyimino, alkenyloxyimino, heteroarylalkoxyimino, heteroarylimino, heterocyclylimino, heterocyclylalkylimino, aminoimino, alkylaminoimino, arylaminoimino, heteroarylaminoimino, cycloalkylaminoimino, bisalkylaminoimino, arylalkylaminoimino, aryl(alkyl)aminoimino, cycloalkyl(alkyl)aminoimino, cycloalkylalkylaminoimino, heterocyclylaminoimino, heteroarylalkoxyalkyl, arylalkoxyalkyl, heterocyclyl-N-alkyl, aryl(alkyl)aminoalkyl, arylalkyl(alkyl)aminoalkyl, alkoxycarbonylalkylaminoalkyl, alkoxycarbonylalkyl(alkyl)aminoalkyl, heteroaryl(alkyl)aminoalkyl, heteroarylalkyl(alkyl)aminoalkyl, cycloalkyl(alkyl)aminoalkyl, cycloalkylaminoalkyl, alkoxy(alkoxy)alkyl, arylalkoxyalkylaryl, heterocyclyl-N-alkylaryl, aryl(alkyl)aminoalkylaryl, arylalkyl(alkyl)aminoalkylaryl, alkoxycarbonylalkylaminoalkylaryl, alkoxycarbonylalkyl(alkyl)aminoalkylaryl, heteroaryl(alkyl)aminoalkylaryl, heteroarylalkyl(alkyl)aminoalkylaryl, cycloalkyl(alkyl)aminoalkylaryl, cycloalkylaminoalkylaryl, alkoxy(alkoxy)alkylaryl,
Q additionally, if X represents an oxygen atom or X represents a sulfur atom, represents haloalkyl, alkoxyhaloalkyl, halocycloalkyl, haloalkoxyhaloalkyl, arylhaloalkyl, alkylthiohaloalkyl, bisalkylaminoalkoxyhaloalkyl,
Z¹ represents hydrogen, hydroxy, alkyl, cycloalkyl, halocycloalkyl, halogen, alkenylalkyl, haloalkyl, alkynyl, alkenyl, cyanoalkyl, nitroalkyl, aminoalkyl, alkylaminoalkyl, bis[alkyl]aminoalkyl, alkynylalkyl, arylalkyl, heteroarylalkyl, heterocyclylalkyl, alkylcarbonyl, alkoxycarbonyl, cycloalkoxycarbonyl, alkylsulfonyl, arylsulfonyl, heteroarylsulfonyl, cycloalkylsulfonyl, alkylsulfinyl, arylsulfinyl, cycloalkylsulfinyl, alkoxycarbonylalkyl, aryl, heteroaryl, heterocyclyl, aminocarbonylalkyl, alkylaminocarbonylalkyl, bisalkylaminocarbonylalkyl, cycloalkylaminocarbonylalkyl, hydroxycarbonylalkyl, amino, alkylamino, arylamino, alkoxy, cycloalkylalkyl
   and
Z² represents hydrogen, alkyl, cycloalkyl, branched or straight-chain haloalkyl, alkynyl, alkenyl, cyanoalkyl, arylalkyl, heteroarylalkyl, alkylcarbonyl, alkoxycarbonyl
   or
Z¹ and Z² together with the atoms to which they are attached form a fully saturated or partially saturated 5- to 7-membered ring which is optionally interrupted by heteroatoms and optionally substituted further
   or
Z¹ and Z² together are part of an optionally substituted sulfilimine or amidine group or form an iminophosphorane.

The compounds of the general formula (I) can form salts by addition of a suitable inorganic or organic acid, for example mineral acids, for example HCl, HBr, H₂SO₄, H₃PO₄ or HNO₃, or organic acids, for example carboxylic acids such as formic acid, acetic acid, propionic acid, oxalic acid, lactic acid or salicylic acid or sulfonic acids, for example p-toluenesulfonic acid, onto a basic group, for example amino, alkylamino, dialkylamino, piperidino, morpholino or pyridino. In such a case, these salts comprise the conjugated base of the acid as the anion. Suitable substituents present in deprotonated form, such as, for example, sulfonic acids or carboxylic acids, may form inner salts with groups which for their part can be protonated, such as amino groups.

The compounds of the formula (I) used in accordance with the invention and salts thereof are referred to hereinafter as “compounds of the general formula (I)”.

Preference is given to the use according to the invention of compounds of the formula (I) in which
R¹, R², R³ independently of one another represent hydrogen, halogen, (C₁-C₈)-alkyl, (C₃-C₈)-cycloalkyl, (C₄-C₈)-cycloalkenyl, (C₃-C₈)-halocycloalkyl, (C₂-C₈)-alkenyl, (C₂-C₈)-alkynyl, optionally substituted phenyl, aryl-(C₁-C₈)-alkyl, aryl-(C₂-C₈)-alkenyl, heteroaryl, heteroaryl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio, (C₁-C₈)-haloalkylthio, (C₁-C₈)-haloalkyl, (C₁-C₈)-alkoxy, (C₁-C₈)-haloalkoxy, (C₃-C₈)-cycloalkoxy, (C₃-C₈)-cycloalkyl-(C₁-C₈)-alkoxy, aryloxy, heteroaryloxy, (C₁-C₈)-alkoxy-(C₁-C₈)-alkoxy, (C₂-C₈)-alkynyl-(C₁-C₈)-alkoxy, (C₂-C₈)-alkenyloxy, bis[(C₁-C₈)-alkyl]amino-(C₁-C₈)-alkoxy, tris[(C₁-C₈)-alkyl]silyl, bis[(C₁-C₈)-alkyl]arylsilyl, bis[(C₁-C₈)-alkyl]-(C₁-C₈)-alkylsilyl, tris[(C₁-C₈)-alkyl]silyl-(C₂-C₈)-alkynyl, aryl-(C₂-C₈)-alkynyl, heteroaryl-(C₂-C₈)-alkynyl, (C₁-C₈)-alkyl-(C₂-C₈)-alkynyl, (C₃-C₈)-cycloalkyl-(C₂-C₈)-alkynyl, (C₁-C₈)-haloalkyl-(C₂-C₈)-alkynyl, heterocyclyl-N—(C₁-C₈)-alkoxy, nitro, cyano, amino, (C₁-C₈)-alkylamino, bis[(C₁-C₈)-alkyl]amino, (C₁-C₈)-alkylcarbonylamino, (C₃-C₈)-cycloalkylcarbonylamino, arylcarbonylamino, (C₁-C₈)-alkoxycarbonylamino, heterocyclyl-(C₁-C₈)-alkoxy, (C₃-C₈)-cycloalkyl-(C₁-C₈)-alkyl, (C₂-C₈)-haloalkenyl, (C₂-C₈)-haloalkynyl, heterocyclyl-(C₂-C₈)-alkynyl, (C₃-C₈)-halocycloalkoxy, (C₂-C₈)-haloalkynyloxy, arylthio, heteroarylthio, (C₁-C₈)-alkylsulfinyl, (C₁-C₈)-haloalkylsulfinyl, arylsulfinyl, heteroarylsulfinyl, (C₁-C₈)-alkylsulfonyl, (C₁-C₈)-haloalkylsulfonyl, arylsulfonyl, heteroarylsulfonyl, thiocyanato, isothiocyanato, (C₃-C₈)-cycloalkylamino, (C₃-C₈)-cycloalkyl[(C₁-C₈)-alkyl]amino, (C₂-C₈)-alkenylamino,
R¹ and R² with the atoms to which they are attached form a fully saturated, partially saturated or unsaturated 5- to 7-membered ring which is optionally interrupted by heteroatoms and optionally substituted further,
R² and R³ with the atoms to which they are attached form a fully saturated, partially saturated or unsaturated 5- to 7-membered ring which is optionally interrupted by heteroatoms and optionally substituted further,
W represents oxygen, sulfur,
X represents oxygen, sulfur, N—R⁴,
R⁴ represents hydrogen, (C₁-C₈)-alkyl, (C₃-C₈)-cycloalkyl, (C₃-C₈)-cycloalkyl-(C₁-C₈)-alkyl, (C₄-C₈)-cycloalkenyl, cyano-(C₁-C₈)-alkyl, (C₂-C₈)-alkenyl-(C₁-C₈)-alkyl, (C₁-C₈)-haloalkyl, (C₂-C₈)-alkynyl-(C₁-C₈)-alkyl, aryl-(C₁-C₈)-alkyl, heteroaryl-(C₁-C₈)-alkyl, heterocyclyl-(C₁-C₈)-alkyl, (C₁-C₈)-alkylcarbonyl, (C₃-C₈)-cycloalkylcarbonyl, arylcarbonyl, heteroarylcarbonyl, (C₁-C₈)-haloalkylcarbonyl, heterocyclylcarbonyl, aryl-(C₁-C₈)-alkylcarbonyl, (C₁-C₈)-alkoxycarbonyl, (C₃-C₈)-cycloalkyl-(C₁-C₈)-alkoxycarbonyl, (C₃-C₈)-cycloalkoxycarbonyl, (C₁-C₈)-alkoxycarbonyl-(C₁-C₈)-alkyl, (C₁-C₈)-alkylsulfonyl, (C₃-C₈)-cycloalkylsulfonyl, arylsulfonyl, heteroarylsulfonyl, (C₁-C₈)-alkoxycarbonylcarbonyl, aryl-(C₁-C₈)-alkoxycarbonylcarbonyl, (C₁-C₈)-alkylaminothiocarbonyl, (C₁-C₈)-alkylaminocarbonyl, (C₃-C₈)-cycloalkylaminocarbonyl, (C₁-C₈)-alkoxy-(C₁-C₈)-alkyl, bis[(C₁-C₈)-alkyl]aminocarbonyl, aryl, heteroaryl, heterocyclyl, (C₃-C₈)-cycloalkoxycarbonyl-(C₁-C₈)-alkyl,
Q represents (C₁-C₈)-alkyl, (C₂-C₈)-alkenyl, (C₃-C₈)-cycloalkyl, (C₃-C₈)-cycloalkyl-(C₁-C₈)-alkyl, (C₄-C₈)-cycloalkenyl, optionally substituted phenyl, aryl-(C₁-C₈)-alkyl, heteroaryl-(C₁-C₈)-alkyl, heterocyclyl-(C₁-C₈)-alkyl, heteroaryl, heterocyclyl, heterocyclylaryl, heterocyclyl heteroaryl, heteroarylheteroaryl, heteroarylaryl, arylaryl, aryloxyaryl, aryl-(C₂-C₈)-alkenyl, heteroaryl-(C₂-C₈)-alkenyl, heterocyclyl-(C₂-C₈)-alkenyl, aryl-(C₂-C₈)-alkynyl, heteroaryl-(C₂-C₈)-alkynyl, heterocyclyl-(C₁-C₈)-alkynyl, (C₃-C₈)-cycloalkyl-(C₂-C₈)-alkynyl, (C₁-C₈)-alkylamino-(C₁-C₈)-alkyl, bis[(C₁-C₈)-alkyl]amino-(C₁-C₈)-alkyl, hydroxy-(C₁-C₈)-alkyl, (C₁-C₈)-alkoxy-(C₁-C₈)-alkyl, tris[(C₁-C₈)-alkyl]silyloxy-(C₁-C₈)-alkyl, bis[(C₁-C₈)-alkyl]arylsilyloxy-(C₁-C₈)-alkyl, bis[(C₁-C₈)-alkyl]-(C₁-C₈)-alkylsilyloxy-(C₁-C₈)-alkyl, bis[(C₁-C₈)-alkyl]amino-(C₁-C₈)-alkoxy-(C₁-C₈)-alkyl, (C₁-C₈)-alkoxy-(C₁-C₈)-alkoxy-(C₁-C₈)-alkyl, aryloxy-(C₁-C₈)-alkyl, heteroaryloxy-(C₁-C₈)-alkyl, (C₁-C₈)-alkylthio-(C₁-C₈)-alkyl, arylthio-(C₁-C₈)-alkyl, heteroarylthio-(C₁-C₈)-alkyl, (C₁-C₈)-alkoxycarbonyl-N-heterocyclyl, aryl-(C₁-C₈)-alkoxycarbonyl-N-heterocyclyl, (C₁-C₈)-alkyl-N-heterocyclyl, (C₁-C₈)-alkylsulfonyl-N-heterocyclyl, arylsulfonyl-N-heterocyclyl, heteroarylsulfonyl-N-heterocyclyl, (C₃-C₈)-cycloalkylsulfonyl-N-heterocyclyl, (C₁-C₈)-haloalkylsulfonyl-N-heterocyclyl, (C₁-C₈)-alkylcarbonyl-N-heterocyclyl, arylcarbonyl-N-heterocyclyl, heteroarylcarbonyl-N-heterocyclyl, (C₃-C₈)-cycloalkylcarbonyl-N-heterocyclyl, (C₁-C₈)-cycloalkyl-N-heterocyclyl, aryl-N-heterocyclyl, aryl-(C₁-C₈)-alkyl-N-heterocyclyl, bis[(C₁-C₈)-alkyl]aminoalkyl-N-heterocyclyl, bis[(C₁-C₈)-alkyl]aminosulfonyl-N-heterocyclyl, heteroaryloxyaryl, heteroaryloxyheteroaryl, aryloxyheteroaryl, (C₁-C₈)-alkylsulfinyl, (C₁-C₈)-alkylthio, (C₁-C₈)-alkylsulfonyl, (C₃-C₈)-cycloalkylsulfinyl, (C₃-C₈)-cycloalkylthio, (C₃-C₈)-cycloalkylsulfonyl, arylsulfinyl, arylthio, arylsulfonyl, amino, (C₁-C₈)-alkylamino, bis[(C₁-C₈)-alkyl]amino, arylamino, aryl-(C₁-C₈)-alkylamino, (C₃-C₈)-cycloalkylamino, formyl, (C₁-C₈)-alkylcarbonyl, arylcarbonyl, imino-(C₁-C₈)-alkyl, (C₁-C₈)-alkylimino-(C₁-C₈)-alkyl, arylimino-(C₁-C₈)-alkyl, (C₁-C₈)-alkoxycarbonyl, (C₃-C₈)-cycloalkoxycarbonyl, (C₃-C₈)-cycloalkyl-(C₁-C₈)-alkoxycarbonyl, aryl-(C₁-C₈)-alkoxycarbonyl, aryl-(C₁-C₈)-alkylaminocarbonyl, aminocarbonyl, (C₁-C₈)-alkylaminocarbonyl, (C₃-C₈)-cycloalkylaminocarbonyl, bis[(C₁-C₈)-alkyl]aminocarbonyl, heterocyclyl-N-carbonyl, imino, (C₁-C₈)-alkylimino, arylimino, (C₃-C₈)-cycloalkylimino, (C₃-C₈)-cycloalkyl-(C₁-C₈)-alkylimino, hydroxyimino, (C₁-C₈)-alkoxyimino, (C₂-C₈)-alkenyloxyimino, (C₃-C₈)-cycloalkoxyimino, (C₃-C₈)-cyloalkyl-(C₁-C₈)-alkoxyimino, aryloxyimino, aryl-(C₁-C₈)-alkoxyimino, heteroaryl-(C₁-C₈)-alkoxyimino, heteroaryloxyimino, heteroarylimino, heterocyclylimino, heterocyclyl-(C₁-C₈)-alkylimino, aminoimino, (C₁-C₈)-alkylaminoimino, arylaminoimino, heteroarylaminoimino, (C₃-C₈)-cycloalkylaminoimino, bis[(C₁-C₈)-alkyl]aminoimino, aryl-(C₁-C₈)-alkylaminoimino, aryl[(C₁-C₈)-alkyl]aminoimino, (C₃-C₈)-cycloalkyl[(C₁-C₈)-alkyl]aminoimino, (C₃-C₈)-cycloalkyl-(C₁-C₈)-alkylaminoimino, heterocyclylaminoimino, heteroaryl-(C₁-C₈)-alkoxy-(C₁-C₈)-alkyl, aryl-(C₁-C₈)-alkoxy-(C₁-C₈)-alkyl, heterocyclyl-N—(C₁-C₈)-alkyl, aryl-[(C₁-C₈)-alkyl]amino-(C₁-C₈)-alkyl, aryl-(C₁-C₈)-alkyl[(C₁-C₈)-alkyl]amino-(C₁-C₈)-alkyl, (C₁-C₈)-alkoxycarbonyl-(C₁-C₈)-alkylamino-(C₁-C₈)-alkyl, (C₁-C₈)-alkoxycarbonyl-(C₁-C₈)-alkyl[(C₁-C₈)-alkyl]amino-(C₁-C₈)-alkyl, heteroaryl-[(C₁-C₈)-alkyl]amino-(C₁-C₈)-alkyl, heteroaryl-(C₁-C₈)-alkyl-[(C₁-C₈)-alkyl]amino-(C₁-C₈)-alkyl, —(C₃-C₈)-cycloalkyl-[(C₁-C₈)-alkyl]amino-(C₁-C₈)-alkyl, (C₃-C₈)-cycloalkylamino-(C₁-C₈)-alkyl, aryl-(C₁-C₈)-alkoxy-(C₁-C₈)-alkylaryl, heterocyclyl-N—(C₁-C₈)-alkylaryl, aryl[(C₁-C₈)-alkyl]amino-(C₁-C₈)-alkylaryl, aryl-(C₁-C₈)-alkyl[(C₁-C₈)-alkyl]amino-(C₁-C₈)-alkylaryl, (C₁-C₈)-alkoxycarbonyl-(C₁-C₈)-alkylamino-(C₁-C₈)-alkylaryl, (C₁-C₈)-alkoxycarbonyl-(C₁-C₈)-alkyl[(C₁-C₈)-alkyl]amino-(C₁-C₈)-alkylaryl, heteroaryl[(C₁-C₈)-alkyl]amino-(C₁-C₈)-alkylaryl, heteroaryl-(C₁-C₈)-alkyl[(C₁-C₈)-alkyl]amino-(C₁-C₈)-alkylaryl, (C₃-C₈)-cycloalkyl[(C₁-C₈)-alkyl]amino-(C₁-C₈)-alkylaryl, (C₃-C₈)-cycloalkylamino-(C₁-C₈)-alkylaryl, (C₁-C₈)-alkoxy[(C₁-C₈)-alkoxy]-(C₁-C₈)-alkylaryl,
Q additionally, if X represents an oxygen atom or X represents a sulfur atom, represents (C₁-C₈)-haloalkyl, (C₁-C₈)-alkoxy-(C₁-C₈)-haloalkyl, (C₃-C₈)-halocycloalkyl, (C₁-C₈)-haloalkoxy-(C₁-C₈)-haloalkyl, aryl-(C₁-C₈)-haloalkyl, (C₁-C₈)-alkylthio-(C₁-C₈)-haloalkyl, bis[(C₁-C₈)-alkyl]amino-(C₁-C₈)-alkoxy-(C₁-C₈)-haloalkyl,
Z¹ represents hydrogen, hydroxy, (C₁-C₈)-alkyl, (C₃-C₈)-cycloalkyl, (C₃-C₈)-halocycloalkyl, halogen, (C₂-C₈)-alkenyl-(C₁-C₈)-alkyl, (C₁-C₈)-haloalkyl, (C₂-C₈)-alkynyl, (C₂-C₈)-alkenyl, cyano-(C₁-C₈)-alkyl, nitro-(C₁-C₈)-alkyl, amino-(C₁-C₈)-alkyl, alkyl-(C₁-C₈)-amino-(C₁-C₈)-alkyl, bis[(C₁-C₈)-alkyl]amino-(C₁-C₈)-alkyl, (C₂-C₈)-alkynyl-(C₁-C₈)-alkyl, aryl-(C₁-C₈)-alkyl, heteroaryl-(C₁-C₈)-alkyl, heterocyclyl-(C₁-C₈)-alkyl, (C₁-C₈)-alkylcarbonyl, (C₁-C₈)-alkoxycarbonyl, (C₃-C₈)-cycloalkoxycarbonyl, (C₁-C₈)-alkylsulfonyl, arylsulfonyl, heteroarylsulfonyl, (C₃-C₈)-cycloalkylsulfonyl, (C₁-C₈)-alkylsulfinyl, arylsulfinyl, (C₃-C₈)-cycloalkylsulfinyl, (C₁-C₈)-alkoxycarbonyl-(C₁-C₈)-alkyl, aryl, heteroaryl, heterocyclyl, aminocarbonyl-(C₁-C₈)-alkyl, (C₁-C₈)-alkylaminocarbonyl-(C₁-C₈)-alkyl, bis[(C₁-C₈)-alkyl]aminocarbonyl-(C₁-C₈)-alkyl, (C₃-C₈)-cycloalkylaminocarbonyl-(C₁-C₈)-alkyl, hydroxycarbonyl-(C₁-C₈)-alkyl, amino, (C₁-C₈)-alkylamino, arylamino, (C₁-C₈)-alkoxy
   and
Z² represents hydrogen, (C₁-C₈)-alkyl, (C₃-C₈)-cycloalkyl, (C₁-C₈)-haloalkyl, (C₂-C₈)-alkynyl, (C₂-C₈)-alkenyl, cyano-(C₁-C₈)-alkyl, aryl-(C₁-C₈)-alkyl, heteroaryl-(C₁-C₈)-alkyl, (C₁-C₈)-alkylcarbonyl, (C₁-C₈)-alkoxycarbonyl
   or
Z¹ and Z² together with the atoms to which they are attached form a fully saturated or partially saturated 5- to 7-membered ring which is optionally interrupted by heteroatoms and optionally substituted further
   or
Z¹ and Z² together form an N-(bis(C₁-C₈)-alkyl)sulfanylidene, N-(aryl-(C₁-C₈)-alkyl)sulfanylidene, N-(bis(C₃-C₈)-cycloalkyl)sulfanylidene, N—((C₁-C₈)-alkyl-(C₃-C₈)-cycloalkyl)sulfanylidene group or an N,N-di-(C₁-C₈)-alkylformylidene group.

Particular preference is given to the use according to the invention of compounds of the formula (I) in which
R¹, R², R³ independently of one another represent hydrogen, iodine, bromine, chlorine, fluorine, (C₁-C₇)-alkyl, (C₃-C₇)-cycloalkyl, (C₄-C₇)-cycloalkenyl, (C₃-C₇)-halocycloalkyl, (C₂-C₇)-alkenyl, (C₂-C₇)-alkynyl, optionally substituted phenyl, aryl-(C₁-C₇)-alkyl, aryl-(C₂-C₇)-alkenyl, heteroaryl, heteroaryl-(C₁-C₇)-alkyl, heterocyclyl, heterocyclyl-(C₁-C₇)-alkyl, (C₁-C₇)-alkoxy-(C₁-C₇)-alkyl, (C₁-C₇)-alkylthio, (C₁-C₇)-haloalkylthio, (C₁-C₇)-haloalkyl, (C₁-C₇)-alkoxy, (C₁-C₇)-haloalkoxy, (C₃-C₇)-cycloalkoxy, (C₃-C₇)-cycloalkyl-(C₁-C₇)-alkoxy, aryloxy, heteroaryloxy, (C₁-C₇)-alkoxy-(C₁-C₇)-alkoxy, (C₂-C₇)-alkynyl-(C₁-C₇)-alkoxy, (C₂-C₇)-alkenyloxy, bis[(C₁-C₇)-alkyl]amino-(C₁-C₇)-alkoxy, tris[(C₁-C₇)-alkyl]silyl, bis[(C₁-C₇)-alkyl]arylsilyl, bis[(C₁-C₇)-alkyl]-(C₁-C₇)-alkylsilyl, tris[(C₁-C₇)-alkyl]silyl-(C₂-C₇)-alkynyl, aryl-(C₂-C₇)-alkynyl, heteroaryl-(C₂-C₇)-alkynyl, (Cᵣ C₇)-alkyl-(C₂-C₇)-alkynyl, (C₃-C₇)-cycloalkyl-(C₂-C₇)-alkynyl, (C₁-C₇)-haloalkyl-(C₂-C₇)-alkynyl, heterocyclyl-N—(C₁-C₇)-alkoxy, nitro, cyano, amino, (C₁-C₇)-alkylamino, bis[(C₁-C₇)-alkyl]amino, (C₁-C₇)-alkylcarbonylamino, (C₃-C₇)-cycloalkylcarbonylamino, arylcarbonylamino, (C₁-C₇)-alkoxycarbonylamino, heterocyclyl-(C₁-C₇)-alkoxy, (C₃-C₇)-cycloalkyl-(C₁-C₇)-alkyl, (C₂-C₇)-haloalkenyl, (C₂-C₇)-haloalkynyl, heterocyclyl-(C₂-C₇)-alkynyl, (C₃-C₇)-halocycloalkoxy, (C₂-C₇)-haloalkynyloxy, arylthio, heteroarylthio, (C₁-C₇)-alkylsulfinyl, (C₁-C₇)-haloalkylsulfinyl, arylsulfinyl, heteroarylsulfinyl, (C₁-C₇)-alkylsulfonyl, (C₁-C₇)-haloalkylsulfonyl, arylsulfonyl, heteroarylsulfonyl, thiocyanato, isothiocyanato, (C₃-C₇)-cycloalkylamino, (C₃-C₇)-cycloalkyl[(C₁-C₇)-alkyl]amino, (C₂-C₇)-alkenylamino,
R¹ and R² with the atoms to which they are attached form a fully saturated, partially saturated or unsaturated 5- to 7-membered ring which is optionally interrupted by heteroatoms and optionally substituted further,
R² and R³ with the atoms to which they are attached form a fully saturated, partially saturated or unsaturated 5- to 7-membered ring which is optionally interrupted by heteroatoms and optionally substituted further,
W represents oxygen, sulfur,
X represents oxygen, sulfur, N—R⁴,
R⁴ represents hydrogen, (C₁-C₇)-alkyl, (C₃-C₇)-cycloalkyl, (C₃-C₇)-cycloalkyl-(C₁-C₇)-alkyl, (C₄-C₇)-cycloalkenyl, cyano-(C₁-C₇)-alkyl, (C₂-C₇)-alkenyl-(C₁-C₇)-alkyl, (C₁-C₇)-haloalkyl, (C₂-C₇)-alkynyl-(C₁-C₇)-alkyl, aryl-(C₁-C₇)-alkyl, heteroaryl-(C₁-C₇)-alkyl, heterocyclyl-(C₁-C₇)-alkyl, (C₁-C₇)-alkylcarbonyl, (C₃-C₇)-cycloalkylcarbonyl, arylcarbonyl, heteroarylcarbonyl, (C₁-C₇)-haloalkylcarbonyl, heterocyclylcarbonyl, aryl-(C₁-C₇)-alkylcarbonyl, (C₁-C₇)-alkoxycarbonyl, (C₃-C₇)-cycloalkyl-(C₁-C₇)-alkoxycarbonyl, (C₃-C₇)-cycloalkoxycarbonyl, (C₁-C₇)-alkoxycarbonyl-(C₁-C₇)-alkyl, (C₁-C₇)-alkylsulfonyl, (C₃-C₇)-cycloalkylsulfonyl, arylsulfonyl, heteroarylsulfonyl, (C₁-C₇)-alkoxycarbonylcarbonyl, aryl-(C₁-C₇)-alkoxycarbonylcarbonyl, (C₁-C₇)-alkylaminothiocarbonyl, (C₁-C₇)-alkylaminocarbonyl, (C₃-C₇)-cycloalkylaminocarbonyl, (C₁-C₇)-alkoxy-(C₁-C₇)-alkyl, bis[(C₁-C₇)-alkyl]aminocarbonyl, aryl, heteroaryl, heterocyclyl, (C₃-C₇)-cycloalkoxycarbonyl-(C₁-C₇)-alkyl,
Q represents (C₁-C₇)-alkyl, (C₂-C₇)-alkenyl, (C₃-C₇)-cycloalkyl, (C₃-C₇)-cycloalkyl-(C₁-C₇)-alkyl, (C₄-C₇)-cycloalkenyl, optionally substituted phenyl, aryl-(C₁-C₇)-alkyl, heteroaryl-(C₁-C₇)-alkyl, heterocyclyl-(C₁-C₇)-alkyl, heteroaryl, heterocyclyl, heterocyclylaryl, heterocyclyl heteroaryl, heteroarylheteroaryl, heteroarylaryl, arylaryl, aryloxyaryl, aryl-(C₂-C₇)-alkenyl, heteroaryl-(C₂-C₇)-alkenyl, heterocyclyl-(C₂-C₇)-alkenyl, aryl-(C₂-C₇)-alkynyl, heteroaryl-(C₂-C₇)-alkynyl, heterocyclyl-(C₁-C₇)-alkynyl, (C₃-C₇)-cycloalkyl-(C₂-C₇)-alkynyl, (C₁-C₇)-alkylamino-(C₁-C₇)-alkyl, bis[(C₁-C₇)-alkyl]amino-(C₁-C₇)-alkyl, hydroxy-(C₁-C₇)-alkyl, (C₁-C₇)-alkoxy-(C₁-C₇)-alkyl, tris[(C₁-C₇)-alkyl]silyloxy-(C₁-C₇)-alkyl, bis[(C₁-C₇)-alkyl]arylsilyloxy-(C₁-C₇)-alkyl, bis[(C₁-C₇)-alkyl]-(C₁-C₇)-alkylsilyloxy-(C₁-C₇)-alkyl, bis[(C₁-C₇)-alkyl]amino-(C₁-C₇)-alkoxy-(C₁-C₇)-alkyl, (C₁-C₇)-alkoxy-(C₁-C₇)-alkoxy-(C₁-C₇)-alkyl, aryloxy-(C₁-C₇)-alkyl, heteroaryloxy-(C₁-C₇)-alkyl, (C₁-C₇)-alkylthio-(C₁-C₇)-alkyl, arylthio-(C₁-C₇)-alkyl, heteroarylthio-(C₁-C₇)-alkyl, (C₁-C₇)-alkoxycarbonyl-N-heterocyclyl, aryl-(C₁-C₇)-alkoxycarbonyl-N-heterocyclyl, (C₁-C₇)-alkyl-N-heterocyclyl, (C₁-C₇)-alkylsulfonyl-N-heterocyclyl, arylsulfonyl-N-heterocyclyl, heteroarylsulfonyl-N-heterocyclyl, (C₃-C₇)-cycloalkylsulfonyl-N-heterocyclyl, (C₁-C₇)-haloalkylsulfonyl-N-heterocyclyl, (C₁-C₇)-alkylcarbonyl-N-heterocyclyl, arylcarbonyl-N-heterocyclyl, heteroarylcarbonyl-N-heterocyclyl, (C₃-C₇)-cycloalkylcarbonyl-N-heterocyclyl, (C₁-C₇)-cycloalkyl-N-heterocyclyl, aryl-N-heterocyclyl, aryl-(C₁-C₇)-alkyl-N-heterocyclyl, bis[(C₁-C₇)-alkyl]aminoalkyl-N-heterocyclyl, bis[(C₁-C₇)-alkyl]aminosulfonyl-N-heterocyclyl, heteroaryloxyaryl, heteroaryloxyheteroaryl, aryloxyheteroaryl, (C₁-C₇)-alkylsulfinyl, (C₁-C₇)-alkylthio, (C₁-C₇)-alkylsulfonyl, (C₃-C₇)-cycloalkylsulfinyl, (C₃-C₇)-cycloalkylthio, (C₃-C₇)-cycloalkylsulfonyl, arylsulfinyl, arylthio, arylsulfonyl, amino, (C₁-C₇)-alkylamino, bis[(C₁-C₇)-alkyl]amino, arylamino, aryl-(C₁-C₇)-alkylamino, (C₃-C₇)-cycloalkylamino, formyl, (C₁-C₇)-alkylcarbonyl, arylcarbonyl, imino-(C₁-C₇)-alkyl, (C₁-C₇)-alkylimino-(C₁-C₇)-alkyl, arylimino-(C₁-C₇)-alkyl, (C₁-C₇)-alkoxycarbonyl, (C₃-C₇)-cycloalkoxycarbonyl, (C₃-C₇)-cycloalkyl-(C₁-C₇)-alkoxycarbonyl, aryl-(C₁-C₇)-alkoxycarbonyl, aryl-(C₁-C₇)-alkylaminocarbonyl, aminocarbonyl, (C₁-C₇)-alkylaminocarbonyl, (C₃-C₇)-cycloalkylaminocarbonyl, bis[(C₁-C₇)-alkyl]aminocarbonyl, heterocyclyl-N-carbonyl, imino, (C₁-C₇)-alkylimino, arylimino, (C₃-C₇)-cycloalkylimino, (C₃-C₇)-cycloalkyl-(C₁-C₇)-alkylimino, hydroxyimino, (C₁-C₇)-alkoxyimino, (C₂-C₇)-alkenyloxyimino, (C₃-C₇)-cycloalkoxyimino, (C₃-C₇)-cyloalkyl-(C₁-C₇)-alkoxyimino, aryloxyimino, aryl-(C₁-C₇)-alkoxyimino, heteroaryl-(C₁-C₇)-alkoxyimino, heteroaryloxyimino, heteroarylimino, heterocyclylimino, heterocyclyl-(C₁-C₇)-alkylimino, aminoimino, (C₁-C₇)-alkylaminoimino, arylaminoimino, heteroarylaminoimino, (C₃-C₇)-cycloalkylaminoimino, bis[(C₁-C₇)-alkyl]aminoimino, aryl-(C₁-C₇)-alkylaminoimino, aryl[(C₁-C₇)-alkyl]aminoimino, (C₃-C₇)-cycloalkyl[(C₁-C₇)-alkyl]aminoimino, (C₃-C₇)-cycloalkyl-(C₁-C₇)-alkylaminoimino, heterocyclylaminoimino, heteroaryl-(C₁-C₇)-alkoxy-(C₁-C₇)-alkyl, aryl-(C₁-C₇)-alkoxy-(C₁-C₇)-alkyl, heterocyclyl-N—(C₁-C₇)-alkyl, aryl-[(C₁-C₇)-alkyl]amino-(C₁-C₇)-alkyl, aryl-(C₁-C₇)-alkyl[(C₁-C₇)-alkyl]amino-(C₁-C₇)-alkyl, (C₁-C₇)-alkoxycarbonyl-(C₁-C₇)-alkylamino-(C₁-C₇)-alkyl, (C₁-C₇)-alkoxycarbonyl-(C₁-C₇)-alkyl[(C₁-C₇)-alkyl]amino-(C₁-C₇)-alkyl, heteroaryl-[(C₁-C₇)-alkyl]amino-(C₁-C₇)-alkyl, heteroaryl-(C₁-C₇)-alkyl-[(C₁-C₇)-alkyl]amino-(C₁-C₇)-alkyl, —(C₃-C₇)-cycloalkyl-[(C₁-C₇)-alkyl]amino-(C₁-C₇)-alkyl, (C₃-C₇)-cycloalkylamino-(C₁-C₁)-alkyl, aryl-(C₁-C₇)-alkoxy-(C₁-C₇)-alkylaryl, heterocyclyl-N—(C₁-C₇)-alkylaryl, aryl[(C₁-C₇)-alkyl]amino-(C₁-C₇)-alkylaryl, aryl-(C₁-C₇)-alkyl[(C₁-C₇)-alkyl]amino-(C₁-C₇)-alkylaryl, (C₁-C₇)-alkoxycarbonyl-(C₁-C₇)-alkylamino-(C₁-C₇)-alkylaryl, (C₁-C₇)-alkoxycarbonyl-(C₁-C₇)-alkyl[(C₁-C₇)-alkyl]amino-(C₁-C₇)-alkylaryl, heteroaryl[(C₁-C₇)-alkyl]amino-(C₁-C₇)-alkylaryl, heteroaryl-(C₁-C₇)-alkyl[(C₁-C₇)-alkyl]amino-(C₁-C₇)-alkylaryl, (C₃-C₇)-cycloalkyl[(C₁-C₇)-alkyl]amino-(C₁-C₇)-alkylaryl, (C₃-C₇)-cycloalkylamino-(C₁-C₇)-alkylaryl, (C₁-C₇)-alkoxy[(C₁-C₇)-alkoxy]-(C₁-C₇)-alkylaryl,
Q additionally, if X represents an oxygen atom or X represents a sulfur atom, represents (C₁-C₇)-haloalkyl, (C₁-C₇)-alkoxy-(C₁-C₇)-haloalkyl, (C₃-C₇)-halocycloalkyl, (C₁-C₇)-haloalkoxy-(C₁-C₇)-haloalkyl, aryl-(C₁-C₇)-haloalkyl, (C₁-C₇)-alkylthio-(C₁-C₇)-haloalkyl, bis[(C₁-C₇)-alkyl]amino-(C₁-C₇)-alkoxy-(C₁-C₇)-haloalkyl,
Z¹ represents hydrogen, hydroxy, (C₁-C₇)-alkyl, (C₃-C₇)-cycloalkyl, (C₃-C₇)-halocycloalkyl, bromine, chlorine, (C₂-C₇)-alkenyl-(C₁-C₇)-alkyl, (C₁-C₇)-haloalkyl, (C₂-C₇)-alkynyl, (C₂-C₇)-alkenyl, cyano-(C₁-C₇)-alkyl, nitro-(C₁-C₇)-alkyl, amino-(C₁-C₇)-alkyl, alkyl-(C₁-C₇)-amino-(C₁-C₇)-alkyl, bis[(C₁-C₇)-alkyl]amino-(C₁-C₇)-alkyl, (C₂-C₇)-alkynyl-(C₁-C₇)-alkyl, aryl-(C₁-C₇)-alkyl, heteroaryl-(C₁-C₇)-alkyl, heterocyclyl-(C₁-C₇)-alkyl, (C₁-C₇)-alkylcarbonyl, (C₁-C₇)-alkoxycarbonyl, (C₃-C₇)-cycloalkoxycarbonyl, (C₁-C₇)-alkylsulfonyl, arylsulfonyl, heteroarylsulfonyl, (C₃-C₇)-cycloalkylsulfonyl, (C₁-C₇)-alkylsulfinyl, arylsulfinyl, (C₃-C₇)-cycloalkylsulfinyl, (C₁-C₇)-alkoxycarbonyl-(C₁-C₇)-alkyl, aryl, heteroaryl, heterocyclyl, aminocarbonyl-(C₁-C₇)-alkyl, (C₁-C₇)-alkylaminocarbonyl-(C₁-C₇)-alkyl, bis[(C₁-C₇)-alkyl]aminocarbonyl-(C₁-C₇)-alkyl, (C₃-C₇)-cycloalkylaminocarbonyl-(C₁-C₇)-alkyl, hydroxycarbonyl-(C₁-C₇)-alkyl, amino, (C₁-C₇)-alkylamino, arylamino, (C₁-C₇)-alkoxy
   and
Z² represents hydrogen, (C₁-C₇)-alkyl, (C₃-C₇)-cycloalkyl, (C₁-C₇)-haloalkyl, (C₂-C₇)-alkynyl, (C₂-C₇)-alkenyl, cyano-(C₁-C₇)-alkyl, aryl-(C₁-C₇)-alkyl, heteroaryl-(C₁-C₇)-alkyl, (C₁-C₇)-alkylcarbonyl, (C₁-C₇)-alkoxycarbonyl
   or
Z¹ and Z² together with the atoms to which they are attached form a fully saturated or partially saturated 5- to 7-membered ring which is optionally interrupted by heteroatoms and optionally substituted further
   or
Z¹ and Z² together form an N-(bis(C₁-C₇)-alkyl)sulfanylidene, N-(aryl-(C₁-C₇)-alkyl)sulfanylidene, N-(bis(C₃-C₇)-cycloalkyl)sulfanylidene, N—((C₁-C₇)-alkyl-(C₃-C₇)-cycloalkyl)sulfanylidene group or an N,N-di-(C₁-C₇)-alkylformylidene group.

Very particular preference is given to the use according to the invention of compounds of the formula (I) in which
R¹, R², R³ independently of one another represent hydrogen, iodine, bromine, chlorine, fluorine, (C₁-C₄)-alkyl, (C₃-C₆)-cycloalkyl, (C₄-C₆)-cycloalkenyl, (C₃-C₆)-halocycloalkyl, (C₂-C₄)-alkenyl, (C₂-C₄)-alkynyl, optionally substituted phenyl, aryl-(C₁-C₄)-alkyl, aryl-(C₂-C₄)-alkenyl, heteroaryl, heteroaryl-(C₁-C₄)-alkyl, heterocyclyl, heterocyclyl-(C₁-C₄)-alkyl, (C₁-C₄)-alkoxy-(C₁-C₄)-alkyl, (C₁-C₄)-alkylthio, (C₁-C₄)-haloalkylthio, (C₁-C₄)-haloalkyl, (C₁-C₄)-alkoxy, (C₁-C₄)-haloalkoxy, (C₃-C₆)-cycloalkoxy, (C₃-C₆)-cycloalkyl-(C₁-C₄)-alkoxy, aryloxy, heteroaryloxy, (C₁-C₄)-alkoxy-(C₁-C₄)-alkoxy, (C₂-C₄)-alkynyl-(C₁-C₄)-alkoxy, (C₂-C₄)-alkenyloxy, bis[(C₁-C₄)-alkyl]amino-(C₁-C₄)-alkoxy, tris[(C₁-C₄)-alkyl]silyl, bis[(C₁-C₄)-alkyl]arylsilyl, bis[(C₁-C₄)-alkyl]-(C₁-C₄)-alkylsilyl, tris[(C₁-C₄)-alkyl]silyl-(C₂-C₄)-alkynyl, aryl-(C₂-C₄)-alkynyl, heteroaryl-(C₂-C₄)-alkynyl, (C₁-C₄)-alkyl-(C₂-C₄)-alkynyl, (C₃-C₆)-cycloalkyl-(C₂-C₄)-alkynyl, (C₁-C₄)-haloalkyl-(C₂-C₄)-alkynyl, heterocyclyl-N—(C₁-C₄)-alkoxy, nitro, cyano, amino, (C₁-C₄)-alkylamino, bis[(C₁-C₄)-alkyl]amino, (C₁-C₄)-alkylcarbonylamino, (C₃-C₆)-cycloalkylcarbonylamino, arylcarbonylamino, (C₁-C₄)-alkoxycarbonylamino,
R¹ and R² with the atoms to which they are attached form a fully saturated, partially saturated or unsaturated 5- to 7-membered ring which is optionally interrupted by heteroatoms and optionally substituted further,
R² and R³ with the atoms to which they are attached form a fully saturated, partially saturated or unsaturated 5- to 7-membered ring which is optionally interrupted by heteroatoms and optionally substituted further,
W represents oxygen, sulfur,
X represents oxygen, sulfur, N—R⁴,
R⁴ represents hydrogen, (C₁-C₄)-alkyl, (C₃-C₆)-cycloalkyl, (C₃-C₆)-cycloalkyl-(C₁-C₄)-alkyl, (C₄-C₆)-cycloalkenyl, cyano-(C₁-C₄)-alkyl, (C₂-C₄)-alkenyl-(C₁-C₄)-alkyl, (C₁-C₄)-haloalkyl, (C₂-C₄)-alkynyl-(C₁-C₄)-alkyl, aryl-(C₁-C₄)-alkyl, heteroaryl-(C₁-C₄)-alkyl, heterocyclyl-(C₁-C₄)-alkyl, (C₁-C₄)-alkylcarbonyl, (C₃-C₆)-cycloalkylcarbonyl, arylcarbonyl, heteroarylcarbonyl, (C₁-C₄)-haloalkylcarbonyl, heterocyclylcarbonyl, aryl-(C₁-C₄)-alkylcarbonyl, (C₁-C₄)-alkoxycarbonyl, (C₃-C₆)-cycloalkyl-(C₁-C₄)-alkoxycarbonyl, (C₃-C₆)-cycloalkoxycarbonyl, (C₁-C₄)-alkoxycarbonyl-(C₁-C₄)-alkyl, (C₁-C₄)-alkylsulfonyl, (C₃-C₆)-cycloalkylsulfonyl, arylsulfonyl, heteroarylsulfonyl, (C₁-C₄)-alkoxycarbonylcarbonyl, aryl-(C₁-C₄)-alkoxycarbonylcarbonyl, (C₁-C₄)-alkylaminothiocarbonyl, (C₁-C₄)-alkylaminocarbonyl, (C₃-C₆)-cycloalkylaminocarbonyl, (C₁-C₄)-alkoxy-(C₁-C₄)-alkyl, bis[(C₁-C₄)-alkyl]aminocarbonyl, aryl, heteroaryl, heterocyclyl, (C₃-C₆)-cycloalkoxycarbonyl-(C₁-C₄)-alkyl, heterocyclyl-(C₁-C₈)-alkoxy, (C₃-C₆)-cycloalkyl-(C₁-C₄)-alkyl, (C₂-C₄)-haloalkenyl, (C₂-C₄)-haloalkynyl, heterocyclyl-(C₂-C₄)-alkynyl, (C₃-C₆)-halocycloalkoxy, (C₂-C₄)-haloalkynyloxy, arylthio, heteroarylthio, (C₁-C₄)-alkylsulfinyl, (C₁-C₄)-haloalkylsulfinyl, arylsulfinyl, heteroarylsulfinyl, (C₁-C₄)-alkylsulfonyl, (C₁-C₄)-haloalkylsulfonyl, arylsulfonyl, heteroarylsulfonyl, thiocyanato, isothiocyanato, (C₃-C₆)-cycloalkylamino, (C₃-C₆)-cycloalkyl[(C₁-C₄)-alkyl]amino, (C₂-C₄)-alkenylamino,
Q represents (C₁-C₄)-alkyl, (C₂-C₄)-alkenyl, (C₃-C₆)-cycloalkyl, (C₃-C₆)-cycloalkyl-(C₁-C₄)-alkyl, (C₄-C₆)-cycloalkenyl, optionally substituted phenyl, aryl-(C₁-C₄)-alkyl, heteroaryl-(C₁-C₄)-alkyl, heterocyclyl-(C₁-C₄)-alkyl, heteroaryl, heterocyclyl, heterocyclylaryl, heterocyclyl heteroaryl, heteroarylheteroaryl, heteroarylaryl, arylaryl, aryloxyaryl, aryl-(C₂-C₄)-alkenyl, heteroaryl-(C₂-C₄)-alkenyl, heterocyclyl-(C₂-C₄)-alkenyl, aryl-(C₂-C₄)-alkynyl, heteroaryl-(C₂-C₄)-alkynyl, heterocyclyl-(C₁-C₄)-alkynyl, (C₃-C₆)-cycloalkyl-(C₂-C₄)-alkynyl, (C₁-C₄)-alkylamino-(C₁-C₄)-alkyl, bis[(C₁-C₄)-alkyl]amino-(C₁-C₄)-alkyl, hydroxy-(C₁-C₄)-alkyl, (C₁-C₄)-alkoxy-(C₁-C₄)-alkyl, tris[(C₁-C₄)-alkyl]silyloxy-(C₁-C₄)-alkyl, bis[(C₁-C₄)-alkyl]arylsilyloxy-(C₁-C₄)-alkyl, bis[(C₁-C₄)-alkyl]-(C₁-C₄)-alkylsilyloxy-(C₁-C₄)-alkyl, bis[(C₁-C₄)-alkyl]amino-(C₁-C₄)-alkoxy-(C₁-C₄)-alkyl, (C₁-C₄)-alkoxy-(C₁-C₄)-alkoxy-(C₁-C₄)-alkyl, aryloxy-(C₁-C₄)-alkyl, heteroaryloxy-(C₁-C₄)-alkyl, (C₁-C₄)-alkylthio-(C₁-C₄)-alkyl, arylthio-(C₁-C₄)-alkyl, heteroarylthio-(C₁-C₄)-alkyl, (C₁-C₄)-alkoxycarbonyl-N-heterocyclyl, aryl-(C₁-C₄)-alkoxycarbonyl-N-heterocyclyl, (C₁-C₄)-alkyl-N-heterocyclyl, (C₁-C₄)-alkylsulfonyl-N-heterocyclyl, arylsulfonyl-N-heterocyclyl, heteroarylsulfonyl-N-heterocyclyl, (C₃-C₆)-cycloalkylsulfonyl-N-heterocyclyl, (C₁-C₄)-haloalkylsulfonyl-N-heterocyclyl, (C₁-C₄)-alkylcarbonyl-N-heterocyclyl, arylcarbonyl-N-heterocyclyl, heteroarylcarbonyl-N-heterocyclyl, (C₃-C₆)-cycloalkylcarbonyl-N-heterocyclyl, (C₁-C₆)-cycloalkyl-N-heterocyclyl, aryl-N-heterocyclyl, aryl-(C₁-C₄)-alkyl-N-heterocyclyl, bis[(C₁-C₄)-alkyl]aminoalkyl-N-heterocyclyl, bis[(C₁-C₄)-alkyl]aminosulfonyl-N-heterocyclyl, heteroaryloxyaryl, heteroaryloxyheteroaryl, aryloxyheteroaryl, (C₁-C₄)-alkylsulfinyl, (C₁-C₄)-alkylthio, (C₁-C₄)-alkylsulfonyl, (C₃-C₆)-cycloalkylsulfinyl, (C₃-C₆)-cycloalkylthio, (C₃-C₆)-cycloalkylsulfonyl, arylsulfinyl, arylthio, arylsulfonyl, amino, (C₁-C₄)-alkylamino, bis[(C₁-C₄)-alkyl]amino, arylamino, aryl-(C₁-C₄)-alkylamino, (C₃-C₆)-cycloalkylamino, formyl, (C₁-C₄)-alkylcarbonyl, arylcarbonyl, imino-(C₁-C₄)-alkyl, (C₁-C₄)-alkylimino-(C₁-C₄)-alkyl, arylimino-(C₁-C₄)-alkyl, (C₁-C₄)-alkoxycarbonyl, (C₃-C₆)-cycloalkoxycarbonyl, (C₃-C₆)-cycloalkyl-(C₁-C₄)-alkoxycarbonyl, aryl-(C₁-C₄)-alkoxycarbonyl, aryl-(C₁-C₄)-alkylaminocarbonyl, aminocarbonyl, (C₁-C₄)-alkylaminocarbonyl, (C₃-C₆)-cycloalkylaminocarbonyl, bis[(C₁-C₄)-alkyl]aminocarbonyl, heterocyclyl-N-carbonyl, imino, (C₁-C₄)-alkylimino, arylimino, (C₃-C₆)-cycloalkylimino, (C₃-C₆)-cycloalkyl-(C₁-C₄)-alkylimino, hydroxyimino, (C₁-C₄)-alkoxyimino, (C₂-C₄)-alkenyloxyimino, (C₃-C₆)-cycloalkoxyimino, (C₃-C₆)-cyloalkyl-(C₁-C₄)-alkoxyimino, aryloxyimino, aryl-(C₁-C₄)-alkoxyimino, heteroaryl-(C₁-C₄)-alkoxyimino, heteroaryloxyimino, heteroarylimino, heterocyclylimino, heterocyclyl-(C₁-C₄)-alkylimino, aminoimino, (C₁-C₄)-alkylaminoimino, arylaminoimino, heteroarylaminoimino, (C₃-C₆)-cycloalkylaminoimino, bis[(C₁-C₄)-alkyl]aminoimino, aryl-(C₁-C₄)-alkylaminoimino, aryl[(C₁-C₄)-alkyl]aminoimino, (C₃-C₆)-cycloalkyl[(C₁-C₄)-alkyl]aminoimino, (C₃-C₆)-cycloalkyl-(C₁-C₄)-alkylaminoimino, heterocyclylaminoimino, heteroaryl-(C₁-C₄)-alkoxy-(C₁-C₄)-alkyl, aryl-(C₁-C₄)-alkoxy-(C₁-C₄)-alkyl, heterocyclyl-N—(C₁-C₄)-alkyl, aryl-[(C₁-C₄)-alkyl]amino-(C₁-C₄)-alkyl, aryl-(C₁-C₄)-alkyl[(C₁-C₄)-alkyl]amino-(C₁-C₄)-alkyl, (C₁-C₄)-alkoxycarbonyl-(C₁-C₄)-alkylamino-(C₁-C₄)-alkyl, (C₁-C₄)-alkoxycarbonyl-(C₁-C₄)-alkyl[(C₁-C₄)-alkyl]amino-(C₁-C₄)-alkyl, heteroaryl-[(C₁-C₄)-alkyl]amino-(C₁-C₄)-alkyl, heteroaryl-(C₁-C₄)-alkyl-[(C₁-C₄)-alkyl]amino-(C₁-C₄)-alkyl, (C₃-C₆)-cycloalkyl-[(C₁-C₄)-alkyl]amino-(C₁-C₄)-alkyl, (C₃-C₆)-cycloalkylamino-(C₁-C₄)-alkyl, aryl-(C₁-C₄)-alkoxy-(C₁-C₄)-alkylaryl, heterocyclyl-N—(C₁-C₄)-alkylaryl, aryl[(C₁-C₄)-alkyl]amino-(C₁-C₄)-alkylaryl, aryl-(C₁-C₄)-alkyl[(C₁-C₄)-alkyl]amino-(C₁-C₄)-alkylaryl, (C₁-C₄)-alkoxycarbonyl-(C₁-C₄)-alkylamino-(C₁-C₄)-alkylaryl, (C₁-C₄)-alkoxycarbonyl-(C₁-C₄)-alkyl[(C₁-C₄)-alkyl]amino-(C₁-C₄)-alkylaryl, heteroaryl[(C₁-C₄)-alkyl]amino-(C₁-C₄)-alkylaryl, heteroaryl-(C₁-C₄)-alkyl[(C₁-C₄)-alkyl]amino-(C₁-C₄)-alkylaryl, (C₃-C₆)-cycloalkyl[(C₁-C₄)-alkyl]amino-(C₁-C₄)-alkylaryl, (C₃-C₆)-cycloalkylamino-(C₁-C₄)-alkylaryl, (C₁-C₄)-alkoxy[(C₁-C₄)-alkoxy]-(C₁-C₄)-alkylaryl,
Q additionally, if X represents an oxygen atom or X represents a sulfur atom, represents (C₁-C₅)-haloalkyl, (C₁-C₄)-alkoxy-(C₁-C₄)-haloalkyl, (C₃-C₆)-halocycloalkyl, (C₁-C₄)-haloalkoxy-(C₁-C₄)-haloalkyl, aryl-(C₁-C₄)-haloalkyl, (Cᵣ C₄)-alkylthio-(C₁-C₄)-haloalkyl, bis[(C₁-C₄)-alkyl]amino-(C₁-C₄)-alkoxy-(C₁-C₄)-haloalkyl,
Z¹ represents hydrogen, hydroxy, (C₁-C₄)-alkyl, (C₃-C₆)-cycloalkyl, (C₃-C₆)-halocycloalkyl, chlorine, (C₂-C₄)-alkenyl-(C₁-C₄)-alkyl, (C₁-C₄)-haloalkyl, (C₂-C₄)-alkynyl, (C₂-C₄)-alkenyl, cyano-(C₁-C₄)-alkyl, nitro-(C₁-C₄)-alkyl, amino-(C₁-C₄)-alkyl, alkyl-(C₁-C₄)-amino-(C₁-C₄)-alkyl, bis[(C₁-C₄)-alkyl]amino-(C₁-C₄)-alkyl, (C₂-C₄)-alkynyl-(C₁-C₄)-alkyl, aryl-(C₁-C₄)-alkyl, heteroaryl-(C₁-C₄)-alkyl, heterocyclyl-(C₁-C₄)-alkyl, (C₁-C₄)-alkylcarbonyl, (C₁-C₄)-alkoxycarbonyl, (C₃-C₆)-cycloalkoxycarbonyl, (C₁-C₄)-alkylsulfonyl, arylsulfonyl, heteroarylsulfonyl, (C₃-C₆)-cycloalkylsulfonyl, (C₁-C₄)-alkylsulfinyl, arylsulfinyl, (C₃-C₆)-cycloalkylsulfinyl, (C₁-C₄)-alkoxycarbonyl-(C₁-C₄)-alkyl, aryl, heteroaryl, heterocyclyl, aminocarbonyl-(C₁-C₄)-alkyl, (C₁-C₄)-alkylaminocarbonyl-(C₁-C₄)-alkyl, bis[(C₁-C₄)-alkyl]aminocarbonyl-(C₁-C₄)-alkyl, (C₃-C₆)-cycloalkylaminocarbonyl-(C₁-C₄)-alkyl, hydroxycarbonyl-(C₁-C₄)-alkyl
   and
Z² represents hydrogen, (C₁-C₄)-alkyl, (C₃-C₆)-cycloalkyl, (C₁-C₄)-haloalkyl, (C₂-C₄)-alkynyl, (C₂-C₄)-alkenyl, cyano-(C₁-C₄)-alkyl, aryl-(C₁-C₄)-alkyl, heteroaryl-(C₁-C₄)-alkyl, (C₁-C₄)-alkylcarbonyl, (C₁-C₄)-alkoxycarbonyl
   or
Z¹ and Z² together with the atoms to which they are attached form a fully saturated or partially saturated 5- to 7-membered ring which is optionally interrupted by heteroatoms and optionally substituted further
   or
Z¹ and Z² together form an N-(bis(C₁-C₆)-alkyl)sulfanylidene, N-(aryl-(C₁-C₆)-alkyl)sulfanylidene, N-(bis(C₃-C₇)-cycloalkyl)sulfanylidene, N—((C₁-C₆)-alkyl-(C₃-C₇)-cycloalkyl)sulfanylidene group or an N,N-di-(C₁-C₆)-alkylformylidene group.

The definitions of radicals stated above in general terms or in areas of preference apply both to the end products of the formula (I) and correspondingly to the starting materials or intermediates required in each case for preparation thereof. These radical definitions can be combined with one another as desired, i.e. including combinations between the given preferred ranges.

The aforementioned haloalkyl-substituted 2-amidobenzoxazoles and 2-amidobenzothiazoles of the general formula (I) are essentially likewise as yet unknown in the prior art. Thus, a further part of the invention is that of haloalkyl-substituted 2-amidobenzoxazoles and 2-amidobenzothiazoles of the general formula (I), or salts thereof,

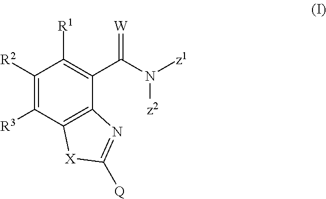

in which
R¹, R², R³ independently of one another represent hydrogen, halogen, (C₁-C₈)-alkyl, (C₃-C₈)-cycloalkyl, (C₄-C₈)-cycloalkenyl, (C₃-C₈)-halocycloalkyl, (C₂-C₈)-alkenyl, (C₂-C₈)-alkynyl, optionally substituted phenyl, aryl-(C₁-C₈)-alkyl, aryl-(C₂-C₈)-alkenyl, heteroaryl, heteroaryl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio, (C₁-C₈)-haloalkylthio, (Cᵣ C₈)-haloalkyl, (C₁-C₈)-alkoxy, (C₁-C₈)-haloalkoxy, (C₃-C₈)-cycloalkoxy, (C₃-C₈)-cycloalkyl-(C₁-C₈)-alkoxy, aryloxy, heteroaryloxy, (C₁-C₈)-alkoxy-(C₁-C₈)-alkoxy, (C₂-C₈)-alkynyl-(C₁-C₈)-alkoxy, (C₂-C₈)-alkenyloxy, bis[(C₁-C₈)-alkyl]amino-(C₁-C₈)-alkoxy, tris[(C₁-C₈)-alkyl]silyl, bis[(C₁-C₈)-alkyl]arylsilyl, bis[(C₁-C₈)-alkyl]-(C₁-C₈)-alkylsilyl, tris[(C₁-C₈)-alkyl]silyl-(C₂-C₈)-alkynyl, aryl-(C₂-C₈)-alkynyl, heteroaryl-(C₂-C₈)-alkynyl, (C₁-C₈)-alkyl-(C₂-C₈)-alkynyl, (C₃-C₈)-cycloalkyl-(C₂-C₈)-alkynyl, (C₁-C₈)-haloalkyl-(C₂-C₈)-alkynyl, heterocyclyl-N—(C₁-C₈)-alkoxy, nitro, cyano, amino, (C₁-C₈)-alkylamino, bis[(C₁-C₈)-alkyl]amino, (C₁-C₈)-alkylcarbonylamino, (C₃-C₈)-cycloalkylcarbonylamino, arylcarbonylamino, (C₁-C₈)-alkoxycarbonylamino, heterocyclyl-(C₁-C₈)-alkoxy, (C₃-C₈)-cycloalkyl-(C₁-C₈)-alkyl, (C₂-C₈)-haloalkenyl, (C₂-C₈)-haloalkynyl, heterocyclyl-(C₂-C₈)-alkynyl, (C₃-C₈)-halocycloalkoxy, (C₂-C₈)-haloalkynyloxy, arylthio, heteroarylthio, (C₁-C₈)-alkylsulfinyl, (C₁-C₈)-haloalkylsulfinyl, arylsulfinyl, heteroarylsulfinyl, (C₁-C₈)-alkylsulfonyl, (C₁-C₈)-haloalkylsulfonyl, arylsulfonyl, heteroarylsulfonyl, thiocyanato, isothiocyanato, (C₃-C₈)-cycloalkylamino, (C₃-C₈)-cycloalkyl[(C₁-C₈)-alkyl]amino, (C₂-C₈)-alkenylamino,
R¹ and R² with the atoms to which they are attached form a fully saturated, partially saturated or unsaturated 5- to 7-membered ring which is optionally interrupted by heteroatoms and optionally substituted further,
R² and R³ with the atoms to which they are attached form a fully saturated, partially saturated or unsaturated 5- to 7-membered ring which is optionally interrupted by heteroatoms and optionally substituted further,
W represents oxygen, sulfur,
X represents oxygen, sulfur,
Q represents (C₁-C₈)-haloalkyl, (C₁-C₈)-alkoxy-(C₁-C₈)-haloalkyl, (C₃-C₈)-halocycloalkyl, (C₁-C₈)-haloalkoxy-(C₁-C₈)-haloalkyl, aryl-(C₁-C₈)-haloalkyl, (C₁-C₈)-alkylthio-(C₁-C₈)-haloalkyl, bis[(C₁-C₈)-alkyl]amino-(C₁-C₈)-alkoxy-(C₁-C₈)-haloalkyl,
Z¹ represents hydrogen, hydroxy, (C₁-C₈)-alkyl, (C₃-C₈)-cycloalkyl, (C₃-C₈)-halocycloalkyl, halogen, (C₂-C₈)-alkenyl-(C₁-C₈)-alkyl, (C₁-C₈)-haloalkyl, (C₂-C₈)-alkynyl, (C₂-C₈)-alkenyl, cyano-(C₁-C₈)-alkyl, nitro-(C₁-C₈)-alkyl, amino-(C₁-C₈)-alkyl, alkyl-(C₁-C₈)-amino-(C₁-C₈)-alkyl, bis[(C₁-C₈)-alkyl]amino-(C₁-C₈)-alkyl, (C₂-C₈)-alkynyl-(C₁-C₈)-alkyl, aryl-(C₁-C₈)-alkyl, heteroaryl-(C₁-C₈)-alkyl, heterocyclyl-(C₁-C₈)-alkyl, (C₁-C₈)-alkylcarbonyl, (C₁-C₈)-alkoxycarbonyl, (C₃-C₈)-cycloalkoxycarbonyl, (C₁-C₈)-alkylsulfonyl, arylsulfonyl, heteroarylsulfonyl, (C₃-C₈)-cycloalkylsulfonyl, (C₁-C₈)-alkylsulfinyl, arylsulfinyl, (C₃-C₈)-cycloalkylsulfinyl, (C₁-C₈)-alkoxycarbonyl-(C₁-C₈)-alkyl, aryl, heteroaryl, heterocyclyl, aminocarbonyl-(C₁-C₈)-alkyl, (C₁-C₈)-alkylaminocarbonyl-(C₁-C₈)-alkyl, bis[(C₁-C₈)-alkyl]aminocarbonyl-(C₁-C₈)-alkyl, (C₃-C₈)-cycloalkylaminocarbonyl-(C₁-C₈)-alkyl, hydroxycarbonyl-(C₁-C₈)-alkyl, amino, (C₁-C₈)-alkylamino, arylamino, (C₁-C₈)-alkoxy
   and
Z² represents hydrogen, (C₁-C₈)-alkyl, (C₃-C₈)-cycloalkyl, (C₁-C₈)-haloalkyl, (C₂-C₈)-alkynyl, (C₂-C₈)-alkenyl, cyano-(C₁-C₈)-alkyl, aryl-(C₁-C₈)-alkyl, heteroaryl-(C₁-C₈)-alkyl, (C₁-C₈)-alkylcarbonyl, (C₁-C₈)-alkoxycarbonyl
   or
Z¹ and Z² together with the atoms to which they are attached form a fully saturated or partially saturated 5- to 7-membered ring which is optionally interrupted by heteroatoms and optionally substituted further
   or
Z¹ and Z² together form an N-(bis(C₁-C₈)-alkyl)sulfanylidene, N-(aryl-(C₁-C₈)-alkyl)sulfanylidene, N-(bis(C₃-C₇)-cycloalkyl)sulfanylidene, N—((C₁-C₈)-alkyl-(C₃-C₇)-cycloalkyl)sulfanylidene group or an N,N-di-(C₁-C₆)-alkylformylidene group.

Preference is given to compounds of the formula (I) according to the invention in which
R¹, R², R³ independently of one another represent hydrogen, halogen, (C₁-C₄)-alkyl, (C₃-C₈)-cycloalkyl, (C₄-C₈)-cycloalkenyl, (C₃-C₈)-halocycloalkyl, (C₂-C₄)-alkenyl, (C₂-C₄)-alkynyl, optionally substituted phenyl, aryl-(C₁-C₄)-alkyl, aryl-(C₂-C₄)-alkenyl, heteroaryl, heteroaryl-(C₁-C₄)-alkyl, heterocyclyl, heterocyclyl-(C₁-C₄)-alkyl, (C₁-C₄)-alkoxy-(C₁-C₄)-alkyl, (C₁-C₄)-alkylthio, (C₁-C₄)-haloalkylthio, (C₁-C₄)-haloalkyl, (C₁-C₄)-alkoxy, (C₁-C₄)-haloalkoxy, (C₃-C₈)-cycloalkoxy, (C₃-C₈)-cycloalkyl-(C₁-C₄)-alkoxy, aryloxy, heteroaryloxy, (C₁-C₄)-alkoxy-(C₁-C₄)-alkoxy, (C₂-C₄)-alkynyl-(C₁-C₄)-alkoxy, (C₂-C₄)-alkenyloxy, bis[(C₁-C₄)-alkyl]amino-(C₁-C₄)-alkoxy, tris[(C₁-C₄)-alkyl]silyl, bis[(C₁-C₄)-alkyl]arylsilyl, bis[(C₁-C₄)-alkyl]-(C₁-C₄)-alkylsilyl, tris[(C₁-C₄)-alkyl]silyl-(C₂-C₄)-alkynyl, aryl-(C₂-C₄)-alkynyl, heteroaryl-(C₂-C₄)-alkynyl, (C₁-C₄)-alkyl-(C₂-C₄)-alkynyl, (C₃-C₈)-cycloalkyl-(C₂-C₄)-alkynyl, (C₁-C₄)-haloalkyl-(C₂-C₄)-alkynyl, heterocyclyl-N—(C₁-C₄)-alkoxy, nitro, cyano, amino, (C₁-C₄)-alkylamino, bis[(C₁-C₄)-alkyl]amino, (C₁-C₄)-alkylcarbonylamino, (C₃-C₈)-cycloalkylcarbonylamino, arylcarbonylamino, (C₁-C₄)-alkoxycarbonylamino, heterocyclyl-(C₁-C₄)-alkoxy, (C₃-C₈)-cycloalkyl-(C₁-C₄)-alkyl, (C₂-C₄)-haloalkenyl, (C₂-C₄)-haloalkynyl, heterocyclyl-(C₂-C₄)-alkynyl, (C₃-C₈)-halocycloalkoxy, (C₂-C₄)-haloalkynyloxy, arylthio, heteroarylthio, (C₁-C₄)-alkylsulfinyl, (C₁-C₄)-haloalkylsulfinyl, arylsulfinyl, heteroarylsulfinyl, (C₁-C₄)-alkylsulfonyl, (C₁-C₄)-haloalkylsulfonyl, arylsulfonyl, heteroarylsulfonyl, thiocyanato, isothiocyanato, (C₃-C₈)-cycloalkylamino, (C₃-C₈)-cycloalkyl[(C₁-C₄)-alkyl]amino, (C₂-C₄)-alkenylamino,
R¹ and R² with the atoms to which they are attached form a fully saturated, partially saturated or unsaturated 5- to 7-membered ring which is optionally interrupted by heteroatoms and optionally substituted further,
R² and R³ with the atoms to which they are attached form a fully saturated, partially saturated or unsaturated 5- to 7-membered ring which is optionally interrupted by heteroatoms and optionally substituted further,
W represents oxygen, sulfur,
X represents oxygen, sulfur,
Q represents (C₁-C₇)-haloalkyl, (C₁-C₄)-alkoxy-(C₁-C₄)-haloalkyl, (C₃-C₈)-halocycloalkyl, (C₁-C₄)-haloalkoxy-(C₁-C₄)-haloalkyl, aryl-(C₁-C₄)-haloalkyl, (C₁-C₄)-alkylthio-(C₁-C₄)-haloalkyl, bis[(C₁-C₄)-alkyl]amino-(C₁-C₄)-alkoxy-(C₁-C₄)-haloalkyl,
Z¹ represents hydrogen, hydroxy, (C₁-C₄)-alkyl, (C₃-C₈)-cycloalkyl, (C₃-C₈)-halocycloalkyl, halogen, (C₂-C₄)-alkenyl-(C₁-C₄)-alkyl, (C₁-C₄)-haloalkyl, (C₂-C₄)-alkynyl, (C₂-C₄)-alkenyl, cyano-(C₁-C₄)-alkyl, nitro-(C₁-C₄)-alkyl, amino-(C₁-C₄)-alkyl, alkyl-(C₁-C₄)-amino-(C₁-C₄)-alkyl, bis[(C₁-C₄)-alkyl]amino-(C₁-C₄)-alkyl, (C₂-C₄)-alkynyl-(C₁-C₄)-alkyl, aryl-(C₁-C₄)-alkyl, heteroaryl-(C₁-C₄)-alkyl, heterocyclyl-(C₁-C₄)-alkyl, (C₁-C₄)-alkylcarbonyl, (C₁-C₄)-alkoxycarbonyl, (C₃-C₈)-cycloalkoxycarbonyl, (C₁-C₄)-alkylsulfonyl, arylsulfonyl, heteroarylsulfonyl, (C₃-C₆)-cycloalkylsulfonyl, (C₁-C₄)-alkylsulfinyl, arylsulfinyl, (C₃-C₈)-cycloalkylsulfinyl, (C₁-C₄)-alkoxycarbonyl-(C₁-C₄)-alkyl, aryl, heteroaryl, heterocyclyl, aminocarbonyl-(C₁-C₄)-alkyl, (C₁-C₄)-alkylaminocarbonyl-(C₁-C₄)-alkyl, bis[(C₁-C₄)-alkyl]aminocarbonyl-(C₁-C₄)-alkyl, (C₃-C₈)-cycloalkylaminocarbonyl-(C₁-C₄)-alkyl, hydroxycarbonyl-(C₁-C₄)-alkyl, amino, (C₁-C₄)-alkylamino, arylamino, (C₁-C₄)-alkoxy
   and
Z² represents hydrogen, (C₁-C₄)-alkyl, (C₃-C₈)-cycloalkyl, (C₁-C₄)-haloalkyl, (C₂-C₄)-alkynyl, (C₂-C₄)-alkenyl, cyano-(C₁-C₄)-alkyl, aryl-(C₁-C₄)-alkyl, heteroaryl-(C₁-C₄)-alkyl, (C₁-C₄)-alkylcarbonyl, (C₁-C₄)-alkoxycarbonyl
   or
Z¹ and Z² together with the atoms to which they are attached form a fully saturated or partially saturated 5- to 7-membered ring which is optionally interrupted by heteroatoms and optionally substituted further
   or
Z¹ and Z² together form an N-(bis(C₁-C₆)-alkyl)sulfanylidene, N-(aryl-(C₁-C₆)-alkyl)sulfanylidene, N-(bis(C₃-C₇)-cycloalkyl)sulfanylidene, N—((C₁-C₆)-alkyl-(C₃-C₇)-cycloalkyl)sulfanylidene group or an N,N-di-(C₁-C₆)-alkylformylidene group.

The aforementioned sulfanylidene-substituted 2-amidobenzimidazoles of the general formula (I) are essentially likewise as yet unknown in the prior art. Thus, a further part of the invention is that of sulfanylidene-substituted 2-amidobenzimidazoles of the general formula (I), or salts thereof,

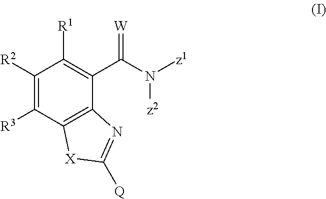

in which
R¹, R², R³ independently of one another represent hydrogen, halogen, (C₁-C₈)-alkyl, (C₃-C₈)-cycloalkyl, (C₄-C₈)-cycloalkenyl, (C₃-C₈)-halocycloalkyl, (C₂-C₈)-alkenyl, (C₂-C₈)-alkynyl, optionally substituted phenyl, aryl-(C₁-C₈)-alkyl, aryl-(C₂-C₈)-alkenyl, heteroaryl, heteroaryl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio, (C₁-C₈)-haloalkylthio, (Cᵣ C₈)-haloalkyl, (C₁-C₈)-alkoxy, (C₁-C₈)-haloalkoxy, (C₃-C₈)-cycloalkoxy, (C₃-C₈)-cycloalkyl-(C₁-C₈)-alkoxy, aryloxy, heteroaryloxy, (C₁-C₈)-alkoxy-(C₁-C₈)-alkoxy, (C₂-C₈)-alkynyl-(C₁-C₈)-alkoxy, (C₂-C₈)-alkenyloxy, bis[(C₁-C₈)-alkyl]amino-(C₁-C₈)-alkoxy, tris[(C₁-C₈)-alkyl]silyl, bis[(C₁-C₈)-alkyl]arylsilyl, bis[(C₁-C₈)-alkyl]-(C₁-C₈)-alkylsilyl, tris[(C₁-C₈)-alkyl]silyl-(C₂-C₈)-alkynyl, aryl-(C₂-C₈)-alkynyl, heteroaryl-(C₂-C₈)-alkynyl, (C₁-C₈)-alkyl-(C₂-C₈)-alkynyl, (C₃-C₈)-cycloalkyl-(C₂-C₈)-alkynyl, (C₁-C₈)-haloalkyl-(C₂-C₈)-alkynyl, heterocyclyl-N—(C₁-C₈)-alkoxy, nitro, cyano, amino, (C₁-C₈)-alkylamino, bis[(C₁-C₈)-alkyl]amino, (C₁-C₈)-alkylcarbonylamino, (C₃-C₈)-cycloalkylcarbonylamino, arylcarbonylamino, (C₁-C₈)-alkoxycarbonylamino, heterocyclyl-(C₁-C₈)-alkoxy, (C₃-C₈)-cycloalkyl-(C₁-C₈)-alkyl, (C₂-C₈)-haloalkenyl, (C₃-C₈)-haloalkynyl, heterocyclyl-(C₂-C₈)-alkynyl, (C₃-C₈)-halocycloalkoxy, (C₂-C₈)-haloalkynyloxy, arylthio, heteroarylthio, (C₁-C₈)-alkylsulfinyl, (C₁-C₈)-haloalkylsulfinyl, arylsulfinyl, heteroarylsulfinyl, (C₁-C₈)-alkylsulfonyl, (C₁-C₈)-haloalkylsulfonyl, arylsulfonyl, heteroarylsulfonyl, thiocyanato, isothiocyanato, (C₃-C₈)-cycloalkylamino, (C₃-C₈)-cycloalkyl[(C₁-C₈)-alkyl]amino, (C₂-C₈)-alkenylamino,
R¹ and R² with the atoms to which they are attached form a fully saturated, partially saturated or unsaturated 5- to 7-membered ring which is optionally interrupted by heteroatoms and optionally substituted further,
R² and R³ with the atoms to which they are attached form a fully saturated, partially saturated or unsaturated 5- to 7-membered ring which is optionally interrupted by heteroatoms and optionally substituted further,
W represents oxygen, sulfur,
X represents N—R⁴,
R⁴ represents hydrogen, (C₁-C₈)-alkyl, (C₃-C₈)-cycloalkyl, (C₃-C₈)-cycloalkyl-(C₁-C₈)-alkyl, (C₄-C₈)-cycloalkenyl, cyano-(C₁-C₈)-alkyl, (C₂-C₈)-alkenyl-(C₁-C₈)-alkyl, (C₁-C₈)-haloalkyl, (C₂-C₈)-alkynyl-(C₁-C₈)-alkyl, aryl-(C₁-C₈)-alkyl, heteroaryl-(C₁-C₈)-alkyl, heterocyclyl-(C₁-C₈)-alkyl, (C₁-C₈)-alkylcarbonyl, (C₃-C₈)-cycloalkylcarbonyl, arylcarbonyl, heteroarylcarbonyl, (C₁-C₈)-haloalkylcarbonyl, heterocyclylcarbonyl, aryl-(C₁-C₈)-alkylcarbonyl, (C₁-C₈)-alkoxycarbonyl, (C₃-C₈)-cycloalkyl-(C₁-C₈)-alkoxycarbonyl, (C₃-C₈)-cycloalkoxycarbonyl, (C₁-C₈)-alkoxycarbonyl-(C₁-C₈)-alkyl, (C₁-C₈)-alkylsulfonyl, (C₃-C₈)-cycloalkylsulfonyl, arylsulfonyl, heteroarylsulfonyl, (C₁-C₈)-alkoxycarbonylcarbonyl, aryl-(C₁-C₈)-alkoxycarbonylcarbonyl, (C₁-C₈)-alkylaminothiocarbonyl, (C₁-C₈)-alkylaminocarbonyl, (C₃-C₈)-cycloalkylaminocarbonyl, (C₁-C₈)-alkoxy-(C₁-C₈)-alkyl, bis[(C₁-C₈)-alkyl]aminocarbonyl, aryl, heteroaryl, heterocyclyl, (C₃-C₈)-cycloalkoxycarbonyl-(C₁-C₈)-alkyl,
Q represents (C₁-C₈)-alkyl, (C₂-C₈)-alkenyl, (C₃-C₈)-cycloalkyl, (C₃-C₈)-cycloalkyl-(C₁-C₈)-alkyl, (C₄-C₈)-cycloalkenyl, optionally substituted phenyl, aryl-(C₁-C₈)-alkyl, heteroaryl-(C₁-C₈)-alkyl, heterocyclyl-(C₁-C₈)-alkyl, heteroaryl, heterocyclyl, heterocyclylaryl, heterocyclyl heteroaryl, heteroaryl heteroaryl, heteroarylaryl, arylaryl, aryloxyaryl, aryl-(C₂-C₈)-alkenyl, heteroaryl-(C₂-C₈)-alkenyl, heterocyclyl-(C₂-C₈)-alkenyl, aryl-(C₂-C₈)-alkynyl, heteroaryl-(C₂-C₈)-alkynyl, heterocyclyl-(C₁-C₈)-alkynyl, (C₃-C₈)-cycloalkyl-(C₂-C₈)-alkynyl, (C₁-C₈)-alkylamino-(C₁-C₈)-alkyl, bis[(C₁-C₈)-alkyl]amino-(C₁-C₈)-alkyl, hydroxy-(C₁-C₈)-alkyl, (C₁-C₈)-alkoxy-(C₁-C₈)-alkyl, tris[(C₁-C₈)-alkyl]silyloxy-(C₁-C₈)-alkyl, bis[(C₁-C₈)-alkyl]arylsilyloxy-(C₁-C₈)-alkyl, bis[(C₁-C₈)-alkyl]-(C₁-C₈)-alkylsilyloxy-(C₁-C₈)-alkyl, bis[(C₁-C₈)-alkyl]amino-(C₁-C₈)-alkoxy-(C₁-C₈)-alkyl, (C₁-C₈)-alkoxy-(C₁-C₈)-alkoxy-(C₁-C₈)-alkyl, aryloxy-(C₁-C₈)-alkyl, heteroaryloxy-(C₁-C₈)-alkyl, (C₁-C₈)-alkylthio-(C₁-C₈)-alkyl, arylthio-(C₁-C₈)-alkyl, heteroarylthio-(C₁-C₈)-alkyl, (C₁-C₈)-alkoxycarbonyl-N-heterocyclyl, aryl-(C₁-C₈)-alkoxycarbonyl-N-heterocyclyl, (C₁-C₈)-alkyl-N-heterocyclyl, (C₁-C₈)-alkylsulfonyl-N-heterocyclyl, arylsulfonyl-N-heterocyclyl, heteroarylsulfonyl-N-heterocyclyl, (C₃-C₈)-cycloalkylsulfonyl-N-heterocyclyl, (C₁-C₈)-haloalkylsulfonyl-N-heterocyclyl, (C₁-C₈)-alkylcarbonyl-N-heterocyclyl, arylcarbonyl-N-heterocyclyl, heteroarylcarbonyl-N-heterocyclyl, (C₃-C₈)-cycloalkylcarbonyl-N-heterocyclyl, (C₁-C₈)-cycloalkyl-N-heterocyclyl, aryl-N-heterocyclyl, aryl-(C₁-C₈)-alkyl-N-heterocyclyl, bis[(C₁-C₈)-alkyl]aminoalkyl-N-heterocyclyl, bis[(C₁-C₈)-alkyl]aminosulfonyl-N-heterocyclyl, heteroaryloxyaryl, heteroaryloxyheteroaryl, aryloxyheteroaryl, (C₁-C₈)-alkylsulfinyl, (C₁-C₈)-alkylthio, (C₁-C₈)-alkylsulfonyl, (C₃-C₈)-cycloalkylsulfinyl, (C₃-C₈)-cycloalkylthio, (C₃-C₈)-cycloalkylsulfonyl, arylsulfinyl, arylthio, arylsulfonyl, amino, (C₁-C₈)-alkylamino, bis[(C₁-C₈)-alkyl]amino, arylamino, aryl-(C₁-C₈)-alkylamino, (C₃-C₈)-cycloalkylamino, formyl, (C₁-C₈)-alkylcarbonyl, arylcarbonyl, imino-(C₁-C₈)-alkyl, (C₁-C₈)-alkylimino-(C₁-C₈)-alkyl, arylimino-(C₁-C₈)-alkyl, (C₁-C₈)-alkoxycarbonyl, (C₃-C₈)-cycloalkoxycarbonyl, (C₃-C₈)-cycloalkyl-(C₁-C₈)-alkoxycarbonyl, aryl-(C₁-C₈)-alkoxycarbonyl, aryl-(C₁-C₈)-alkylaminocarbonyl, aminocarbonyl, (C₁-C₈)-alkylaminocarbonyl, (C₃-C₈)-cycloalkylaminocarbonyl, bis[(C₁-C₈)-alkyl]aminocarbonyl, heterocyclyl-N-carbonyl, imino, (C₁-C₈)-alkylimino, arylimino, (C₃-C₈)-cycloalkylimino, (C₃-C₈)-cycloalkyl-(C₁-C₈)-alkylimino, hydroxyimino, (C₁-C₈)-alkoxyimino, (C₂-C₈)-alkenyloxyimino, (C₃-C₈)-cycloalkoxyimino, (C₃-C₈)-cyloalkyl-(C₁-C₈)-alkoxyimino, aryloxyimino, aryl-(C₁-C₈)-alkoxyimino, heteroaryl-(C₁-C₈)-alkoxyimino, heteroaryloxyimino, heteroarylimino, heterocyclylimino, heterocyclyl-(C₁-C₈)-alkylimino, aminoimino, (C₁-C₈)-alkylaminoimino, arylaminoimino, heteroarylaminoimino, (C₃-C₈)-cycloalkylaminoimino, bis[(C₁-C₈)-alkyl]aminoimino, aryl-(C₁-C₈)-alkylaminoimino, aryl[(C₁-C₈)-alkyl]aminoimino, (C₃-C₈)-cycloalkyl[(C₁-C₈)-alkyl]aminoimino, (C₃-C₈)-cycloalkyl-(C₁-C₈)-alkylaminoimino, heterocyclylaminoimino, heteroaryl-(C₁-C₈)-alkoxy-(C₁-C₈)-alkyl, aryl-(C₁-C₈)-alkoxy-(C₁-C₈)-alkyl, heterocyclyl-N—(C₁-C₈)-alkyl, aryl-[(C₁-C₈)-alkyl]amino-(C₁-C₈)-alkyl, aryl-(C₁-C₈)-alkyl[(C₁-C₈)-alkyl]amino-(C₁-C₈)-alkyl, (C₁-C₈)-alkoxycarbonyl-(C₁-C₈)-alkylamino-(C₁-C₈)-alkyl, (C₁-C₈)-alkoxycarbonyl-(C₁-C₈)-alkyl[(C₁-C₈)-alkyl]amino-(C₁-C₈)-alkyl, heteroaryl-[(C₁-C₈)-alkyl]amino-(C₁-C₈)-alkyl, heteroaryl-(C₁-C₈)-alkyl-[(C₁-C₈)-alkyl]amino-(C₁-C₈)-alkyl, —(C₃-C₈)-cycloalkyl-[(C₁-C₈)-alkyl]amino-(C₁-C₈)-alkyl, (C₃-C₈)-cycloalkylamino-(C₁-C₈)-alkyl, aryl-(C₁-C₈)-alkoxy-(C₁-C₈)-alkylaryl, heterocyclyl-N—(C₁-C₈)-alkylaryl, aryl[(C₁-C₈)-alkyl]amino-(C₁-C₈)-alkylaryl, aryl-(C₁-C₈)-alkyl[(C₁-C₈)-alkyl]amino-(C₁-C₈)-alkylaryl, (C₁-C₈)-alkoxycarbonyl-(C₁-C₈)-alkylamino-(C₁-C₈)-alkylaryl, (C₁-C₈)-alkoxycarbonyl-(C₁-C₈)-alkyl[(C₁-C₈)-alkyl]amino-(C₁-C₈)-alkylaryl, heteroaryl[(C₁-C₈)-alkyl]amino-(C₁-C₈)-alkylaryl, heteroaryl-(C₁-C₈)-alkyl[(C₁-C₈)-alkyl]amino-(C₁-C₈)-alkylaryl, (C₃-C₈)-cycloalkyl[(C₁-C₈)-alkyl]amino-(C₁-C₈)-alkylaryl, (C₃-C₈)-cycloalkylamino-(C₁-C₈)-alkylaryl, (C₁-C₈)-alkoxy[(C₁-C₈)-alkoxy]-(C₁-C₈)-alkylaryl
   and
Z¹ and Z² together form an N-(bis(C₁-C₈)-alkyl)sulfanylidene, N-(aryl-(C₁-C₈)-alkyl)sulfanylidene, N-(bis(C₃-C₈)-cycloalkyl)sulfanylidene, N—((C₁-C₈)-alkyl-(C₃-C₈)-cycloalkyl)sulfanylidene group or an N,N-di-(C₁-C₈)-alkylformylidene group.

Preference is given to compounds of the formula (I) according to the invention in which
R¹, R², R³ independently of one another represent hydrogen, halogen, (C₁-C₄)-alkyl, (C₃-C₈)-cycloalkyl, (C₄-C₈)-cycloalkenyl, (C₃-C₈)-halocycloalkyl, (C₂-C₄)-alkenyl, (C₂-C₄)-alkynyl, optionally substituted phenyl, aryl-(C₁-C₄)-alkyl, aryl-(C₂-C₄)-alkenyl, heteroaryl, heteroaryl-(C₁-C₄)-alkyl, heterocyclyl, heterocyclyl-(C₁-C₄)-alkyl, (C₁-C₄)-alkoxy-(C₁-C₄)-alkyl, (C₁-C₄)-alkylthio, (C₁-C₄)-haloalkylthio, (C₁-C₄)-haloalkyl, (C₁-C₄)-alkoxy, (C₁-C₄)-haloalkoxy, (C₃-C₈)-cycloalkoxy, (C₃-C₈)-cycloalkyl-(C₁-C₄)-alkoxy, aryloxy, heteroaryloxy, (C₁-C₄)-alkoxy-(C₁-C₄)-alkoxy, (C₂-C₄)-alkynyl-(C₁-C₄)-alkoxy, (C₂-C₄)-alkenyloxy, bis[(C₁-C₄)-alkyl]amino-(C₁-C₄)-alkoxy, tris[(C₁-C₄)-alkyl]silyl, bis[(C₁-C₄)-alkyl]arylsilyl, bis[(C₁-C₄)-alkyl]-(C₁-C₄)-alkylsilyl, tris[(C₁-C₄)-alkyl]silyl-(C₂-C₄)-alkynyl, aryl-(C₂-C₄)-alkynyl, heteroaryl-(C₂-C₄)-alkynyl, (C₁-C₄)-alkyl-(C₂-C₄)-alkynyl, (C₃-C₈)-cycloalkyl-(C₂-C₄)-alkynyl, (C₁-C₄)-haloalkyl-(C₂-C₄)-alkynyl, heterocyclyl-N—(C₁-C₄)-alkoxy, nitro, cyano, amino, (C₁-C₄)-alkylamino, bis[(C₁-C₄)-alkyl]amino, (C₁-C₄)-alkylcarbonylamino, (C₃-C₈)-cycloalkylcarbonylamino, arylcarbonylamino, (C₁-C₄)-alkoxycarbonylamino, heterocyclyl-(C₁-C₄)-alkoxy, (C₃-C₈)-cycloalkyl-(C₁-C₄)-alkyl, (C₂-C₄)-haloalkenyl, (C₂-C₄)-haloalkynyl, heterocyclyl-(C₂-C₄)-alkynyl, (C₃-C₄)-halocycloalkoxy, (C₂-C₄)-haloalkynyloxy, arylthio, heteroarylthio, (C₁-C₄)-alkylsulfinyl, (C₁-C₄)-haloalkylsulfinyl, arylsulfinyl, heteroarylsulfinyl, (C₁-C₄)-alkylsulfonyl, (C₁-C₄)-haloalkylsulfonyl, arylsulfonyl, heteroarylsulfonyl, thiocyanato, isothiocyanato, (C₃-C₈)-cycloalkylamino, (C₃-C₈)-cycloalkyl[(C₁-C₄)-alkyl]amino, (C₂-C₄)-alkenylamino,
R¹ and R² with the atoms to which they are attached form a fully saturated, partially saturated or unsaturated 5- to 7-membered ring which is optionally interrupted by heteroatoms and optionally substituted further,
R² and R³ with the atoms to which they are attached form a fully saturated, partially saturated or unsaturated 5- to 7-membered ring which is optionally interrupted by heteroatoms and optionally substituted further,
W represents oxygen, sulfur,
X represents N—R⁴,
R⁴ represents hydrogen, (C₁-C₄)-alkyl, (C₃-C₈)-cycloalkyl, (C₃-C₈)-cycloalkyl-(C₁-C₄)-alkyl, (C₄-C₈)-cycloalkenyl, cyano-(C₁-C₄)-alkyl, (C₂-C₄)-alkenyl-(C₁-C₄)-alkyl, (C₁-C₄)-haloalkyl, (C₂-C₄)-alkynyl-(C₁-C₄)-alkyl, aryl-(C₁-C₄)-alkyl, heteroaryl-(C₁-C₄)-alkyl, heterocyclyl-(C₁-C₄)-alkyl, (C₁-C₄)-alkylcarbonyl, (C₃-C₈)-cycloalkylcarbonyl, arylcarbonyl, heteroarylcarbonyl, (C₁-C₄)-haloalkylcarbonyl, heterocyclylcarbonyl, aryl-(C₁-C₄)-alkylcarbonyl, (C₁-C₄)-alkoxycarbonyl, (C₃-C₈)-cycloalkyl-(C₁-C₄)-alkoxycarbonyl, (C₃-C₈)-cycloalkoxycarbonyl, (C₁-C₄)-alkoxycarbonyl-(C₁-C₄)-alkyl, (C₁-C₄)-alkylsulfonyl, (C₃-C₈)-cycloalkylsulfonyl, arylsulfonyl, heteroarylsulfonyl, (C₁-C₄)-alkoxycarbonylcarbonyl, aryl-(C₁-C₄)-alkoxycarbonylcarbonyl, (C₁-C₄)-alkylaminothiocarbonyl, (C₁-C₄)-alkylaminocarbonyl, (C₃-C₈)-cycloalkylaminocarbonyl, (C₁-C₄)-alkoxy-(C₁-C₄)-alkyl, bis[(C₁-C₄)-alkyl]aminocarbonyl, aryl, heteroaryl, heterocyclyl, (C₃-C₈)-cycloalkoxycarbonyl-(C₁-C₄)-alkyl,
Q represents (C₁-C₄)-alkyl, (C₂-C₄)-alkenyl, (C₃-C₈)-cycloalkyl, (C₃-C₈)-cycloalkyl-(C₁-C₄)-alkyl, (C₄-C₈)-cycloalkenyl, optionally substituted phenyl, aryl-(C₁-C₄)-alkyl, heteroaryl-(C₁-C₄)-alkyl, heterocyclyl-(C₁-C₄)-alkyl, heteroaryl, heterocyclyl, heterocyclylaryl, heterocyclyl heteroaryl, heteroarylheteroaryl, heteroarylaryl, arylaryl, aryloxyaryl, aryl-(C₂-C₄)-alkenyl, heteroaryl-(C₂-C₄)-alkenyl, heterocyclyl-(C₂-C₄)-alkenyl, aryl-(C₂-C₄)-alkynyl, heteroaryl-(C₂-C₄)-alkynyl, heterocyclyl-(C₁-C₄)-alkynyl, (C₃-C₈)-cycloalkyl-(C₂-C₄)-alkynyl, (C₁-C₄)-alkylamino-(C₁-C₄)-alkyl, bis[(C₁-C₄)-alkyl]amino-(C₁-C₄)-alkyl, hydroxy-(C₁-C₄)-alkyl, (C₁-C₄)-alkoxy-(C₁-C₄)-alkyl, tris[(C₁-C₄)-alkyl]silyloxy-(C₁-C₄)-alkyl, bis[(C₁-C₄)-alkyl]arylsilyloxy-(C₁-C₄)-alkyl, bis[(C₁-C₄)-alkyl]-(C₁-C₄)-alkylsilyloxy-(C₁-C₄)-alkyl, bis[(C₁-C₄)-alkyl]amino-(C₁-C₄)-alkoxy-(C₁-C₄)-alkyl, (C₁-C₄)-alkoxy-(C₁-C₄)-alkoxy-(C₁-C₄)-alkyl, aryloxy-(C₁-C₄)-alkyl, heteroaryloxy-(C₁-C₄)-alkyl, (C₁-C₄)-alkylthio-(C₁-C₄)-alkyl, arylthio-(C₁-C₄)-alkyl, heteroarylthio-(C₁-C₄)-alkyl, (C₁-C₄)-alkoxycarbonyl-N-heterocyclyl, aryl-(C₁-C₄)-alkoxycarbonyl-N-heterocyclyl, (C₁-C₄)-alkyl-N-heterocyclyl, (C₁-C₄)-alkylsulfonyl-N-heterocyclyl, arylsulfonyl-N-heterocyclyl, heteroarylsulfonyl-N-heterocyclyl, (C₃-C₈)-cycloalkylsulfonyl-N-heterocyclyl, (C₁-C₄)-haloalkylsulfonyl-N-heterocyclyl, (C₁-C₄)-alkylcarbonyl-N-heterocyclyl, arylcarbonyl-N-heterocyclyl, heteroarylcarbonyl-N-heterocyclyl, (C₃-C₈)-cycloalkylcarbonyl-N-heterocyclyl, (C₁-C₈)-cycloalkyl-N-heterocyclyl, aryl-N-heterocyclyl, aryl-(C₁-C₄)-alkyl-N-heterocyclyl, bis[(C₁-C₄)-alkyl]aminoalkyl-N-heterocyclyl, bis[(C₁-C₄)-alkyl]aminosulfonyl-N-heterocyclyl, heteroaryloxyaryl, heteroaryloxyheteroaryl, aryloxyheteroaryl, (C₁-C₄)-alkylsulfinyl, (C₁-C₄)-alkylthio, (C₁-C₄)-alkylsulfonyl, (C₃-C₈)-cycloalkylsulfinyl, (C₃-C₈)-cycloalkylthio, (C₃-C₈)-cycloalkylsulfonyl, arylsulfinyl, arylthio, arylsulfonyl, amino, (C₁-C₄)-alkylamino, bis[(C₁-C₄)-alkyl]amino, arylamino, aryl-(C₁-C₄)-alkylamino, (C₃-C₈)-cycloalkylamino, formyl, (C₁-C₄)-alkylcarbonyl, arylcarbonyl, imino-(C₁-C₄)-alkyl, (C₁-C₄)-alkylimino-(C₁-C₄)-alkyl, arylimino-(C₁-C₄)-alkyl, (C₁-C₄)-alkoxycarbonyl, (C₃-C₈)-cycloalkoxycarbonyl, (C₃-C₈)-cycloalkyl-(C₁-C₄)-alkoxycarbonyl, aryl-(C₁-C₄)-alkoxycarbonyl, aryl-(C₁-C₄)-alkylaminocarbonyl, aminocarbonyl, (C₁-C₄)-alkylaminocarbonyl, (C₃-C₈)-cycloalkylaminocarbonyl, bis[(C₁-C₄)-alkyl]aminocarbonyl, heterocyclyl-N-carbonyl, imino, (C₁-C₄)-alkylimino, arylimino, (C₃-C₈)-cycloalkylimino, (C₃-C₈)-cycloalkyl-(C₁-C₄)-alkylimino, hydroxyimino, (C₁-C₄)-alkoxyimino, (C₂-C₄)-alkenyloxyimino, (C₃-C₈)-cycloalkoxyimino, (C₃-C₈)-cyloalkyl-(C₁-C₄)-alkoxyimino, aryloxyimino, aryl-(C₁-C₄)-alkoxyimino, heteroaryl-(C₁-C₄)-alkoxyimino, heteroaryloxyimino, heteroarylimino, heterocyclylimino, heterocyclyl-(C₁-C₄)-alkylimino, aminoimino, (C₁-C₄)-alkylaminoimino, arylaminoimino, heteroarylaminoimino, (C₃-C₈)-cycloalkylaminoimino, bis[(C₁-C₄)-alkyl]aminoimino, aryl-(C₁-C₄)-alkylaminoimino, aryl[(C₁-C₄)-alkyl]aminoimino, (C₃-C₈)-cycloalkyl[(C₁-C₄)-alkyl]aminoimino, (C₃-C₈)-cycloalkyl-(C₁-C₄)-alkylaminoimino, heterocyclylaminoimino, heteroaryl-(C₁-C₄)-alkoxy-(C₁-C₄)-alkyl, aryl-(C₁-C₄)-alkoxy-(C₁-C₄)-alkyl, heterocyclyl-N—(C₁-C₄)-alkyl, aryl-[(C₁-C₄)-alkyl]amino-(C₁-C₄)-alkyl, aryl-(C₁-C₄)-alkyl[(C₁-C₄)-alkyl]amino-(C₁-C₄)-alkyl, (C₁-C₄)-alkoxycarbonyl-(C₁-C₄)-alkylamino-(C₁-C₄)-alkyl, (C₁-C₄)-alkoxycarbonyl-(C₁-C₄)-alkyl[(C₁-C₄)-alkyl]amino-(C₁-C₄)-alkyl, heteroaryl-[(C₁-C₄)-alkyl]amino-(C₁-C₄)-alkyl, heteroaryl-(C₁-C₄)-alkyl-[(C₁-C₄)-alkyl]amino-(C₁-C₄)-alkyl, —(C₃-C₈)-cycloalkyl-[(C₁-C₄)-alkyl]amino-(C₁-C₄)-alkyl, (C₃-C₈)-cycloalkylamino-(C₁-C₄)-alkyl, aryl-(C₁-C₄)-alkoxy-(C₁-C₄)-alkylaryl, heterocyclyl-N—(C₁-C₄)-alkylaryl, aryl[(C₁-C₄)-alkyl]amino-(C₁-C₄)-alkylaryl, aryl-(C₁-C₄)-alkyl[(C₁-C₄)-alkyl]amino-(C₁-C₄)-alkylaryl, (C₁-C₄)-alkoxycarbonyl-(C₁-C₄)-alkylamino-(C₁-C₄)-alkylaryl, (C₁-C₄)-alkoxycarbonyl-(C₁-C₄)-alkyl[(C₁-C₄)-alkyl]amino-(C₁-C₄)-alkylaryl, heteroaryl[(C₁-C₄)-alkyl]amino-(C₁-C₄)-alkylaryl, heteroaryl-(C₁-C₄)-alkyl[(C₁-C₄)-alkyl]amino-(C₁-C₄)-alkylaryl, (C₃-C₈)-cycloalkyl[(C₁-C₄)-alkyl]amino-(C₁-C₄)-alkylaryl, (C₃-C₈)-cycloalkylamino-(C₁-C₄)-alkylaryl, (C₁-C₄)-alkoxy[(C₁-C₄)-alkoxy]-(C₁-C₄)-alkylaryl
   and
Z¹ and Z² together form an N-(bis(C₁-C₄)-alkyl)sulfanylidene, N-(aryl-(C₁-C₄)-alkyl)sulfanylidene, N-(bis(C₃-C₈)-cycloalkyl)sulfanylidene, N—((C₁-C₄)-alkyl-(C₃-C₈)-cycloalkyl)sulfanylidene group or an N,N-di-(C₁-C₄)-alkylformylidene group.

With regard to the compounds according to the invention, the terms used above and further below will be elucidated. These are familiar to the person skilled in the art and have especially the definitions elucidated hereinafter:

According to the invention, “arylsulfonyl” represents optionally substituted phenylsulfonyl or optionally substituted polycyclic arylsulfonyl, here especially optionally substituted naphthylsulfonyl, for example substituted by fluorine, chlorine, bromine, iodine, cyano, nitro, alkyl, haloalkyl, haloalkoxy, amino, alkylamino, alkylcarbonylamino, dialkylamino or alkoxy groups.

According to the invention, “cycloalkylsulfonyl”—alone or as a constituent of a chemical group—represents optionally substituted cycloalkylsulfonyl, preferably having 3 to 6 carbon atoms, for example cyclopropylsulfonyl, cyclobutylsulfonyl, cyclopentylsulfonyl or cyclohexylsulfonyl.

According to the invention, “alkylsulfonyl”—alone or as part of a chemical group—represents straight-chain or branched alkylsulfonyl, preferably having 1 to 8 or having 1 to 6 carbon atoms, for example methylsulfonyl, ethylsulfonyl, n-propylsulfonyl, isopropylsulfonyl, n-butylsulfonyl, isobutylsulfonyl, sec-butylsulfonyl and tert-butylsulfonyl.

According to the invention, “heteroarylsulfonyl” represents optionally substituted pyridylsulfonyl, pyrimidinylsulfonyl, pyrazinylsulfonyl or optionally substituted polycyclic heteroarylsulfonyl, here in particular optionally substituted quinolinylsulfonyl, for example substituted by fluorine, chlorine, bromine, iodine, cyano, nitro, alkyl, haloalkyl, haloalkoxy, amino, alkylamino, alkylcarbonylamino, dialkylamino or alkoxy groups.

According to the invention, “alkylthio”—alone or as part of a chemical group—represents straight-chain or branched S-alkyl, preferably having 1 to 8 or having 1 to 6 carbon atoms, for example methylthio, ethylthio, n-propylthio, Isopropylthio, n-butylthio, isobutylthio, sec-butylthio and tert-butylthio. Alkenylthio is an alkenyl radical attached via a sulfur atom, alkynylthio is an alkynyl radical attached via a sulfur atom, cycloalkylthio is a cycloalkyl radical attached via a sulfur atom, and cycloalkenylthio is a cycloalkenyl radical attached via a sulfur atom.

“Alkoxy” is an alkyl radical attached via an oxygen atom, alkenyloxy is an alkenyl radical attached via an oxygen atom, alkynyloxy is an alkynyl radical attached via an oxygen atom, cycloalkyloxy is a cycloalkyl radical attached via an oxygen atom, and cycloalkenyloxy is a cycloalkenyl radical attached via an oxygen atom.

The term “aryl” means an optionally substituted mono-, bi- or polycyclic aromatic system having preferably 6 to 14, especially 6 to 10, ring carbon atoms, for example phenyl, naphthyl, anthryl, phenanthrenyl and the like, preferably phenyl.

The term “optionally substituted aryl” also includes polycyclic systems, such as tetrahydronaphtyl, indenyl, indanyl, fluorenyl, biphenylyl, where the bonding site is on the aromatic system. In systematic terms, “aryl” is generally also encompassed by the term “optionally substituted phenyl”. Here, preferred aryl substituents are, for example, hydrogen, halogen, alkyl, cycloalkyl, cycloalkylalkyl, cycloalkenyl, halocycloalkyl, alkenyl, alkynyl, aryl, arylalkyl, arylalkenyl, heteroaryl, heteroarylalkyl, heterocyclyl, heterocyclylalkyl, alkoxyalkyl, alkylthio, haloalkylthio, haloalkyl, alkoxy, haloalkoxy, cycloalkoxy, cycloalkylalkoxy, aryloxy, heteroaryloxy, alkoxyalkoxy, alkynylalkoxy, alkenyloxy, bisalkylaminoalkoxy, tris[alkyl]silyl, bis[alkyl]arylsilyl, bis[alkyl]alkylsilyl, tris[alkyl]silylalkynyl, arylalkynyl, heteroarylalkynyl, alkylalkynyl, cycloalkylalkynyl, haloalkylalkynyl, heterocyclyl-N-alkoxy, nitro, cyano, amino, alkylamino, bisalkylamino, alkylcarbonylamino, cycloalkylcarbonylamino, arylcarbonylamino, alkoxycarbonylamino, alkoxycarbonylalkylamino, arylalkoxycarbonylalkylamino, hydroxycarbonyl, alkoxycarbonyl, aminocarbonyl, alkylaminocarbonyl, cycloalkylaminocarbonyl, bisalkylaminocarbonyl, heteroarylalkoxy, arylalkoxy.

A heterocyclic radical (heterocyclyl) contains at least one heterocyclic ring (=carbocyclic ring in which at least one carbon atom has been replaced by a heteroatom, preferably by a heteroatom from the group of N, O, S, P) which is saturated, unsaturated, partly saturated or heteroaromatic and may be unsubstituted or substituted, in which case the bonding site is localized on a ring atom. When the heterocyclyl radical or the heterocyclic ring is optionally substituted, it may be fused to other carbocyclic or heterocyclic rings. In the case of optionally substituted heterocyclyl, polycyclic systems are also included, for example 8-azabicyclo[3.2.1]octanyl, 8-azabicyclo[2.2.2]octanyl or 1-azabicyclo[2.2.1]heptyl. In the case of optionally substituted heterocyclyl, spirocyclic systems are also included, such as, for example, 1-oxa-5-azaspiro[2.3]hexyl. Unless defined differently, the heterocyclic ring contains preferably 3 to 9 ring atoms and in particular 3 to 6 ring atoms and one or more, preferably 1 to 4 and in particular 1, 2 or 3 heteroatoms in the heterocyclic ring, preferably from the group consisting of N, O and S, although no two oxygen atoms should be directly adjacent, for example, with one heteroatom from the group consisting of N, O and S, 1- or 2- or 3-pyrrolidinyl, 3,4-dihydro-2H-pyrrol-2- or 3-yl, 2,3-dihydro-1H-pyrrol-1- or 2- or 3- or 4- or 5-yl; 2,5-dihydro-1H-pyrrol-1- or 2- or 3-yl, 1- or 2- or 3- or 4-piperidinyl; 2,3,4,5-tetrahydropyridin-2- or 3- or 4- or 5-yl or 6-yl; 1,2,3,6-tetrahydropyridin-1- or 2- or 3- or 4- or 5- or 6-yl; 1,2,3,4-tetrahydropyridin-1- or 2- or 3- or 4- or 5- or 6-yl; 1,4-dihydropyridin-1- or 2- or 3- or 4-yl; 2,3-dihydropyridin-2- or 3- or 4- or 5- or 6-yl; 2,5-dihydropyridin-2- or 3- or 4- or 5- or 6-yl, 1- or 2- or 3- or 4-azepanyl; 2,3,4,5-tetrahydro-1H-azepin-1- or 2- or 3- or 4- or 5- or 6- or 7-yl; 2,3,4,7-tetrahydro-1H-azepin-1- or 2- or 3- or 4- or 5- or 6- or 7-yl; 2,3,6,7-tetrahydro-1H-azepin-1- or 2- or 3- or 4-yl; 3,4,5,6-tetrahydro-2H-azepin-2- or 3- or 4- or 5- or 6- or 7-yl; 4,5-dihydro-1H-azepin-1- or 2- or 3- or 4-yl; 2,5-dihydro-1H-azepin-1- or -2- or 3- or 4- or 5- or 6- or 7-yl; 2,7-dihydro-1H-azepin-1- or -2- or 3- or 4-yl; 2,3-dihydro-1H-azepin-1- or -2- or 3- or 4- or 5- or 6- or 7-yl; 3,4-dihydro-2H-azepin-2- or 3- or 4- or 5- or 6- or 7-yl; 3,6-dihydro-2H-azepin-2- or 3- or 4- or 5- or 6- or 7-yl; 5,6-dihydro-2H-azepin-2- or 3- or 4- or 5- or 6- or 7-yl; 4,5-dihydro-3H-azepin-2- or 3- or 4- or 5- or 6- or 7-yl; 1H-azepin-1- or -2- or 3- or 4- or 5- or 6- or 7-yl; 2H-azepin-2- or 3- or 4- or 5- or 6- or 7-yl; 3H-azepin-2- or 3- or 4- or 5- or 6- or 7-yl; 4H-azepin-2- or 3- or 4- or 5- or 6- or 7-yl, 2- or 3-oxolanyl (=2- or 3-tetrahydrofuranyl); 2,3-dihydrofuran-2- or 3- or 4- or 5-yl; 2,5-dihydrofuran-2- or 3-yl, 2- or 3- or 4-oxanyl (=2- or 3- or 4-tetrahydropyranyl); 3,4-dihydro-2H-pyran-2- or 3- or 4- or 5- or 6-yl; 3,6-dihydro-2H-pyran-2- or 3- or 4- or 5- or 6-yl; 2H-pyran-2- or 3- or 4- or 5- or 6-yl; 4H-pyran-2- or 3- or 4-yl, 2- or 3- or 4-oxepanyl; 2,3,4,5-tetrahydrooxepin-2- or 3- or 4- or 5- or 6- or 7-yl; 2,3,4,7-tetrahydrooxepin-2- or 3- or 4- or 5- or 6- or 7-yl; 2,3,6,7-tetrahydrooxepin-2- or 3- or 4-yl; 2,3-dihydrooxepin-2- or 3- or 4- or 5- or 6- or 7-yl; 4,5-dihydrooxepin-2- or 3- or 4-yl; 2,5-dihydrooxepin-2- or 3- or 4- or 5- or 6- or 7-yl; oxepin-2- or 3- or 4- or 5- or 6- or 7-yl; 2- or 3-tetrahydrothiophenyl; 2,3-dihydrothiophen-2- or 3- or 4- or 5-yl; 2,5-dihydrothiophen-2- or 3-yl; tetrahydro-2H-thiopyran-2- or 3- or 4-yl; 3,4-dihydro-2H-thiopyran-2- or 3- or 4- or 5- or 6-yl; 3,6-dihydro-2H-thiopyran-2- or 3- or 4- or 5- or 6-yl; 2H-thiopyran-2- or 3- or 4- or 5- or 6-yl; 4H-thiopyran-2- or 3- or 4-yl. Preferred 3-membered and 4-membered heterocycles are, for example, 1- or 2-aziridinyl, oxiranyl, thiiranyl, 1- or 2- or 3-azetidinyl, 2- or 3-oxetanyl, 2- or 3-thietanyl, 1,3-dioxetan-2-yl. Further examples of “heterocyclyl” are a partially or fully hydrogenated heterocyclic radical having two heteroatoms from the group consisting of N, O and S, such as, for example, 1- or 2- or 3- or 4-pyrazolidinyl; 4,5-dihydro-3H-pyrazol-3- or 4- or 5-yl; 4,5-dihydro-1H-pyrazol-1- or 3- or 4- or 5-yl; 2,3-dihydro-1H-pyrazol-1- or 2- or 3- or 4- or 5-yl; 1- or 2- or 3- or 4-imidazolidinyl; 2,3-dihydro-1H-imidazol-1- or 2- or 3- or 4-yl; 2,5-dihydro-1H-imidazol-1- or 2- or 4- or 5-yl; 4,5-dihydro-1H-imidazol-1- or 2- or 4- or 5-yl; hexahydropyridazin-1- or 2- or 3- or 4-yl; 1,2,3,4-tetrahydropyridazin-1- or 2- or 3- or 4- or 5- or 6-yl; 1,2,3,6-tetrahydropyridazin-1- or 2- or 3- or 4- or 5- or 6-yl; 1,4,5,6-tetrahydropyridazin-1- or 3- or 4- or 5- or 6-yl; 3,4,5,6-tetrahydropyridazin-3- or 4- or 5-yl; 4,5-dihydropyridazin-3- or 4-yl; 3,4-dihydropyridazin-3- or 4- or 5- or 6-yl; 3,6-dihydropyridazin-3- or 4-yl; 1,6-dihydropyriazin-1- or 3- or 4- or 5- or 6-yl; hexahydropyrimidin-1- or 2- or 3- or 4-yl; 1,4,5,6-tetrahydropyrimidin-1- or 2- or 4- or 5- or 6-yl; 1,2,5,6-tetrahydropyrimidin-1- or 2- or 4- or 5- or 6-yl; 1,2,3,4-tetrahydropyrimidin-1- or 2- or 3- or 4- or 5- or 6-yl; 1,6-dihydropyrimidin-1- or 2- or 4- or 5- or 6-yl; 1,2-dihydropyrimidin-1- or 2- or 4- or 5- or 6-yl; 2,5-dihydropyrimidin-2- or 4- or 5-yl; 4,5-dihydropyrimidin-4- or 5- or 6-yl; 1,4-dihydropyrimidin-1- or 2- or 4- or 5- or 6-yl; 1- or 2- or 3-piperazinyl; 1,2,3,6-tetrahydropyrazin-1- or 2- or 3- or 5- or 6-yl; 1,2,3,4-tetrahydropyrazin-1- or 2- or 3- or 4- or 5- or 6-yl; 1,2-dihydropyrazin-1- or 2- or 3- or 5- or 6-yl; 1,4-dihydropyrazin-1- or 2- or 3-yl; 2,3-dihydropyrazin-2- or 3- or 5- or 6-yl; 2,5-dihydropyrazin-2- or 3-yl; 1,3-dioxolan-2- or 4- or 5-yl; 1,3-dioxol-2- or 4-yl; 1,3-dioxan-2- or 4- or 5-yl; 4H-1,3-dioxin-2- or 4- or 5- or 6-yl; 1,4-dioxan-2- or 3- or 5- or 6-yl; 2,3-dihydro-1,4-dioxin-2- or 3- or 5- or 6-yl; 1,4-dioxin-2- or 3-yl; 1,2-dithiolan-3- or 4-yl; 3H-1,2-dithiol-3- or 4- or 5-yl; 1,3-dithiolan-2- or 4-yl; 1,3-dithiol-2- or 4-yl; 1,2-dithian-3- or 4-yl; 3,4-dihydro-1,2-dithiin-3- or 4- or 5- or 6-yl; 3,6-dihydro-1,2-dithiin-3- or 4-yl; 1,2-dithiin-3- or 4-yl; 1,3-dithian-2- or 4- or 5-yl; 4H-1,3-dithiin-2- or 4- or 5- or 6-yl; isoxazolidin-2- or 3- or 4- or 5-yl; 2,3-dihydroisoxazol-2- or 3- or 4- or 5-yl; 2,5-dihydroisoxazol-2- or 3- or 4- or 5-yl; 4,5-dihydroisoxazol-3- or 4- or 5-yl; 1,3-oxazolidin-2- or 3- or 4- or 5-yl; 2,3-dihydro-1,3-oxazol-2- or 3- or 4- or 5-yl; 2,5-dihydro-1,3-oxazol-2- or 4- or 5-yl; 4,5-dihydro-1,3-oxazol-2- or 4- or 5-yl; 1,2-oxazinan-2- or 3- or 4- or 5- or 6-yl; 3,4-dihydro-2H-1,2-oxazin-2- or 3- or 4- or 5- or 6-yl; 3,6-dihydro-2H-1,2-oxazin-2- or 3- or 4- or 5- or 6-yl; 5,6-dihydro-2H-1,2-oxazin-2- or 3- or 4- or 5- or 6-yl; 5,6-dihydro-4H-1,2-oxazin-3- or 4- or 5- or 6-yl; 2H-1,2-oxazin-2- or 3- or 4- or 5- or 6-yl; 6H-1,2-oxazin-3- or 4- or 5- or 6-yl; 4H-1,2-oxazin-3- or 4- or 5- or 6-yl; 1,3-oxazinan-2- or 3- or 4- or 5- or 6-yl; 3,4-dihydro-2H-1,3-oxazin-2- or 3- or 4- or 5- or 6-yl; 3,6-dihydro-2H-1,3-oxazin-2- or 3- or 4- or 5- or 6-yl; 5,6-dihydro-2H-1,3-oxazin-2- or 4- or 5- or 6-yl; 5,6-dihydro-4H-1,3-oxazin-2- or 4- or 5- or 6-yl; 2H-1,3-oxazin-2- or 4- or 5- or 6-yl; 6H-1,3-oxazin-2- or 4- or 5- or 6-yl; 4H-1,3-oxazin-2- or 4- or 5- or 6-yl; morpholin-2- or 3- or 4-yl; 3,4-dihydro-2H-1,4-oxazin-2- or 3- or 4- or 5- or 6-yl; 3,6-dihydro-2H-1,4-oxazin-2- or 3- or 5- or 6-yl; 2H-1,4-oxazin-2- or 3- or 5- or 6-yl; 4H-1,4-oxazin-2- or 3-yl; 1,2-oxazepan-2- or 3- or 4- or 5- or 6- or 7-yl; 2,3,4,5-tetrahydro-1,2-oxazepin-2- or 3- or 4- or 5- or 6- or 7-yl; 2,3,4,7-tetrahydro-1,2-oxazepin-2- or 3- or 4- or 5- or 6- or 7-yl; 2,3,6,7-tetrahydro-1,2-oxazepin-2- or 3- or 4- or 5- or 6- or 7-yl; 2,5,6,7-tetrahydro-1,2-oxazepin-2- or 3- or 4- or 5- or 6- or 7-yl; 4,5,6,7-tetrahydro-1,2-oxazepin-3- or 4- or 5- or 6- or 7-yl; 2,3-dihydro-1,2-oxazepin-2- or 3- or 4- or 5- or 6- or 7-yl; 2,5-dihydro-1,2-oxazepin-2- or 3- or 4- or 5- or 6- or 7-yl; 2,7-dihydro-1,2-oxazepin-2- or 3- or 4- or 5- or 6- or 7-yl; 4,5-dihydro-1,2-oxazepin-3- or 4- or 5- or 6- or 7-yl; 4,7-dihydro-1,2-oxazepin-3- or 4- or 5- or 6- or 7-yl; 6,7-dihydro-1,2-oxazepin-3- or 4- or 5- or 6- or 7-yl; 1,2-oxazepin-3- or 4- or 5- or 6- or 7-yl; 1,3-oxazepan-2- or 3- or 4- or 5- or 6- or 7-yl; 2,3,4,5-tetrahydro-1,3-oxazepin-2- or 3- or 4- or 5- or 6- or 7-yl; 2,3,4,7-tetrahydro-1,3-oxazepin-2- or 3- or 4- or 5- or 6- or 7-yl; 2,3,6,7-tetrahydro-1,3-oxazepin-2- or 3- or 4- or 5- or 6- or 7-yl; 2,5,6,7-tetrahydro-1,3-oxazepin-2- or 4- or 5- or 6- or 7-yl; 4,5,6,7-tetrahydro-1,3-oxazepin-2- or 4- or 5- or 6- or 7-yl; 2,3-dihydro-1,3-oxazepin-2- or 3- or 4- or 5- or 6- or 7-yl; 2,5-dihydro-1,3-oxazepin-2- or 4- or 5- or 6- or 7-yl; 2,7-dihydro-1,3-oxazepin-2- or 4- or 5- or 6- or 7-yl; 4,5-dihydro-1,3-oxazepin-2- or 4- or 5- or 6- or 7-yl; 4,7-dihydro-1,3-oxazepin-2- or 4- or 5- or 6- or 7-yl; 6,7-dihydro-1,3-oxazepin-2- or 4- or 5- or 6- or 7-yl; 1,3-oxazepin-2- or 4- or 5- or 6- or 7-yl; 1,4-oxazepan-2- or 3- or 5- or 6- or 7-yl; 2,3,4,5-tetrahydro-1,4-oxazepin-2- or 3- or 4- or 5- or 6- or 7-yl; 2,3,4,7-tetrahydro-1,4-oxazepin-2- or 3- or 4- or 5- or 6- or 7-yl; 2,3,6,7-tetrahydro-1,4-oxazepin-2- or 3- or 5- or 6- or 7-yl; 2,5,6,7-tetrahydro-1,4-oxazepin-2- or 3- or 5- or 6- or 7-yl; 4,5,6,7-tetrahydro-1,4-oxazepin-2- or 3- or 4- or 5- or 6- or 7-yl; 2,3-dihydro-1,4-oxazepin-2- or 3- or 5- or 6- or 7-yl; 2,5-dihydro-1,4-oxazepin-2- or 3- or 5- or 6- or 7-yl; 2,7-dihydro-1,4-oxazepin-2- or 3- or 5- or 6- or 7-yl; 4,5-dihydro-1,4-oxazepin-2- or 3- or 4- or 5- or 6- or 7-yl; 4,7-dihydro-1,4-oxazepin-2- or 3- or 4- or 5- or 6- or 7-yl; 6,7-dihydro-1,4-oxazepin-2- or 3- or 5- or 6- or 7-yl; 1,4-oxazepin-2- or 3- or 5- or 6- or 7-yl; isothiazolidin-2- or 3- or 4- or 5-yl; 2,3-dihydroisothiazol-2- or 3- or 4- or 5-yl; 2,5-dihydroisothiazol-2- or 3- or 4- or 5-yl; 4,5-dihydroisothiazol-3- or 4- or 5-yl; 1,3-thiazolidin-2- or 3- or 4- or 5-yl; 2,3-dihydro-1,3-thiazol-2- or 3- or 4- or 5-yl; 2,5-dihydro-1,3-thiazol-2- or 4- or 5-yl; 4,5-dihydro-1,3-thiazol-2- or 4- or 5-yl; 1,3-thiazinan-2- or 3- or 4- or 5- or 6-yl; 3,4-dihydro-2H-1,3-thiazin-2- or 3- or 4- or 5- or 6-yl; 3,6-dihydro-2H-1,3-thiazin-2- or 3- or 4- or 5- or 6-yl; 5,6-dihydro-2H-1,3-thiazin-2- or 4- or 5- or 6-yl; 5,6-dihydro-4H-1,3-thiazin-2- or 4- or 5- or 6-yl; 2H-1,3-thiazin-2- or 4- or 5- or 6-yl; 6H-1,3-thiazin-2- or 4- or 5- or 6-yl; 4H-1,3-thiazin-2- or 4- or 5- or 6-yl. Further examples of “heterocyclyl” are a partly or fully hydrogenated heterocyclic radical having 3 heteroatoms from the group of N, O and S, for example 1,4,2-dioxazolidin-2- or 3- or 5-yl; 1,4,2-dioxazol-3- or 5-yl; 1,4,2-dioxazinan-2- or -3- or 5- or 6-yl; 5,6-dihydro-1,4,2-dioxazin-3- or 5- or 6-yl; 1,4,2-dioxazin-3- or 5- or 6-yl; 1,4,2-dioxazepan-2- or 3- or 5- or 6- or 7-yl; 6,7-dihydro-5H-1,4,2-dioxazepin-3- or 5- or 6- or 7-yl; 2,3-dihydro-7H-1,4,2-dioxazepin-2- or 3- or 5- or 6- or 7-yl; 2,3-dihydro-5H-1,4,2-dioxazepin-2- or 3- or 5- or 6- or 7-yl; 5H-1,4,2-dioxazepin-3- or 5- or 6- or 7-yl; 7H-1,4,2-dioxazepin-3- or 5- or 6- or 7-yl. Structural examples of heterocycles which are optionally substituted further are also listed below:

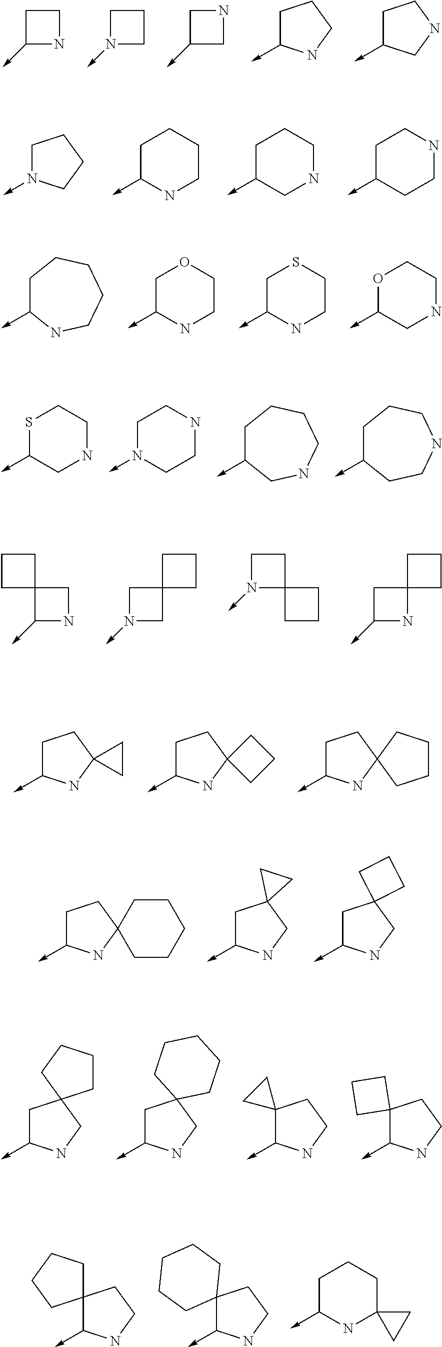

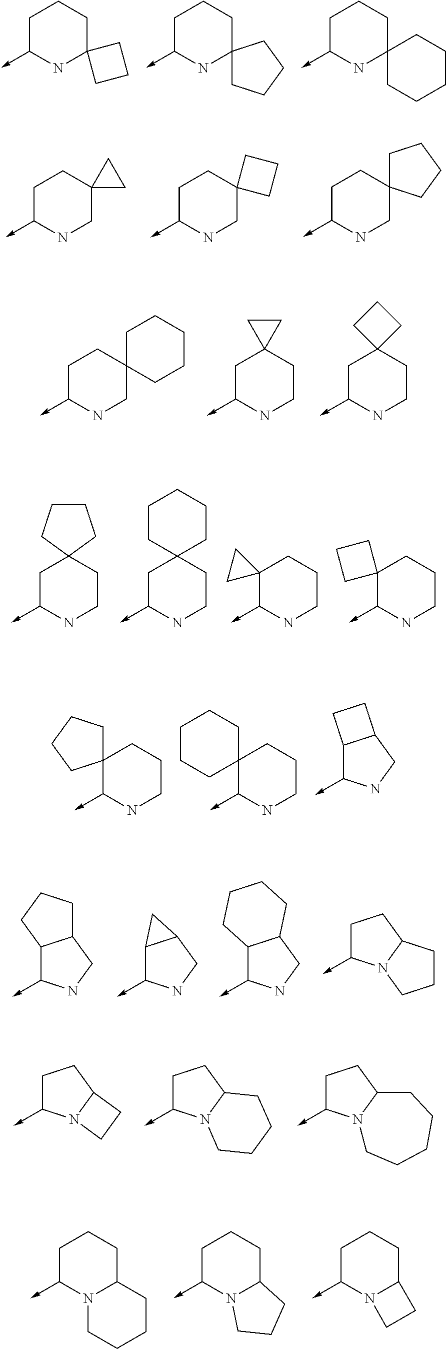

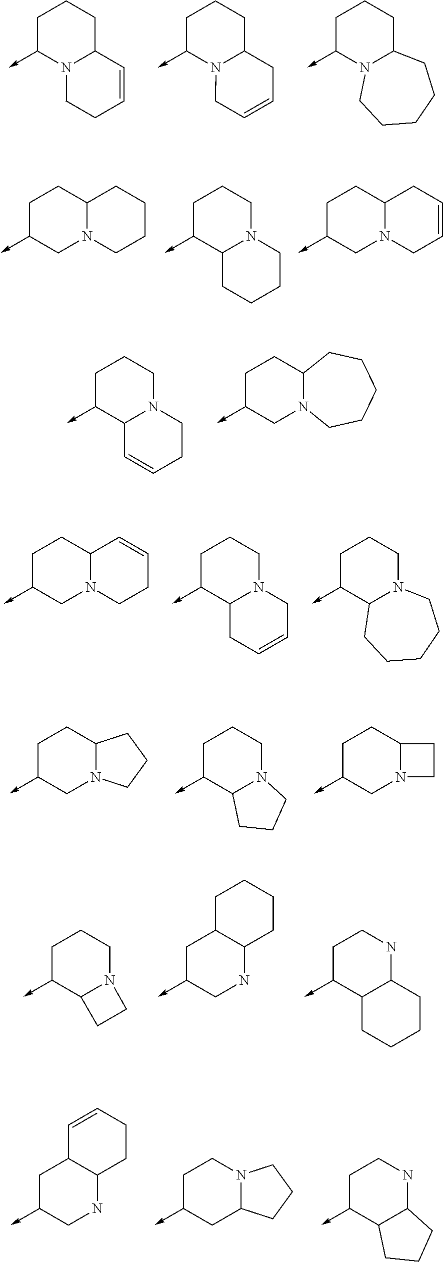

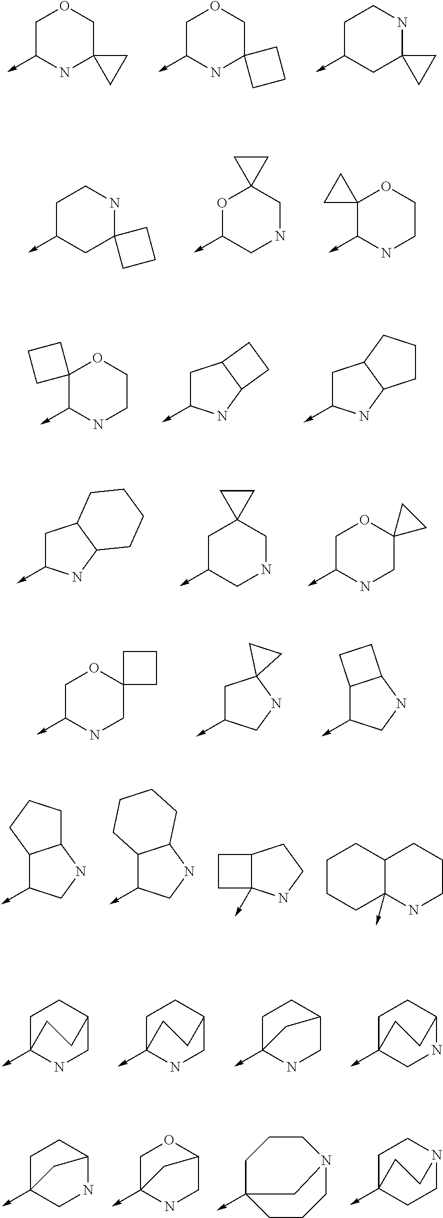

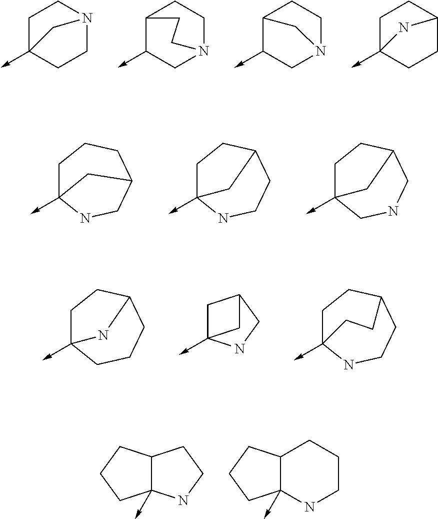

The heterocycles listed above are preferably substituted, for example, by hydrogen, halogen, alkyl, haloalkyl, hydroxy, alkoxy, cycloalkoxy, aryloxy, alkoxyalkyl, alkoxyalkoxy, cycloalkyl, halocycloalkyl, aryl, arylalkyl, heteroaryl, heterocyclyl, alkenyl, alkylcarbonyl, cycloalkylcarbonyl, arylcarbonyl, heteroarylcarbonyl, alkoxycarbonyl, hydroxycarbonyl, cycloalkoxycarbonyl, cycloalkylalkoxycarbonyl, alkoxycarbonylalkyl, arylalkoxycarbonyl, arylalkoxycarbonylalkyl, alkynyl, alkynylalkyl, alkylalkynyl, trisalkylsilylalkynyl, nitro, amino, cyano, haloalkoxy, haloalkylthio, alkylthio, hydrothio, hydroxyalkyl, oxo, heteroarylalkoxy, arylalkoxy, heterocyclylalkoxy, heterocyclylalkylthio, heterocyclyloxy, heterocyclylthio, heteroaryloxy, bisalkylamino, alkylamino, cycloalkylamino, hydroxycarbonylalkylamino, alkoxycarbonylalkylamino, arylalkoxycarbonylalkylamino, alkoxycarbonylalkyl(alkyl)amino, aminocarbonyl, alkylaminocarbonyl, bisalkylaminocarbonyl, cycloalkylaminocarbonyl, hydroxycarbonylalkylaminocarbonyl, alkoxycarbonylalkylaminocarbonyl, arylalkoxycarbonylalkylaminocarbonyl.

When a base structure is substituted “by one or more radicals” from a list of radicals (=group) or a generically defined group of radicals, this in each case includes simultaneous substitution by a plurality of identical and/or structurally different radicals.

In the case of a partly or fully saturated nitrogen heterocycle, this may be joined to the remainder of the molecule either via carbon or via the nitrogen.

Suitable substituents for a substituted heterocyclic radical are the substituents specified later on below, and additionally also oxo and thioxo. The oxo group as a substituent on a ring carbon atom is then, for example, a carbonyl group in the heterocyclic ring. As a result, lactones and lactams are preferably also included. The oxo group may also be present on the ring heteroatoms, which can exist in various oxidation states, for example on N and S, in which case they form, for example, the divalent groups N(O), S(O) (also SO for short) and S(O)2 (also SO2 for short) in the heterocyclic ring. In the case of —N(O)- and —S(O)-groups, both enantiomers in each case are included.

According to the invention, the expression “heteroaryl” represents heteroaromatic compounds, i.e. fully unsaturated aromatic heterocyclic compounds, preferably 5- to 7-membered rings having 1 to 4, preferably 1 or 2, identical or different heteroatoms, preferably O, S or N. Inventive heteroaryls are, for example, 1H-pyrrol-1-yl; 1H-pyrrol-2-yl; 1H-pyrrol-3-yl; furan-2-yl; furan-3-yl; thien-2-yl; thien-3-yl, 1H-imidazol-1-yl; 1H-imidazol-2-yl; 1H-imidazol-4-yl; 1H-imidazol-5-yl; 1H-pyrazol-1-yl; 1H-pyrazol-3-yl; 1H-pyrazol-4-yl; 1H-pyrazol-5-yl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 1H-1,2,4-triazol-1-yl, 1H-1,2,4-triazol-3-yl, 4H-1,2,4-triazol-4-yl, 1,2,4-oxadiazol-3-yl, 1,2,4-oxadiazol-5-yl, 1,3,4-oxadiazol-2-yl, 1,2,3-oxadiazol-4-yl, 1,2,3-oxadiazol-5-yl, 1,2,5-oxadiazol-3-yl, azepinyl, pyridin-2-yl, pyridin-3-yl, pyridin-4-yl, pyrazin-2-yl, pyrazin-3-yl, pyrimidin-2-yl, pyrimidin-4-yl, pyrimidin-5-yl, pyridazin-3-yl, pyridazin-4-yl, 1,3,5-triazin-2-yl, 1,2,4-triazin-3-yl, 1,2,4-triazin-5-yl, 1,2,4-triazin-6-yl, 1,2,3-triazin-4-yl, 1,2,3-triazin-5-yl, 1,2,4-, 1,3,2-, 1,3,6- and 1,2,6-oxazinyl, isoxazol-3-yl, isoxazol-4-yl, isoxazol-5-yl, 1,3-oxazol-2-yl, 1,3-oxazol-4-yl, 1,3-oxazol-5-yl, isothiazol-3-yl, isothiazol-4-yl, isothiazol-5-yl, 1,3-thiazol-2-yl, 1,3-thiazol-4-yl, 1,3-thiazol-5-yl, oxepinyl, thiepinyl, 1,2,4-triazolonyl and 1,2,4-diazepinyl, 2H-1,2,3,4-tetrazol-5-yl, 1H-1,2,3,4-tetrazol-5-yl, 1,2,3,4-oxatriazol-5-yl, 1,2,3,4-thiatriazol-5-yl, 1,2,3,5-oxatriazol-4-yl, 1,2,3,5-thiatriazol-4-yl. The heteroaryl groups according to the invention may also be substituted by one or more identical or different radicals. If two adjacent carbon atoms are part of a further aromatic ring, the systems are fused heteroaromatic systems, such as benzofused or polyannulated heteroaromatics. Preferred examples are quinolines (e.g. quinolin-2-yl, quinolin-3-yl, quinolin-4-yl, quinolin-5-yl, quinolin-6-yl, quinolin-7-yl, quinolin-8-yl); isoquinolines (e.g. isoquinolin-1-yl, isoquinolin-3-yl, isoquinolin-4-yl, isoquinolin-5-yl, isoquinolin-6-yl, isoquinolin-7-yl, isoquinolin-8-yl); quinoxaline; quinazoline; cinnoline; 1,5-naphthyridine; 1,6-naphthyridine; 1,7-naphthyridine; 1,8-naphthyridine; 2,6-naphthyridine; 2,7-naphthyridine; phthalazine; pyridopyrazines; pyridopyrimidines; pyridopyridazines; pteridines; pyrimidopyrimidines. Examples of heteroaryl are also 5- or 6-membered benzofused rings from the group of 1H-indol-1-yl, 1H-indol-2-yl, 1H-indol-3-yl, 1H-indol-4-yl, 1H-indol-5-yl, 1H-indol-6-yl, 1H-indol-7-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, 2H-indazol-7-yl, 2H-isoindol-2-yl, 2H-isoindol-1-yl, 2H-isoindol-3-yl, 2H-isoindol-4-yl, 2H-isoindol-5-yl, 2H-isoindol-6-yl; 2H-isoindol-7-yl, 1H-benzimidazol-1-yl, 1H-benzimidazol-2-yl, 1H-benzimidazol-4-yl, 1H-benzimidazol-5-yl, 1H-benzimidazol-6-yl, 1H-benzimidazol-7-yl, 1,3-benzoxazol-2-yl, 1,3-benzoxazol-4-yl, 1,3-benzoxazol-5-yl, 1,3-benzoxazol-6-yl, 1,3-benzoxazol-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,2-benzisoxazol-3-yl, 1,2-benzisoxazol-4-yl, 1,2-benzisoxazol-5-yl, 1,2-benzisoxazol-6-yl, 1,2-benzisoxazol-7-yl, 1,2-benzisothiazol-3-yl, 1,2-benzisothiazol-4-yl, 1,2-benzisothiazol-5-yl, 1,2-benzisothiazol-6-yl, 1,2-benzisothiazol-7-yl.

The term “halogen” means, for example, fluorine, chlorine, bromine or iodine. If the term is used for a radical, “halogen” means, for example, a fluorine, chlorine, bromine or iodine atom.

According to the invention, “alkyl” means a straight-chain or branched open-chain, saturated hydrocarbon radical which is optionally mono- or polysubstituted, preferably unsubstituted. Preferred substituents are halogen atoms, alkoxy, haloalkoxy, cyano, alkylthio, haloalkylthio, amino or nitro groups, particular preference being given to methoxy, methyl, fluoroalkyl, cyano, nitro, fluorine, chlorine, bromine or iodine.

“Haloalkyl”, “-alkenyl” and “-alkynyl” mean alkyl, alkenyl and alkynyl, respectively, partially or fully substituted by identical or different halogen atoms, for example monohaloalkyl such as, for example, CH₂CH₂Cl, CH₂CH₂Br, CHClCH₃, CH₂Cl, CH₂F; perhaloalkyl such as, for example, CCl₃, CClF₂, CFCl₂, CF₂CClF₂, CF₂CClFCF₃; polyhaloalkyl such as, for example, CH₂CHFCl, CF₂CClFH, CF₂CBrFH, CH₂CF₃; here, the term perhaloalkyl also includes the term perfluoroalkyl.

Partly fluorinated alkyl means a straight-chain or branched, saturated hydrocarbon which is mono- or polysubstituted by fluorine, where the fluorine atoms in question may be present as substituents on one or more different carbon atoms of the straight-chain or branched hydrocarbyl chain, for example CHFCH₃, CH₂CH₂F, CH₂CH₂CF₃, CHF₂, CH₂F, CHFCF₂CF₃.

Partly fluorinated haloalkyl means a straight-chain or branched, saturated hydrocarbon which is substituted by different halogen atoms with at least one fluorine atom, where any other halogen atoms optionally present are selected from the group consisting of fluorine, chlorine or bromine, iodine. The corresponding halogen atoms may be present as substituents on one or more different carbon atoms of the straight-chain or branched hydrocarbyl chain. Partly fluorinated haloalkyl also includes full substitution of the straight or branched chain by halogen including at least one fluorine atom.

Haloalkoxy is, for example, OCF₃, OCHF₂, OCH₂F, OCF₂CF₃, OCH₂CF₃ and OCH₂CH₂Cl; the situation is equivalent for haloalkenyl and other halogen-substituted radicals.

The expression “(C₁-C₄)-alkyl” mentioned here by way of example is a brief notation for straight-chain or branched alkyl having one to 4 carbon atoms according to the range stated for carbon atoms, i.e. encompasses the methyl, ethyl, 1-propyl, 2-propyl, 1-butyl, 2-butyl, 2-methylpropyl or tert-butyl radicals. General alkyl radicals with a larger specified range of carbon atoms, e.g. “(C₁-C₆)-alkyl”, correspondingly also encompass straight-chain or branched alkyl radicals with a greater number of carbon atoms, i.e. according to the example also the alkyl radicals having 5 and 6 carbon atoms.

Unless stated specifically, preference is given to the lower carbon skeletons, for example having from 1 to 6 carbon atoms, or having from 2 to 6 carbon atoms in the case of unsaturated groups, in the case of the hydrocarbon radicals such as alkyl, alkenyl and alkynyl radicals, including in composite radicals. Alkyl radicals, including in composite radicals such as alkoxy, haloalkyl, etc., are, for example, methyl, ethyl, n-propyl or i-propyl, n-, i-, t- or 2-butyl, pentyls, hexyls such as n-hexyl, i-hexyl and 1,3-dimethylbutyl, heptyls such as n-heptyl, 1-methylhexyl and 1,4-dimethylpentyl; alkenyl and alkynyl radicals are defined as the possible unsaturated radicals corresponding to the alkyl radicals, where at least one double bond or triple bond is present. Preference is given to radicals having one double bond or triple bond.

The term “alkenyl” also includes, in particular, straight-chain or branched open-chain hydrocarbyl radicals having more than one double bond, such as 1,3-butadienyl and 1,4-pentadienyl, but also allenyl or cumulenyl radicals having one or more cumulated double bonds, for example allenyl (1,2-propadienyl), 1,2-butadienyl and 1,2,3-pentatrienyl. Alkenyl is, for example, vinyl which may optionally be substituted by further alkyl radicals, for example prop-1-en-1-yl, but-1-en-1-yl, allyl, 1-methylprop-2-en-1-yl, 2-methylprop-2-en-1-yl, but-2-en-1-yl, 1-methylbut-3-en-1-yl and 1-methylbut-2-en-1-yl, 2-methylprop-1-en-1-yl, 1-methylprop-1-en-1-yl, 1-methylprop-2-en-1-yl, 2-methylprop-2-en-1-yl, but-2-en-1-yl, but-3-en-1-yl, 1-methylbut-3-en-1-yl or 1-methylbut-2-en-1-yl, pentenyl, 2-methylpentenyl or hexenyl.

The term “alkynyl” also includes, in particular, straight-chain or branched open-chain hydrocarbyl radicals having more than one triple bond, or else having one or more triple bonds and one or more double bonds, for example 1,3-butatrienyl or 3-penten-1-yn-1-yl. (C₂-C₆)-alkynyl is, for example, ethynyl, propargyl, 1-methylprop-2-yn-1-yl, 2-butynyl, 2-pentynyl or 2-hexynyl, preferably propargyl, but-2-yn-1-yl, but-3-yn-1-yl or 1-methylbut-3-yn-1-yl.

The term “cycloalkyl” means a carbocyclic saturated ring system having preferably 3-8 ring carbon atoms, for example cyclopropyl, cyclobutyl, cyclopentyl or cyclohexyl. In the case of optionally substituted cycloalkyl, cyclic systems with substituents are included, also including substituents with a double bond on the cycloalkyl radical, for example an alkylidene group such as methylidene. Optionally substituted cycloalkyl also includes polycyclic aliphatic systems, for example bicyclo[1.1.0]butan-1-yl, bicyclo[1.1.0]butan-2-yl, bicyclo[2.1.0]pentan-1-yl, bicyclo[2.1.0]pentan-2-yl, bicyclo[2.1.0]pentan-5-yl, bicyclo[2.2.1]hept-2-yl (norbornyl), bicyclo[2.2.2]octan-2-yl, adamantan-1-yl and adamantan-2-yl. The term “(C₃-C₇)-cycloalkyl” is a brief notation for cycloalkyl having three to 7 carbon atoms, corresponding to the range specified for carbon atoms.

In the case of substituted cycloalkyl, spirocyclic aliphatic systems are also included, for example spiro[2.2]pent-1-yl, spiro[2.3]hex-1-yl, spiro[2.3]hex-4-yl, 3-spiro[2.3]hex-5-yl.

“Cycloalkenyl” means a carbocyclic, nonaromatic, partly unsaturated ring system having preferably 4-8 carbon atoms, e.g. 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl, also including substituents with a double bond on the cycloalkenyl radical, for example an alkylidene group such as methylidene. In the case of optionally substituted cycloalkenyl, the elucidations for substituted cycloalkyl apply correspondingly.

The term “alkylidene”, for example including in the form of (C₁-C₁₀)-alkylidene, means the radical of a straight-chain or branched open-chain hydrocarbon radical attached via a double bond. Possible bonding sites for alkylidene are naturally only positions on the base structure where two hydrogen atoms can be replaced by the double bond; radicals are, for example, ═CH₂, ═CH—CH₃, ═C(CH₃)—CH₃, ═C(CH₃)—C₂H₅ or ═C(C₂H₅)—C₂H₅. Cycloalkylidene is a carbocyclic radical attached via a double bond.

The term “stannyl” represents a further-substituted radical containing a tin atom; “germanyl” analogously represents a further-substituted radical containing a germanium atom. “Zirconyl” represents a further-substituted radical containing a zirconium atom. “Hafnyl” represents a further-substituted radical containing a hafnium atom. “Boryl”, “borolanyl” and “borinanyl” represent further-substituted and optionally cyclic groups each containing a boron atom. “Plumbanyl” represents a further-substituted radical containing a lead atom. “Hydrargyl” represents a further-substituted radical containing a mercury atom. “Alanyl” represents a further-substituted radical containing an aluminum atom. “Magnesyl” represents a further-substituted radical containing a magnesium atom. “Zincyl” represents a further-substituted radical containing a zinc atom.

Depending on the nature of the substituents and the manner in which they are attached, the compounds of the general formula (I) may be present as stereoisomers. The formula (I) embraces all possible stereoisomers defined by the specific three-dimensional form thereof, such as enantiomers, diastereomers, Z and E isomers. When, for example, one or more alkenyl groups are present, diastereomers (Z and E isomers) may occur. When, for example, one or more asymmetric carbon atoms are present, enantiomers and diastereomers may occur. Stereoisomers can be obtained from the mixtures obtained in the preparation by customary separation methods. The chromatographic separation can be effected either on the analytical scale to find the enantiomeric excess or the diastereomeric excess, or else on the preparative scale to produce test specimens for biological testing. It is likewise possible to selectively prepare stereoisomers by using stereoselective reactions with use of optically active starting materials and/or auxiliaries. The invention thus also relates to all stereoisomers which are embraced by the general formula (I) but are not shown in their specific stereomeric form, and to mixtures thereof.

### Synthesis:

Substituted 2-amidobenzimidazoles can be prepared by known processes (cf. J. Med. Chem. 2000, 43, 4084; Bioorg. Med. Chem. 2008, 16, 6965; Bioorg. Med. Chem. 2008, 16, 3955; Org. Proc. Res. Develop. 2007, 11, 693; J. Med. Chem. 2009, 52, 514; J. Heterocyclic Chem. 2001, 38, 979; WO2000026192; WO2003106430; WO9704771; WO2000029384, WO2000032579). Various literature preparation routes were used to form the core structure, and some were optimized (see scheme 1). Selected detailed synthesis examples are detailed in the next section. The synthesis routes used and examined proceed from commercially available or easily preparable 2-amino-3-nitrobenzoic acids or 2,3-diaminobenzonitriles. The relevant 2-amino-3-nitrobenzoic acid with optional additional substitution can be converted with the aid of thionyl chloride and ammonia to the corresponding 2-amino-3-nitrobenzamide, which is reduced either with hydrogen in the presence of palladium on carbon in a suitable solvent or with tin(II) chloride to give an optionally further-substituted 2,3-diaminobenzamide. The 2,3-diaminobenzamide thus obtained can be converted in the subsequent step via various reaction variants, for example condensation with a carboxylic acid, with an aldehyde or an amide oxime, to the desired benzimidazole derivative. Alternatively, the corresponding benzimidazole can also be formed by condensation of a 2,3-diaminobenzoic acid with a carboxylic acid or by N-acylation of a 2-amino-3-nitrobenzoic ester and subsequent reduction with hydrogen in the presence of palladium on carbon, and the carboxyl function can be converted to the desired amide in the subsequent step. A further reaction route to the synthesis of the compounds according to the invention is the condensation of an optionally substituted 2,3-diaminobenzonitrile with a corresponding carboxylic acid and the subsequent reaction with a hydroxide base (e.g. potassium hydroxide) in a protic solvent (e.g. ethanol). The radicals R¹, R², R³ and Q mentioned in Scheme 1 below have the meanings defined above.

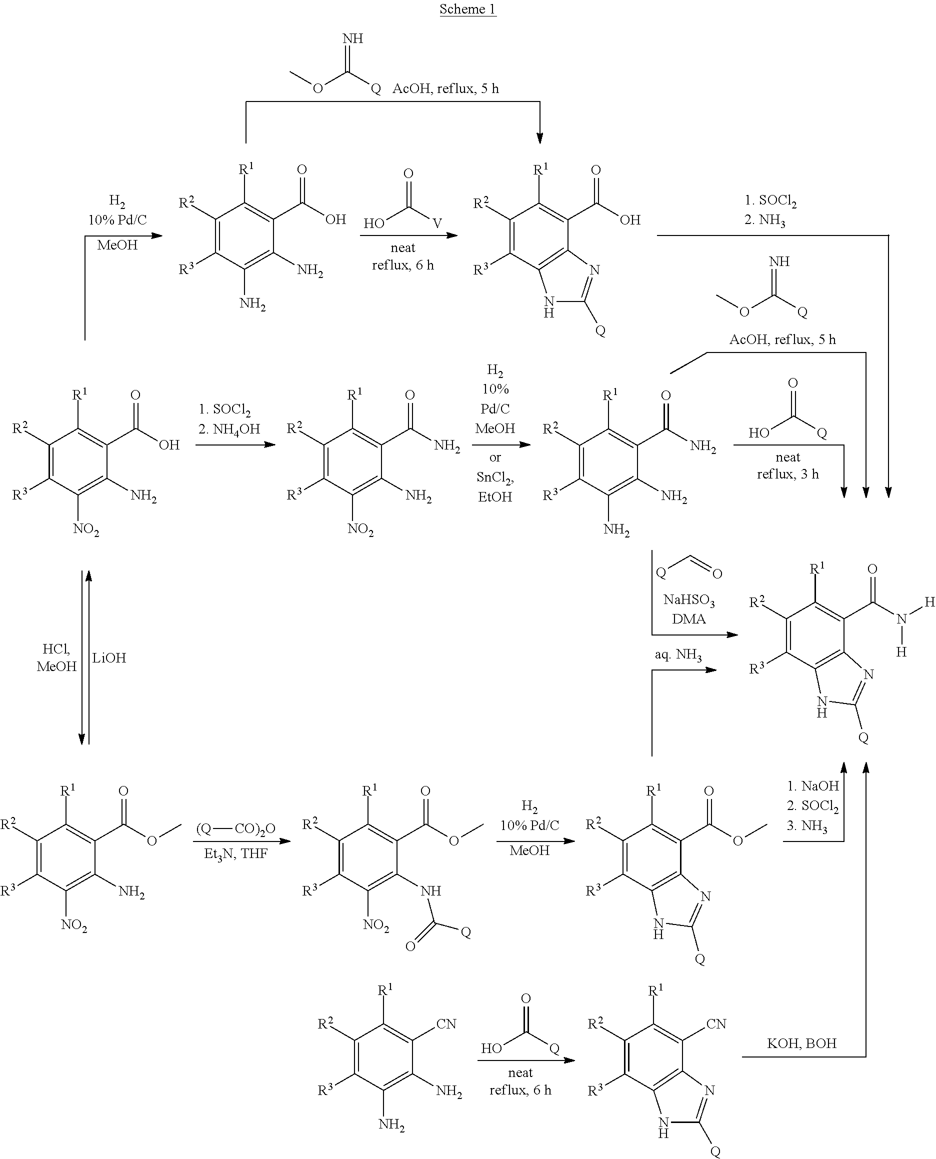

The resulting carboxyl-substituted benzimidazoles can be converted with the aid of thionyl chloride in a suitable solvent and subsequent reaction with a substituted amine or a substituted sulfonamide to correspondingly N-substituted benzimidazoles. The functionalization of a benzimidazole nitrogen atom is possible by deprotonation with a suitable base, for example sodium hydride in an aprotic solvent, and subsequent reaction with a suitable electrophile, for example an acyl chloride, an alkyl halide or a chloroformate. The amide group of the fluoroalkyl-substituted 2-amidobenzimidazoles prepared in accordance with the invention can also be converted to the corresponding thioamide with the aid of 2,4-bis(4-methoxyphenyl)-1,3,2,4-dithiadiphosphetane 2,4-disulfide, or to the corresponding substituted sulfilimines in a two-stage synthesis by reaction with tert-butyl hypochlorite and AlBN in an aprotic solvent (e.g. carbon tetrachloride) and subsequent reaction with a dialkyl sulfide in the presence of a base (e.g. triethylamine) in a suitable solvent (e.g. toluene) (see scheme 2). The radicals R¹, R², R³ and Q listed in Scheme 2 below have the meanings defined above; in addition, Scheme 2 shows, in an exemplary manner, the substituents methyl, ethyl and isopropyl as representatives of the groups according to the invention. The preparation and the use of the compounds according to the invention is illustrated by the examples which follow.

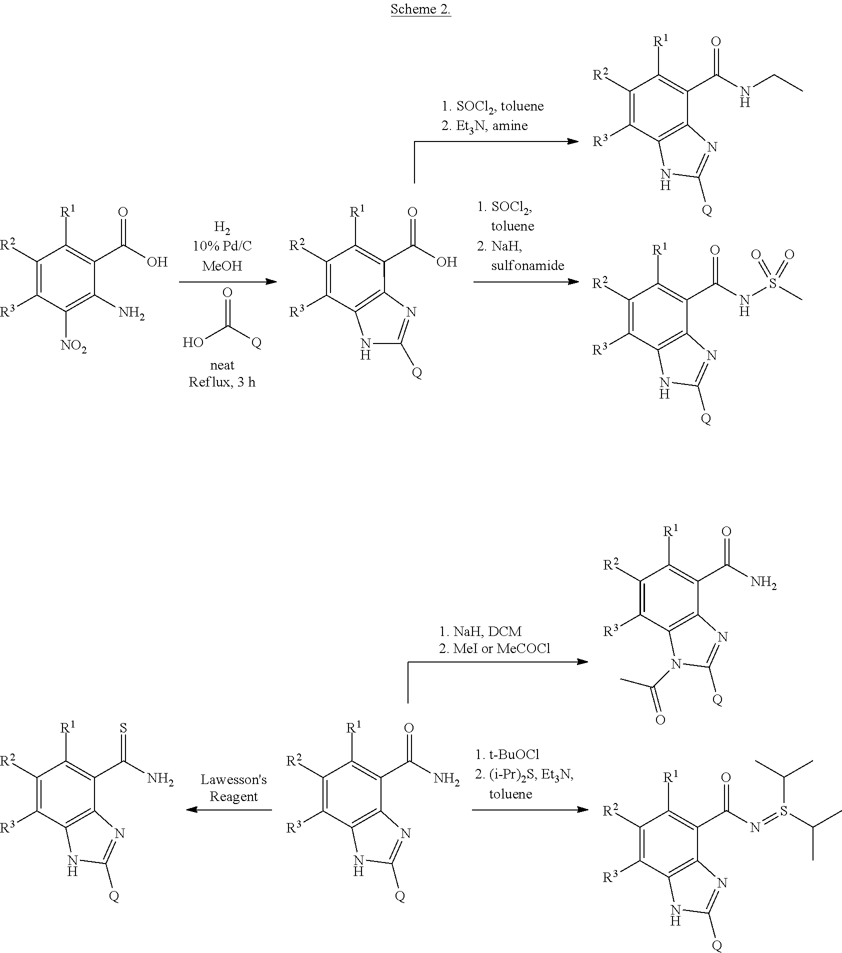

Substituted 2-amidobenzoxazoles can likewise be prepared by known processes (cf. Bioorg. Med. Chem. 2006, 14, 6106; WO2010083220; US20090197863; WO9524379). The synthesis routes used and examined proceed from commercially available or easily preparable 2-amino-3-hydroxybenzoic acids or their analogous esters (Scheme 3). Schema 3 shows the synthesis sequence in an exemplary manner using an ethyl ester, without limiting the radical definition according to the invention. Here, the respective ethyl 2-amino-3-hydroxybenzoate, which is optionally substituted further, is converted using a suitable anhydride in THF or by condensation with a suitable carboxylic acid into the corresponding benzoxazole, which is optionally substituted further. In the next step, the ethyl ester is cleaved with the aid of a suitable hydroxide base (e.g. LiOH, KOH or NaOH), giving the benzoxazolylcarboxylic acid, which is optionally substituted further, which is converted using thionyl chloride and subsequent reaction of the acid chloride with ammonia into the 2-amidobenzoxazole according to the invention, which is optionally substituted further. The radicals R¹, R², R³ and Q mentioned in Scheme 3 below have the meanings defined above.

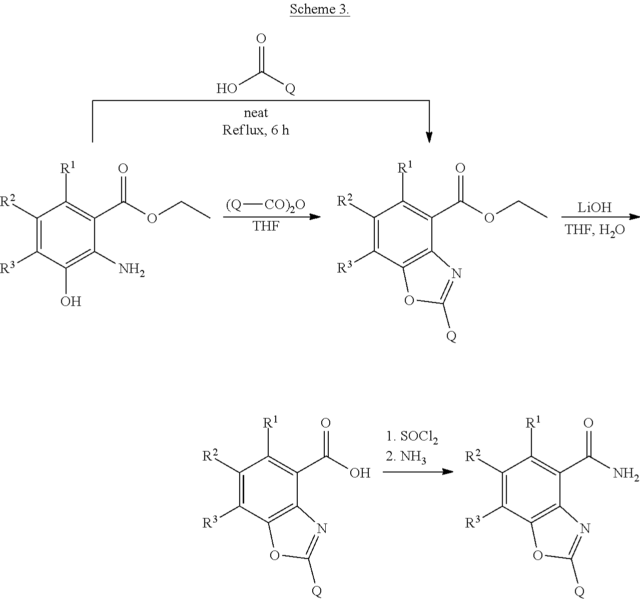

Substituted 2-amidobenzothiazoles can be prepared analogously to the synthesis routes described above, also following processes known from the literature (cf. Bioorg. Med. Chem. 2006, 14, 6106; WO2010083199). Here, 2-nitro-3-chlorobenzoic acids which are optionally substituted further are initially converted with the aid of sodium sulfide hydrate in a suitable polar-protic solvent (e.g. methanol or water) into the corresponding 2-amino-3-hydrothiobenzoic acids (Scheme 4). Here, the 2-amino-3-hydrothiobenzoic acid in question, which is optionally substituted further, is converted with a suitable anhydride in THF or by direct condensation with a suitable carboxylic acid into the corresponding benzothiazolylcarboxylic acid, which is optionally substituted further, which is then, by using thionyl chloride or another suitable chlorinating agent (e.g. oxalyl chloride) and subsequent reaction of the acid chloride with ammonia, converted into the 2-amidobenzothiazole according to the invention, which is optionally substituted further. The radicals R¹, R², R³ and Q mentioned in Scheme 4 below likewise have the meanings defined above.

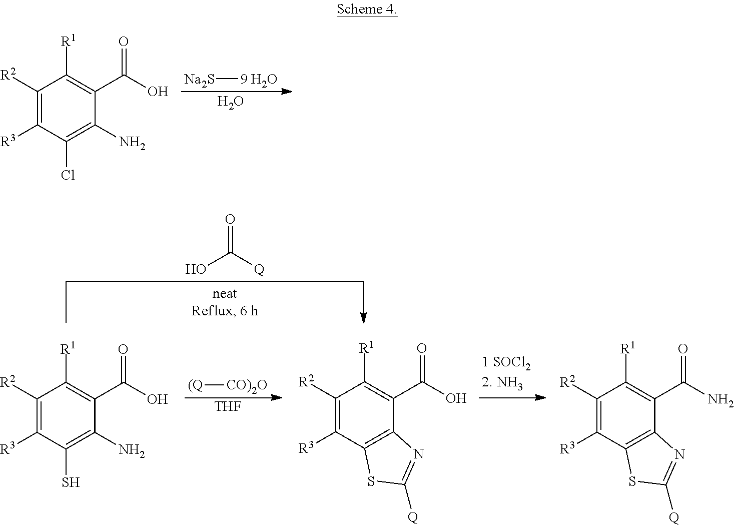

The ¹H NMR, ¹³C NMR and ¹⁹F-NMR spectroscopic data which are reported for the chemical examples described in the paragraphs which follow (400 MHz for ¹H NMR and 150 MHz for ¹³C NMR and 375 MHz for ¹⁹F-NMR, solvent: CDCl₃, CD₃OD or d₆-DMSO, internal standard: tetramethylsilane δ=0.00 ppm), were obtained on a Bruker instrument, and the signals listed have the meanings given below: br=broad; s=singlet, d=doublet, t=triplet, dd=doublet of doublets, ddd=doublet of a doublet of doublets, m=multiplet, q=quartet, quint=quintet, sext=sextet, sept=septet, dq=doublet of quartets, dt=doublet of triplets, tt=triplet of triplets.

### SYNTHESIS EXAMPLES

### No. A1-13: 2-(4-Bromo-3-fluorophenyl)-1,3-benzimidazole-4-carboxamide

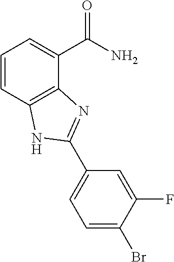 [SMILES: NC(=O)c1cccc2[nH]c(-c3ccc(Br)c(F)c3)nc12]

Methyl 2-amino-3-nitrobenzoate (1.30 g, 6.63 mmol) was dissolved in abs. THF (tetrahydrofuran) (10 ml), triethylamine (2.77 ml, 19.88 mmol) was added and the mixture was stirred at room temperature under argon for 20 min. Thereafter, a solution of 3-fluoro-4-bromobenzoyl chloride (19.88 mmol) in abs. THF (5 ml) was slowly added dropwise and the reaction mixture was stirred at room temperature for 4 h. After the addition of water, the aqueous phase was extracted repeatedly with ethyl acetate. The combined organic phases were then extracted once again with water, dried over magnesium sulfate, filtered and concentrated. Purification of the resulting crude product by column chromatography gave methyl 2-amino-3-[(4-bromo-3-fluorobenzoyl)amino]benzoate. Methyl 2-amino-3-[(4-bromo-3-fluorobenzoyl)amino]benzoate (5.26 mmol) was then dissolved in methanol (50 ml) and added in a metal vessel to palladium on carbon (water-moist catalyst, 10% Pd, 0.02 equiv., 84 mg, 0.079 mmol) in methanol (30 ml). In a laboratory reactor, hydrogen was introduced into the metal vessel and the resulting reaction mixture was stirred at room temperature at a pressure of 2 bar for 5 h. After complete conversion, the catalyst was filtered off through Celite and washed with methanol. The solvent was carefully distilled out of the filtrate under reduced pressure and the residue was purified by column chromatography (silica gel, gradient with n-heptane and ethyl acetate). This gave methyl 2-(4-bromo-3-fluorophenyl)-1H-benzimidazole-4-carboxylate (49% of theory), which in the next step was partially dissolved in THF (1 ml), and water (7 ml) and sodium hydroxide (163 mg, 4.08 mmol) were added. The resulting reaction mixture was stirred under reflux for 3 h. After cooling to room temperature, a pH of 2-3 was established by adding dil. HCl and the precipitate formed was filtered off with suction, washed with heptane and dried. In this way, 2-(4-bromo-3-fluorophenyl)-1H-benzimidazole-4-carboxylic acid (75% of theory) was obtained, which was then dissolved in dichloromethane (6 ml), and oxalyl chloride (1.73 mmol) and a catalytic amount of N,N-dimethylformamide were added. The reaction mixture was stirred at room temperature for 15 min and then at 70° C. for 2 h, and thereafter was concentrated completely. After addition of toluene, the mixture was concentrated again and the acid chloride thus obtained, without further purification, was dissolved in dioxane (6 ml). Then ammonia (g) was introduced while cooling and the mixture was stirred at room temperature for 1 h. The reaction mixture was concentrated completely and the residue was purified by column chromatography (silica gel, gradient with n-heptane and ethyl acetate). This gave 2-(4-bromo-3-fluorophenyl)-1,3-benzimidazole-4-carboxamide (620 mg, 81% of theory). ¹H-NMR (400 MHz, d₆-DMSO δ, ppm) 13.55 (s, 1H, NH), 9.23 (s, 1H, NH), 8.25 (d, 1H), 8.05 (d, 1H), 7.90 (m, 2H), 7.75 (m, 2H), 7.41 (t, 1H).

### No. A1-181: tert-Butyl 4-(4-carbamoyl-1H-benzimidazol-2-yl)piperidine-1-carboxylate

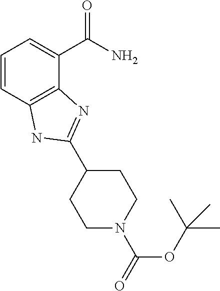 [SMILES: CC(C)(C)OC(=O)N1CCC(c2nc3c(C(N)=O)cccc3[nH]2)CC1]

At room temperature, 2,3-diaminobenzamide (500 mg, 3.30 mmol) and 1-N-Boc-4-piperidinecarboxaldehyde (776 mg, 3.63 mmol) were initially charged in DMA (dimethylamide). With vigorous stirring, sodium bisulfite (585 mg, 5.62 mmol) was added at room temperature, and the reaction solution was then stirred at 130° C. for 5 h. After cooling to room temperature, water was added and the reaction mixture was repeatedly extracted thoroughly with dichloromethane The combined organic phases were then dried over magnesium sulfate, filtered off, concentrated under reduced pressure and then purified by column chromatography (gradient ethyl acetate/heptane). This gave tert-butyl 4-(4-carbamoyl-1H-benzimidazol-2-yl)piperidine-1-carboxylate (1.0 g, 88% of theory) in the form of a colorless solid. ¹H-NMR (400 MHz, CDCl₃ δ, ppm) 9.70 (br. s, 1H, NH), 8.10 (s, 1H), 7.60 (s, 1H), 7.30 (t, 1H), 5.90 (br. s, 1H, NH), 3.12 (m, 1H), 2.10 (m, 2H), 1.90 (m, 2H), 1.65 (m, 4H), 1.50 (s, 9H).

### No. A1-130: 2-(Piperidin-4-yl)-1H-benzimidazole-4-carboxamide

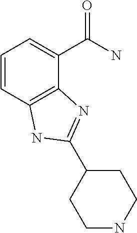 [SMILES: NC(=O)c1cccc2[nH]c(C3CCNCC3)nc12]

At room temperature and under argon, tert-butyl 4-(4-carbamoyl-1H-benzimidazol-2-yl)piperidine-1-carboxylate (1.0 g, 2.9 mmol) was dissolved in abs. dichloromethane (10 ml), and trifluoroacetic acid (2.5 ml) was added slowly with vigorous stirring. This reaction solution was stirred for another 1 h, with the conversion being controlled continuously by TLC and anl. HPLC. Subsequently, aqueous sodium bicarbonate solution was added carefully with stirring until a pH of 9 had been reached. Together, the aqueous and organic phases were concentrated completely under reduced pressure on a rotary evaporator, and the solid that remained was triturated with ethanol. The ethanol phase was dried over sodium sulfate, filtered and concentrated under reduced pressure. This gave 2-(piperidin-4-yl)-1H-benzimidazole-4-carboxamide (300 mg, 40% of theory) in the form of a colorless solid. ¹H-NMR (400 MHz, CD₃OD δ, ppm) 7.88 (d, 1H), 7.69 (d, 1H), 7.29 (t, 1H), 3.15 (m, 3H), 2.80 (m, 2H), 2.10 (m, 2H), 1.90 (m, 2H).

### No. B1-18: 2-(2,4-Dichlorophenyl)-1,3-benzoxazole-4-carboxamide

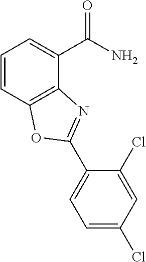 [SMILES: NC(=O)c1cccc2oc(-c3ccc(Cl)cc3Cl)nc12]

At room temperature, 8 g (40.58 mmol) of methyl 3-hydroxy-2-nitrobenzoate were dissolved in 160 ml of a mixture (1:1) of acetic acid and ethanol. 9.971 g (178.55 mmol) of iron powder were added to the solution, and the mixture was heated at the boil for 2 h. The mixture was cooled to room temperature, 50 ml of water were added and the mixture was extracted twice with 100 ml of ethyl acetate. The organic phase was washed with dilute sodium bicarbonate solution until neutral and dried over sodium sulfate. The solvent was removed under reduced pressure, giving 6.50 g (95% of theory) of the desired methyl 2-amino-3-hydroxybenzoate. ¹H-NMR (400 MHz, DMSO-d₆ δ in ppm) 9.66 (s, 1H), 7.20 (d, 1H), 6.81 (d, 1H), 6.39 (t, 1H), 6.09 (br. s, 2H), 3.78 (s, 3H). 700 mg (4.19 mmol) of methyl 2-amino-3-hydroxybenzoate, 877 mg (4.19 mmol) of 2,4-dichlorobenzoyl chloride and 210 mg (0.838 mmol) of 4-methylbenzenesulfonic acid monohydrate were suspended 10 ml of xylene in a microwave vial. The vial was sealed with a septum cap and the mixture was heated in a Biotage Initiator Sixty microwave at 160° C. for 25 min. After cooling to room temperature, the solvent was removed under reduced pressure and the crude product was purified by column chromatography (n-heptane:ethyl acetate 4:1→ethyl acetate), giving 500 mg (35% of theory) of methyl 2-(2,4-dichlorophenyl)-1,3-benzoxazole-4-carboxylate. ¹H-NMR (CDCl₃ δ in ppm): 8.21 (d, 1H), 8.08 (d, 1H), 7.81 (dd, 1H), 7.59 (d, 1H), 7.48 (t, 1H), 7.42 (dd, 1H), 4.05 (s, 3H). 450 mg (1.40 mmol) of methyl 2-(2,4-dichlorophenyl)-1,3-benzoxazole-4-carboxylate and 0.838 ml of 2 N aqueous sodium hydroxide solution were dissolved in 10 ml of THF and 2 ml of water. The solution was stirred at room temperature overnight, resulting in the precipitation of a solid. The suspension was acidified with 2 N hydrochloric acid and the resulting solid was filtered off with suction. The solid was air-dried, giving 300 mg (66% of theory) of the desired 2-(2,4-dichlorophenyl)-1,3-benzoxazole-4-carboxylic acid. ¹H-NMR (CDCl₃ δ in ppm): 11.62 (br. s, 1H), 8.23-8.18 (m, 2H), 7.88 (d, 1H), 7.67 (d, 1H), 7.60 (t, 1H), 7.49 (dd, 1H). 250 mg (0.81 mmol) of 2-(2,4-dichlorophenyl)-1,3-benzoxazole-4-carboxylic acid, 132 mg (0.97 mmol) of 1-hydroxy-1H-benzotriazole, 171 mg (0.89 mmol) of 1-(3-dimethylaminopropyl)-3-ethylcarbodiimide hydrochloride and 10 mg of DMAP were dissolved in 5 ml of dichloromethane. The mixture was stirred at room temperature for 15 min, and 1.78 ml of a 0.5 M solution of ammonia in 1,4-dioxane were then added dropwise. The mixture was stirred at room temperature for 2 h and the organic phase was washed twice with 0.5 N hydrochloric acid and once with dilute sodium bicarbonate solution. The solvent was removed under reduced pressure and the residue was suspended in acetonitrile and then heated in an ultrasonic bath. The solid was filtered off with suction and air-dried. This gives 120 mg (45% of theory) of 2-(2,4-dichlorophenyl)-1,3-benzoxazole-4-carboxamide. ¹H-NMR (DMSO-d₆ δ in ppm): 8.42 (br. s, 1H), 8.33 (d, 1H), 8.07-8.01 (m, 3H), 7.98 (d, 1H), 7.71 (d, 1H), 7.62 (dd, 1H).

### No. B1-321: 2-(Pentafluoroethyl)-1,3-benzoxazole-4-carboxamide

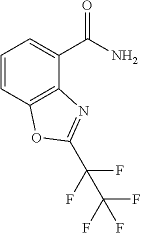 [SMILES: NC(=O)c1cccc2oc(C(F)(F)C(F)(F)F)nc12]

Ethyl 2-amino-3-hydroxybenzoate (250 mg, 1.38 mmol) was dissolved in abs. THF (tetrahydrofuran) (3 ml) and, under argon, cooled to a temperature of −78° C. A solution of pentafluoropropionic anhydride (471 mg, 1.52 mmol) in abs. THF (2 ml) was then slowly added dropwise and the reaction mixture was stirred at −78° C. for 30 min and then at room temperature for 1 h. After the addition of water, the aqueous phase was extracted repeatedly with dichloromethane. The combined organic phases were then extracted once again with water, dried over magnesium sulfate, filtered off and concentrated. Purification of the resulting crude product by column chromatography gave ethyl 2-(pentafluoroethyl)-1,3-benzoxazole-4-carboxylate (400 mg, 94% of theory). The ethyl 2-(pentafluoroethyl)-1,3-benzoxazole-4-carboxylate (200 mg, 0.65 mmol) was then dissolved in abs. THF (1 ml). After the addition of water (5 ml) and sodium hydroxide (65 mg, 1.62 mmol), the resulting reaction mixture was stirred under reflux conditions for 3 h. After cooling to room temperature, dilute hydrochloric acid was added carefully such that a slightly acidic pH was obtained. The aqueous phase was repeatedly extracted intensively with ethyl acetate. The combined organic phases were then extracted once again with water, dried over magnesium sulfate, filtered off and concentrated. In this manner, 2-(pentafluoroethyl)-1,3-benzoxazole-4-carboxylic acid (190 mg, 99% of theory) was obtained, a partial amount of which (130 mg, 0.46 mmol) was then dissolved in dichloromethane (2 ml), and oxalyl chloride (0.03 ml, 0.39 mmol) and a catalytic amount of N,N-dimethylformamide were added. The reaction mixture was stirred at room temperature for 15 min and then at 70° C. for 3 h and was subsequently evaporated to dryness. After addition of toluene, the mixture was concentrated again and the acid chloride thus obtained, without further purification, was dissolved in tetrahydrofuran (5 ml). Then ammonia (g) was introduced while cooling and the mixture was stirred at room temperature for 1 h. The reaction mixture was evaporated to dryness and the residue was purified by column chromatography (silica gel, gradient with n-heptane and ethyl acetate). This gave 2-(pentafluoroethyl)-1,3-benzoxazol-4-carboxamide in the form of a colorless solid (21 mg, 16% of theory). ¹H-NMR (400 MHz, CDCl₃ δ, ppm) 8.37 (d, 1H), 8.36 (br. s, 1H, NH), 7.88 (d, 1H), 7.71 (dd, 1H), 5.93 (br. s, 1H, NH).

### No. C1-321: 2-(Pentafluoroethyl)-1,3-benzothiazole-4-carboxamide

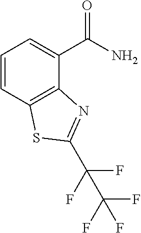 [SMILES: NC(=O)c1cccc2sc(C(F)(F)C(F)(F)F)nc12]

3-Chloro-2-nitrobenzoic acid (500 mg, 2.48 mmol) and sodium sulfide nonahydrate (1.61 g, 6.69 mmol) were dissolved in water and stirred under reflux conditions for several hours. After cooling to room temperature, the reaction mixture was adjusted to pH 5 using dilute hydrochloric acid. The resulting precipitate was filtered off with suction, washed repeatedly with water and dried thoroughly, giving 2-amino-3-hydrothiobenzoic acid in the form of a colorless solid (250 mg, 59% of theory). 2-Amino-3-hydrothiobenzoic acid (250 mg, 1.48 mmol) was dissolved in abs. THF (tetrahydrofuran) (5 ml) and, under argon, cooled to a temperature of −78° C. A solution of pentafluoropropionic anhydride (560 mg, 1.77 mmol) in abs. THF (3 ml) was then slowly added dropwise and the reaction mixture was stirred at −78° C. for 30 min and then at room temperature for 1 h. The solvent was removed on a rotary evaporator and the residue was taken up in dichloromethane. After the addition of water, the aqueous phase was extracted repeatedly with dichloromethane. The combined organic phases were then extracted once again with water, dried over magnesium sulfate, filtered off and concentrated. Purification of the resulting crude product by column chromatography gave 2-(pentafluoroethyl)-1,3-benzothiazole-4-carboxtylic acid (230 mg, 52% of theory) which was then dissolved in dichloromethane (4 ml), and oxalyl chloride (0.06 ml, 0.78 mmol) and a catalytic amount of N,N-dimethylformamide were added. The reaction mixture was stirred at room temperature for 15 min and then at 70° C. for 3 h and was subsequently evaporated to dryness. After addition of toluene, the mixture was concentrated again and the acid chloride thus obtained, without further purification, was dissolved in tetrahydrofuran (5 ml). Then ammonia (g) was introduced while cooling and the mixture was stirred at room temperature for 1 h. The reaction mixture was evaporated to dryness and the residue was purified by column chromatography (silica gel, gradient with n-heptane and ethyl acetate). This gave 2-(pentafluoroethyl)-1,3-benzothiazole-4-carboxamide in the form of a colorless solid (60 mg, 28% of theory). ¹H-NMR (400 MHz, CDCl₃ δ, ppm) 9.11 (br. s, 1H, NH), 8.59 (d, 1H), 8.19 (d, 1H), 7.73 (dd, 1H), 6.03 (br. s, 1H, NH); ¹⁹F-NMR (375 MHz, d₆-DMSO δ, ppm) −82.7, −108.9.

In analogy to the preparation examples cited above and recited in the tables below, and taking account of the general details of the preparation of substituted 2-amidobenzimidazoles, 2-amidobenzoxazoles and 2-amidobenzothiazoles of the general formula (I), the following compounds are obtained:

A1. Compounds A1-1 to A1-1000 of the general formula (I) in which R¹, R² and R³ represent hydrogen and Q, W, Z¹, Z² and R⁴ correspond to the definitions (Nos 1 to 1000; corresponding to compounds A1-1 to A1-1000) in Table 1 below.

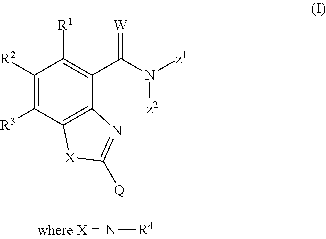

TABLE 1

| No. | Q | W | R⁴ | Z¹ | Z² |
|---|---|---|---|---|---|
| 1 | 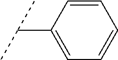 | O | H | H | H |
| 2 | 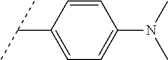 | O | H | H | H |
| 3 | 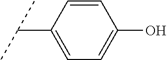 | O | H | H | H |
| 4 | 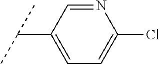 | O | H | H | H |
| 5 | 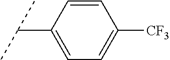 | O | H | H | H |
| 6 | 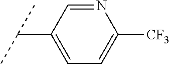 | O | H | H | H |
| 7 | 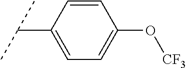 | O | H | H | H |
| 8 | 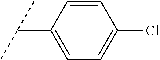 | O | H | H | H |
| 9 | 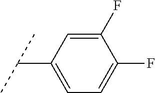 | O | H | H | H |
| 10 | 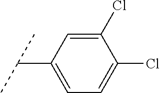 | O | H | H | H |
| 11 | 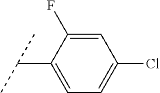 | O | H | H | H |
| 12 | 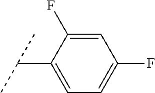 | O | H | H | H |
| 13 | 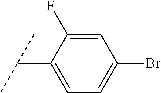 | O | H | H | H |
| 14 | 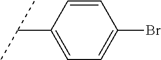 | O | H | H | H |
| 15 | 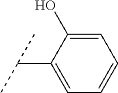 | O | H | H | H |
| 16 | 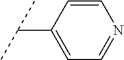 | O | H | H | H |
| 17 | 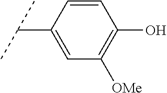 | O | H | H | H |
| 18 | 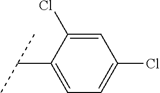 | O | H | H | H |
| 19 | 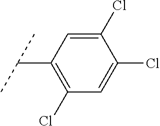 | O | H | H | H |
| 20 | 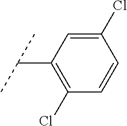 | O | H | H | H |
| 21 | 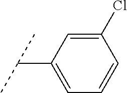 | O | H | H | H |
| 22 | 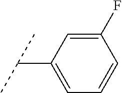 | O | H | H | H |
| 23 | 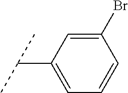 | O | H | H | H |
| 24 | 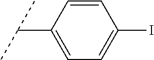 | O | H | H | H |
| 25 | 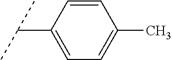 | O | H | H | H |
| 26 | 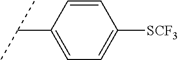 | O | H | H | H |
| 27 | 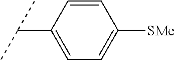 | O | H | H | H |
| 28 | 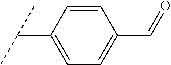 | O | H | H | H |
| 29 | 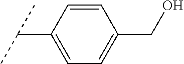 | O | H | H | H |
| 30 | 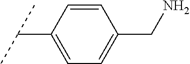 | O | H | H | H |
| 31 | 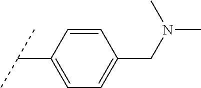 | O | H | H | H |
| 32 | 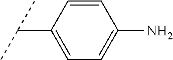 | O | H | H | H |
| 33 | 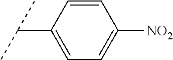 | O | H | H | H |
| 34 | 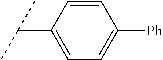 | O | H | H | H |
| 35 | 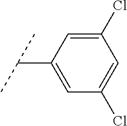 | O | H | H | H |
| 36 | 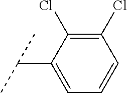 | O | H | H | H |
| 37 | 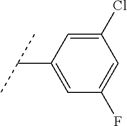 | O | H | H | H |
| 38 | 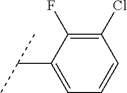 | O | H | H | H |
| 39 | 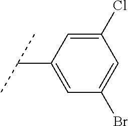 | O | H | H | H |
| 40 | 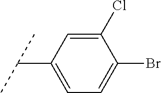 | O | H | H | H |
| 41 | 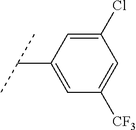 | O | H | H | H |
| 42 | 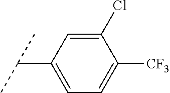 | O | H | H | H |
| 43 | 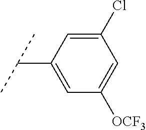 | O | H | H | H |
| 44 | 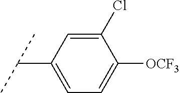 | O | H | H | H |
| 45 | 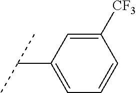 | O | H | H | H |
| 46 | 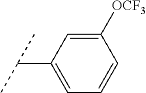 | O | H | H | H |
| 47 | 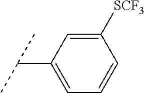 | O | H | H | H |
| 48 | 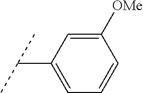 | O | H | H | H |
| 49 | 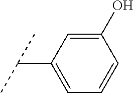 | O | H | H | H |
| 50 | 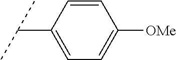 | O | H | H | H |
| 51 | 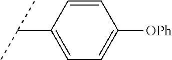 | O | H | H | H |
| 52 | 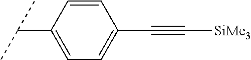 | O | H | H | H |
| 53 | 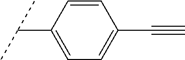 | O | H | H | H |
| 54 | 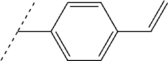 | O | H | H | H |
| 55 | 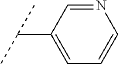 | O | H | H | H |
| 56 | 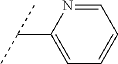 | O | H | H | H |
| 57 | 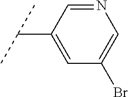 | O | H | H | H |
| 58 | 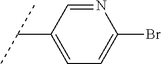 | O | H | H | H |
| 59 | 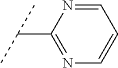 | O | H | H | H |
| 60 | 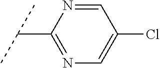 | O | H | H | H |
| 61 | 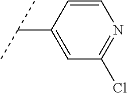 | O | H | H | H |
| 62 | 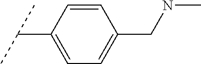 | O | H | H | H |
| 63 | 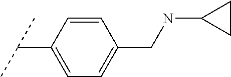 | O | H | H | H |
| 64 | 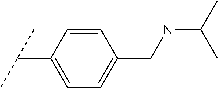 | O | H | H | H |
| 65 | 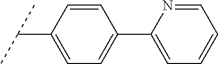 | O | H | H | H |
| 66 | 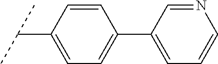 | O | H | H | H |
| 67 | 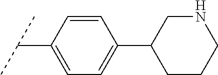 | O | H | H | H |
| 68 | 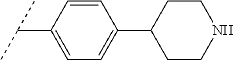 | O | H | H | H |
| 69 | 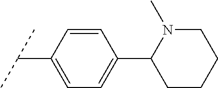 | O | H | H | H |
| 70 | 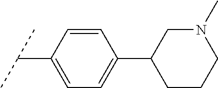 | O | H | H | H |
| 71 | 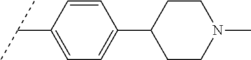 | O | H | H | H |
| 72 | 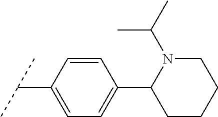 | O | H | H | H |
| 73 | 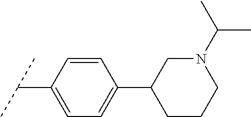 | O | H | H | H |
| 74 | 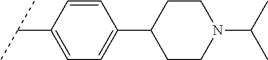 | O | H | H | H |
| 75 | 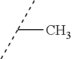 | O | H | H | H |
| 76 | 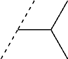 | O | H | H | H |
| 77 | 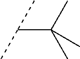 | O | H | H | H |
| 78 | 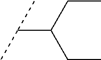 | O | H | H | H |
| 79 | 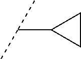 | O | H | H | H |
| 80 | 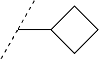 | O | H | H | H |
| 81 | 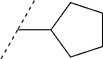 | O | H | H | H |
| 82 | 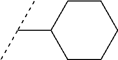 | O | H | H | H |
| 83 | 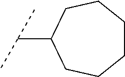 | O | H | H | H |
| 84 | 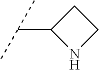 | O | H | H | H |
| 85 | 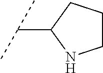 | O | H | H | H |
| 86 | 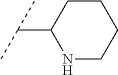 | O | H | H | H |
| 87 | 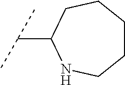 | O | H | H | H |
| 88 | 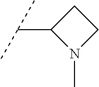 | O | H | H | H |
| 89 | 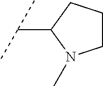 | O | H | H | H |
| 90 | 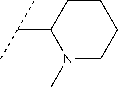 | O | H | H | H |
| 91 | 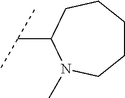 | O | H | H | H |
| 92 | 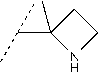 | O | H | H | H |
| 93 | 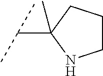 | O | H | H | H |
| 94 | 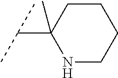 | O | H | H | H |
| 95 | 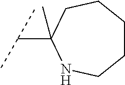 | O | H | H | H |
| 96 | 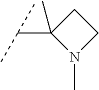 | O | H | H | H |
| 97 | 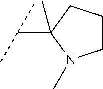 | O | H | H | H |
| 98 | 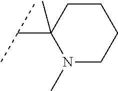 | O | H | H | H |
| 99 | 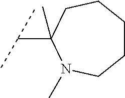 | O | H | H | H |
| 100 | 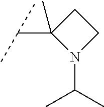 | O | H | H | H |
| 101 | 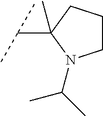 | O | H | H | H |
| 102 | 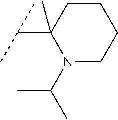 | O | H | H | H |
| 103 | 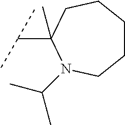 | O | H | H | H |
| 104 | 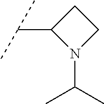 | O | H | H | H |
| 105 | 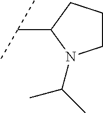 | O | H | H | H |
| 106 | 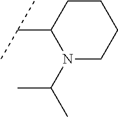 | O | H | H | H |
| 107 | 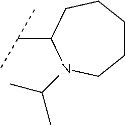 | O | H | H | H |
| 108 | 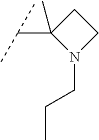 | O | H | H | H |
| 109 | 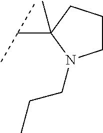 | O | H | H | H |
| 110 | 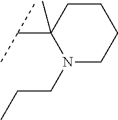 | O | H | H | H |
| 111 | 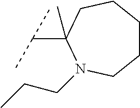 | O | H | H | H |
| 112 | 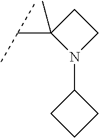 | O | H | H | H |
| 113 | 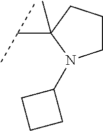 | O | H | H | H |
| 114 | 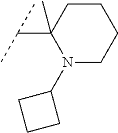 | O | H | H | H |
| 115 | 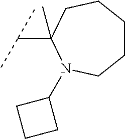 | O | H | H | H |
| 116 | 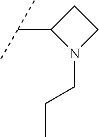 | O | H | H | H |
| 117 | 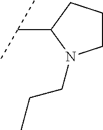 | O | H | H | H |
| 118 | 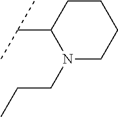 | O | H | H | H |
| 119 | 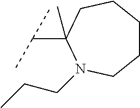 | O | H | H | H |
| 120 | 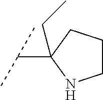 | O | H | H | H |
| 121 | 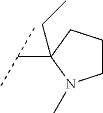 | O | H | H | H |
| 122 | 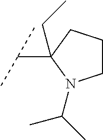 | O | H | H | H |
| 123 | 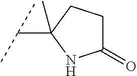 | O | H | H | H |
| 124 | 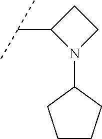 | O | H | H | H |
| 125 | 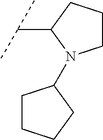 | O | H | H | H |
| 126 | 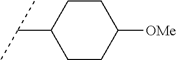 | O | H | H | H |
| 127 | 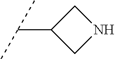 | O | H | H | H |
| 128 | 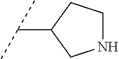 | O | H | H | H |
| 129 | 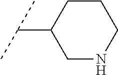 | O | H | H | H |
| 130 | 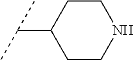 | O | H | H | H |
| 131 | 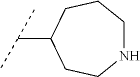 | O | H | H | H |
| 132 | 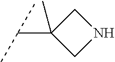 | O | H | H | H |
| 133 | 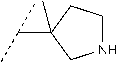 | O | H | H | H |
| 134 | 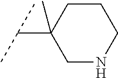 | O | H | H | H |
| 135 | 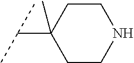 | O | H | H | H |
| 136 | 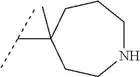 | O | H | H | H |
| 137 | 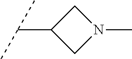 | O | H | H | H |
| 138 | 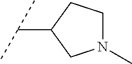 | O | H | H | H |
| 139 | 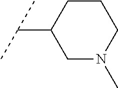 | O | H | H | H |
| 140 | 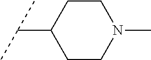 | O | H | H | H |
| 141 | 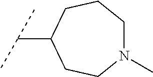 | O | H | H | H |
| 142 | 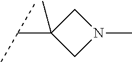 | O | H | H | H |
| 143 | 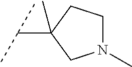 | O | H | H | H |
| 144 | 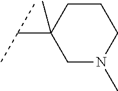 | O | H | H | H |
| 145 | 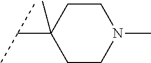 | O | H | H | H |
| 146 | 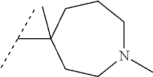 | O | H | H | H |
| 147 | 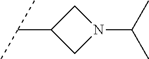 | O | H | H | H |
| 148 | 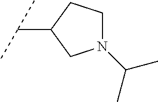 | O | H | H | H |
| 149 | 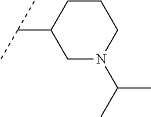 | O | H | H | H |
| 150 | 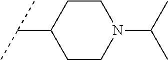 | O | H | H | H |
| 151 | 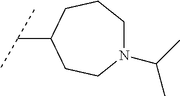 | O | H | H | H |
| 152 | 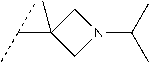 | O | H | H | H |
| 153 | 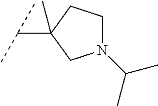 | O | H | H | H |
| 154 | 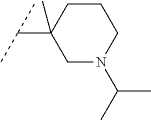 | O | H | H | H |
| 155 | 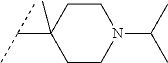 | O | H | H | H |
| 156 | 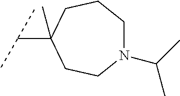 | O | H | H | H |
| 157 | 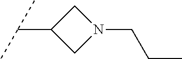 | O | H | H | H |
| 158 | 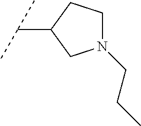 | O | H | H | H |
| 159 | 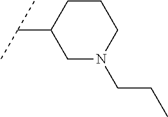 | O | H | H | H |
| 160 | 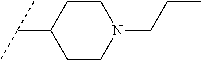 | O | H | H | H |
| 161 | 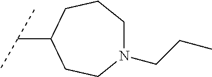 | O | H | H | H |
| 162 | 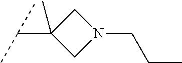 | O | H | H | H |
| 163 | 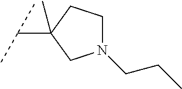 | O | H | H | H |
| 164 | 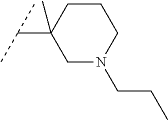 | O | H | H | H |
| 165 | 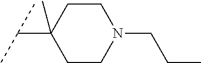 | O | H | H | H |
| 166 | 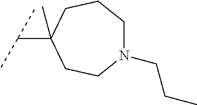 | O | H | H | H |
| 167 | 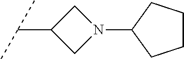 | O | H | H | H |
| 168 | 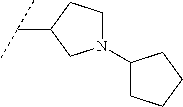 | O | H | H | H |
| 169 | 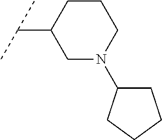 | O | H | H | H |
| 170 | 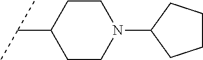 | O | H | H | H |
| 171 | 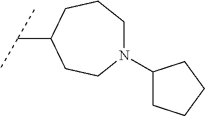 | O | H | H | H |
| 172 | 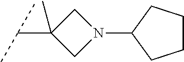 | O | H | H | H |
| 173 | 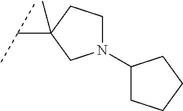 | O | H | H | H |
| 174 | 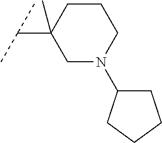 | O | H | H | H |
| 175 | 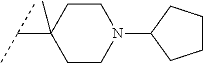 | O | H | H | H |
| 176 | 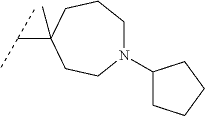 | O | H | H | H |
| 177 | 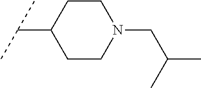 | O | H | H | H |
| 178 | 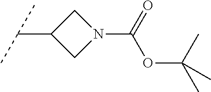 | O | H | H | H |
| 179 | 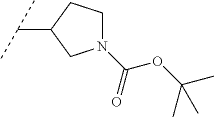 | O | H | H | H |
| 180 | 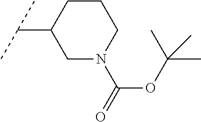 | O | H | H | H |
| 181 | 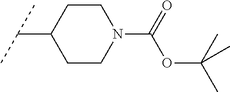 | O | H | H | H |
| 182 | 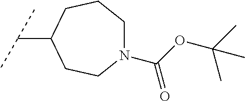 | O | H | H | H |
| 183 | 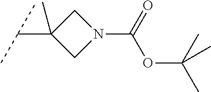 | O | H | H | H |
| 184 | 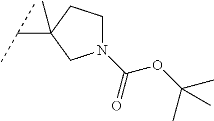 | O | H | H | H |
| 185 | 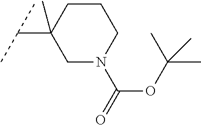 | O | H | H | H |
| 186 | 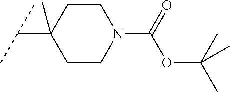 | O | H | H | H |
| 187 | 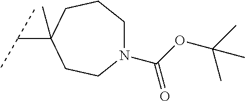 | O | H | H | H |
| 188 | 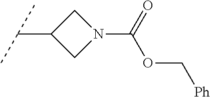 | O | H | H | H |
| 189 | 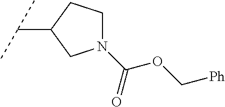 | O | H | H | H |
| 190 | 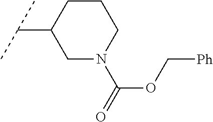 | O | H | H | H |
| 191 | 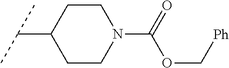 | O | H | H | H |
| 192 | 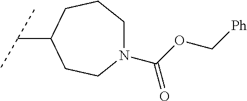 | O | H | H | H |
| 193 | 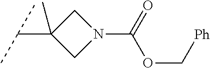 | O | H | H | H |
| 194 | 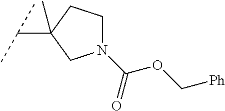 | O | H | H | H |
| 195 | 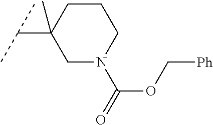 | O | H | H | H |
| 196 | 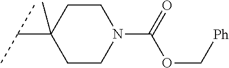 | O | H | H | H |
| 197 | 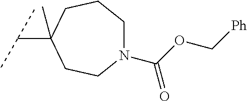 | O | H | H | H |
| 198 | 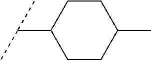 | O | H | H | H |
| 199 | 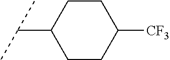 | O | H | H | H |
| 200 | 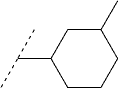 | O | H | H | H |
| 201 | 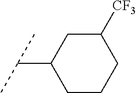 | O | H | H | H |
| 202 | 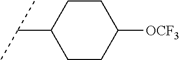 | O | H | H | H |
| 203 | 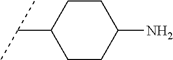 | O | H | H | H |
| 204 | 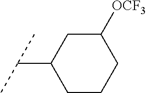 | O | H | H | H |
| 205 | 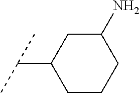 | O | H | H | H |
| 206 | 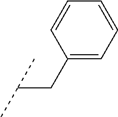 | O | H | H | H |
| 207 | 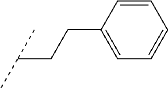 | O | H | H | H |
| 208 | 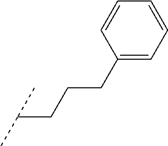 | O | H | H | H |
| 209 | 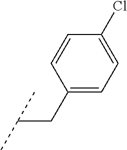 | O | H | H | H |
| 210 | 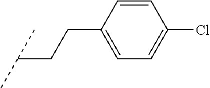 | O | H | H | H |
| 211 | 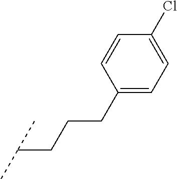 | O | H | H | H |
| 212 | 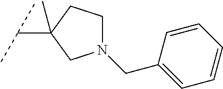 | O | H | H | H |
| 213 | 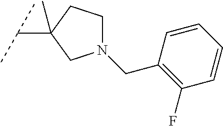 | O | H | H | H |
| 214 | 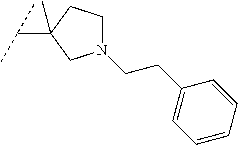 | O | H | H | H |
| 215 | 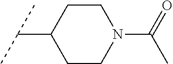 | O | H | H | H |
| 216 | 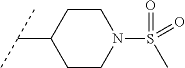 | O | H | H | H |
| 217 | 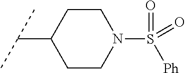 | O | H | H | H |
| 218 | 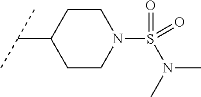 | O | H | H | H |
| 219 | 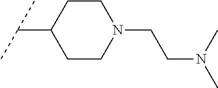 | O | H | H | H |
| 220 | 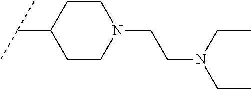 | O | H | H | H |
| 221 | 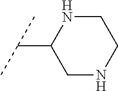 | O | H | H | H |
| 222 | 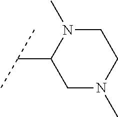 | O | H | H | H |
| 223 | 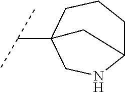 | O | H | H | H |
| 224 | 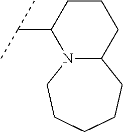 | O | H | H | H |
| 225 | 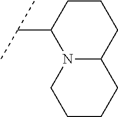 | O | H | H | H |
| 226 | 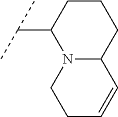 | O | H | H | H |
| 227 | 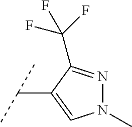 | O | H | H | H |
| 228 | 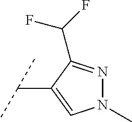 | O | H | H | H |
| 229 | 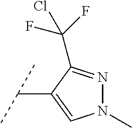 | O | H | H | H |
| 230 | 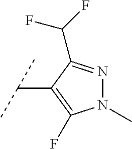 | O | H | H | H |
| 231 | 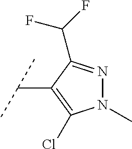 | O | H | H | H |
| 232 | 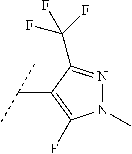 | O | H | H | H |
| 233 | 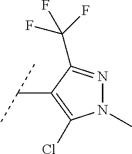 | O | H | H | H |
| 234 | 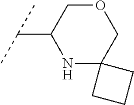 | O | H | H | H |
| 235 | 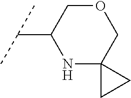 | O | H | H | H |
| 236 | 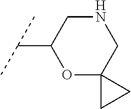 | O | H | H | H |
| 237 | 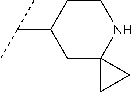 | O | H | H | H |
| 238 | 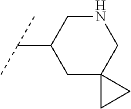 | O | H | H | H |
| 239 | 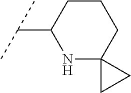 | O | H | H | H |
| 240 | 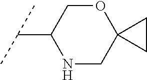 | O | H | H | H |
| 241 | 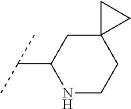 | O | H | H | H |
| 242 | 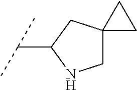 | O | H | H | H |
| 243 | 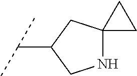 | O | H | H | H |
| 244 | 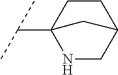 | O | H | H | H |
| 245 | 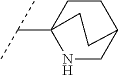 | O | H | H | H |
| 246 | 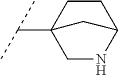 | O | H | H | H |
| 247 | 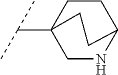 | O | H | H | H |
| 248 | 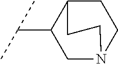 | O | H | H | H |
| 249 | 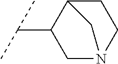 | O | H | H | H |
| 250 | 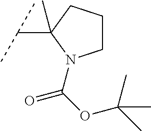 | O | H | H | H |
| 251 | 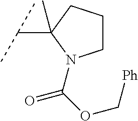 | O | H | H | H |
| 252 | 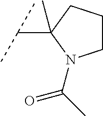 | O | H | H | H |
| 253 | 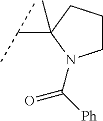 | O | H | H | H |
| 254 | 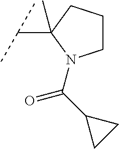 | O | H | H | H |
| 255 | 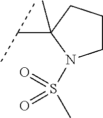 | O | H | H | H |
| 256 | 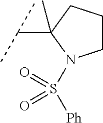 | O | H | H | H |
| 257 | 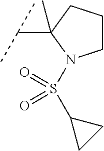 | O | H | H | H |
| 258 | 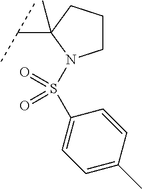 | O | H | H | H |
| 259 | 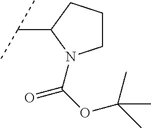 | O | H | H | H |
| 260 | 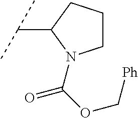 | O | H | H | H |
| 261 | 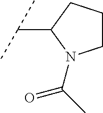 | O | H | H | H |
| 262 | 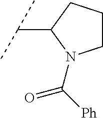 | O | H | H | H |
| 263 | 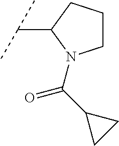 | O | H | H | H |
| 264 | 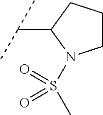 | O | H | H | H |
| 265 | 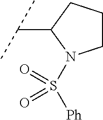 | O | H | H | H |
| 266 | 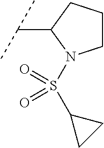 | O | H | H | H |
| 267 | 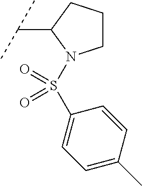 | O | H | H | H |
| 268 | 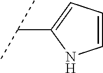 | O | H | H | H |
| 269 | 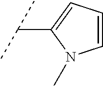 | O | H | H | H |
| 270 | 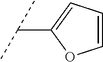 | O | H | H | H |
| 271 | 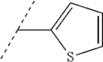 | O | H | H | H |
| 272 | 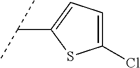 | O | H | H | H |
| 273 | 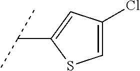 | O | H | H | H |
| 274 | 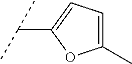 | O | H | H | H |
| 275 | 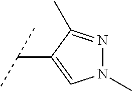 | O | H | H | H |
| 276 | 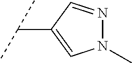 | O | H | H | H |
| 277 | 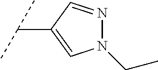 | O | H | H | H |
| 278 | 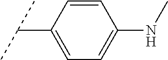 | O | H | H | H |
| 279 | 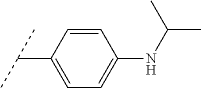 | O | H | H | H |
| 280 | 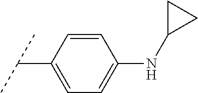 | O | H | H | H |
| 281 | 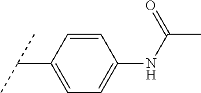 | O | H | H | H |
| 282 | 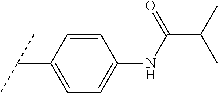 | O | H | H | H |
| 283 | 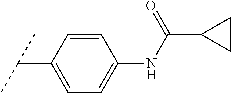 | O | H | H | H |
| 284 | 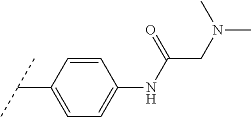 | O | H | H | H |
| 285 | 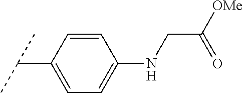 | O | H | H | H |
| 286 | 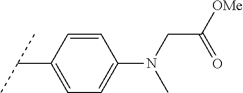 | O | H | H | H |
| 287 | 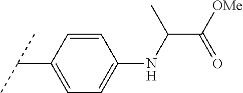 | O | H | H | H |
| 288 | 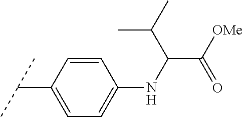 | O | H | H | H |
| 289 | 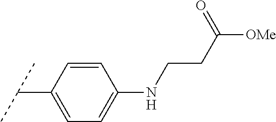 | O | H | H | H |
| 290 | 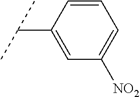 | O | H | H | H |
| 291 | 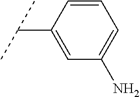 | O | H | H | H |
| 292 | 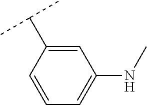 | O | H | H | H |
| 293 | 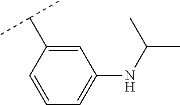 | O | H | H | H |
| 294 | 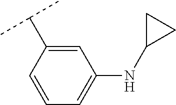 | O | H | H | H |
| 295 | 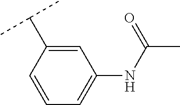 | O | H | H | H |
| 296 | 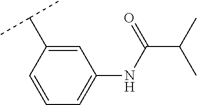 | O | H | H | H |
| 297 | 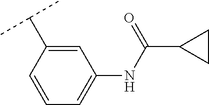 | O | H | H | H |
| 298 | 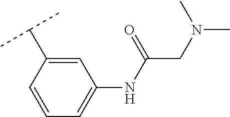 | O | H | H | H |
| 299 | 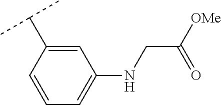 | O | H | H | H |
| 300 | 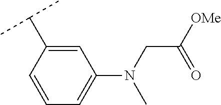 | O | H | H | H |
| 301 | 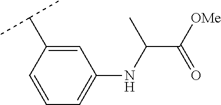 | O | H | H | H |
| 302 | 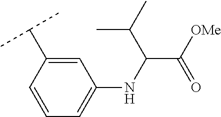 | O | H | H | H |
| 303 | 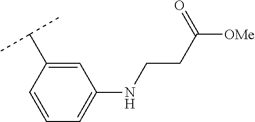 | O | H | H | H |
| 304 | 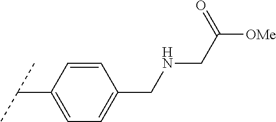 | O | H | H | H |
| 305 | 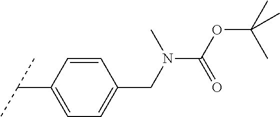 | O | H | H | H |
| 306 | 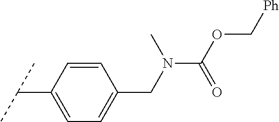 | O | H | H | H |
| 307 | 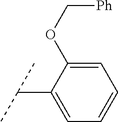 | O | H | H | H |
| 308 | 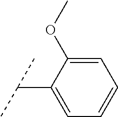 | O | H | H | H |
| 309 | 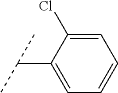 | O | H | H | H |
| 310 | 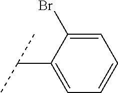 | O | H | H | H |
| 311 | 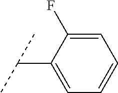 | O | H | H | H |
| 312 | 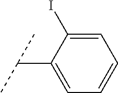 | O | H | H | H |
| 313 | 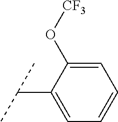 | O | H | H | H |
| 314 | 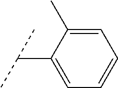 | O | H | H | H |
| 315 | 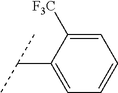 | O | H | H | H |
| 316 | 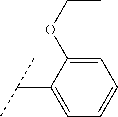 | O | H | H | H |
| 317 | 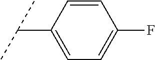 | O | H | H | H |
| 318 | 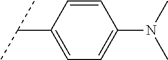 | O | CH₃ | H | H |
| 319 | 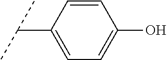 | O | CH₃ | H | H |
| 320 | 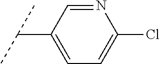 | O | CH₃ | H | H |
| 11 321 | 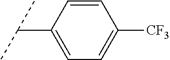 | O | CH₃ | H | H |
| 322 | 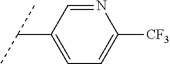 | O | CH₃ | H | H |
| 323 | 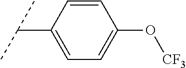 | O | CH₃ | H | H |
| 324 | 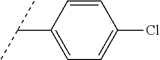 | O | CH₃ | H | H |
| 325 | 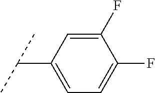 | O | CH₃ | H | H |
| 326 | 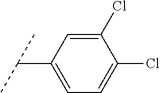 | O | CH₃ | H | H |
| 327 | 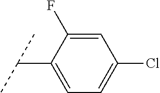 | O | CH₃ | H | H |
| 328 | 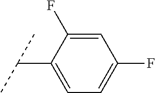 | O | CH₃ | H | H |
| 329 | 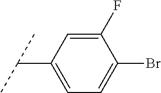 | O | CH₃ | H | H |
| 330 | 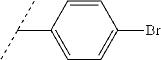 | O | CH₃ | H | H |
| 331 | 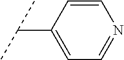 | O | CH₃ | H | H |
| 332 | 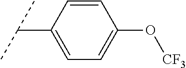 | O | CH₃ | H | H |
| 333 | 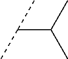 | O | CH₃ | H | H |
| 334 | 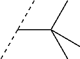 | O | CH₃ | H | H |
| 335 | 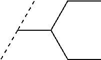 | O | CH₃ | H | H |
| 336 | 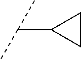 | O | CH₃ | H | H |
| 337 | 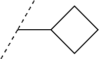 | O | CH₃ | H | H |
| 338 | 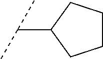 | O | CH₃ | H | H |
| 339 | 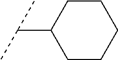 | O | CH₃ | H | H |
| 340 | 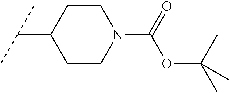 | O | CH₃ | H | H |
| 341 | 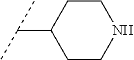 | O | CH₃ | H | H |
| 342 | 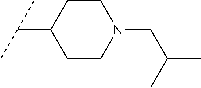 | O | CH₃ | H | H |
| 343 | 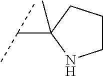 | O | CH₃ | H | H |
| 344 | 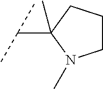 | O | CH₃ | H | H |
| 345 | 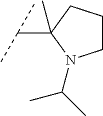 | O | CH₃ | H | H |
| 346 | 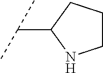 | O | CH₃ | H | H |
| 347 | 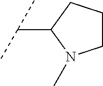 | O | CH₃ | H | H |
| 348 | 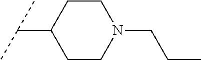 | O | CH₃ | H | H |
| 349 | 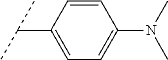 | O | 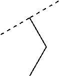 | H | H |
| 350 | 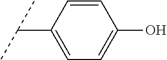 | O | 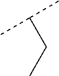 | H | H |
| 351 | 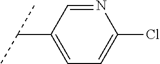 | O | 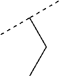 | H | H |
| 352 | 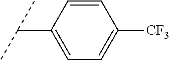 | O | 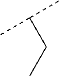 | H | H |
| 353 | 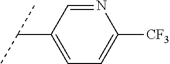 | O | 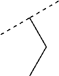 | H | H |
| 354 | 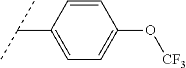 | O | 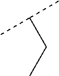 | H | H |
| 355 | 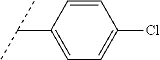 | O | 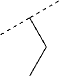 | H | H |
| 356 | 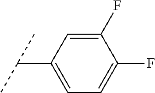 | O | 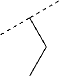 | H | H |
| 357 | 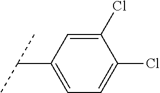 | O | 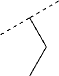 | H | H |
| 358 | 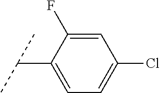 | O | 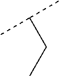 | H | H |
| 359 | 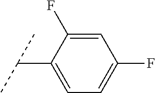 | O | 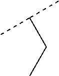 | H | H |
| 360 | 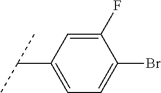 | O | 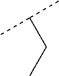 | H | H |
| 361 | 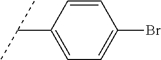 | O | 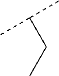 | H | H |
| 362 | 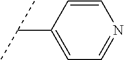 | O | 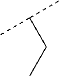 | H | H |
| 363 | 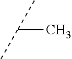 | O | 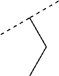 | H | H |
| 364 | 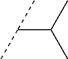 | O | 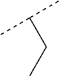 | H | H |
| 365 | 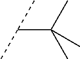 | O | 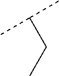 | H | H |
| 366 | 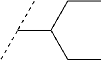 | O | 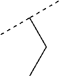 | H | H |
| 367 | 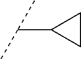 | O | 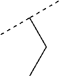 | H | H |
| 368 | 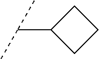 | O | 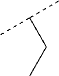 | H | H |
| 369 | 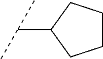 | O | 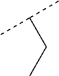 | H | H |
| 370 | 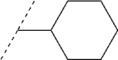 | O | 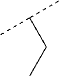 | H | H |
| 371 | 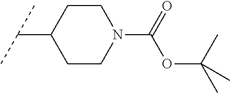 | O | 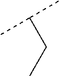 | H | H |
| 372 | 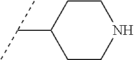 | O | 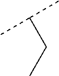 | H | H |
| 373 | 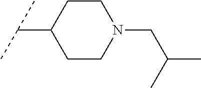 | O | 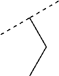 | H | H |
| 374 | 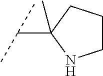 | O | 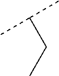 | H | H |
| 375 | 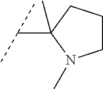 | O | 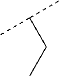 | H | H |
| 376 | 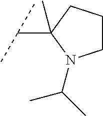 | O | 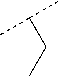 | H | H |
| 377 | 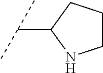 | O | 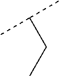 | H | H |
| 378 | 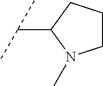 | O | 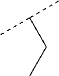 | H | H |
| 379 | 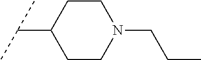 | O | 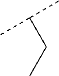 | H | H |
| 380 | 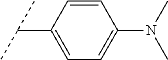 | S | H | H | H |
| 381 | 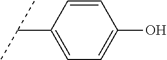 | S | H | H | H |
| 382 | 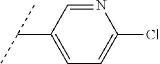 | S | H | H | H |
| 383 | 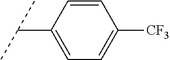 | S | H | H | H |
| 384 | 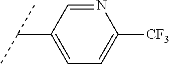 | S | H | H | H |
| 385 | 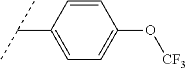 | S | H | H | H |
| 386 | 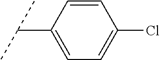 | S | H | H | H |
| 387 | 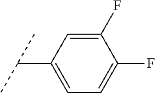 | S | H | H | H |
| 388 | 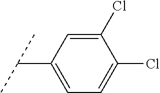 | S | H | H | H |
| 389 | 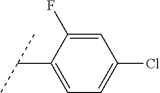 | S | H | H | H |
| 390 | 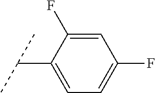 | S | H | H | H |
| 391 | 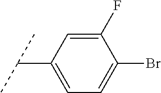 | S | H | H | H |
| 392 | 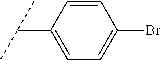 | S | H | H | H |
| 393 | 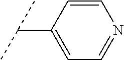 | S | H | H | H |
| 394 | 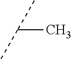 | S | H | H | H |
| 395 | 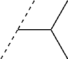 | S | H | H | H |
| 396 | 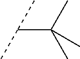 | S | H | H | H |
| 397 | 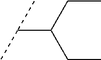 | S | H | H | H |
| 398 | 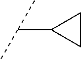 | S | H | H | H |
| 399 | 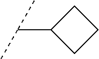 | S | H | H | H |
| 400 | 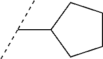 | S | H | H | H |
| 401 | 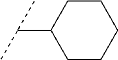 | S | H | H | H |
| 402 | 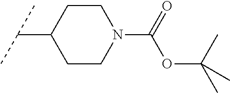 | S | H | H | H |
| 403 | 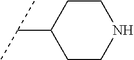 | S | H | H | H |
| 404 | 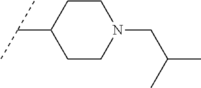 | S | H | H | H |
| 405 | 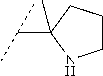 | S | H | H | H |
| 406 | 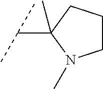 | S | H | H | H |
| 407 | 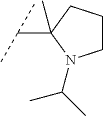 | S | H | H | H |
| 408 | 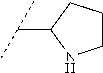 | S | H | H | H |
| 409 | 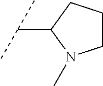 | S | H | H | H |
| 410 | 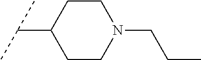 | S | H | H | H |
| 411 | 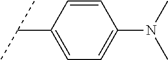 | O | 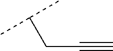 | H | H |
| 412 | 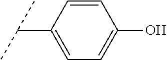 | O | 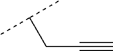 | H | H |
| 413 | 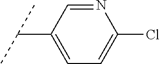 | O | 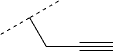 | H | H |
| 414 | 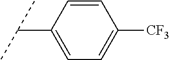 | O | 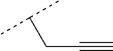 | H | H |
| 415 | 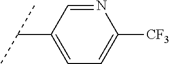 | O | 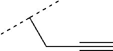 | H | H |
| 416 | 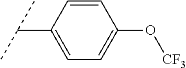 | O | 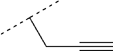 | H | H |
| 417 | 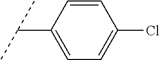 | O | 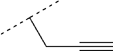 | H | H |
| 418 | 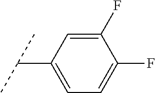 | O | 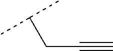 | H | H |
| 419 | 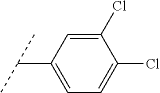 | O | 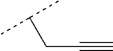 | H | H |
| 420 | 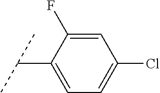 | O | 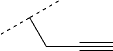 | H | H |
| 421 | 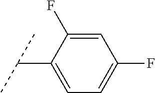 | O | 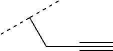 | H | H |
| 422 | 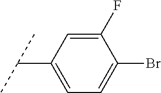 | O | 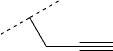 | H | H |
| 423 | 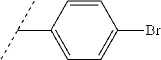 | O | 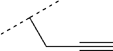 | H | H |
| 424 | 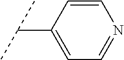 | O | 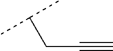 | H | H |
| 425 | 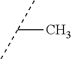 | O | 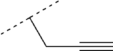 | H | H |
| 426 | 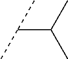 | O | 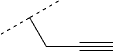 | H | H |
| 427 | 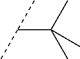 | O | 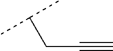 | H | H |
| 428 | 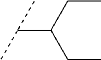 | O | 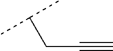 | H | H |
| 429 | 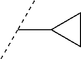 | O | 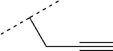 | H | H |
| 430 | 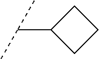 | O | 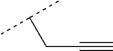 | H | H |
| 431 | 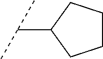 | O | 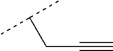 | H | H |
| 432 | 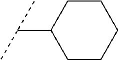 | O | 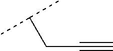 | H | H |
| 433 | 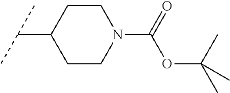 | O | 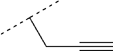 | H | H |
| 434 | 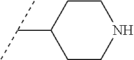 | O | 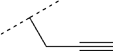 | H | H |
| 435 | 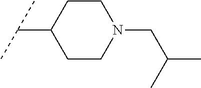 | O | 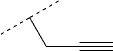 | H | H |
| 436 | 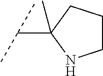 | O | 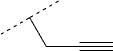 | H | H |
| 437 | 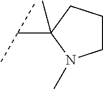 | O | 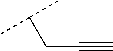 | H | H |
| 438 | 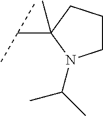 | O | 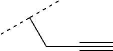 | H | H |
| 439 | 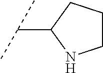 | O | 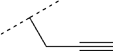 | H | H |
| 440 | 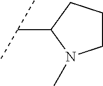 | O | 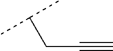 | H | H |
| 441 | 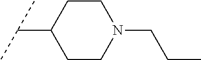 | O | 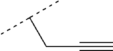 | H | H |
| 442 | 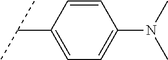 | O | 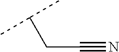 | H | H |
| 443 | 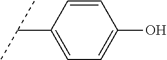 | O | 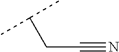 | H | H |
| 444 | 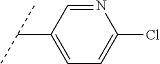 | O | 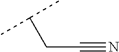 | H | H |
| 445 | 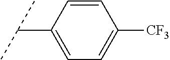 | O | 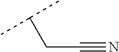 | H | H |
| 446 | 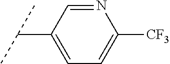 | O | 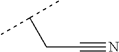 | H | H |
| 447 | 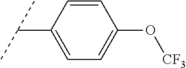 | O | 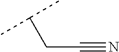 | H | H |
| 448 | 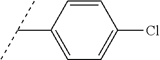 | O | 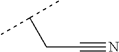 | H | H |
| 449 | 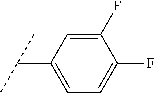 | O | 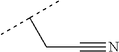 | H | H |
| 450 | 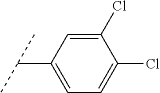 | O | 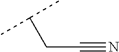 | H | H |
| 451 | 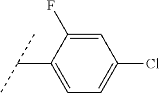 | O | 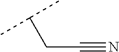 | H | H |
| 452 | 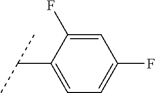 | O | 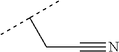 | H | H |
| 453 | 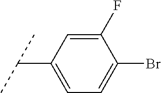 | O | 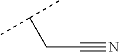 | H | H |
| 454 | 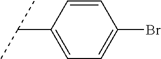 | O | 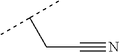 | H | H |
| 455 | 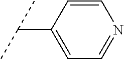 | O | 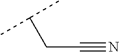 | H | H |
| 456 | 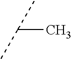 | O | 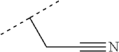 | H | H |
| 457 | 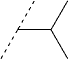 | O | 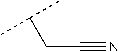 | H | H |
| 11 458 | 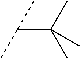 | O | 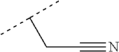 | H | H |
| 459 | 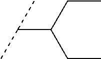 | O | 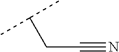 | H | H |
| 460 | 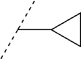 | O | 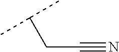 | H | H |
| 461 | 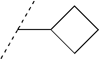 | O | 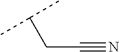 | H | H |
| 462 | 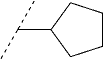 | O | 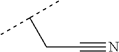 | H | H |
| 463 | 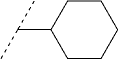 | O | 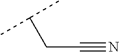 | H | H |
| 464 | 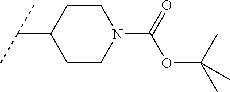 | O | 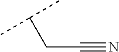 | H | H |
| 465 | 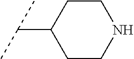 | O | 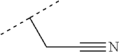 | H | H |
| 466 | 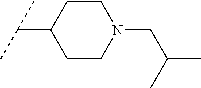 | O | 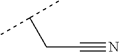 | H | H |
| 467 | 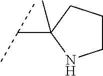 | O | 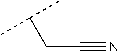 | H | H |
| 468 | 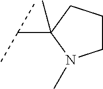 | O | 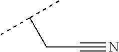 | H | H |
| 469 | 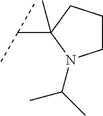 | O | 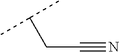 | H | H |
| 470 | 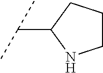 | O | 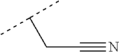 | H | H |
| 471 | 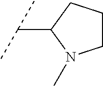 | O | 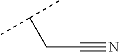 | H | H |
| 472 | 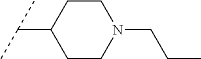 | O | 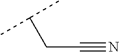 | H | H |
| 473 | 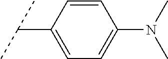 | O | 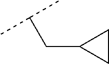 | H | H |
| 474 | 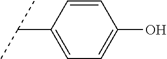 | O | 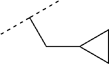 | H | H |
| 475 | 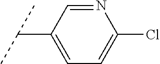 | O | 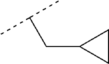 | H | H |
| 476 | 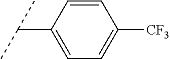 | O | 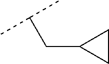 | H | H |
| 477 | 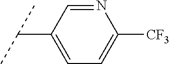 | O | 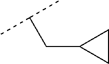 | H | H |
| 478 | 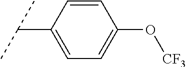 | O | 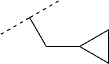 | H | H |
| 479 | 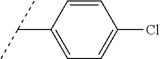 | O | 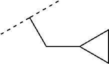 | H | H |
| 480 | 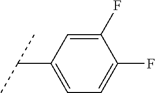 | O | 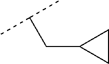 | H | H |
| 481 | 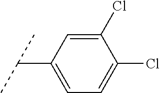 | O | 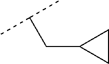 | H | H |
| 482 | 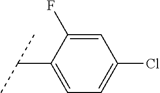 | O | 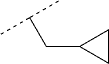 | H | H |
| 483 | 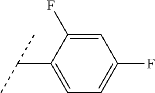 | O | 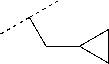 | H | H |
| 484 | 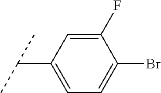 | O | 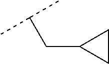 | H | H |
| 485 | 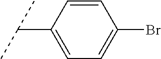 | O | 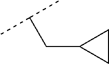 | H | H |
| 486 | 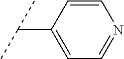 | O | 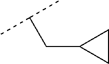 | H | H |
| 487 | 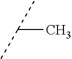 | O | 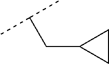 | H | H |
| 488 | 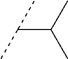 | O | 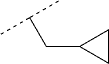 | H | H |
| 489 | 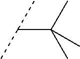 | O | 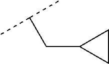 | H | H |
| 490 | 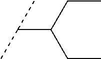 | O | 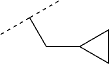 | H | H |
| 491 | 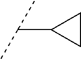 | O | 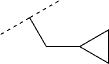 | H | H |
| 492 | 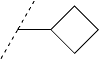 | O | 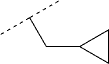 | H | H |
| 493 | 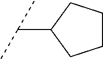 | O | 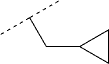 | H | H |
| 494 | 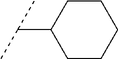 | O | 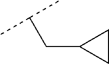 | H | H |
| 496 | 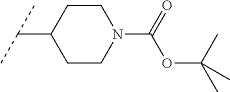 | O | 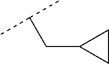 | H | H |
| 497 | 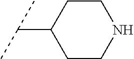 | O | 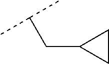 | H | H |
| 498 | 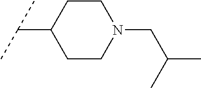 | O | 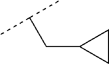 | H | H |
| 499 | 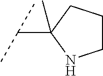 | O | 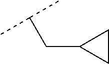 | H | H |
| 500 | 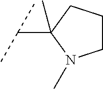 | O | 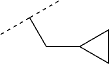 | H | H |
| 501 | 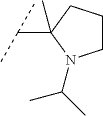 | O | 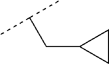 | H | H |
| 502 | 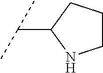 | O | 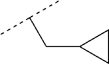 | H | H |
| 503 | 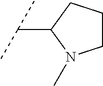 | O | 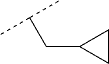 | H | H |
| 504 | 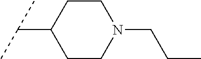 | O | 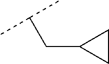 | H | H |
| 505 | 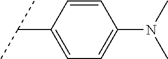 | O | 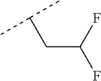 | H | H |
| 506 | 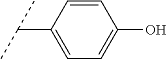 | O | 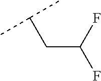 | H | H |
| 507 | 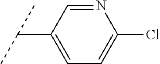 | O | 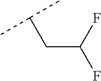 | H | H |
| 508 | 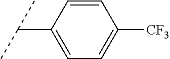 | O | 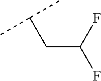 | H | H |
| 509 | 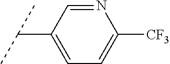 | O | 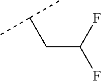 | H | H |
| 510 | 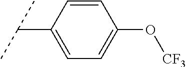 | O | 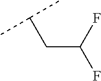 | H | H |
| 511 | 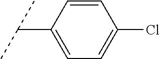 | O | 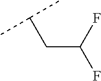 | H | H |
| 512 | 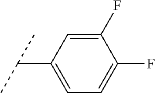 | O | 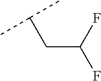 | H | H |
| 513 | 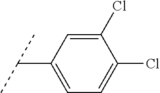 | O | 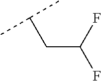 | H | H |
| 514 | 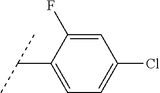 | O | 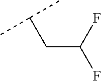 | H | H |
| 515 | 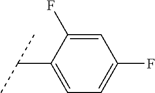 | O | 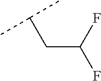 | H | H |
| 516 | 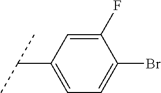 | O | 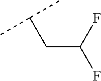 | H | H |
| 517 | 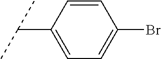 | O | 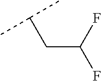 | H | H |
| 518 | 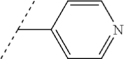 | O | 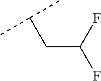 | H | H |
| 519 | 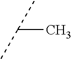 | O | 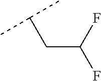 | H | H |
| 520 | 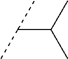 | O | 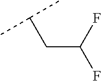 | H | H |
| 521 | 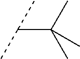 | O | 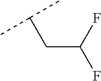 | H | H |
| 522 | 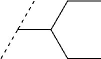 | O | 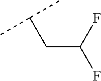 | H | H |
| 523 | 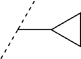 | O | 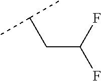 | H | H |
| 524 | 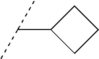 | O | 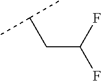 | H | H |
| 525 | 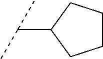 | O | 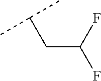 | H | H |
| 526 | 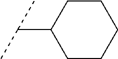 | O | 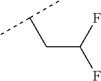 | H | H |
| 527 | 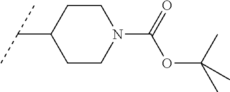 | O | 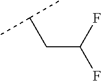 | H | H |
| 528 | 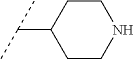 | O | 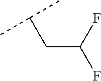 | H | H |
| 529 | 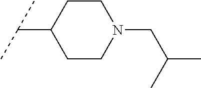 | O | 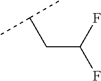 | H | H |
| 530 | 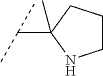 | O | 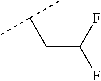 | H | H |
| 531 | 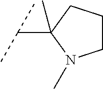 | O | 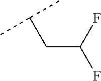 | H | H |
| 532 | 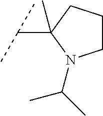 | O | 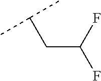 | H | H |
| 533 | 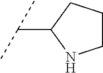 | O | 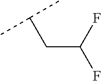 | H | H |
| 534 | 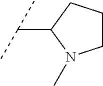 | O | 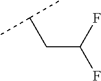 | H | H |
| 535 | 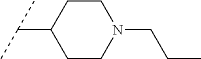 | O | 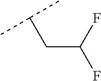 | H | H |
| 536 | 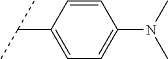 | O | 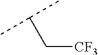 | H | H |
| 537 | 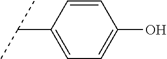 | O | 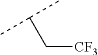 | H | H |
| 538 | 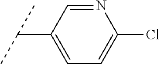 | O | 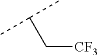 | H | H |
| 539 | 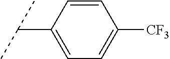 | O | 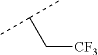 | H | H |
| 540 | 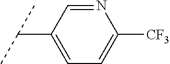 | O | 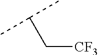 | H | H |
| 541 | 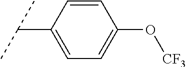 | O | 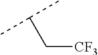 | H | H |
| 542 | 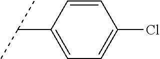 | O | 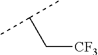 | H | H |
| 543 | 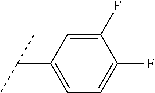 | O | 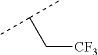 | H | H |
| 544 | 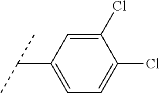 | O | 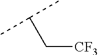 | H | H |
| 545 | 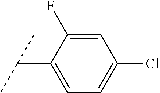 | O | 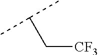 | H | H |
| 546 | 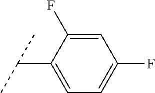 | O | 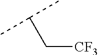 | H | H |
| 547 | 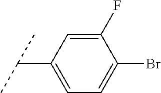 | O | 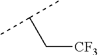 | H | H |
| 548 | 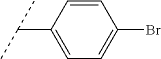 | O | 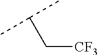 | H | H |
| 549 | 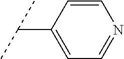 | O | 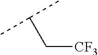 | H | H |
| 550 | 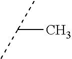 | O | 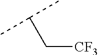 | H | H |
| 551 | 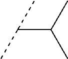 | O | 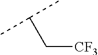 | H | H |
| 552 | 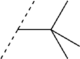 | O | 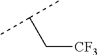 | H | H |
| 553 | 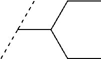 | O | 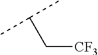 | H | H |
| 554 | 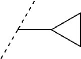 | O | 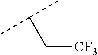 | H | H |
| 555 | 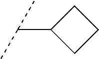 | O | 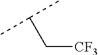 | H | H |
| 556 | 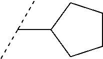 | O | 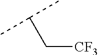 | H | H |
| 557 | 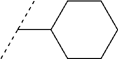 | O | 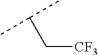 | H | H |
| 558 | 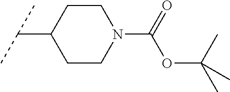 | O | 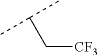 | H | H |
| 559 | 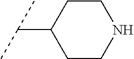 | O | 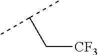 | H | H |
| 560 | 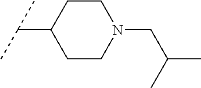 | O | 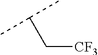 | H | H |
| 561 | 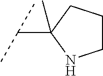 | O | 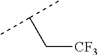 | H | H |
| 562 | 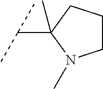 | O | 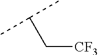 | H | H |
| 563 | 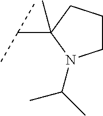 | O | 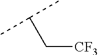 | H | H |
| 564 | 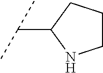 | O | 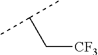 | H | H |
| 565 | 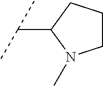 | O | 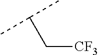 | H | H |
| 566 | 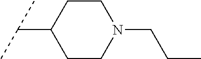 | O | 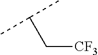 | H | H |
| 567 | 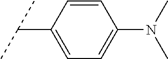 | O | 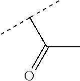 | H | H |
| 568 | 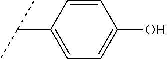 | O | 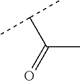 | H | H |
| 569 | 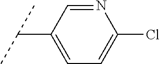 | O | 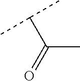 | H | H |
| 570 | 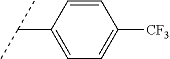 | O | 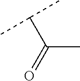 | H | H |
| 571 | 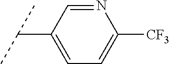 | O | 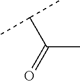 | H | H |
| 572 | 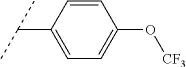 | O | 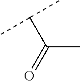 | H | H |
| 573 | 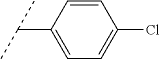 | O | 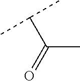 | H | H |
| 574 | 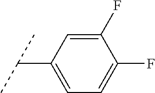 | O | 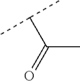 | H | H |
| 575 | 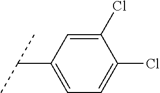 | O | 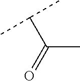 | H | H |
| 576 | 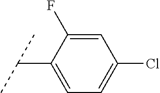 | O | 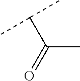 | H | H |
| 577 | 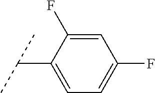 | O | 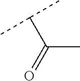 | H | H |
| 578 | 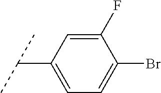 | O | 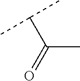 | H | H |
| 579 | 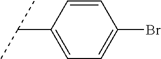 | O | 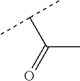 | H | H |
| 580 | 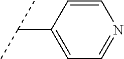 | O | 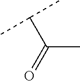 | H | H |
| 581 | 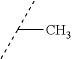 | O | 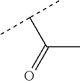 | H | H |
| 582 | 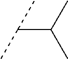 | O | 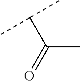 | H | H |
| 583 | 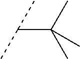 | O | 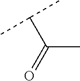 | H | H |
| 584 | 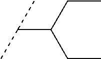 | O | 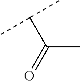 | H | H |
| 585 | 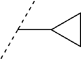 | O | 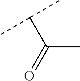 | H | H |
| 586 | 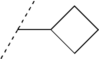 | O | 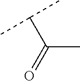 | H | H |
| 587 | 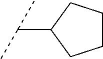 | O | 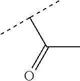 | H | H |
| 588 | 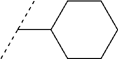 | O | 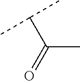 | H | H |
| 589 | 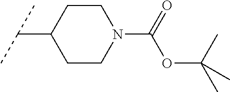 | O | 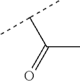 | H | H |
| 590 | 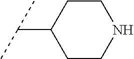 | O | 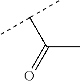 | H | H |
| 591 | 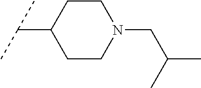 | O | 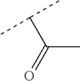 | H | H |
| 592 | 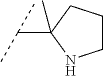 | O | 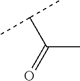 | H | H |
| 593 | 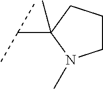 | O | 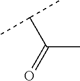 | H | H |
| 594 | 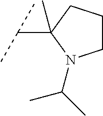 | O | 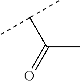 | H | H |
| 595 | 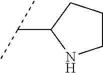 | O | 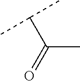 | H | H |
| 596 | 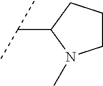 | O | 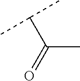 | H | H |
| 597 | 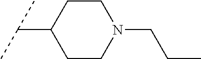 | O | 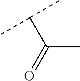 | H | H |
| 598 | 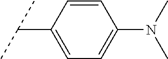 | O | 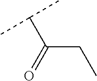 | H | H |
| 599 | 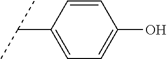 | O | 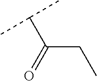 | H | H |
| 600 | 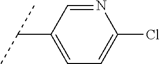 | O | 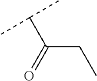 | H | H |
| 601 | 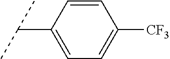 | O | 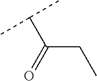 | H | H |
| 602 | 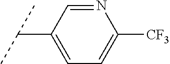 | O | 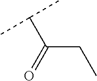 | H | H |
| 603 | 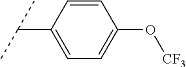 | O | 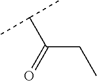 | H | H |
| 604 | 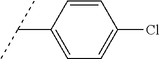 | O | 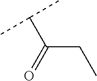 | H | H |
| 605 | 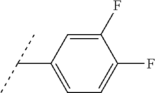 | O | 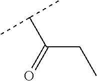 | H | H |
| 606 | 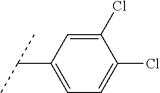 | O | 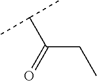 | H | H |
| 607 | 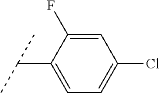 | O | 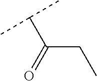 | H | H |
| 608 | 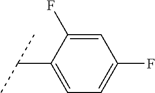 | O | 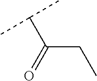 | H | H |
| 609 | 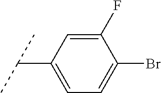 | O | 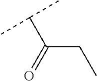 | H | H |
| 610 | 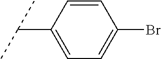 | O | 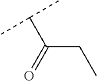 | H | H |
| 611 | 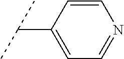 | O | 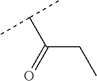 | H | H |
| 612 | 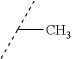 | O | 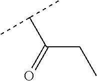 | H | H |
| 613 | 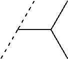 | O | 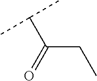 | H | H |
| 614 | 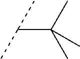 | O | 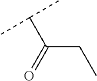 | H | H |
| 615 | 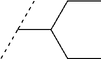 | O | 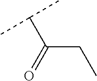 | H | H |
| 616 | 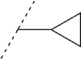 | O | 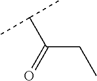 | H | H |
| 617 | 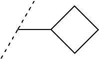 | O | 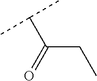 | H | H |
| 618 | 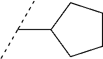 | O | 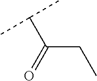 | H | H |
| 619 | 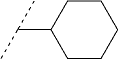 | O | 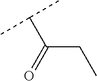 | H | H |
| 620 | 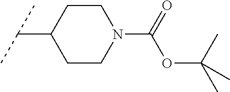 | O | 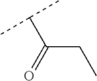 | H | H |
| 621 | 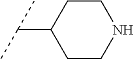 | O | 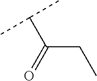 | H | H |
| 622 | 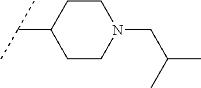 | O | 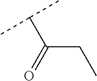 | H | H |
| 623 | 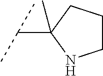 | O | 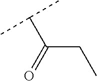 | H | H |
| 624 | 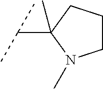 | O | 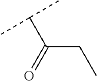 | H | H |
| 625 | 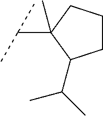 | O | 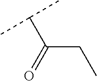 | H | H |
| 626 | 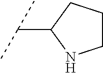 | O | 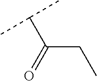 | H | H |
| 627 | 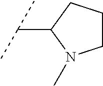 | O | 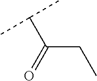 | H | H |
| 628 | 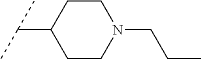 | O | 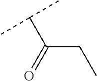 | H | H |
| 629 | 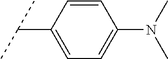 | O | 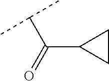 | H | H |
| 630 | 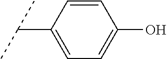 | O | 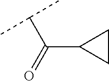 | H | H |
| 631 | 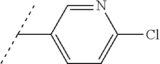 | O | 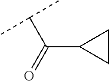 | H | H |
| 632 | 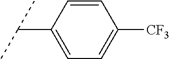 | O | 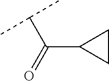 | H | H |
| 633 | 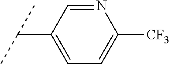 | O | 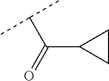 | H | H |
| 634 | 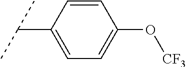 | O | 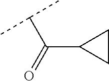 | H | H |
| 635 | 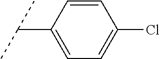 | O | 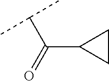 | H | H |
| 636 | 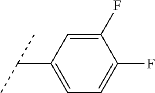 | O | 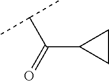 | H | H |
| 637 | 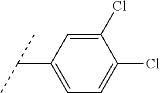 | O | 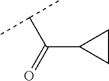 | H | H |
| 638 | 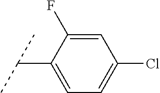 | O | 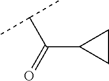 | H | H |
| 639 | 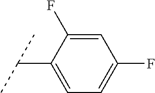 | O | 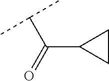 | H | H |
| 640 | 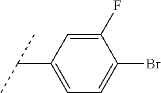 | O | 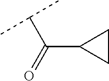 | H | H |
| 641 | 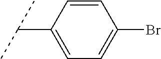 | O | 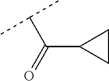 | H | H |
| 642 | 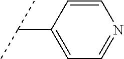 | O | 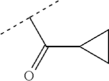 | H | H |
| 643 | 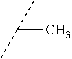 | O | 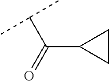 | H | H |
| 644 | 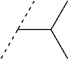 | O | 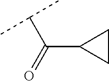 | H | H |
| 645 | 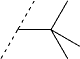 | O | 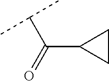 | H | H |
| 646 | 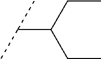 | O | 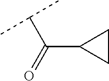 | H | H |
| 647 | 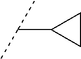 | O | 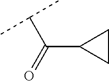 | H | H |
| 648 | 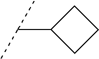 | O | 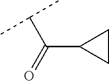 | H | H |
| 649 | 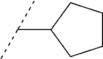 | O | 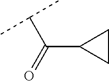 | H | H |
| 650 | 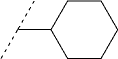 | O | 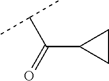 | H | H |
| 651 | 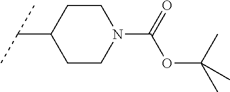 | O | 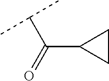 | H | H |
| 652 | 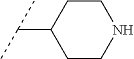 | O | 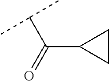 | H | H |
| 653 | 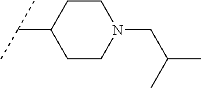 | O | 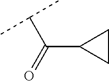 | H | H |
| 654 | 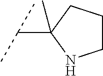 | O | 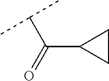 | H | H |
| 655 | 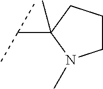 | O | 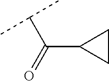 | H | H |
| 656 | 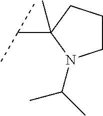 | O | 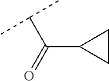 | H | H |
| 657 | 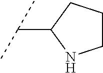 | O | 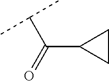 | H | H |
| 658 | 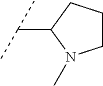 | O | 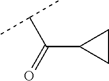 | H | H |
| 659 | 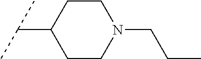 | O | 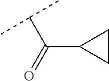 | H | H |
| 660 | 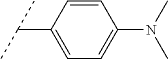 | O | 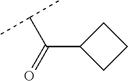 | H | H |
| 661 | 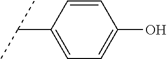 | O | 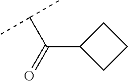 | H | H |
| 662 | 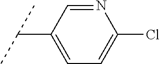 | O | 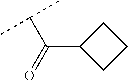 | H | H |
| 663 | 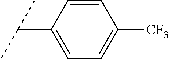 | O | 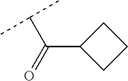 | H | H |
| 664 | 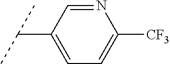 | O | 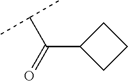 | H | H |
| 665 | 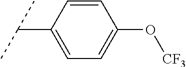 | O | 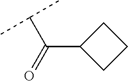 | H | H |
| 666 | 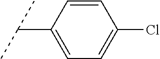 | O | 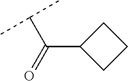 | H | H |
| 667 | 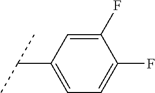 | O | 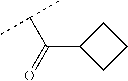 | H | H |
| 668 | 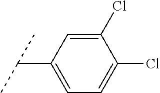 | O | 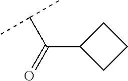 | H | H |
| 669 | 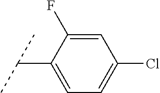 | O | 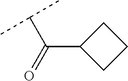 | H | H |
| 670 | 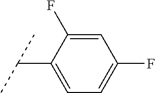 | O | 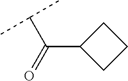 | H | H |
| 671 | 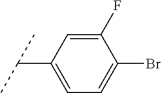 | O | 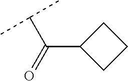 | H | H |
| 672 | 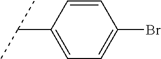 | O | 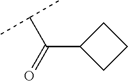 | H | H |
| 673 | 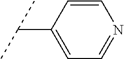 | O | 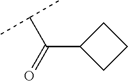 | H | H |
| 674 | 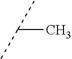 | O | 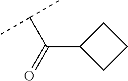 | H | H |
| 675 | 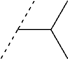 | O | 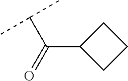 | H | H |
| 676 | 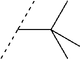 | O | 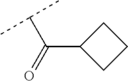 | H | H |
| 677 | 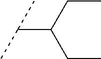 | O | 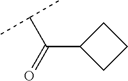 | H | H |
| 678 | 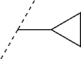 | O | 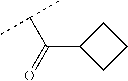 | H | H |
| 679 | 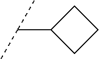 | O | 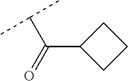 | H | H |
| 680 | 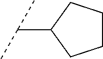 | O | 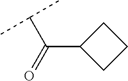 | H | H |
| 681 | 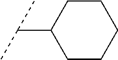 | O | 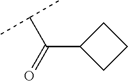 | H | H |
| 682 | 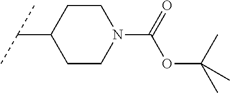 | O | 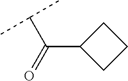 | H | H |
| 683 | 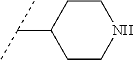 | O | 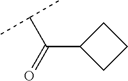 | H | H |
| 684 | 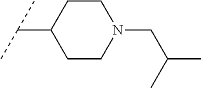 | O | 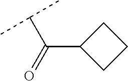 | H | H |
| 685 | 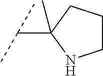 | O | 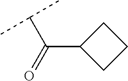 | H | H |
| 686 | 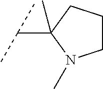 | O | 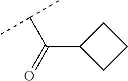 | H | H |
| 687 | 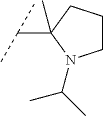 | O | 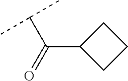 | H | H |
| 688 | 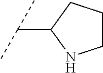 | O | 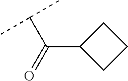 | H | H |
| 689 | 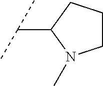 | O | 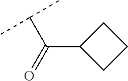 | H | H |
| 690 | 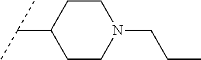 | O | 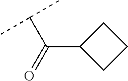 | H | H |
| 691 | 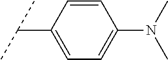 | O | 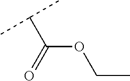 | H | H |
| 692 | 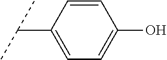 | O | 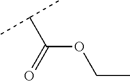 | H | H |
| 693 | 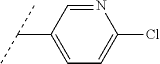 | O | 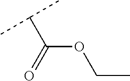 | H | H |
| 694 | 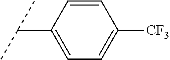 | O | 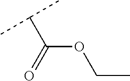 | H | H |
| 695 | 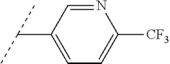 | O | 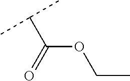 | H | H |
| 696 | 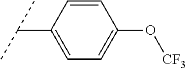 | O | 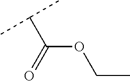 | H | H |
| 697 | 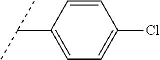 | O | 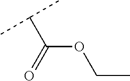 | H | H |
| 698 | 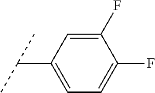 | O | 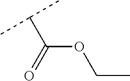 | H | H |
| 699 | 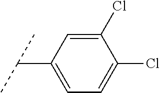 | O | 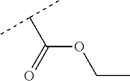 | H | H |
| 700 | 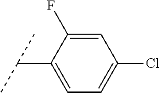 | O | 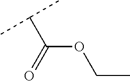 | H | H |
| 701 | 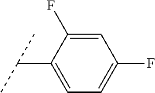 | O | 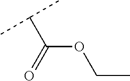 | H | H |
| 702 | 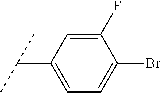 | O | 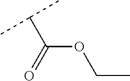 | H | H |
| 703 | 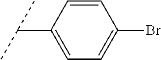 | O | 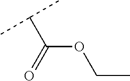 | H | H |
| 704 | 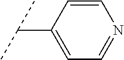 | O | 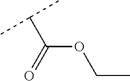 | H | H |
| 705 | 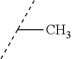 | O | 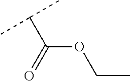 | H | H |
| 706 | 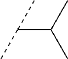 | O | 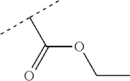 | H | H |
| 707 | 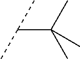 | O | 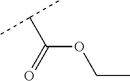 | H | H |
| 708 | 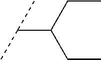 | O | 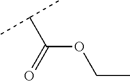 | H | H |
| 709 | 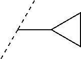 | O | 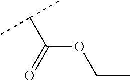 | H | H |
| 710 | 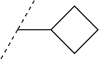 | O | 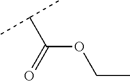 | H | H |
| 711 | 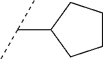 | O | 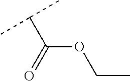 | H | H |
| 712 | 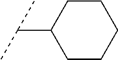 | O | 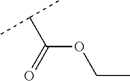 | H | H |
| 713 | 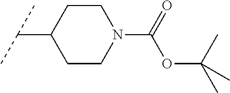 | O | 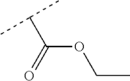 | H | H |
| 714 | 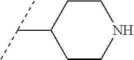 | O | 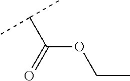 | H | H |
| 715 | 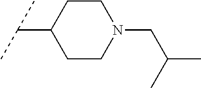 | O | 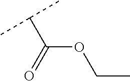 | H | H |
| 716 | 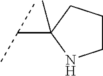 | O | 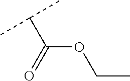 | H | H |
| 717 | 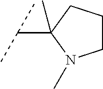 | O | 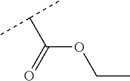 | H | H |
| 718 | 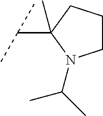 | O | 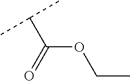 | H | H |
| 719 | 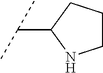 | O | 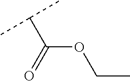 | H | H |
| 720 | 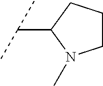 | O | 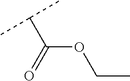 | H | H |
| 721 | 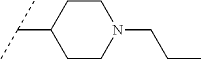 | O | 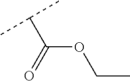 | H | H |
| 722 | 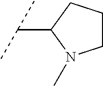 | O | H | 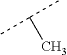 | H |
| 723 | 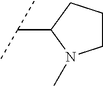 | O | H | 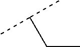 | H |
| 724 | 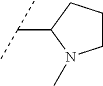 | O | H | 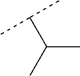 | H |
| 725 | 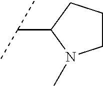 | O | H | 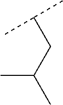 | H |
| 726 | 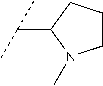 | O | H | 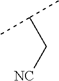 | H |
| 727 | 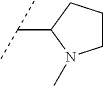 | O | H | 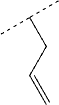 | H |
| 728 | 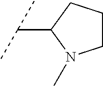 | O | H | 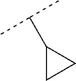 | H |
| 729 | 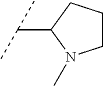 | O | H | 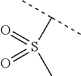 | H |
| 730 | 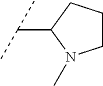 | O | H | 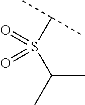 | H |
| 731 | 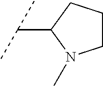 | O | H | 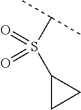 | H |
| 732 | 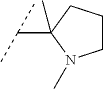 | O | H | 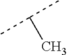 | H |
| 733 | 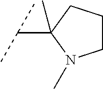 | O | H | 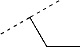 | H |
| 734 | 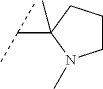 | O | H | 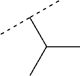 | H |
| 735 | 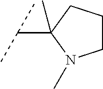 | O | H | 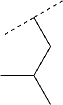 | H |
| 736 | 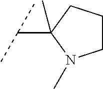 | O | H | 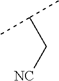 | H |
| 737 | 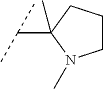 | O | H | 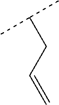 | H |
| 738 | 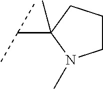 | O | H | 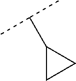 | H |
| 739 | 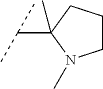 | O | H | 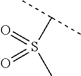 | H |
| 740 | 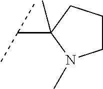 | O | H | 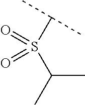 | H |
| 741 | 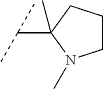 | O | H | 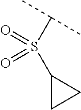 | H |
| 742 | 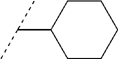 | O | H | 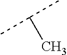 | H |
| 743 | 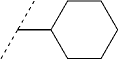 | O | H | 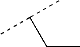 | H |
| 744 | 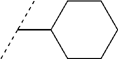 | O | H | 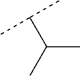 | H |
| 745 | 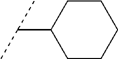 | O | H | 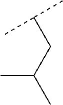 | H |
| 746 | 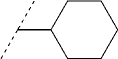 | O | H | 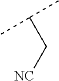 | H |
| 747 | 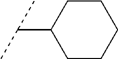 | O | H | 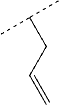 | H |
| 748 | 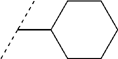 | O | H | 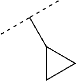 | H |
| 749 | 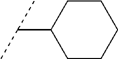 | O | H | 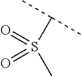 | H |
| 750 | 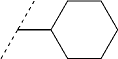 | O | H | 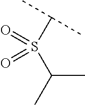 | H |
| 751 | 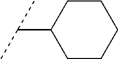 | O | H | 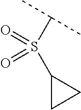 | H |
| 752 | 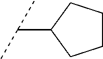 | O | H | 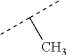 | H |
| 753 | 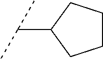 | O | H | 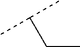 | H |
| 754 | 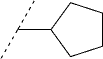 | O | H | 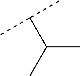 | H |
| 755 | 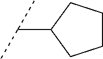 | O | H | 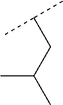 | H |
| 756 | 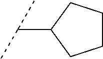 | O | H | 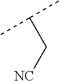 | H |
| 757 | 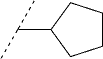 | O | H | 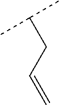 | H |
| 758 | 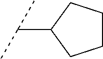 | O | H | 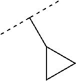 | H |
| 759 | 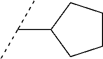 | O | H | 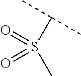 | H |
| 760 | 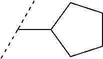 | O | H | 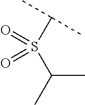 | H |
| 761 | 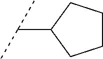 | O | H | 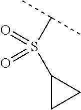 | H |
| 762 | 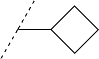 | O | H | 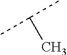 | H |
| 763 | 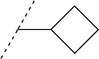 | O | H | 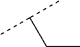 | H |
| 764 | 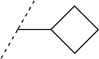 | O | H | 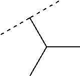 | H |
| 765 | 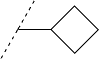 | O | H | 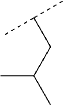 | H |
| 766 | 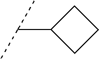 | O | H | 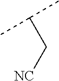 | H |
| 767 | 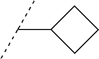 | O | H | 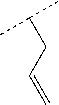 | H |
| 768 | 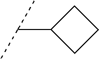 | O | H | 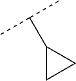 | H |
| 769 | 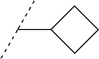 | O | H | 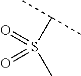 | H |
| 770 | 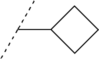 | O | H | 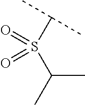 | H |
| 771 | 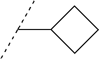 | O | H | 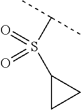 | H |
| 772 | 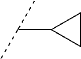 | O | H | 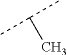 | H |
| 773 | 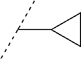 | O | H | 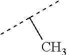 | H |
| 774 | 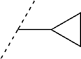 | O | H | 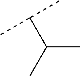 | H |
| 775 | 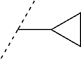 | O | H | 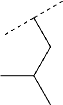 | H |
| 776 | 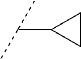 | O | H | 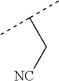 | H |
| 777 | 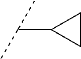 | O | H | 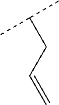 | H |
| 778 | 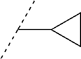 | O | H | 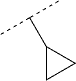 | H |
| 779 | 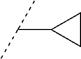 | O | H | 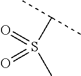 | H |
| 780 | 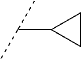 | O | H | 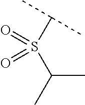 | H |
| 781 | 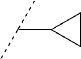 | O | H | 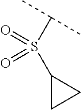 | H |
| 782 | 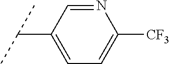 | O | H | 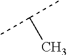 | H |
| 783 | 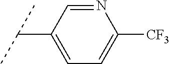 | O | H | 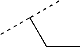 | H |
| 784 | 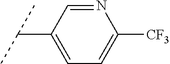 | O | H | 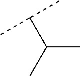 | H |
| 785 | 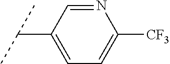 | O | H | 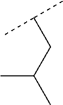 | H |
| 786 | 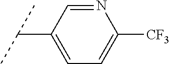 | O | H | 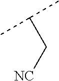 | H |
| 787 | 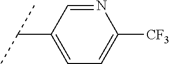 | O | H | 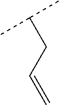 | H |
| 788 | 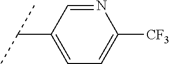 | O | H | 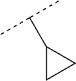 | H |
| 789 | 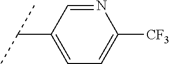 | O | H | 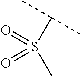 | H |
| 790 | 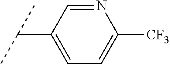 | O | H | 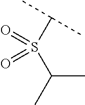 | H |
| 791 | 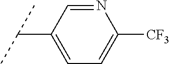 | O | H | 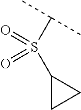 | H |
| 792 | 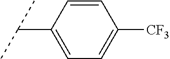 | O | H | 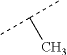 | H |
| 793 | 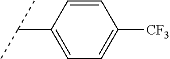 | O | H | 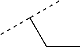 | H |
| 794 | 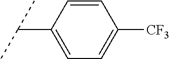 | O | H | 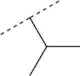 | H |
| 795 | 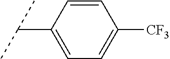 | O | H | 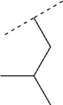 | H |
| 796 | 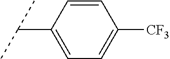 | O | H | 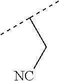 | H |
| 797 | 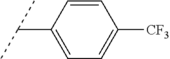 | O | H | 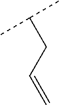 | H |
| 798 | 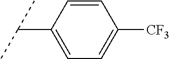 | O | H | 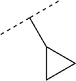 | H |
| 799 | 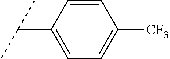 | O | H | 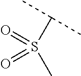 | H |
| 800 | 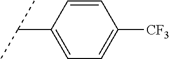 | O | H | 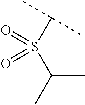 | H |
| 801 | 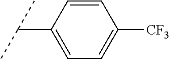 | O | H | 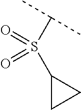 | H |
| 802 | 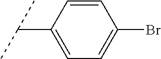 | O | H | 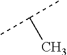 | H |
| 803 | 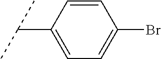 | O | H | 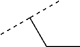 | H |
| 804 | 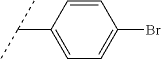 | O | H | 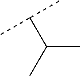 | H |
| 805 | 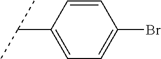 | O | H | 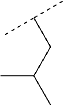 | H |
| 806 | 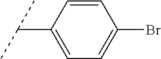 | O | H | 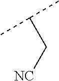 | H |
| 807 | 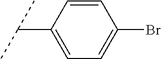 | O | H | 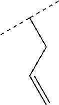 | H |
| 808 | 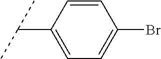 | O | H | 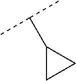 | H |
| 809 | 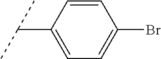 | O | H | 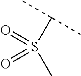 | H |
| 810 | 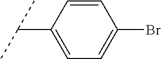 | O | H | 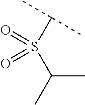 | H |
| 811 | 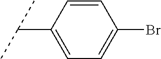 | O | H | 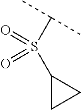 | H |
| 812 | 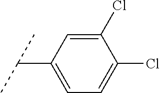 | O | H | 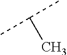 | H |
| 813 | 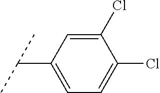 | O | H | 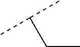 | H |
| 814 | 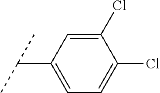 | O | H | 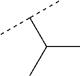 | H |
| 815 | 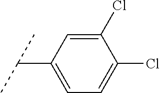 | O | H | 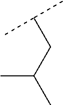 | H |
| 816 | 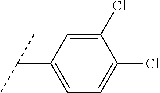 | O | H | 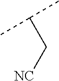 | H |
| 817 | 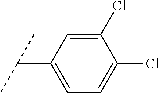 | O | H | 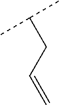 | H |
| 818 | 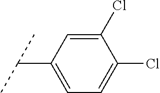 | O | H | 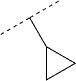 | H |
| 819 | 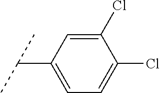 | O | H | 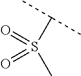 | H |
| 820 | 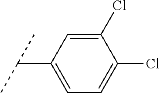 | O | H | 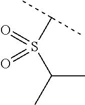 | H |
| 821 | 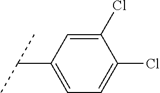 | O | H | 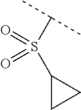 | H |
| 822 | 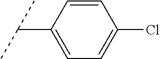 | O | H | 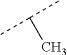 | H |
| 823 | 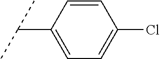 | O | H | 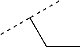 | H |
| 824 | 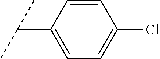 | O | H | 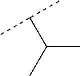 | H |
| 825 | 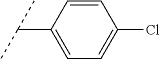 | O | H | 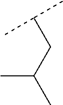 | H |
| 826 | 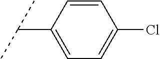 | O | H | 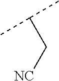 | H |
| 827 | 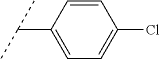 | O | H | 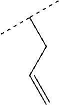 | H |
| 828 | 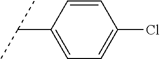 | O | H | 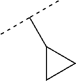 | H |
| 829 | 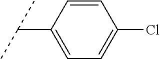 | O | H | 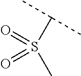 | H |
| 830 | 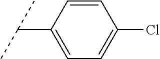 | O | H | 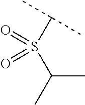 | H |
| 831 | 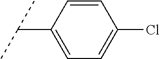 | O | H | 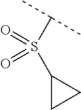 | H |
| 832 | 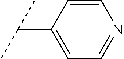 | O | H | 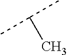 | H |
| 833 | 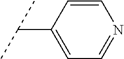 | O | H | 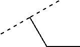 | H |
| 834 | 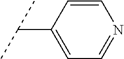 | O | H | 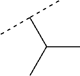 | H |
| 835 | 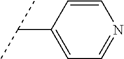 | O | H | 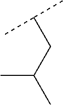 | H |
| 836 | 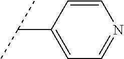 | O | H | 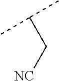 | H |
| 837 | 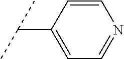 | O | H | 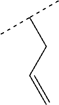 | H |
| 838 | 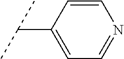 | O | H | 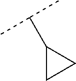 | H |
| 839 | 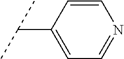 | O | H | 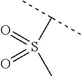 | H |
| 840 | 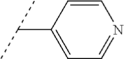 | O | H | 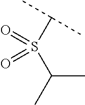 | H |
| 841 | 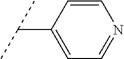 | O | H | 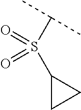 | H |
| 842 | 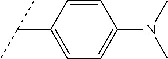 | O | H | 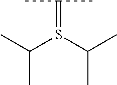 | |
| 843 | 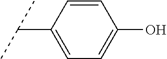 | O | H | 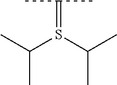 | |
| 844 | 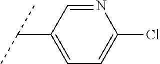 | O | H | 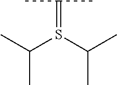 | |
| 845 | 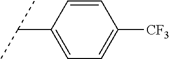 | O | H | 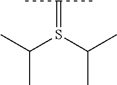 | |
| 846 | 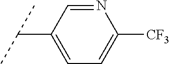 | O | H | 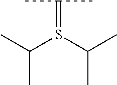 | |
| 847 | 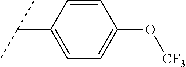 | O | H | 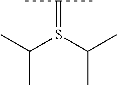 | |
| 848 | 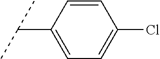 | O | H | 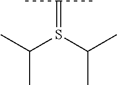 | |
| 849 | 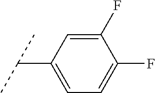 | O | H | 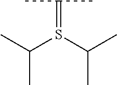 | |
| 850 | 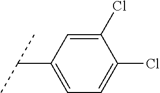 | O | H | 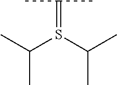 | |
| 851 | 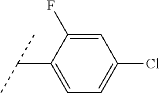 | O | H | 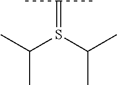 | |
| 852 | 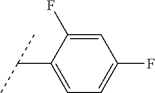 | O | H | 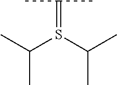 | |
| 853 | 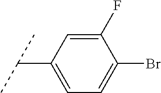 | O | H | 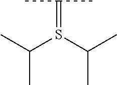 | |
| 854 | 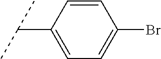 | O | H | 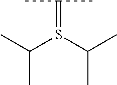 | |
| 855 | 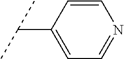 | O | H | 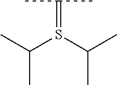 | |
| 856 | 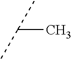 | O | H | 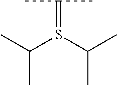 | |
| 857 | 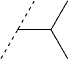 | O | H | 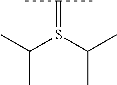 | |
| 858 | 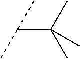 | O | H | 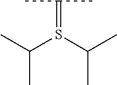 | |
| 859 | 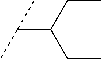 | O | H | 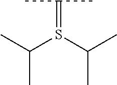 | |
| 860 | 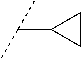 | O | H | 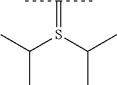 | |
| 861 | 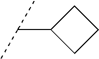 | O | H | 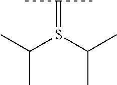 | |
| 862 | 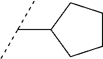 | O | H | 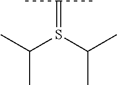 | |
| 863 | 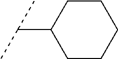 | O | H | 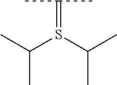 | |
| 864 | 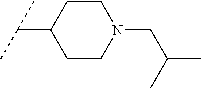 | O | H | 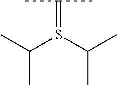 | |
| 865 | 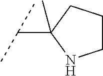 | O | H | 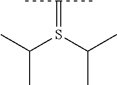 | |
| 866 | 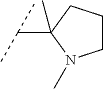 | O | H | 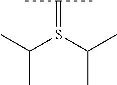 | |
| 867 | 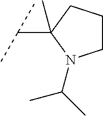 | O | H | 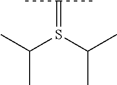 | |
| 868 | 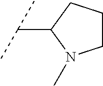 | O | H | 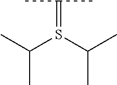 | |
| 869 | 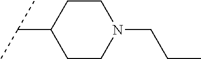 | O | H | 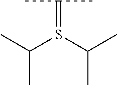 | |
| 870 | 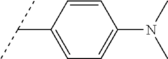 | O | H | 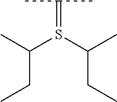 | |
| 871 | 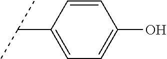 | O | H | 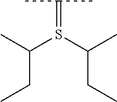 | |
| 872 | 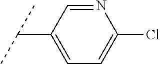 | O | H | 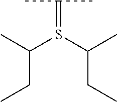 | |
| 873 | 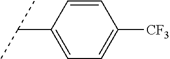 | O | H | 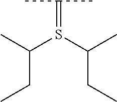 | |
| 874 | 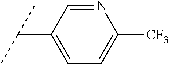 | O | H | 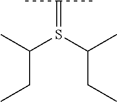 | |
| 875 | 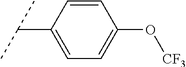 | O | H | 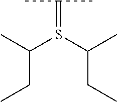 | |
| 876 | 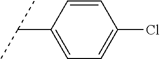 | O | H | 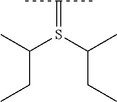 | |
| 877 | 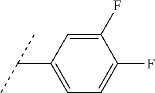 | O | H | 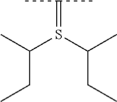 | |
| 878 | 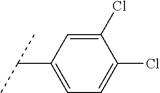 | O | H | 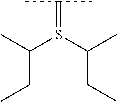 | |
| 879 | 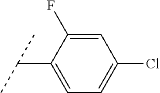 | O | H | 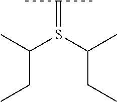 | |
| 880 | 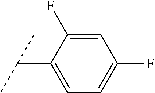 | O | H | 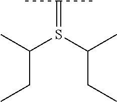 | |
| 881 | 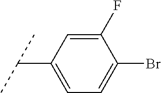 | O | H | 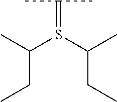 | |
| 882 | 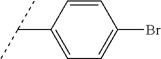 | O | H | 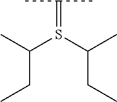 | |
| 883 | 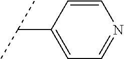 | O | H | 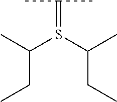 | |
| 884 | 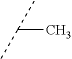 | O | H | 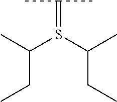 | |
| 885 | 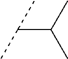 | O | H | 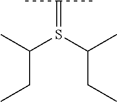 | |
| 886 | 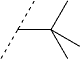 | O | H | 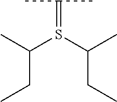 | |
| 887 | 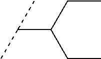 | O | H | 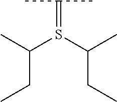 | |
| 888 | 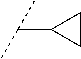 | O | H | 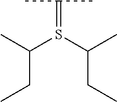 | |
| 889 | 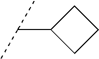 | O | H | 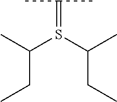 | |
| 890 | 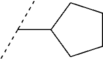 | O | H | 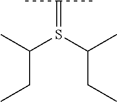 | |
| 891 | 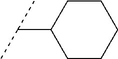 | O | H | 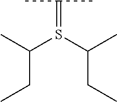 | |
| 892 | 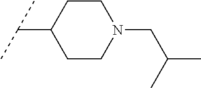 | O | H | 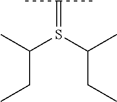 | |
| 893 | 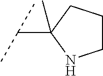 | O | H | 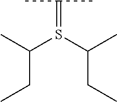 | |
| 894 | 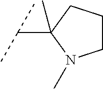 | O | H | 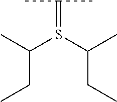 | |
| 895 | 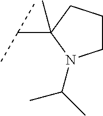 | O | H | 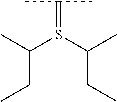 | |
| 896 | 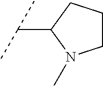 | O | H | 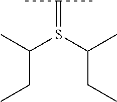 | |
| 897 | 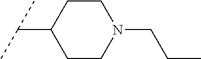 | O | H | 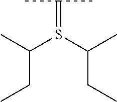 | |
| 898 | 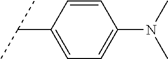 | O | H | 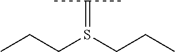 | |
| 899 | 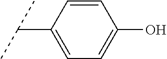 | O | H | 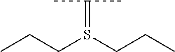 | |
| 900 | 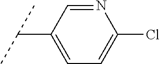 | O | H | 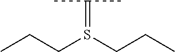 | |
| 901 | 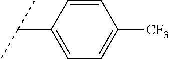 | O | H | 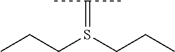 | |
| 902 | 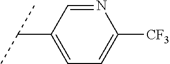 | O | H | 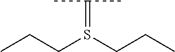 | |
| 903 | 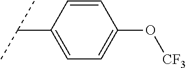 | O | H | 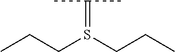 | |
| 904 | 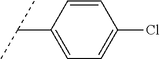 | O | H | 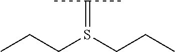 | |
| 905 | 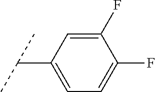 | O | H | 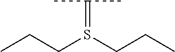 | |
| 906 | 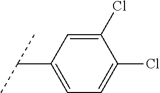 | O | H | 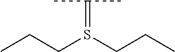 | |
| 907 | 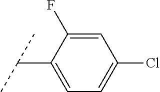 | O | H | 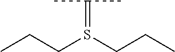 | |
| 908 | 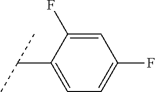 | O | H | 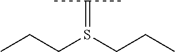 | |
| 909 | 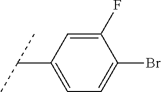 | O | H | 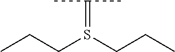 | |
| 910 | 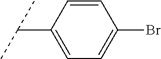 | O | H | 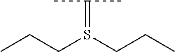 | |
| 911 | 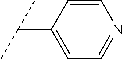 | O | H | 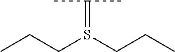 | |
| 912 | 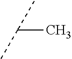 | O | H | 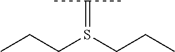 | |
| 913 | 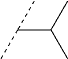 | O | H | 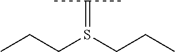 | |
| 914 | 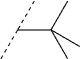 | O | H | 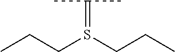 | |
| 915 | 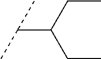 | O | H | 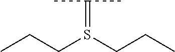 | |
| 916 | 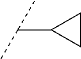 | O | H | 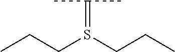 | |
| 917 | 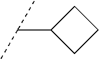 | O | H | 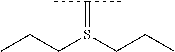 | |
| 918 | 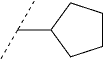 | O | H | 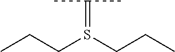 | |
| 919 | 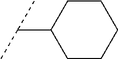 | O | H | 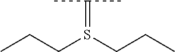 | |
| 920 | 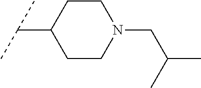 | O | H | 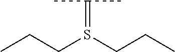 | |
| 921 | 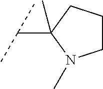 | O | H | 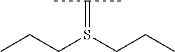 | |
| 922 | 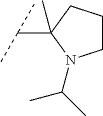 | O | H | 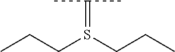 | |
| 923 | 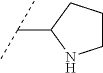 | O | H | 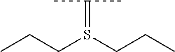 | |
| 924 | 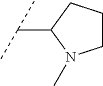 | O | H | 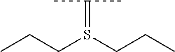 | |
| 925 | 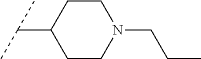 | O | H | 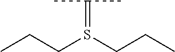 | |
| 926 | 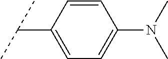 | O | H | 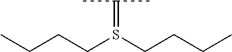 | |
| 927 | 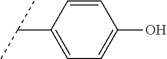 | O | H | 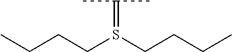 | |
| 928 | 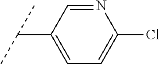 | O | H | 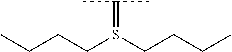 | |
| 929 | 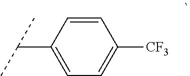 | O | H | 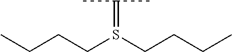 | |
| 930 | 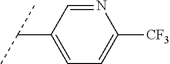 | O | H | 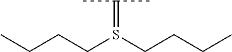 | |
| 931 | 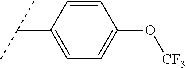 | O | H | 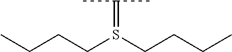 | |
| 932 | 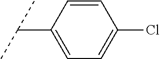 | O | H | 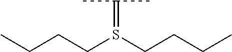 | |
| 933 | 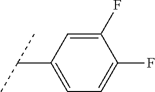 | O | H | 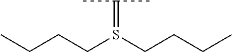 | |
| 934 | 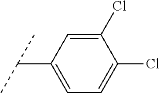 | O | H | 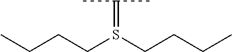 | |
| 935 | 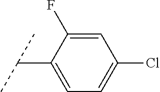 | O | H | 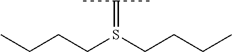 | |
| 936 | 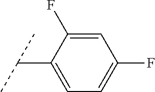 | O | H | 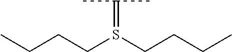 | |
| 937 | 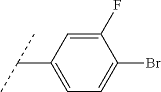 | O | H | 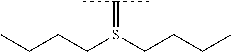 | |
| 938 | 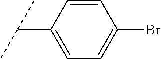 | O | H | 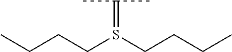 | |
| 939 | 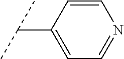 | O | H | 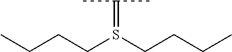 | |
| 940 | 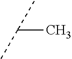 | O | H | 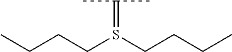 | |
| 941 | 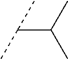 | O | H | 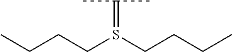 | |
| 942 | 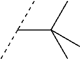 | O | H | 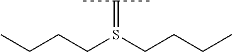 | |
| 943 | 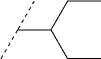 | O | H | 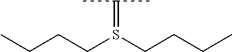 | |
| 944 | 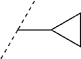 | O | H | 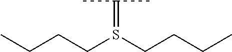 | |
| 945 | 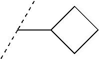 | O | H | 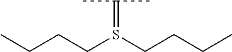 | |
| 946 | 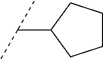 | O | H | 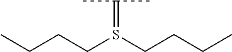 | |
| 947 | 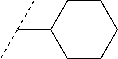 | O | H | 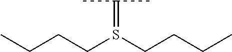 | |
| 948 | 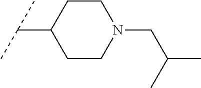 | O | H | 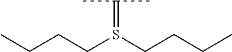 | |
| 949 | 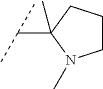 | O | H | 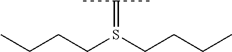 | |
| 950 | 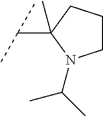 | O | H | 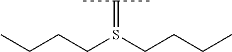 | |
| 951 | 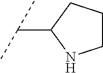 | O | H | 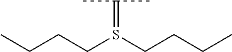 | |
| 952 | 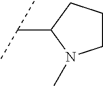 | O | H | 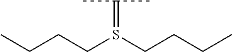 | |
| 953 | 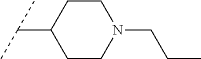 | O | H | 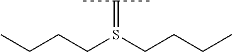 | |
| 954 | 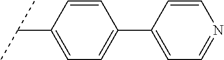 | O | H | H | H |
| 955 | 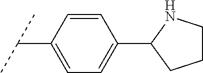 | O | H | H | H |
| 956 | 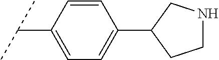 | O | H | H | H |
| 957 | 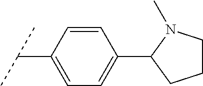 | O | H | H | H |
| 958 | 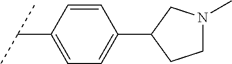 | O | H | H | H |
| 959 | 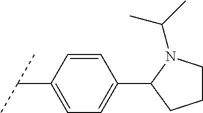 | O | H | H | H |
| 960 | 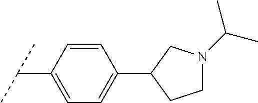 | O | H | H | H |
| 961 | 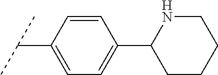 | O | H | H | H |
| 962 | 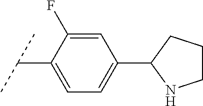 | O | H | H | H |
| 963 | 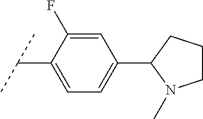 | O | H | H | H |
| 964 | 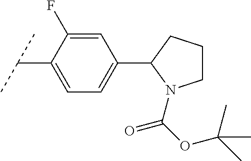 | O | H | H | H |
| 965 | 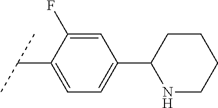 | O | H | H | H |
| 966 | 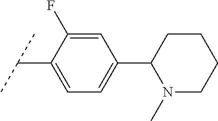 | O | H | H | H |
| 967 | 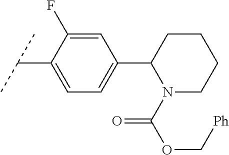 | O | H | H | H |
| 968 | 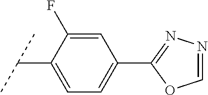 | O | H | H | H |
| 969 | 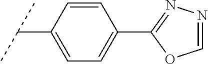 | O | H | H | H |
| 970 | 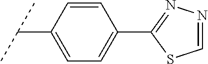 | O | H | H | H |
| 971 | 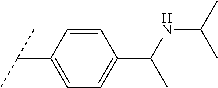 | O | H | H | H |
| 972 | 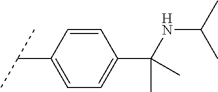 | O | H | H | H |
| 973 | 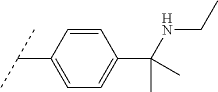 | O | H | H | H |
| 974 | 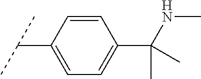 | O | H | H | H |
| 975 | 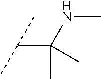 | O | H | H | H |
| 976 | 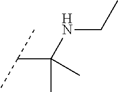 | O | H | H | H |
| 977 | 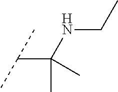 | O | H | H | H |
| 978 | 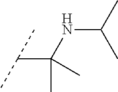 | O | H | H | H |
| 979 | 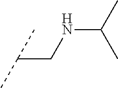 | O | H | H | H |
| 980 | 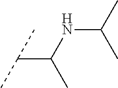 | O | H | H | H |
| 981 | 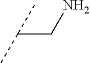 | O | H | H | H |
| 982 | 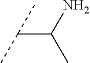 | O | H | H | H |
| 983 | 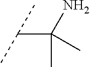 | O | H | H | H |
| 984 | 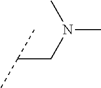 | O | H | H | H |
| 985 | 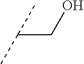 | O | H | H | H |
| 986 | 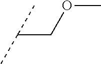 | O | H | H | H |
| 987 | 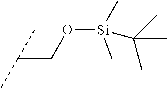 | O | H | H | H |
| 988 | 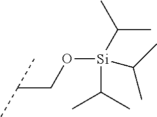 | O | H | H | H |
| 989 | 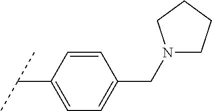 | O | H | H | H |
| 990 | 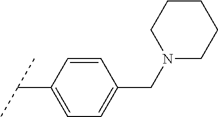 | O | H | H | H |
| 991 | 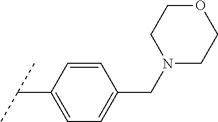 | O | H | H | H |
| 992 | 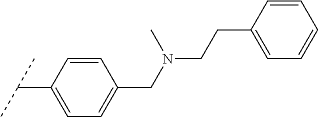 | O | H | H | H |
| 993 | 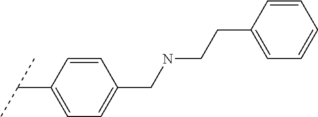 | O | H | H | H |
| 994 | 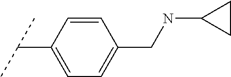 | O | H | H | H |
| 995 | 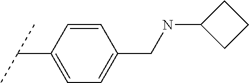 | O | H | H | H |
| 996 | 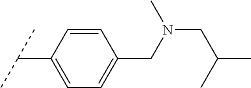 | O | H | H | H |
| 997 | 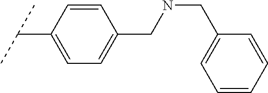 | O | H | H | H |
| 998 | 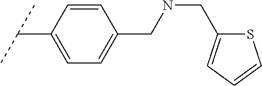 | O | H | H | H |
| 999 | 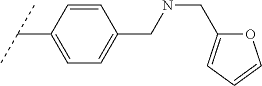 | O | H | H | H |
| 1000 | 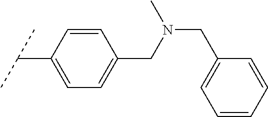 | O | H | H | H |

A2. Compounds A2-1 to A2-1000 of the general formula (I) in which R¹ and R³ represent hydrogen, R² represents trifluoromethyl, X represents N—R⁴ and Q, W, Z¹, Z² and R⁴ for the individual compound in question correspond to the radical definitions given in Table 1 (Nos 1 to 1000; corresponding to compounds A2-1 to A2-1000).
A3. Compounds A3-1 to A3-1000 of the general formula (I) in which R¹ and R³ represent hydrogen, R² represents methyl, X represents N—R⁴ and O, W, Z¹, Z² and R⁴ for the individual compound in question correspond to the radical definitions given in Table 1 (Nos 1 to 1000; corresponding to compounds A3-1 to A3-1000).
A4. Compounds A4-1 to A4-1000 of the general formula (I) in which R¹ and R³ represent hydrogen, R² represents fluorine, X represents N—R⁴ and Q, W, Z¹, Z² and R⁴ for the individual compound in question correspond to the radical definitions given in Table 1 (Nos 1 to 1000; corresponding to compounds A4-1 to A4-1000).
A5. Compounds A5-1 to A5-1000 of the general formula (I) in which R¹ and R³ represent hydrogen, R² represents chlorine, X represents N—R⁴ and Q, W, Z¹, Z² and R⁴ for the individual compound in question correspond to the radical definitions given in Table 1 (Nos 1 to 1000; corresponding to compounds A5-1 to A5-1000).
A6. Compounds A6-1 to A6-1000 of the general formula (I) in which R¹ and R³ represent hydrogen, R² represents bromine, X represents N—R⁴ and Q, W, Z¹, Z² and R⁴ for the individual compound in question correspond to the radical definitions given in Table 1 (Nos 1 to 1000; corresponding to compounds A6-1 to A6-1000).
A7. Compounds A7-1 to A7-1000 of the general formula (I) in which R¹ and R³ represent hydrogen, R² represents iodine, X represents N—R⁴ and Q, W, Z¹, Z² and R⁴ for the individual compound in question correspond to the radical definitions given in Table 1 (Nos 1 to 1000; corresponding to compounds A7-1 to A7-1000).
A8. Compounds A8-1 to A8-1000 of the general formula (I) in which R¹ and R³ represent hydrogen, R² represents ethyl, X represents N—R⁴ and Q, W, Z¹, Z² and R⁴ for the individual compound in question correspond to the radical definitions given in Table 1 (Nos 1 to 1000; corresponding to compounds A8-1 to A8-1000).
A9. Compounds A9-1 to A9-1000 of the general formula (I) in which R¹ and R³ represent hydrogen, R² represents cyclopropyl, X represents N—R⁴ and Q, W, Z¹, Z² and R⁴ for the individual compound in question correspond to the radical definitions given in Table 1 (Nos 1 to 1000; corresponding to compounds A9-1 to A9-1000).
A10. Compounds A10-1 to A10-1000 of the general formula (I) in which R¹ and R³ represent hydrogen, R² represents trimethylsilylethynyl, X represents N—R⁴ and Q, W, Z¹, Z² and R⁴ for the individual compound in question correspond to the radical definitions given in Table 1 (Nos 1 to 1000; corresponding to compounds A10-1 to A10-1000).
A11. Compounds A11-1 to A11-1000 of the general formula (I) in which R¹ and R³ represent hydrogen, R² represents ethynyl, X represents N—R⁴ and Q, W, Z¹, Z² and R⁴ for the individual compound in question correspond to the radical definitions given in Table 1 (Nos 1 to 1000; corresponding to compounds A11-1 to A11-1000).
A12. Compounds A12-1 to A12-1000 of the general formula (I) in which R¹ and R³ represent hydrogen, R² represents phenyl, X represents N—R⁴ and O, W, Z¹, Z² and R⁴ for the individual compound in question correspond to the radical definitions given in Table 1 (Nos 1 to 1000; corresponding to compounds A12-1 to A12-1000).
A13. Compounds A13-1 to A13-1000 of the general formula (I) in which R¹ and R³ represent hydrogen, R² represents isopropyl, X represents N—R⁴ and Q, W, Z¹, Z² and R⁴ for the individual compound in question correspond to the radical definitions given in Table 1 (Nos 1 to 1000; corresponding to compounds A13-1 to A13-1000).
A14. Compounds A14-1 to A14-1000 of the general formula (I) in which R¹ and R³ represent hydrogen, R² represents difluoromethyl, X represents N—R⁴ and Q, W, Z¹, Z² and R⁴ for the individual compound in question correspond to the radical definitions given in Table 1 (Nos 1 to 1000; corresponding to compounds A14-1 to A14-1000).
A15. Compounds A15-1 to A15-1000 of the general formula (I) in which R¹ and R² represent hydrogen, R³ represents fluorine, X represents N—R⁴ and Q, W, Z¹, Z² and R⁴ for the individual compound in question correspond to the radical definitions given in Table 1 (Nos 1 to 1000; corresponding to compounds A15-1 to A15-1000).
A16. Compounds A16-1 to A16-1000 of the general formula (I) in which R¹ and R² represent hydrogen, R³ represents chlorine, X represents N—R⁴ and Q, W, Z¹, Z² and R⁴ for the individual compound in question correspond to the radical definitions given in Table 1 (Nos 1 to 1000; corresponding to compounds A16-1 to A16-1000).
A17. Compounds A17-1 to A17-1000 of the general formula (I) in which R¹ and R² represent hydrogen, R³ represents bromine, X represents N—R⁴ and Q, W, Z¹, Z² and R⁴ for the individual compound in question correspond to the radical definitions given in Table 1 (Nos 1 to 1000; corresponding to compounds A17-1 to A17-1000).
A18. Compounds A18-1 to A18-1000 of the general formula (I) in which R¹ and R² represent hydrogen, R³ represents trifluoromethyl, X represents N—R⁴ and Q, W, Z¹, Z² and R⁴ for the individual compound in question correspond to the radical definitions given in Table 1 (Nos 1 to 1000; corresponding to compounds A18-1 to A18-1000).
A19. Compounds A19-1 to A19-1000 of the general formula (I) in which R¹ and R² represent hydrogen, R³ represents methyl, X represents N—R⁴ and O, W, Z¹, Z² and R⁴ for the individual compound in question correspond to the radical definitions given in Table 1 (Nos 1 to 1000; corresponding to compounds A19-1 to A19-1000).
A20. Compounds A20-1 to A20-1000 of the general formula (I) in which R² and R³ represent hydrogen, R¹ represents fluorine, X represents N—R⁴ and Q, W, Z¹, Z² and R⁴ for the individual compound in question correspond to the radical definitions given in Table 1 (Nos 1 to 1000; corresponding to compounds A20-1 to A20-1000).
A21. Compounds A21-1 to A21-1000 of the general formula (I) in which R² and R³ represent hydrogen, R¹ represents chlorine, X represents N—R⁴ and Q, W, Z¹, Z² and R⁴ for the individual compound in question correspond to the radical definitions given in Table 1 (Nos 1 to 1000; corresponding to compounds A21-1 to A21-1000).
A22. Compounds A22-1 to A22-1000 of the general formula (I) in which R² and R³ represent hydrogen, R¹ represents bromine, X represents N—R⁴ and Q, W, Z¹, Z² and R⁴ for the individual compound in question correspond to the radical definitions given in Table 1 (Nos 1 to 1000; corresponding to compounds A22-1 to A22-1000).
A23. Compounds A23-1 to A23-1000 of the general formula (I) in which R² and R³ represent hydrogen, R¹ represents trifluoromethyl, X represents N—R⁴ and Q, W, Z¹, Z² and R⁴ for the individual compound in question correspond to the radical definitions given in Table 1 (Nos 1 to 1000; corresponding to compounds A23-1 to A23-1000).
A24. Compounds A24-1 to A24-1000 of the general formula (I) in which R² and R³ represent hydrogen, R¹ represents methyl, and Q, W, Z¹, Z² and R⁴ for the individual compound in question correspond to the radical definitions given in Table 1 (Nos 1 to 1000; corresponding to compounds A24-1 to A24-1000).
A25. Compounds A25-1 to A25-1000 of the general formula (I) in which R¹ and R² represent fluorine, R³ represents hydrogen, X represents N—R⁴ and Q, W, Z¹, Z² and R⁴ for the individual compound in question correspond to the radical definitions given in Table 1 (Nos 1 to 1000; corresponding to compounds A25-1 to A25-1000).
A26. Compounds A26-1 to A26-1000 of the general formula (I) in which R¹ and R² represent methyl, R³ represents hydrogen, X represents N—R⁴ and Q, W, Z¹, Z² and R⁴ for the individual compound in question correspond to the radical definitions given in Table 1 (Nos 1 to 1000; corresponding to compounds A26-1 to A26-1000).
A27. Compounds A27-1 to A27-1000 of the general formula (I) in which R¹ and R² with the atoms to which they are attached form a fused phenyl ring, R³ represents hydrogen, X represents N—R⁴ and Q, W, Z¹, Z² and R⁴ for the individual compound in question correspond to the radical definitions given in Table 1 (Nos 1 to 1000; corresponding to compounds A27-1 to A27-1000).
A28. Compounds A28-1 to A28-1000 of the general formula (I) in which R² and R³ with the atoms to which they are attached form a fused phenyl ring, R¹ represents hydrogen, and Q, W, Z₁, Z² and R⁴ for the individual compound in question correspond to the radical definitions given in Table 1 (Nos 1 to 1000; corresponding to compounds A28-1 to A28-1000).
A29. Compounds A29-1 to A29-1000 of the general formula (I) in which R¹ and R³ represent hydrogen, R² represents methoxy, X represents N—R⁴ and Q, W, Z¹, Z² and R⁴ for the individual compound in question correspond to the radical definitions given in Table 1 (Nos 1 to 1000; corresponding to compounds A29-1 to A29-1000).
A30. Compounds A30-1 to A30-1000 of the general formula (I) in which R¹ and R³ represent hydrogen, R² represents trifluoromethoxy, X represents N—R⁴ and Q, W, Z¹, Z² and R⁴ for the individual compound in question correspond to the radical definitions given in Table 1 (Nos 1 to 1000; corresponding to compounds A30-1 to A30-1000).
A31. Compounds A31-1 to A31-1000 of the general formula (I) in which R¹ and R³ represent hydrogen, R² represents trifluoromethylthio, X represents N—R⁴ and Q, W, Z¹, Z² and R⁴ for the individual compound in question correspond to the radical definitions given in Table 1 (Nos 1 to 1000; corresponding to compounds A31-1 to A31-1000).
A32. Compounds A32-1 to A32-1000 of the general formula (I) in which R¹ and R³ represent hydrogen, R² represents difluoromethoxy, X represents N—R⁴ and Q, W, Z¹, Z² and R⁴ for the individual compound in question correspond to the radical definitions given in Table 1 (Nos 1 to 1000; corresponding to compounds A32-1 to A32-1000).
A33. Compounds A33-1 to A33-1000 of the general formula (I) in which R¹ and R³ represent hydrogen, R² represents ethenyl, X represents N—R⁴ and Q, W, Z¹, Z² and R⁴ for the individual compound in question correspond to the radical definitions given in Table 1 (Nos 1 to 1000; corresponding to compounds A33-1 to A33-1000).
A34. Compounds A34-1 to A34-1000 of the general formula (I) in which R¹ and R³ represent hydrogen, R² represents cyclobutyl, X represents N—R⁴ and Q, W, Z¹, Z² and R⁴ for the individual compound in question correspond to the radical definitions given in Table 1 (Nos 1 to 1000; corresponding to compounds A34-1 to A34-1000).
A35. Compounds A35-1 to A35-1000 of the general formula (I) in which R¹ and R³ represent hydrogen, R² represents cyclopentyl, X represents N—R⁴ and Q, W, Z¹, Z² and R⁴ for the individual compound in question correspond to the radical definitions given in Table 1 (Nos 1 to 1000; corresponding to compounds A35-1 to A35-1000).
A36. Compounds A36-1 to A36-1000 of the general formula (I) in which R¹ and R³ represent hydrogen, R² represents 4-chlorophenyl, X represents N—R⁴ and Q, W, Z¹, Z² and R⁴ for the individual compound in question correspond to the radical definitions given in Table 1 (Nos 1 to 1000; corresponding to compounds A36-1 to A36-1000).
A37. Compounds A37-1 to A37-1000 of the general formula (I) in which R¹ and R³ represent hydrogen, R² represents 2-thiophenyl, X represents N—R⁴ and Q, W, Z¹, Z² and R⁴ for the individual compound in question correspond to the radical definitions given in Table 1 (Nos 1 to 1000; corresponding to compounds A37-1 to A37-1000).
A38. Compounds A38-1 to A38-1000 of the general formula (I) in which R¹ and R³ represent hydrogen, R² represents 2-furanyl, X represents N—R⁴ and Q, W, Z¹, Z² and R⁴ for the individual compound in question correspond to the radical definitions given in Table 1 (Nos 1 to 1000; corresponding to compounds A38-1 to A38-1000).
A39. Compounds A39-1 to A39-1000 of the general formula (I) in which R¹ and R³ represent hydrogen, R² represents cyclohexyl, X represents N—R⁴ and Q, W, Z¹, Z² and R⁴ for the individual compound in question correspond to the radical definitions given in Table 1 (Nos 1 to 1000; corresponding to compounds A39-1 to A39-1000).
A40. Compounds A40-1 to A40-1000 of the general formula (I) in which R¹ and R³ represent hydrogen, R² represents 2-tetrahydrofuranyl, X represents N—R⁴ and Q, W, Z¹, Z² and R⁴ for the individual compound in question correspond to the radical definitions given in Table 1 (Nos 1 to 1000; corresponding to compounds A40-1 to A40-1000).
A41. Compounds A41-1 to A41-1000 of the general formula (I) in which R¹ and R³ represent hydrogen, R² represents methoxyethoxy, X represents N—R⁴ and Q, W, Z¹, Z² and R⁴ for the individual compound in question correspond to the radical definitions given in Table 1 (Nos 1 to 1000; corresponding to compounds A41-1 to A41-1000).
B1. Compounds B1-1 to B1-700 of the general formula (I) in which R¹, R² and R³ represent hydrogen and Q, W, Z¹ and Z² correspond to the definitions (Nos 1 to 700; corresponding to compounds B1-1 to B1-700) in Table 2 below

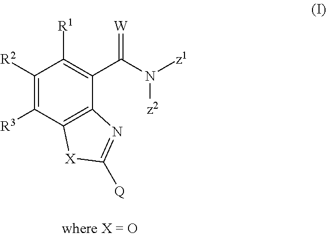

TABLE 2

| No. | Q | W | Z¹ | Z² |
|---|---|---|---|---|
| 1 | 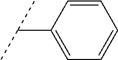 | O | H | H |
| 2 | 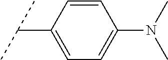 | O | H | H |
| 3 | 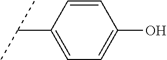 | O | H | H |
| 4 | 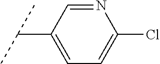 | O | H | H |
| 5 | 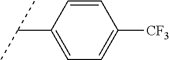 | O | H | H |
| 6 | 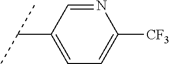 | O | H | H |
| 7 | 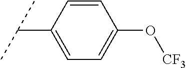 | O | H | H |
| 8 | 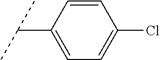 | O | H | H |
| 9 | 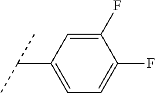 | O | H | H |
| 10 | 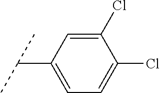 | O | H | H |
| 11 | 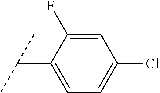 | O | H | H |
| 12 | 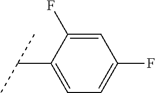 | O | H | H |
| 13 | 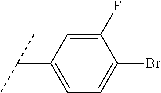 | O | H | H |
| 14 | 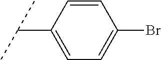 | O | H | H |
| 15 | 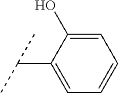 | O | H | H |
| 16 | 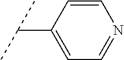 | O | H | H |
| 17 | 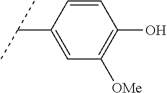 | O | H | H |
| 18 | 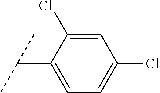 | O | H | H |
| 19 | 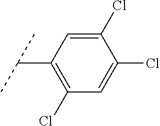 | O | H | H |
| 20 | 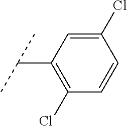 | O | H | H |
| 21 | 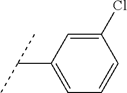 | O | H | H |
| 22 | 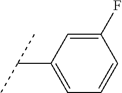 | O | H | H |
| 23 | 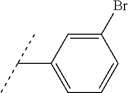 | O | H | H |
| 24 | 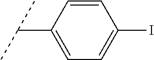 | O | H | H |
| 25 | 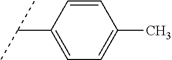 | O | H | H |
| 26 | 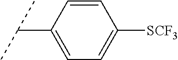 | O | H | H |
| 27 | 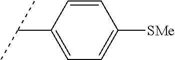 | O | H | H |
| 28 | 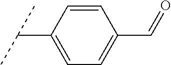 | O | H | H |
| 29 | 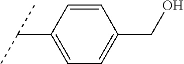 | O | H | H |
| 30 | 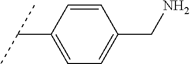 | O | H | H |
| 31 | 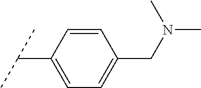 | O | H | H |
| 32 | 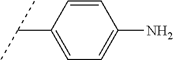 | O | H | H |
| 33 | 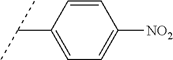 | O | H | H |
| 34 | 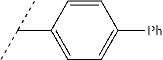 | O | H | H |
| 35 | 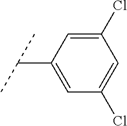 | O | H | H |
| 36 | 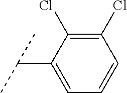 | O | H | H |
| 37 | 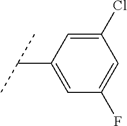 | O | H | H |
| 38 | 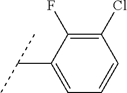 | O | H | H |
| 39 | 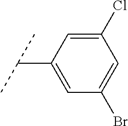 | O | H | H |
| 40 | 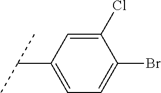 | O | H | H |
| 41 | 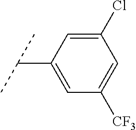 | O | H | H |
| 42 | 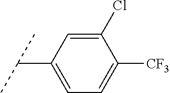 | O | H | H |
| 43 | 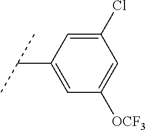 | O | H | H |
| 44 | 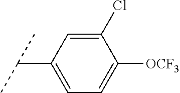 | O | H | H |
| 45 | 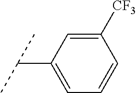 | O | H | H |
| 46 | 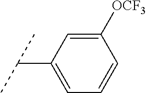 | O | H | H |
| 47 | 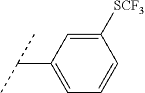 | O | H | H |
| 48 | 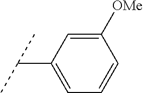 | O | H | H |
| 49 | 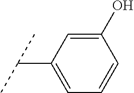 | O | H | H |
| 50 | 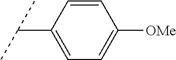 | O | H | H |
| 51 | 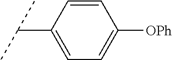 | O | H | H |
| 52 | 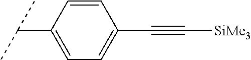 | O | H | H |
| 53 | 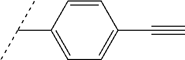 | O | H | H |
| 54 | 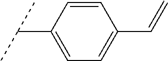 | O | H | H |
| 55 | 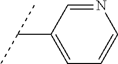 | O | H | H |
| 56 | 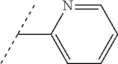 | O | H | H |
| 57 | 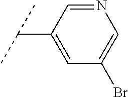 | O | H | H |
| 58 | 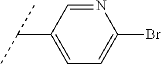 | O | H | H |
| 59 | 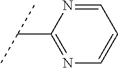 | O | H | H |
| 60 | 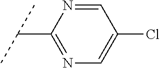 | O | H | H |
| 61 | 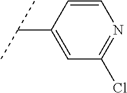 | O | H | H |
| 62 | 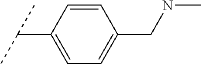 | O | H | H |
| 63 | 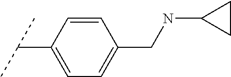 | O | H | H |
| 64 | 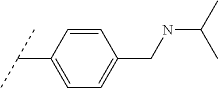 | O | H | H |
| 65 | 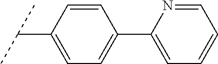 | O | H | H |
| 66 | 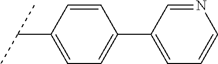 | O | H | H |
| 67 | 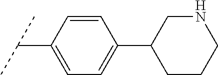 | O | H | H |
| 68 | 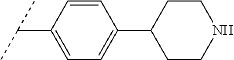 | O | H | H |
| 69 | 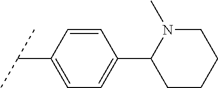 | O | H | H |
| 70 | 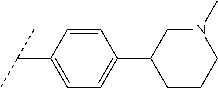 | O | H | H |
| 71 | 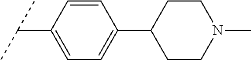 | O | H | H |
| 72 | 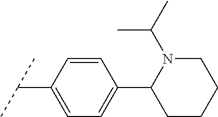 | O | H | H |
| 73 | 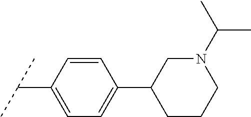 | O | H | H |
| 74 | 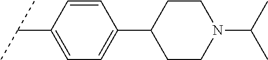 | O | H | H |
| 75 | 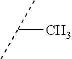 | O | H | H |
| 76 | 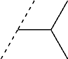 | O | H | H |
| 77 | 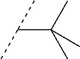 | O | H | H |
| 78 | 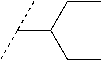 | O | H | H |
| 79 | 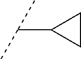 | O | H | H |
| 80 | 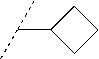 | O | H | H |
| 81 | 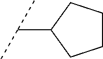 | O | H | H |
| 82 | 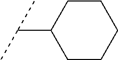 | O | H | H |
| 83 | 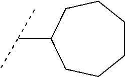 | O | H | H |
| 84 | 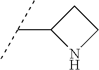 | O | H | H |
| 85 | 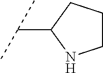 | O | H | H |
| 86 | 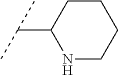 | O | H | H |
| 87 | 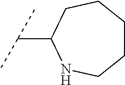 | O | H | H |
| 88 | 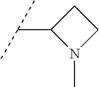 | O | H | H |
| 89 | 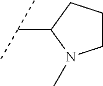 | O | H | H |
| 90 | 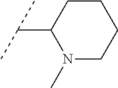 | O | H | H |
| 91 | 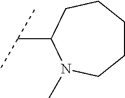 | O | H | H |
| 92 | 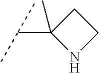 | O | H | H |
| 93 | 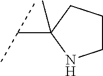 | O | H | H |
| 94 | 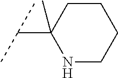 | O | H | H |
| 95 | 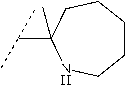 | O | H | H |
| 96 | 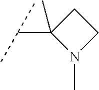 | O | H | H |
| 97 | 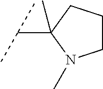 | O | H | H |
| 98 | 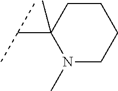 | O | H | H |
| 99 | 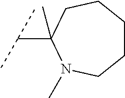 | O | H | H |
| 100 | 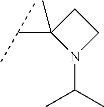 | O | H | H |
| 101 | 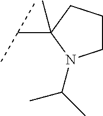 | O | H | H |
| 102 | 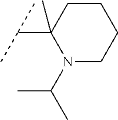 | O | H | H |
| 103 | 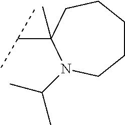 | O | H | H |
| 104 | 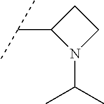 | O | H | H |
| 105 | 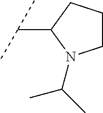 | O | H | H |
| 106 | 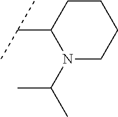 | O | H | H |
| 107 | 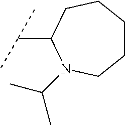 | O | H | H |
| 108 | 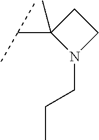 | O | H | H |
| 109 | 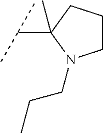 | O | H | H |
| 110 | 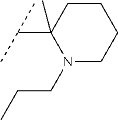 | O | H | H |
| 111 | 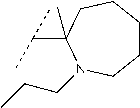 | O | H | H |
| 112 | 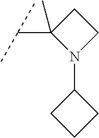 | O | H | H |
| 113 | 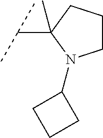 | O | H | H |
| 114 | 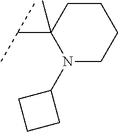 | O | H | H |
| 115 | 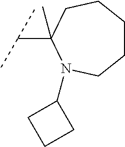 | O | H | H |
| 116 | 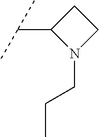 | O | H | H |
| 117 | 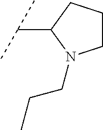 | O | H | H |
| 118 | 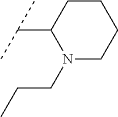 | O | H | H |
| 119 | 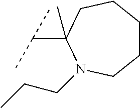 | O | H | H |
| 120 | 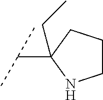 | O | H | H |
| 121 | 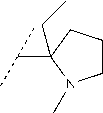 | O | H | H |
| 122 | 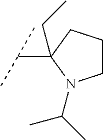 | O | H | H |
| 123 | 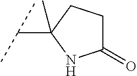 | O | H | H |
| 124 | 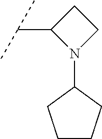 | O | H | H |
| 125 | 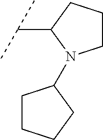 | O | H | H |
| 126 | 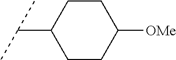 | O | H | H |
| 127 | 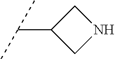 | O | H | H |
| 128 | 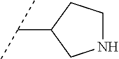 | O | H | H |
| 129 | 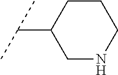 | O | H | H |
| 130 | 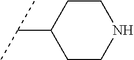 | O | H | H |
| 131 | 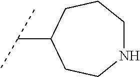 | O | H | H |
| 132 | 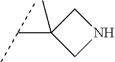 | O | H | H |
| 133 | 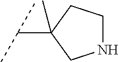 | O | H | H |
| 134 | 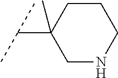 | O | H | H |
| 135 | 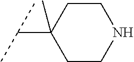 | O | H | H |
| 136 | 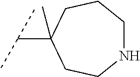 | O | H | H |
| 137 | 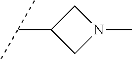 | O | H | H |
| 138 | 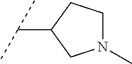 | O | H | H |
| 139 | 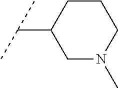 | O | H | H |
| 140 | 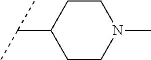 | O | H | H |
| 141 | 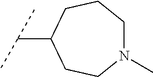 | O | H | H |
| 142 | 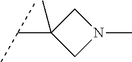 | O | H | H |
| 143 | 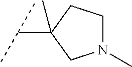 | O | H | H |
| 144 | 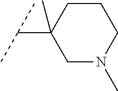 | O | H | H |
| 145 | 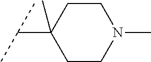 | O | H | H |
| 146 | 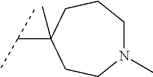 | O | H | H |
| 147 | 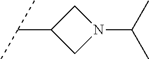 | O | H | H |
| 148 | 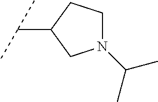 | O | H | H |
| 149 | 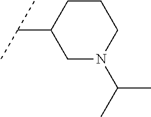 | O | H | H |
| 150 | 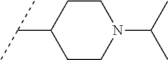 | O | H | H |
| 151 | 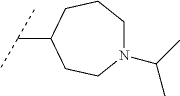 | O | H | H |
| 152 | 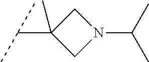 | O | H | H |
| 153 | 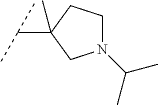 | O | H | H |
| 154 | 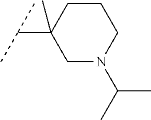 | O | H | H |
| 155 | 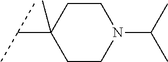 | O | H | H |
| 156 | 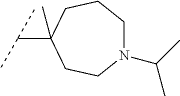 | O | H | H |
| 157 | 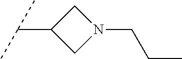 | O | H | H |
| 158 | 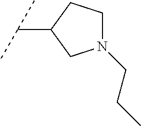 | O | H | H |
| 159 | 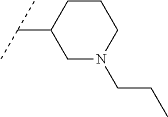 | O | H | H |
| 160 | 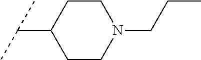 | O | H | H |
| 161 | 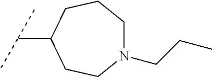 | O | H | H |
| 162 | 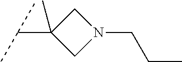 | O | H | H |
| 163 | 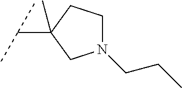 | O | H | H |
| 164 | 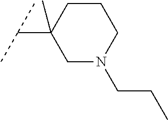 | O | H | H |
| 165 | 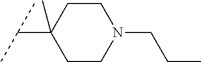 | O | H | H |
| 166 | 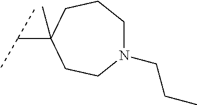 | O | H | H |
| 167 | 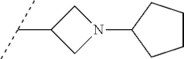 | O | H | H |
| 168 | 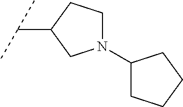 | O | H | H |
| 169 | 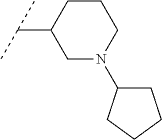 | O | H | H |
| 170 | 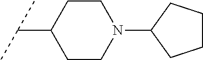 | O | H | H |
| 171 | 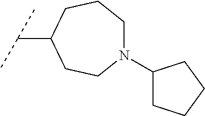 | O | H | H |
| 172 | 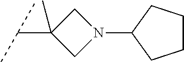 | O | H | H |
| 173 | 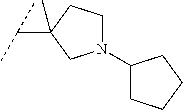 | O | H | H |
| 174 | 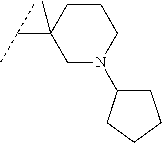 | O | H | H |
| 175 | 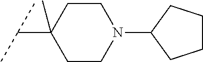 | O | H | H |
| 176 | 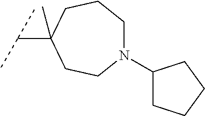 | O | H | H |
| 177 | 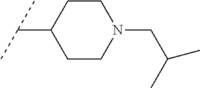 | O | H | H |
| 178 | 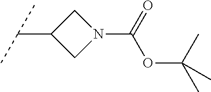 | O | H | H |
| 179 | 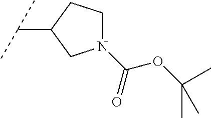 | O | H | H |
| 180 | 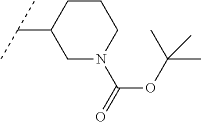 | O | H | H |
| 181 | 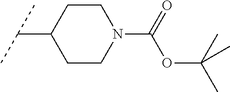 | O | H | H |
| 182 | 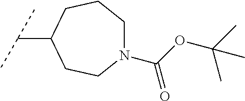 | O | H | H |
| 183 | 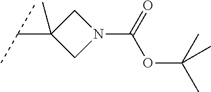 | O | H | H |
| 184 | 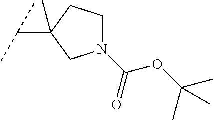 | O | H | H |
| 185 | 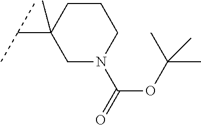 | O | H | H |
| 186 | 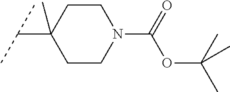 | O | H | H |
| 187 | 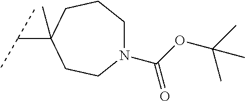 | O | H | H |
| 188 | 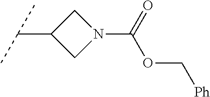 | O | H | H |
| 189 | 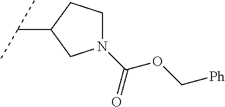 | O | H | H |
| 190 | 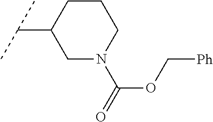 | O | H | H |
| 191 | 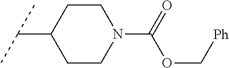 | O | H | H |
| 192 | 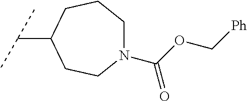 | O | H | H |
| 193 | 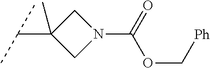 | O | H | H |
| 194 | 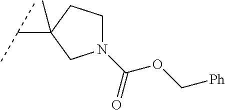 | O | H | H |
| 195 | 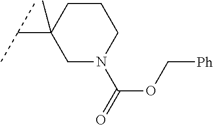 | O | H | H |
| 196 | 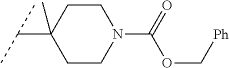 | O | H | H |
| 197 | 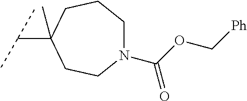 | O | H | H |
| 198 | 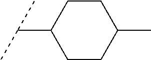 | O | H | H |
| 199 | 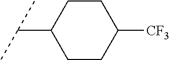 | O | H | H |
| 200 | 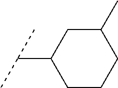 | O | H | H |
| 201 | 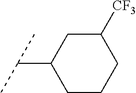 | O | H | H |
| 202 | 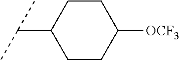 | O | H | H |
| 203 | 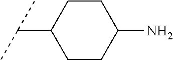 | O | H | H |
| 204 | 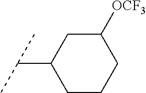 | O | H | H |
| 205 | 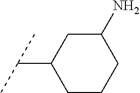 | O | H | H |
| 206 | 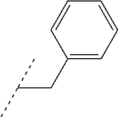 | O | H | H |
| 207 | 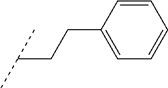 | O | H | H |
| 208 | 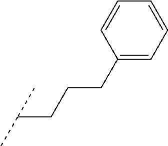 | O | H | H |
| 209 | 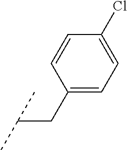 | O | H | H |
| 210 | 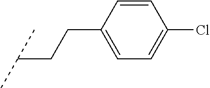 | O | H | H |
| 211 | 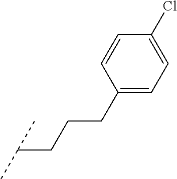 | O | H | H |
| 212 | 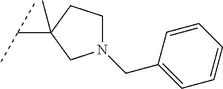 | O | H | H |
| 213 | 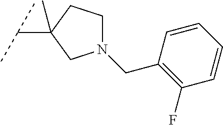 | O | H | H |
| 214 | 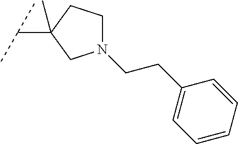 | O | H | H |
| 215 | 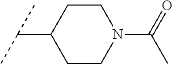 | O | H | H |
| 216 | 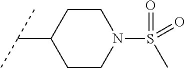 | O | H | H |
| 217 | 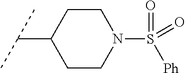 | O | H | H |
| 218 | 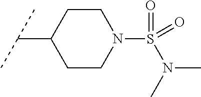 | O | H | H |
| 219 | 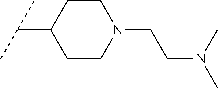 | O | H | H |
| 220 | 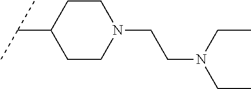 | O | H | H |
| 221 | 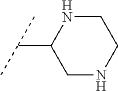 | O | H | H |
| 222 | 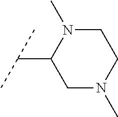 | O | H | H |
| 223 | 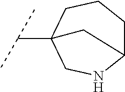 | O | H | H |
| 224 | 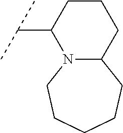 | O | H | H |
| 225 | 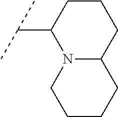 | O | H | H |
| 226 | 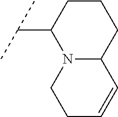 | O | H | H |
| 227 | 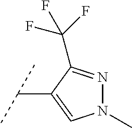 | O | H | H |
| 228 | 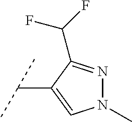 | O | H | H |
| 229 | 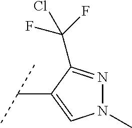 | O | H | H |
| 230 | 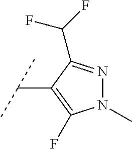 | O | H | H |
| 231 | 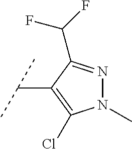 | O | H | H |
| 232 | 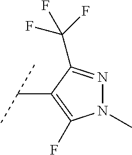 | O | H | H |
| 233 | 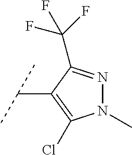 | O | H | H |
| 234 | 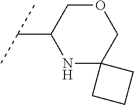 | O | H | H |
| 235 | 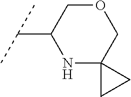 | O | H | H |
| 236 | 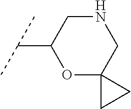 | O | H | H |
| 237 | 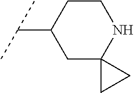 | O | H | H |
| 238 | 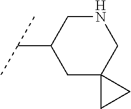 | O | H | H |
| 239 | 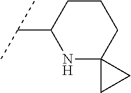 | O | H | H |
| 240 | 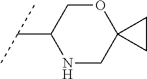 | O | H | H |
| 241 | 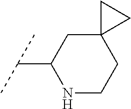 | O | H | H |
| 242 | 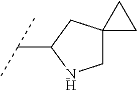 | O | H | H |
| 243 | 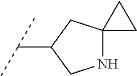 | O | H | H |
| 244 | 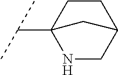 | O | H | H |
| 245 | 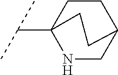 | O | H | H |
| 246 | 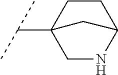 | O | H | H |
| 247 | 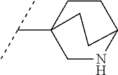 | O | H | H |
| 248 | 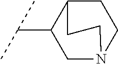 | O | H | H |
| 249 | 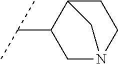 | O | H | H |
| 250 | 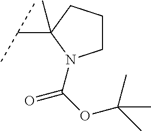 | O | H | H |
| 251 | 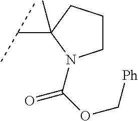 | O | H | H |
| 252 | 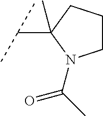 | O | H | H |
| 253 | 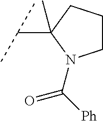 | O | H | H |
| 254 | 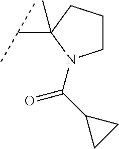 | O | H | H |
| 255 | 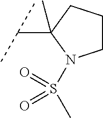 | O | H | H |
| 256 | 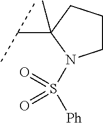 | O | H | H |
| 257 | 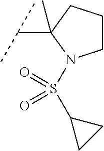 | O | H | H |
| 258 | 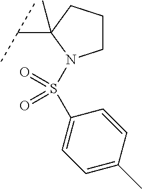 | O | H | H |
| 259 | 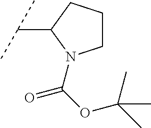 | O | H | H |
| 260 | 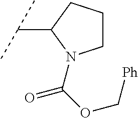 | O | H | H |
| 261 | 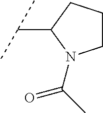 | O | H | H |
| 262 | 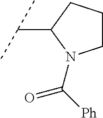 | O | H | H |
| 263 | 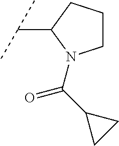 | O | H | H |
| 264 | 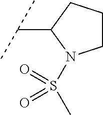 | O | H | H |
| 265 | 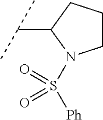 | O | H | H |
| 266 | 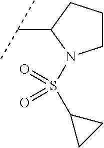 | O | H | H |
| 267 | 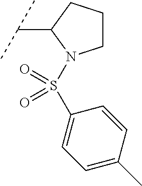 | O | H | H |
| 268 | 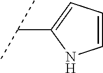 | O | H | H |
| 269 | 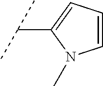 | O | H | H |
| 270 | 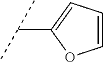 | O | H | H |
| 271 | 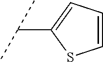 | O | H | H |
| 272 | 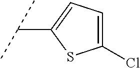 | O | H | H |
| 273 | 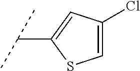 | O | H | H |
| 274 | 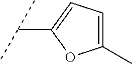 | O | H | H |
| 275 | 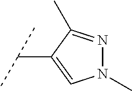 | O | H | H |
| 276 | 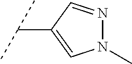 | O | H | H |
| 277 | 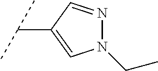 | O | H | H |
| 278 | 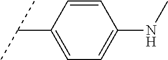 | O | H | H |
| 279 | 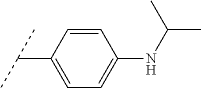 | O | H | H |
| 280 | 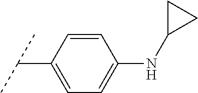 | O | H | H |
| 281 | 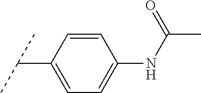 | O | H | H |
| 282 | 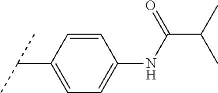 | O | H | H |
| 283 | 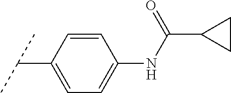 | O | H | H |
| 284 | 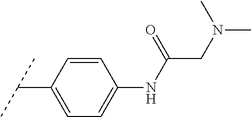 | O | H | H |
| 285 | 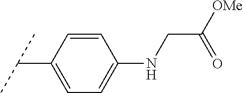 | O | H | H |
| 286 | 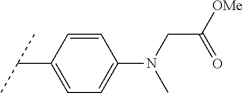 | O | H | H |
| 287 | 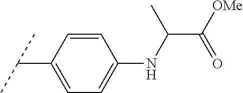 | O | H | H |
| 288 | 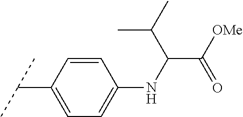 | O | H | H |
| 289 | 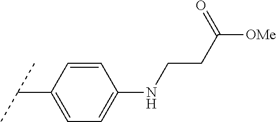 | O | H | H |
| 290 | 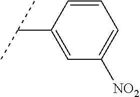 | O | H | H |
| 291 | 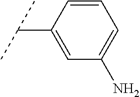 | O | H | H |
| 292 | 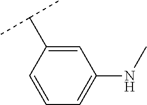 | O | H | H |
| 293 | 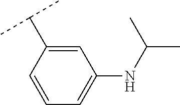 | O | H | H |
| 294 | 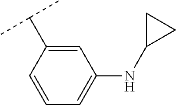 | O | H | H |
| 295 | 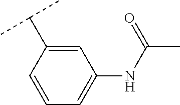 | O | H | H |
| 296 | 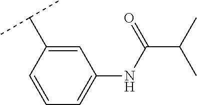 | O | H | H |
| 297 | 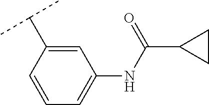 | O | H | H |
| 298 | 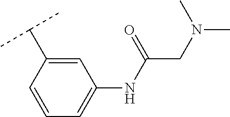 | O | H | H |
| 299 | 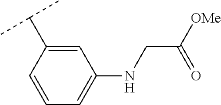 | O | H | H |
| 300 | 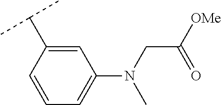 | O | H | H |
| 301 | 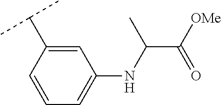 | O | H | H |
| 302 | 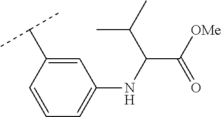 | O | H | H |
| 303 | 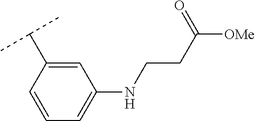 | O | H | H |
| 304 | 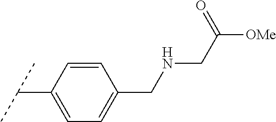 | O | H | H |
| 305 | 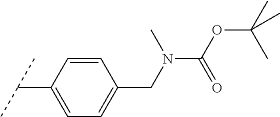 | O | H | H |
| 306 | 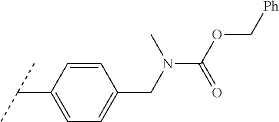 | O | H | H |
| 307 | 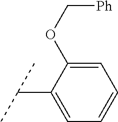 | O | H | H |
| 308 | 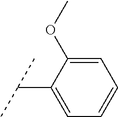 | O | H | H |
| 309 | 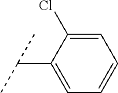 | O | H | H |
| 310 | 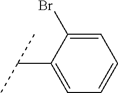 | O | H | H |
| 311 | 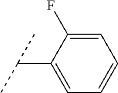 | O | H | H |
| 312 | 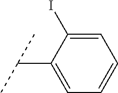 | O | H | H |
| 313 | 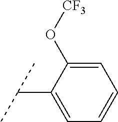 | O | H | H |
| 314 | 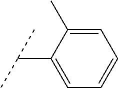 | O | H | H |
| 315 | 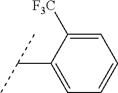 | O | H | H |
| 316 | 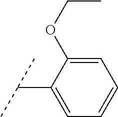 | O | H | H |
| 317 | 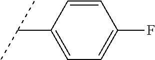 | O | H | H |
| 318 | 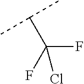 | O | H | H |
| 319 | 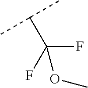 | O | H | H |
| 320 | 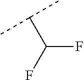 | O | H | H |
| 321 | 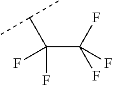 | O | H | H |
| 322 | 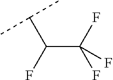 | O | H | H |
| 323 | 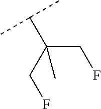 | O | H | H |
| 324 | 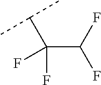 | O | H | H |
| 325 | 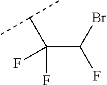 | O | H | H |
| 326 | 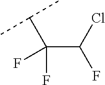 | O | H | H |
| 327 | 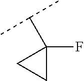 | O | H | H |
| 328 | 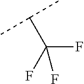 | O | H | H |
| 329 | 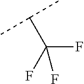 | O | 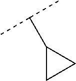 | H |
| 330 | 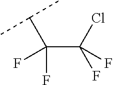 | O | H | H |
| 331 | 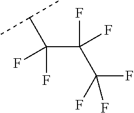 | O | H | H |
| 332 | 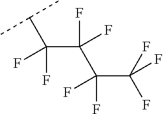 | O | H | H |
| 333 | 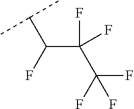 | O | H | H |
| 334 | 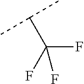 | O | 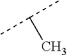 | H |
| 335 | 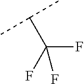 | O | 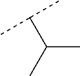 | H |
| 336 | 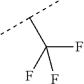 | O | 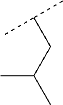 | H |
| 337 | 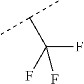 | O | 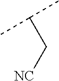 | H |
| 338 | 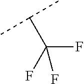 | O | 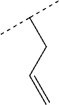 | H |
| 339 | 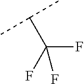 | O | 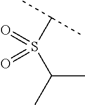 | H |
| 340 | 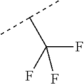 | O | 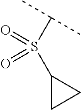 | H |
| 347 | 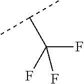 | O | 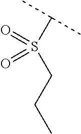 | H |
| 348 | 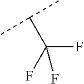 | O | 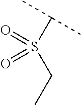 | H |
| 349 | 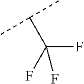 | O | 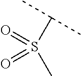 | H |
| 350 | 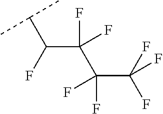 | O | H | H |
| 351 | 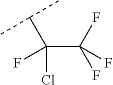 | O | H | H |
| 352 | 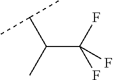 | O | H | H |
| 353 | 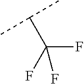 | S | H | H |
| 354 | 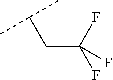 | O | H | H |
| 355 | 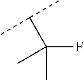 | O | H | H |
| 356 | 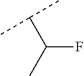 | O | H | H |
| 357 | 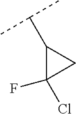 | O | H | H |
| 358 | 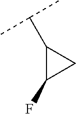 | O | H | H |
| 359 | 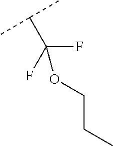 | O | H | H |
| 360 | 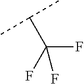 | O | 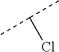 | H |
| 361 | 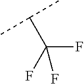 | O | 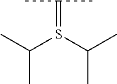 | |
| 362 | 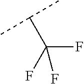 | O | 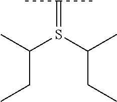 | |
| 363 | 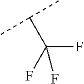 | O | 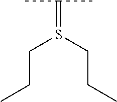 | |
| 364 | 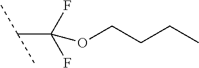 | O | H | H |
| 365 | 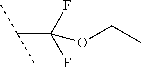 | O | H | H |
| 366 | 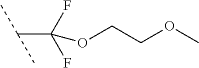 | O | H | H |
| 381 | 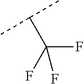 | O | 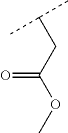 | H |
| 382 | 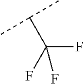 | O | 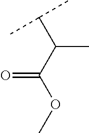 | H |
| 383 | 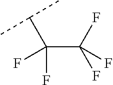 | S | H | H |
| 384 | 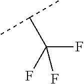 | O | 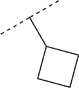 | H |
| 385 | 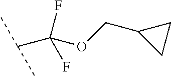 | O | H | H |
| 386 | 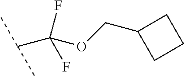 | O | H | H |
| 387 | 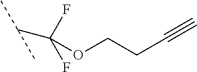 | O | H | H |
| 388 | 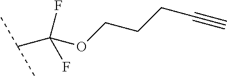 | O | H | H |
| 389 | 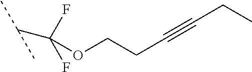 | O | H | H |
| 390 | 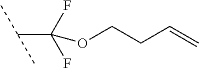 | O | H | H |
| 391 | 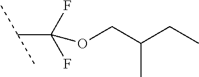 | O | H | H |
| 392 | 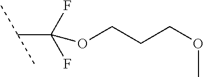 | O | H | H |
| 393 | 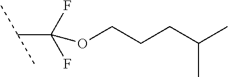 | O | H | H |
| 394 | 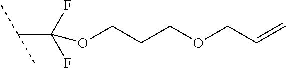 | O | H | H |
| 395 | 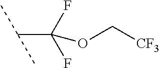 | O | H | H |
| 396 | 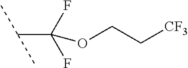 | O | H | H |
| 397 | 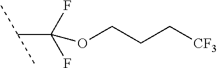 | O | H | H |
| 398 | 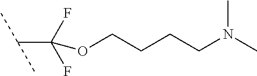 | O | H | H |
| 399 | 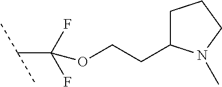 | O | H | H |
| 400 | 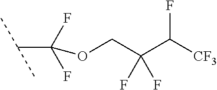 | O | H | H |
| 401 | 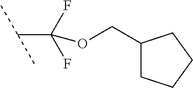 | O | H | H |
| 402 | 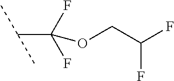 | O | H | H |
| 403 | 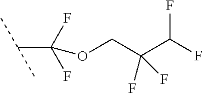 | O | H | H |
| 404 | 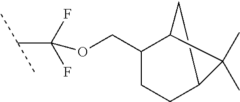 | O | H | H |
| 405 | 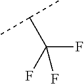 | O | 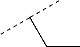 | H |
| 406 | 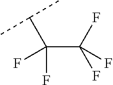 | O | 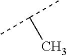 | H |
| 407 | 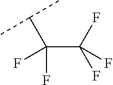 | O | 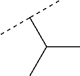 | H |
| 409 | 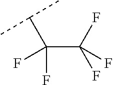 | O | 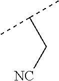 | H |
| 410 | 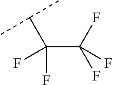 | O | 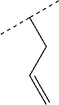 | H |
| 411 | 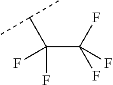 | O | 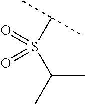 | H |
| 412 | 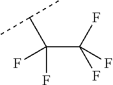 | O | 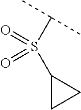 | H |
| 413 | 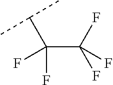 | O | 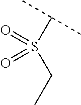 | H |
| 414 | 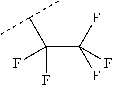 | O | 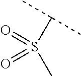 | H |
| 415 | 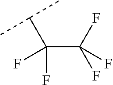 | O | 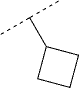 | H |
| 416 | 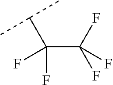 | O | 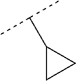 | H |
| 417 | 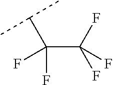 | O | 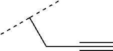 | H |
| 418 | 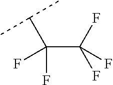 | O | 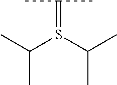 | |
| 419 | 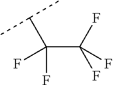 | O | 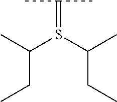 | |
| 420 | 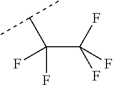 | O | 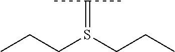 | |
| 421 | 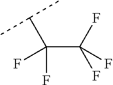 | O | 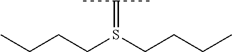 | |
| 422 | 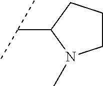 | O | 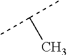 | H |
| 423 | 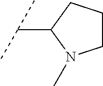 | O | 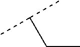 | H |
| 424 | 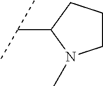 | O | 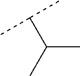 | H |
| 425 | 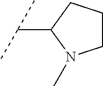 | O | 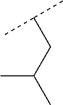 | H |
| 426 | 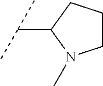 | O | 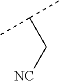 | H |
| 427 | 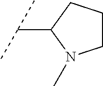 | O | 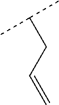 | H |
| 428 | 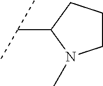 | O | 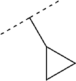 | H |
| 429 | 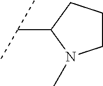 | O | 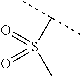 | H |
| 430 | 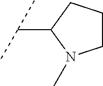 | O | 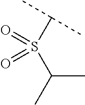 | H |
| 431 | 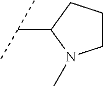 | O | 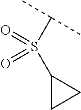 | H |
| 432 | 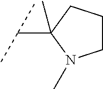 | O | 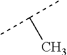 | H |
| 433 | 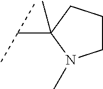 | O | 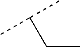 | H |
| 434 | 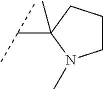 | O | 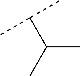 | H |
| 435 | 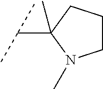 | O | 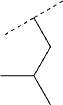 | H |
| 436 | 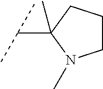 | O | 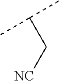 | H |
| 437 | 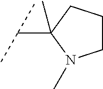 | O | 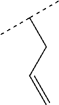 | H |
| 438 | 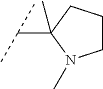 | O | 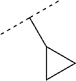 | H |
| 439 | 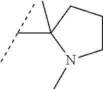 | O | 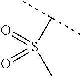 | H |
| 440 | 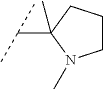 | O | 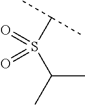 | H |
| 441 | 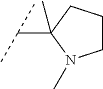 | O | 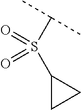 | H |
| 442 | 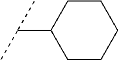 | O | 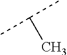 | H |
| 443 | 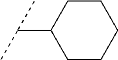 | O | 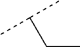 | H |
| 444 | 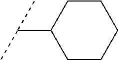 | O | 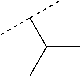 | H |
| 445 | 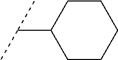 | O | 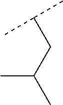 | H |
| 446 | 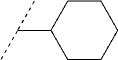 | O | 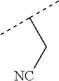 | H |
| 447 | 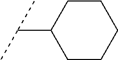 | O | 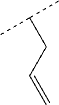 | H |
| 448 | 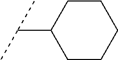 | O | 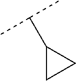 | H |
| 449 | 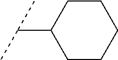 | O | 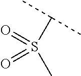 | H |
| 450 | 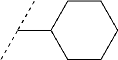 | O | 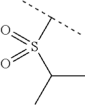 | H |
| 451 | 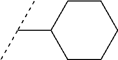 | O | 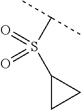 | H |
| 452 | 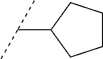 | O | 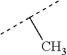 | H |
| 453 | 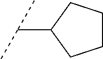 | O | 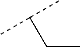 | H |
| 454 | 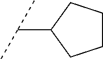 | O | 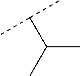 | H |
| 455 | 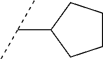 | O | 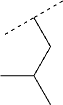 | H |
| 456 | 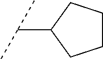 | O | 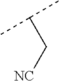 | H |
| 457 | 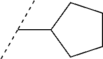 | O | 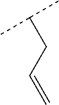 | H |
| 458 | 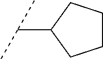 | O | 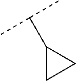 | H |
| 459 | 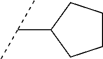 | O | 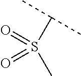 | H |
| 460 | 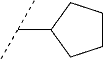 | O | 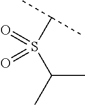 | H |
| 461 | 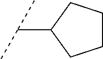 | O | 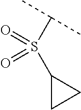 | H |
| 462 | 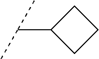 | O | 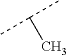 | H |
| 463 | 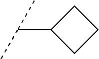 | O | 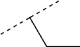 | H |
| 464 | 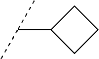 | O | 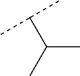 | H |
| 465 | 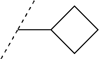 | O | 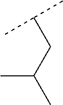 | H |
| 466 | 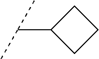 | O | 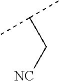 | H |
| 467 | 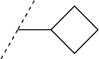 | O | 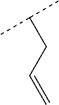 | H |
| 468 | 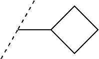 | O | 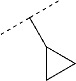 | H |
| 469 | 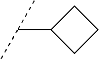 | O | 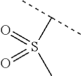 | H |
| 470 | 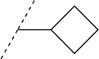 | O | 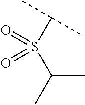 | H |
| 472 | 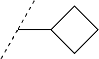 | O | 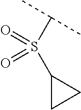 | H |
| 472 | 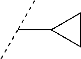 | O | 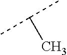 | H |
| 473 | 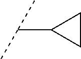 | O | 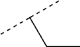 | H |
| 474 | 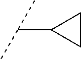 | O | 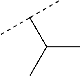 | H |
| 475 | 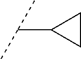 | O | 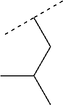 | H |
| 476 | 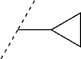 | O | 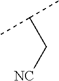 | H |
| 477 | 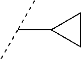 | O | 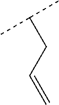 | H |
| 478 | 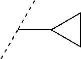 | O | 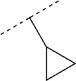 | H |
| 479 | 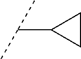 | O | 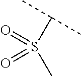 | H |
| 480 | 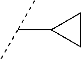 | O | 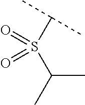 | H |
| 481 | 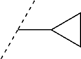 | O | 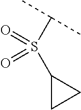 | H |
| 482 | 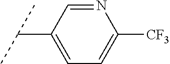 | O | 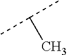 | H |
| 483 | 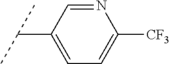 | O | 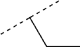 | H |
| 484 | 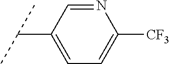 | O | 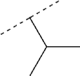 | H |
| 485 | 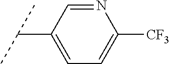 | O | 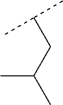 | H |
| 486 | 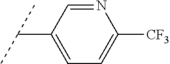 | O | 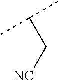 | H |
| 487 | 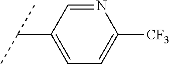 | O | 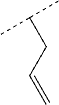 | H |
| 488 | 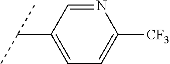 | O | 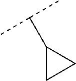 | H |
| 489 | 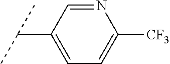 | O | 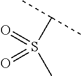 | H |
| 490 | 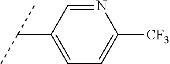 | O | 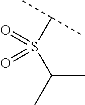 | H |
| 491 | 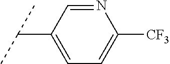 | O | 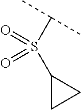 | H |
| 492 | 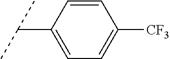 | O | 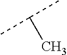 | H |
| 493 | 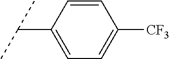 | O | 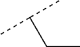 | H |
| 494 | 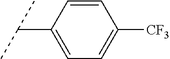 | O | 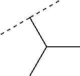 | H |
| 495 | 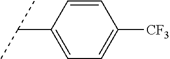 | O | 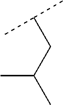 | H |
| 496 | 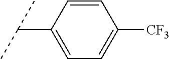 | O | 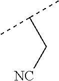 | H |
| 497 | 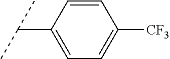 | O | 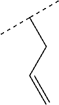 | H |
| 498 | 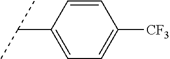 | O | 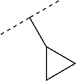 | H |
| 499 | 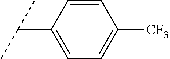 | O | 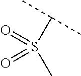 | H |
| 500 | 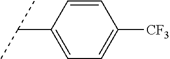 | O | 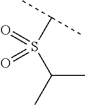 | H |
| 501 | 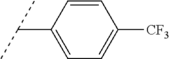 | O | 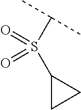 | H |
| 502 | 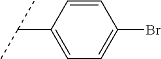 | O | 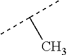 | H |
| 503 | 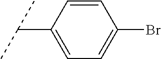 | O | 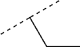 | H |
| 504 | 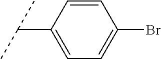 | O | 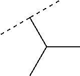 | H |
| 505 | 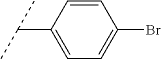 | O | 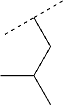 | H |
| 506 | 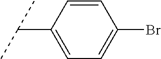 | O | 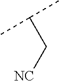 | H |
| 507 | 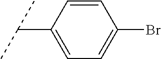 | O | 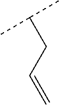 | H |
| 508 | 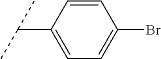 | O | 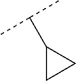 | H |
| 509 | 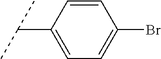 | O | 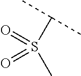 | H |
| 510 | 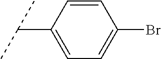 | O | 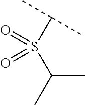 | H |
| 511 | 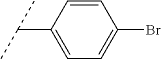 | O | 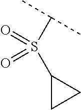 | H |
| 512 | 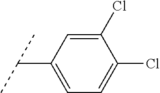 | O | 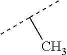 | H |
| 513 | 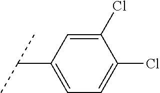 | O | 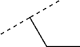 | H |
| 514 | 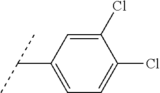 | O | 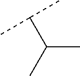 | H |
| 515 | 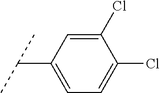 | O | 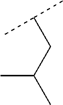 | H |
| 516 | 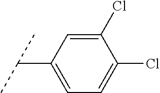 | O | 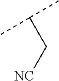 | H |
| 517 | 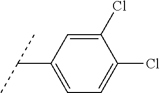 | O | 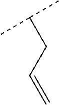 | H |
| 518 | 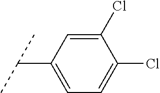 | O | 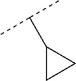 | H |
| 519 | 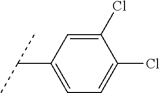 | O | 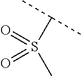 | H |
| 520 | 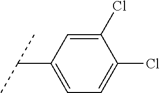 | O | 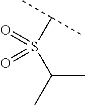 | H |
| 521 | 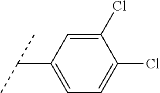 | O | 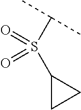 | H |
| 522 | 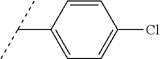 | O | 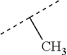 | H |
| 523 | 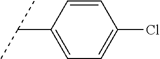 | O | 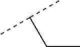 | H |
| 524 | 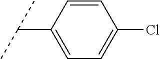 | O | 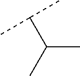 | H |
| 525 | 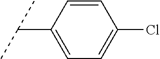 | O | 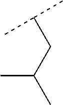 | H |
| 526 | 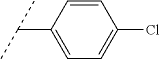 | O | 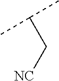 | H |
| 527 | 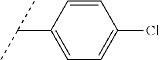 | O | 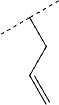 | H |
| 528 | 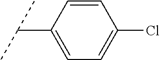 | O | 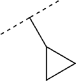 | H |
| 529 | 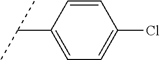 | O | 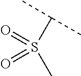 | H |
| 530 | 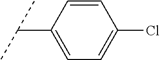 | O | 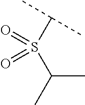 | H |
| 531 | 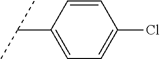 | O | 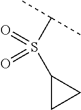 | H |
| 532 | 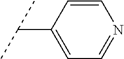 | O | 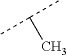 | H |
| 533 | 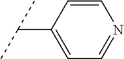 | O | 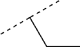 | H |
| 534 | 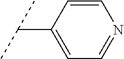 | O | 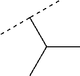 | H |
| 535 | 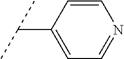 | O | 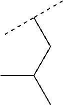 | H |
| 536 | 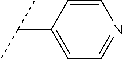 | O | 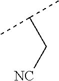 | H |
| 537 | 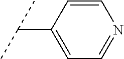 | O | 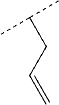 | H |
| 538 | 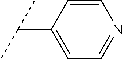 | O | 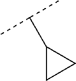 | H |
| 539 | 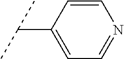 | O | 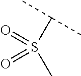 | H |
| 540 | 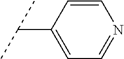 | O | 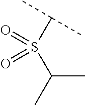 | H |
| 541 | 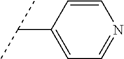 | O | 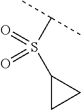 | H |
| 542 | 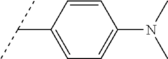 | O | 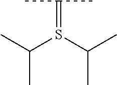 | |
| 543 | 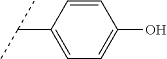 | O | 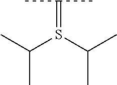 | |
| 544 | 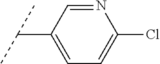 | O | 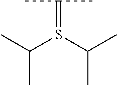 | |
| 545 | 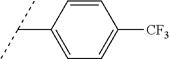 | O | 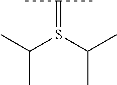 | |
| 546 | 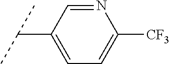 | O | 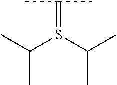 | |
| 547 | 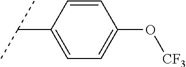 | O | 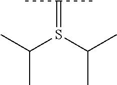 | |
| 548 | 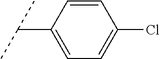 | O | 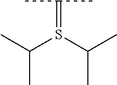 | |
| 549 | 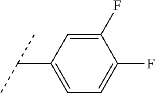 | O | 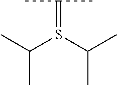 | |
| 550 | 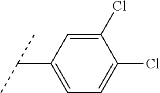 | O | 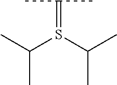 | |
| 551 | 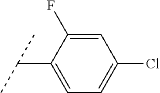 | O | 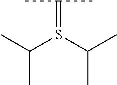 | |
| 552 | 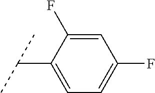 | O | 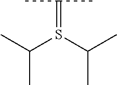 | |
| 553 | 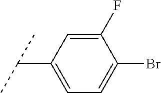 | O | 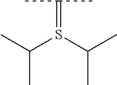 | |
| 554 | 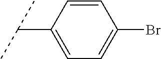 | O | 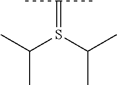 | |
| 555 | 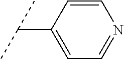 | O | 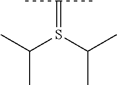 | |
| 556 | 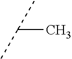 | O | 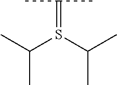 | |
| 557 | 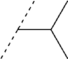 | O | 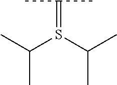 | |
| 558 | 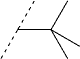 | O | 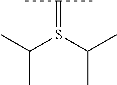 | |
| 559 | 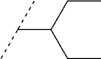 | O | 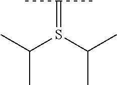 | |
| 560 | 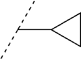 | O | 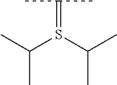 | |
| 561 | 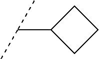 | O | 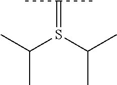 | |
| 562 | 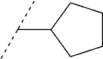 | O | 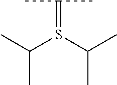 | |
| 563 | 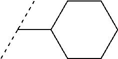 | O | 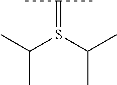 | |
| 564 | 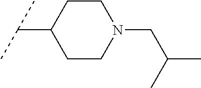 | O | 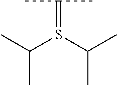 | |
| 565 | 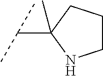 | O | 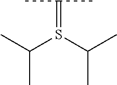 | |
| 566 | 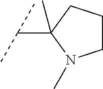 | O | 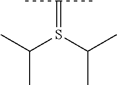 | |
| 567 | 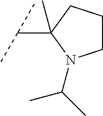 | O | 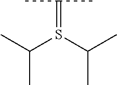 | |
| 568 | 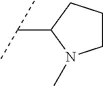 | O | 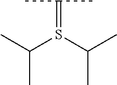 | |
| 569 | 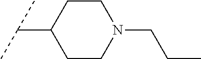 | O | 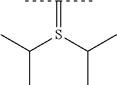 | |
| 570 | 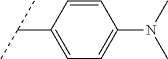 | O | 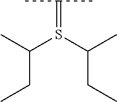 | |
| 571 | 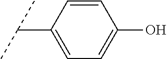 | O | 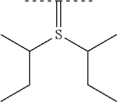 | |
| 572 | 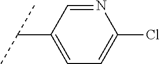 | O | 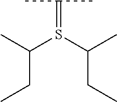 | |
| 573 | 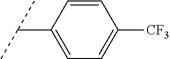 | O | 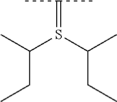 | |
| 574 | 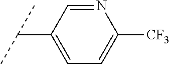 | O | 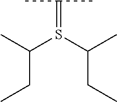 | |
| 575 | 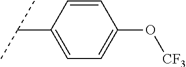 | O | 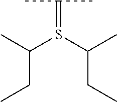 | |
| 576 | 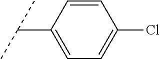 | O | 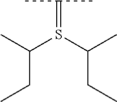 | |
| 577 | 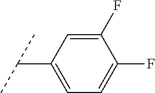 | O | 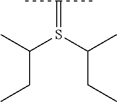 | |
| 578 | 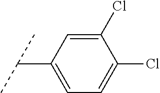 | O | 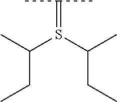 | |
| 579 | 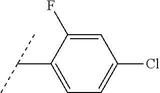 | O | 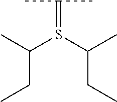 | |
| 580 | 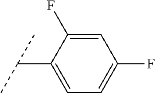 | O | 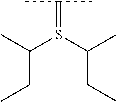 | |
| 581 | 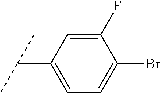 | O | 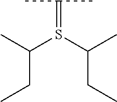 | |
| 582 | 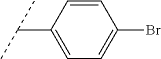 | O | 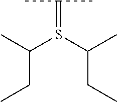 | |
| 583 | 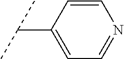 | O | 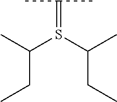 | |
| 584 | 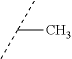 | O | 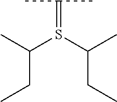 | |
| 585 | 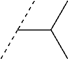 | O | 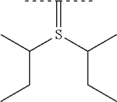 | |
| 586 | 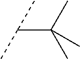 | O | 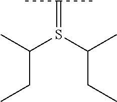 | |
| 587 | 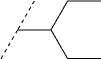 | O | 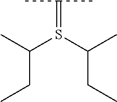 | |
| 588 | 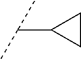 | O | 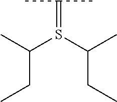 | |
| 589 | 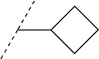 | O | 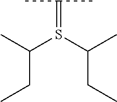 | |
| 590 | 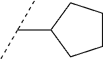 | O | 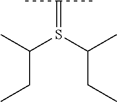 | |
| 591 | 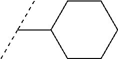 | O | 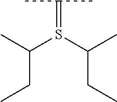 | |
| 592 | 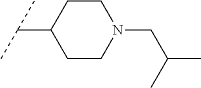 | O | 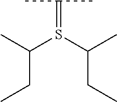 | |
| 593 | 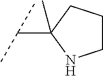 | O | 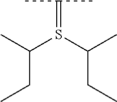 | |
| 594 | 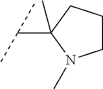 | O | 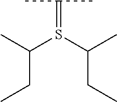 | |
| 595 | 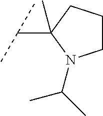 | O | 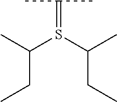 | |
| 596 | 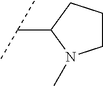 | O | 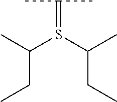 | |
| 597 | 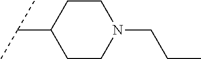 | O | 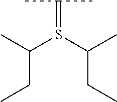 | |
| 598 | 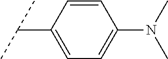 | O | 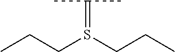 | |
| 599 | 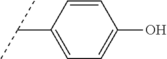 | O | 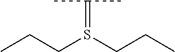 | |
| 600 | 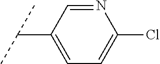 | O | 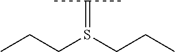 | |
| 601 | 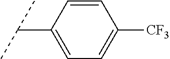 | O | 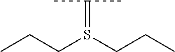 | |
| 602 | 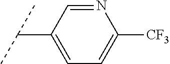 | O | 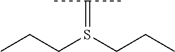 | |
| 603 | 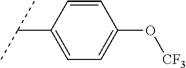 | O | 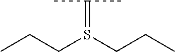 | |
| 604 | 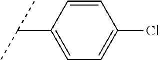 | O | 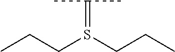 | |
| 605 | 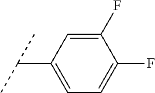 | O | 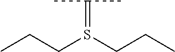 | |
| 606 | 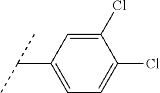 | O | 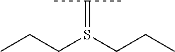 | |
| 607 | 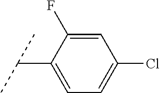 | O | 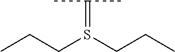 | |
| 608 | 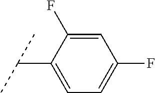 | O | 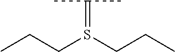 | |
| 609 | 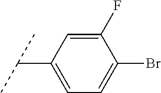 | O | 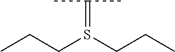 | |
| 610 | 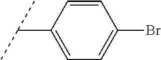 | O | 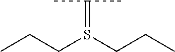 | |
| 611 | 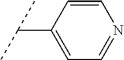 | O | 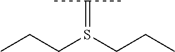 | |
| 612 | 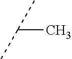 | O | 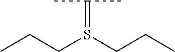 | |
| 613 | 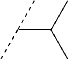 | O | 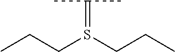 | |
| 614 | 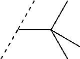 | O | 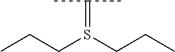 | |
| 615 | 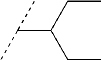 | O | 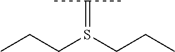 | |
| 616 | 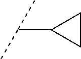 | O | 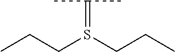 | |
| 617 | 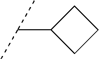 | O | 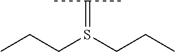 | |
| 618 | 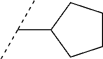 | O | 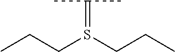 | |
| 619 | 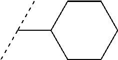 | O | 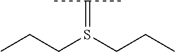 | |
| 620 | 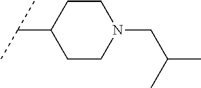 | O | 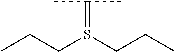 | |
| 621 | 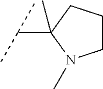 | O | 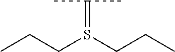 | |
| 622 | 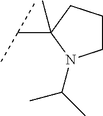 | O | 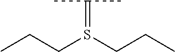 | |
| 623 | 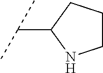 | O | 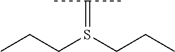 | |
| 624 | 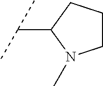 | O | 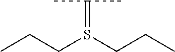 | |
| 625 | 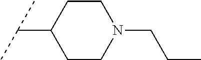 | O | 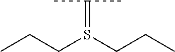 | |
| 626 | 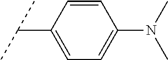 | O | 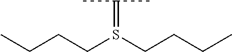 | |
| 627 | 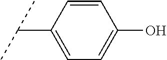 | O | 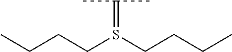 | |
| 628 | 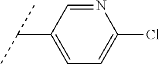 | O | 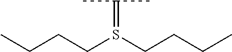 | |
| 629 | 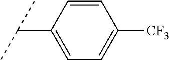 | O | 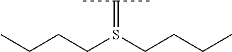 | |
| 630 | 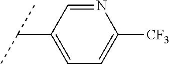 | O | 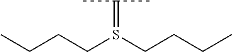 | |
| 631 | 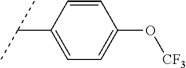 | O | 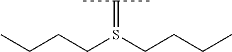 | |
| 632 | 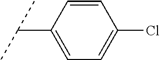 | O | 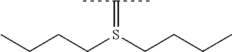 | |
| 633 | 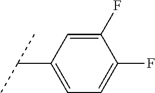 | O | 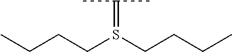 | |
| 634 | 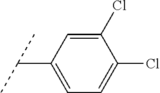 | O | 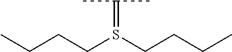 | |
| 635 | 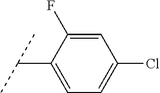 | O | 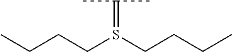 | |
| 636 | 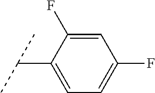 | O | 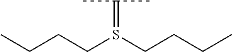 | |
| 637 | 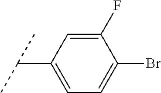 | O | 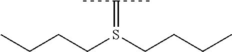 | |
| 638 | 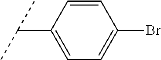 | O | 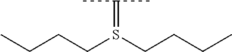 | |
| 639 | 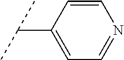 | O | 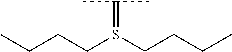 | |
| 640 | 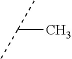 | O | 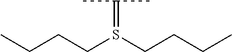 | |
| 641 | 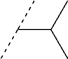 | O | 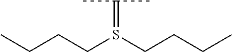 | |
| 642 | 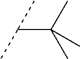 | O | 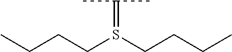 | |
| 643 | 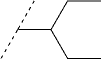 | O | 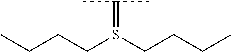 | |
| 644 | 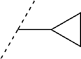 | O | 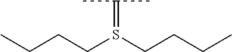 | |
| 645 | 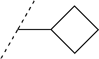 | O | 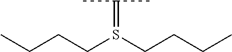 | |
| 646 | 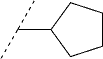 | O | 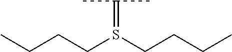 | |
| 647 | 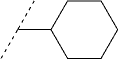 | O | 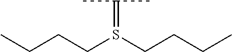 | |
| 648 | 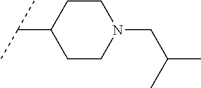 | O | 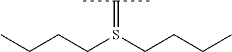 | |
| 649 | 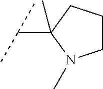 | O | 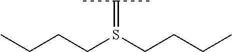 | |
| 650 | 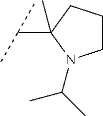 | O | 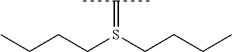 | |
| 651 | 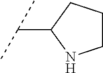 | O | 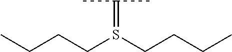 | |
| 652 | 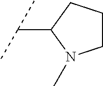 | O | 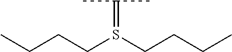 | |
| 653 | 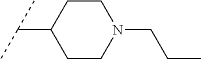 | O | 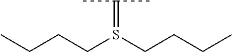 | |
| 654 | 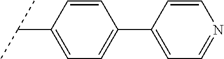 | O | H | H |
| 655 | 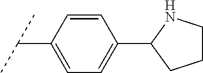 | O | H | H |
| 656 | 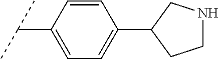 | O | H | H |
| 657 | 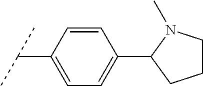 | O | H | H |
| 658 | 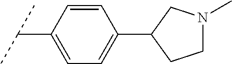 | O | H | H |
| 659 | 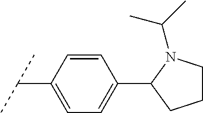 | O | H | H |
| 660 | 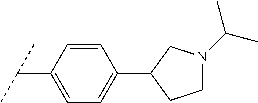 | O | H | H |
| 661 | 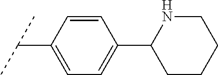 | O | H | H |
| 662 | 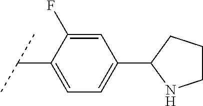 | O | H | H |
| 663 | 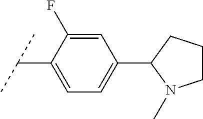 | O | H | H |
| 664 | 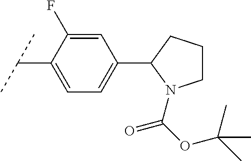 | O | H | H |
| 665 | 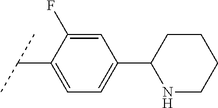 | O | H | H |
| 666 | 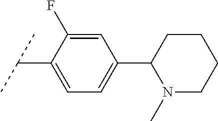 | O | H | H |
| 667 | 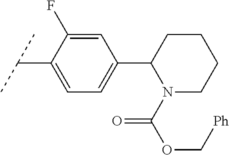 | O | H | H |
| 668 | 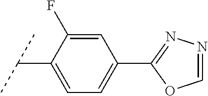 | O | H | H |
| 669 | 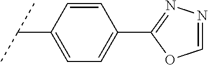 | O | H | H |
| 670 | 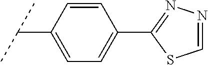 | O | H | H |
| 671 | 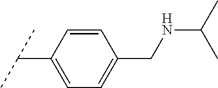 | O | H | H |
| 672 | 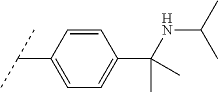 | O | H | H |
| 673 | 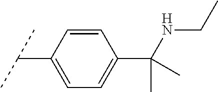 | O | H | H |
| 674 | 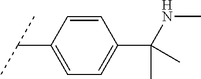 | O | H | H |
| 675 | 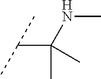 | O | H | H |
| 676 | 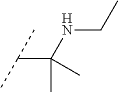 | O | H | H |
| 677 | 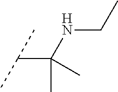 | O | H | H |
| 678 | 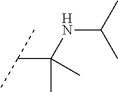 | O | H | H |
| 679 | 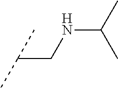 | O | H | H |
| 680 | 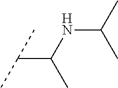 | O | H | H |
| 681 | 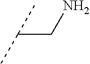 | O | H | H |
| 682 | 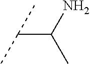 | O | H | H |
| 683 | 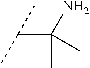 | O | H | H |
| 684 | 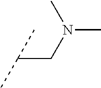 | O | H | H |
| 685 | 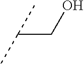 | O | H | H |
| 686 | 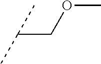 | O | H | H |
| 687 | 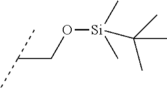 | O | H | H |
| 688 | 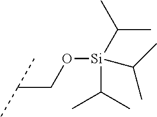 | O | H | H |
| 689 | 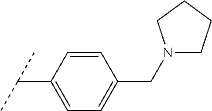 | O | H | H |
| 690 | 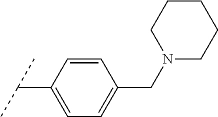 | O | H | H |
| 691 | 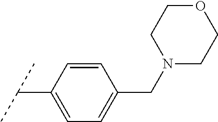 | O | H | H |
| 692 | 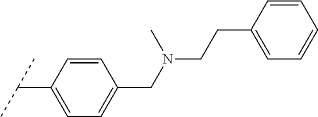 | O | H | H |
| 693 | 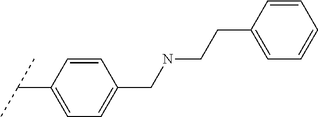 | O | H | H |
| 694 | 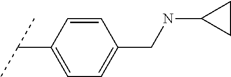 | O | H | H |
| 695 | 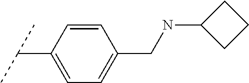 | O | H | H |
| 696 | 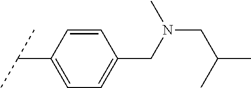 | O | H | H |
| 697 | 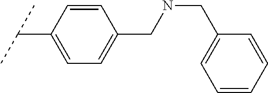 | O | H | H |
| 698 | 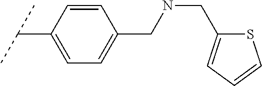 | O | H | H |
| 699 | 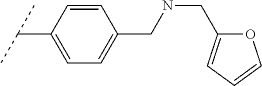 | O | H | H |
| 700 | 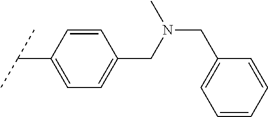 | O | H | H |

B2. Compounds B2-1 to B2-700 of the general formula (I) in which R¹ and R³ represent hydrogen, R² represents fluorine, X represents O and Q, W, Z¹, Z² for the individual compound in question correspond to the radical definitions given in Table 2 (Nos 1 to 700; corresponding to compounds B2-1 to B2-700).
B3. Compounds B3-1 to B3-700 of the general formula (I) in which R¹ and R³ represent hydrogen, R² represents chlorine, X represents O and Q, W, Z¹, Z² for the individual compound in question correspond to the radical definitions given in Table 2 (Nos 1 to 700; corresponding to compounds B3-1 to B3-700).
B4. Compounds B4-1 to B4-700 of the general formula (I) in which R¹ and R³ represent hydrogen, R² represents bromine, X represents O and Q, W, Z¹, Z² for the individual compound in question correspond to the radical definitions given in Table 2 (Nos 1 to 700; corresponding to compounds B4-1 to B4-700).
B5. Compounds B5-1 to B5-700 of the general formula (I) in which R¹ and R³ represent hydrogen, R² represents iodine, X represents O and Q, W, Z¹, Z² for the individual compound in question correspond to the radical definitions given in Table 2 (Nos 1 to 700; corresponding to compounds B5-1 to B5-700).
B6. Compounds B6-1 to B6-700 of the general formula (I) in which R¹ and R³ represent hydrogen, R² represents trifluoromethyl, X represents O and Q, W, Z¹, Z² for the individual compound in question correspond to the radical definitions given in Table 2 (Nos 1 to 700; corresponding to compounds B6-1 to B6-700).
B7. Compounds B7-1 to B7-700 of the general formula (I) in which R¹ and R³ represent hydrogen, R² represents methyl, X represents O and Q, W, Z¹, Z² for the individual compound in question correspond to the radical definitions given in Table 2 (Nos 1 to 700; corresponding to compounds B7-1 to B7-700).
B8. Compounds B8-1 to B8-700 of the general formula (I) in which R¹ and R³ represent hydrogen, R² represents methoxy, X represents O and Q, W, Z¹, Z² for the individual compound in question correspond to the radical definitions given in Table 2 (Nos 1 to 700; corresponding to compounds B8-1 to B8-700).
B9. Compounds B9-1 to B9-700 of the general formula (I) in which R¹ and R³ represent hydrogen, R² represents trifluoromethoxy, X represents O and Q, W, Z¹, Z² for the individual compound in question correspond to the radical definitions given in Table 2 (Nos 1 to 700; corresponding to compounds B9-1 to B9-700).
B10. Compounds B10-1 to B10-700 of the general formula (I) in which R¹ and R² represent hydrogen, R³ represents fluorine, X represents 0 and Q, W, Z¹, Z² for the individual compound in question correspond to the radical definitions given in Table 2 (Nos 1 to 700; corresponding to compounds B10-1 to B10-700).
B11. Compounds B11-1 to B11-700 of the general formula (I) in which R¹ and R² represent hydrogen, R³ represents chlorine, X represents 0 and Q, W, Z¹, Z² for the individual compound in question correspond to the radical definitions given in Table 2 (Nos 1 to 700; corresponding to compounds B11-1 to B11-700).
B12. Compounds B12-1 to B12-700 of the general formula (I) in which R² and R³ represent hydrogen, R¹ represents fluorine, X represents 0 and Q, W, Z¹, Z² for the individual compound in question correspond to the radical definitions given in Table 2 (Nos 1 to 700; corresponding to compounds B12-1 to B12-700).
B13. Compounds B13-1 to B13-700 of the general formula (I) in which R² and R³ represent hydrogen, R¹ represents chlorine, X represents 0 and Q, W, Z¹, Z² for the individual compound in question correspond to the radical definitions given in Table 2 (Nos 1 to 700; corresponding to compounds B13-1 to B13-700).
C1. Compounds C1-1 to C1-700 of the general formula (I) in which R¹, R² and R³ represent hydrogen and Q, W, Z¹ and Z² correspond to the definitions (Nos 1 to 700; corresponding to compounds C1-1 to C1-700) in Table 2 above.

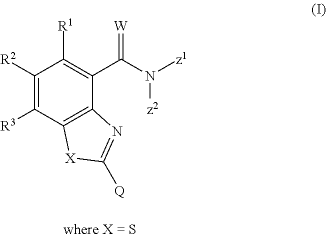

C2. Compounds C2-1 to C2-700 of the general formula (I) in which R¹ and R³ represent hydrogen, R² represents fluorine, X represents S and Q, W, Z¹, Z² for the individual compound in question correspond to the radical definitions given in Table 2 (Nos 1 to 700; corresponding to compounds C2-1 to C2-700).
C3. Compounds C3-1 to C3-700 of the general formula (I) in which R¹ and R³ represent hydrogen, R² represents chlorine, X represents S and Q, W, Z¹, Z² for the individual compound in question correspond to the radical definitions given in Table 2 (Nos 1 to 700; corresponding to compounds C3-1 to C3-700).
C4. Compounds C4-1 to C4-700 of the general formula (I) in which R¹ and R³ represent hydrogen, R² represents bromine, X represents S and Q, W, Z¹, Z² for the individual compound in question correspond to the radical definitions given in Table 2 (Nos 1 to 700; corresponding to compounds C4-1 to C4-700).
C5. Compounds C5-1 to C5-700 of the general formula (I) in which R¹ and R³ represent hydrogen, R² represents iodine, X represents S and Q, W, Z¹, Z² for the individual compound in question correspond to the radical definitions given in Table 2 (Nos 1 to 700; corresponding to compounds C5-1 to C5-700).
C6. Compounds C6-1 to C6-700 of the general formula (I) in which R¹ and R³ represent hydrogen, R² represents trifluoromethyl, X represents S and Q, W, Z¹, Z² for the individual compound in question correspond to the radical definitions given in Table 2 (Nos 1 to 700; corresponding to compounds C6-1 to C6-700).
C7. Compounds C7-1 to C7-700 of the general formula (I) in which R¹ and R³ represent hydrogen, R² represents methyl, X represents S and Q, W, Z¹, Z² for the individual compound in question correspond to the radical definitions given in Table 2 (Nos 1 to 700; corresponding to compounds C7-1 to C7-700).
C8. Compounds C8-1 to C8-700 of the general formula (I) in which R¹ and R³ represent hydrogen, R² represents methoxy, X represents S and Q, W, Z¹, Z² for the individual compound in question correspond to the radical definitions given in Table 2 (Nos 1 to 700; corresponding to compounds C8-1 to C8-700).
C9. Compounds C9-1 to C9-700 of the general formula (I) in which R¹ and R³ represent hydrogen, R² represents trifluoromethoxy, X represents S and Q, W, Z¹, Z² for the individual compound in question correspond to the radical definitions given in Table 2 (Nos 1 to 700; corresponding to compounds 08-1 to C8-700).
C10. Compounds C10-1 to C10-700 of the general formula (I) in which R¹ and R² represent hydrogen, R³ represents fluorine, X represents S and Q, W, Z¹, Z² for the individual compound in question correspond to the radical definitions given in Table 2 (Nos 1 to 700; corresponding to compounds 010-1 to 010-700).
C11. Compounds C11-1 to C11-700 of the general formula (I) in which R¹ and R² represent hydrogen, R³ represents chlorine, X represents S and Q, W, Z¹, Z² for the individual compound in question correspond to the radical definitions given in Table 2 (Nos 1 to 700; corresponding to compounds 011-1 to 011-700).
C12. Compounds C12-1 to C12-700 of the general formula (I) in which R² and R³ represent hydrogen, R¹ represents fluorine, X represents S and Q, W, Z¹, Z² for the individual compound in question correspond to the radical definitions given in Table 2 (Nos 1 to 700; corresponding to compounds 012-1 to C12-700).
C13. Compounds C13-1 to C13-700 of the general formula (I) in which R² and R³ represent hydrogen, R¹ represents chlorine, X represents S and Q, W, Z¹, Z² for the individual compound in question correspond to the radical definitions given in Table 2 (Nos 1 to 700; corresponding to compounds 013-1 to C13-700).

### Spectroscopic Data of Selected Table Examples

### Example No. A1-2

¹H-NMR (400 MHz, d₆-DMSO δ, ppm) 13.02 (s, 1H, NH), 9.43 (d, 1H, NH), 8.05 (d, 2H), 7.71 (d, 1H, NH), 7.62 (d, 1H), 7.23 (t, 1H), 6.83 (d, 2H), 3.02 (s, 6H).

### Example No. A1-3

¹H-NMR (400 MHz, d₆-DMSO δ, ppm) 13.11 (s, 1H, NH), 10.12 (d, 1H), 9.40 (d, 1H, NH), 8.08 (d, 2H), 7.82 (d, 1H), 7.72 (d, 1H, NH), 7.68 (d, 1H), 7.29 (t, 1H), 6.95 (d, 2H).

### Example No. A1-4

¹H-NMR (400 MHz, d₆-DMSO δ, ppm) 13.65 (s, 1H, NH), 9.27 (d, 2H), 8.67 (d, 1H), 7.91 (d, 1H), 7.80 (m, 3H), 7.40 (t, 1H).

### Example No. A1-5

¹H-NMR (400 MHz, d₆-DMSO δ, ppm) 13.63 (s, 1H, NH), 9.31 (d, 1H, NH), 8.43 (d, 2H), 8.02 (d, 2H), 7.85 (m, 3H), 7.41 (t, 1H).

### Example No. A1-6

¹H-NMR (400 MHz, d₆-DMSO δ, ppm) 13.83 (s, 1H, NH), 9.61 (s, 1H), 9.23 (s, 1H), 8.88 (d, 1H), 8.16 (d, 1H), 7.93 (d, 1H), 7.84 (m, 2H), 7.45 (t, 1H).

### Example No. A1-7

¹H-NMR (400 MHz, d₆-DMSO δ, ppm) 13.52 (s, 1H, NH), 9.23 (d, 1H, NH), 8.38 (d, 2H), 7.90 (d, 1H), 7.82 (d, 1H, NH), 7.78 (d, 1H), 7.6 (d, 2H), 7.38 (t, 1H).

### Example No. A1-8

¹H-NMR (400 MHz, CDCl₃ δ, ppm) 9.85 (br. s, 1H, NH), 9.71 (br. s, 1H, NH), 8.2 (d, 1H), 8.02 (m, 2H), 7.65 (d, 1H), 7.49 (m, 3H), 5.95 (br. s, 1H).

### Example No. A1-9

¹H-NMR (400 MHz, d₆-DMSO δ, ppm) 13.10 (s, 1H, NH), 9.25 (s, 1H), 8.38 (m, 1H), 7.90 (d, 1H), 7.75 (m, 2H), 7.55 (m, 1H), 7.35 (m, 2H).

### Example No. A1-10

¹H-NMR (400 MHz, d₆-DMSO δ, ppm) 13.55 (s, 1H, NH), 9.21 (s, 1H, NH), 8.49 (s, 1H), 8.25 (d, 1H), 7.85 (m, 2H), 7.75 (m, 1H), 7.42 (t, 1H).

### Example No. A1-11

¹H-NMR (400 MHz, d₆-DMSO δ, ppm) 9.31 (s, 1H), 8.40 (t, 1H), 7.95 (d, 1H), 7.83 (m, 2H), 7.61 (d, 1H), 7.42 (m, 3H).

### Example No. A1-12

¹H-NMR (400 MHz, d₆-DMSO δ, ppm) 13.10 (s, 1H, NH), 9.24 (s, 1H, NH), 8.38 (m, 1H), 7.91 (d, 1H), 7.82 (m, 2H), 7.75 (m, 1H), 7.35 (m, 2H).

### Example No. A1-14

¹H-NMR (400 MHz, d₆-DMSO δ, ppm) 13.48 (s, 1H, NH), 9.28 (s, 1H, NH), 8.23 (d, 2H), 7.89 (d, 1H), 7.80 (m, 4H), 7.37 (t, 1H).

### Example No. A1-15

¹H-NMR (400 MHz, d₆-DMSO δ, ppm) 13.75 (br. s, 1H, NH), 9.24 (br. s, 1H, NH), 8.31 (d, 2H), 8.22 (d, 2H), 7.92 (d, 1H), 7.80 (m, 2H), 7.44 (t, 1H).

### Example No. A1-16

¹H-NMR (400 MHz, d₆-DMSO δ, ppm) 8.30 (d, 1H, NH), 7.9 (d, 1H), 7.80 (d, 1H), 7.35 (m, 2H), 7.10 (d, 1H), 7.00 (t, 1H).

### Example No. A1-17

¹H-NMR (400 MHz, CD₃OD δ, ppm) 7.82 (d, 1H), 7.75 (d, 1H), 7.68 (s, 1H), 7.55 (d, 1H), 7.25 (t, 1H), 6.85 (d, 1H), 3.90 (s, 3H).

### Example No. A1-76

¹H-NMR (400 MHz, d₆-DMSO δ, ppm) 12.65 (s, 1H, NH), 9.37 (d, 1H, NH), 7.8 (d, 1H), 7.68 (br. s, 1H, NH), 7.62 (d, 1H), 7.25 (t, 1H), 3.22 (m, 1H), 1.39 (d, 6H).

### Example No. A1-77

¹H-NMR (400 MHz, d₆-DMSO δ, ppm) 12.6 (s, 1H, NH), 9.4 (d, 1H, NH), 7.8 (d, 1H), 7.68 (br. s, 1H, NH), 7.62 (d, 1H), 7.26 (t, 1H), 1.43 (s, 9H).

### Example No. A1-78

¹H-NMR (400 MHz, CDCl₃ δ, ppm) 9.90 (br. s, 1H, NH), 8.10 (s, 1H), 7.55 (s, 1H), 7.27 (t, 1H), 5.94 (br. s, 1H, NH), 2.80 (m, 1H), 1.85 (m, 4H), 0.90 (t, 6H).

### Example No. A1-79

¹H-NMR (400 MHz, CD₃OD δ, ppm) 7.74 (d, 1H), 7.48 (d, 1H), 7.13 (t, 1H), 2.10 (s, 1H), 1.08 (d, 4H).

### Example No. A1-81

¹H-NMR (400 MHz, CDCl₃ δ, ppm) 9.60 (br. s, 1H, NH), 8.10 (s, 1H), 7.60 (s, 1H), 7.25 (t, 1H), 5.90 (br. s, 1H, NH), 3.35 (m, 1H), 2.20 (m, 2H), 2.00 (m, 2H), 1.85 (m, 2H), 1.73 (m, 2H).

### Example No. A1-82

¹H-NMR (400 MHz, d₆-DMSO δ, ppm) 12.6 (s, 1H, NH), 9.35 (d, 1H, NH), 7.8 (d, 1H), 7.62 (m, 2H), 7.25 (t, 1H), 2.9 (m, 1H), 2.07 (d, 2H), 1.8 (m, 2H), 1.7-1.55 (m, 3H), 1.43-1.23 (m, 3H).

### Example No. A1-177

¹H-NMR (400 MHz, CD₃OD δ, ppm) 7.90 (d, 1H), 7.70 (d, 1H), 7.30 (t, 1H), 3.44 (d, 2H), 3.24 (m, 1H), 2.80 (t, 2H), 2.70 (d, 2H), 2.35-2.20 (m, 4H), 2.10 (m, 1H), 1.04 (d, 6H).

### Example No. A1-208

¹H-NMR (400 MHz, CDCl₃ δ, ppm) 9.65 (br. s, 1H, NH), 8.95 (br. s, 1H, NH), 8.10 (d, 1H), 7.90 (d, 1H), 7.52 (t, 1H), 7.35-7.20 (m, 5H), 2.95 (m, 2H), 2.77 (t, 2H), 2.23 (m, 2H).

### Example No. A1-227

¹H-NMR (400 MHz, CD₃OD δ, ppm) 8.30 (s, 1H), 7.95 (d, 1H), 7.70 (d, 1H), 7.35 (t, 1H), 4.03 (s, 3H).

### Example No. A1-228

¹H-NMR (400 MHz, CD₃OD δ, ppm) 8.27 (s, 1H), 7.95 (d, 1H), 7.70 (d, 1H), 7.50+6.90 (s, 1H), 7.30 (t, 1H), 4.03 (s, 3H).

### Example No. A1-491

¹H-NMR (400 MHz, CDCl₃ δ, ppm) 9.65 (br. s, 1H, NH), 8.08 (d, 1H), 7.45 (d, 1H), 7.3 (t, 1H), 5.89 (br. s, 1H, NH), 4.2 (d, 2H), 2.04 (m, 1H), 1.3 (m, 3H), 1.18 (m, 2H), 0.63 (m, 2H), 0.44 (m, 2H).

### Example No. A2-76

¹H-NMR (400 MHz, CDCl₃ δ, ppm) 9.70 (br. s, 1H, NH), 8.40 (s, 1H), 8.15+7.65 (br. s, 1H, NH), 7.80 (s, 1H), 6.00 (br. s, 2H), 3.30 (m, 1H), 1.60 (d, 6H).

### Example No. A2-81

¹H-NMR (400 MHz, d₆-DMSO δ, ppm) 13.25 (s, 1H, NH), 8.25 (s, 1H, NH), 8.08 (d, 2H), 3.40 (m, 1H), 2.12 (m, 2H), 1.94 (m, 2H), 1.75 (m, 2H) 1.70 (m, 2H).

### Example No. A2-82

¹H-NMR (400 MHz, CDCl₃ δ, ppm) 9.70 (br. s, 1H, NH), 8.40 (s, 1H), 8.15+7.65 (br. s, 1H, NH), 7.80 (s, 1H), 6.00 (br. s, 1H), 3.30 (m, 1H), 2.20 (m, 2H), 1.90 (m, 2H), 1.80 (m, 1H), 1.70 (m, 2H), 1.50 (m, 2H), 1.40 (m, 1H).

### Example No. B1-5

¹H-NMR (400 MHz, CDCl₃ δ, ppm) 8.85 (br. s, 1H, NH), 8.42 (d, 1H), 8.25 (d, 1H), 7.84 (d, 2H), 7.79 (d, 1H), 7.53 (t, 1H), 5.92 (br. s, 1H, NH).

### Example No. B1-7

¹H-NMR (400 MHz, DMSO-d₆ δ, ppm) 8.48-8.43 (m, 2H), 8.01-7.98 (m, 2H), 7.67-7.53 (m, 3H), 7.49 (t, 1H).

### Example No. B1-8

¹H-NMR (400 MHz, DMSO-d₆ δ, ppm) 8.43 (br. s, 1H), 8.32 (d, 2H), 8.01-7.97 (m, 3H), 7.72-7.68 (d, 2H), 7.56 (t, 1H)

### Example No. B1-11

¹H-NMR (400 MHz, DMSO-d₆ δ, ppm) 8.43-8.39 (m, 2H), 8.07-8.00 (m, 3H), 7.81 (d, 1H), 7.62-7.57 (m, 2H)

### Example No. B1-14

¹H-NMR (400 MHz, DMSO-d₆ δ, ppm) 8.45 (br. s, 1H), 8.23 (d, 2H), 8.03-7.99 (m, 3H), 7.86 (d, 2H), 7.57 (t, 1H).

### Example No. B1-36

¹H-NMR (400 MHz, DMSO-d₆ δ, ppm) 8.38 (br. s, 1H), 8.25 (d, 1H), 8.09-7.96 (m, 4H), 7.66-7.61 (m, 2H).

### Example No. B1-50

¹H-NMR (400 MHz, DMSO-d₆ δ, ppm) 8.49 (br. s, 1H, NH), 8.24 (d, 2H), 7.99-7.94 (m, 3H), 7.50 (t, 1H), 7.18 (d, 2H), 3.88 (s, 3H).

### Example No. B1-75

¹H-NMR (400 MHz, DMSO-d₆ δ, ppm) 8.31 (br. s, 1H), 8.06 (d, 1H), 7.90-7.84 (m, 2H), 7.51 (t, 1H), 2.70 (s, 3H).

### Example No. B1-76

¹H-NMR (400 MHz, CDCl₃ δ, ppm) 8.90 (br. s, 1H, NH), 8.13 (d, 1H), 7.63 (d, 1H), 7.40 (t, 1H), 5.85 (br. s, 1H, NH), 3.29 (m, 1H), 1.49 (d, 6H).

### Example No. B1-77

¹H-NMR (400 MHz, CDCl₃ δ, ppm) 8.94 (br. s, 1H, NH), 8.25 (d, 1H), 7.65 (d, 1H), 7.41 (t, 1H), 5.82 (br. s, 1H, NH), 1.53 (s, 9H).

### Example No. B1-82

¹H-NMR (400 MHz, CDCl₃ δ, ppm) 8.90 (br. s, 1H, NH), 8.13 (d, 1H), 7.63 (d, 1H), 7.40 (t, 1H), 5.83 (br. s, 1H, NH), 3.0 (m, 1H), 2.20 (m, 2H), 1.90 (m, 2H), 1.75-1.65 (m, 3H), 1.45-1.30 (m, 3H).

### Example No. B1-328

¹H-NMR (400 MHz, CDCl₃ δ, ppm) 10.12 (br. s, 1H, NH), 7.83 (d, 1H), 7.54 (t, 1H), 7.40 (d, 1H), 7.19 (br. s, 1H, NH).

### Example No. B1-331

¹H-NMR (400 MHz, CDCl₃ δ, ppm) 9.50 (br. s, 1H, NH), 7.82 (d, 1H), 7.57 (m, 1H), 7.40 (d, 1H), 7.13 (br. s, 1H, NH).

### Example No. C1-328

¹H-NMR (400 MHz, CDCl₃ δ, ppm) 9.18 (br. s, 1H, NH), 8.60 (d, 1H), 8.18 (d, 1H), 7.74 (t, 1H), 6.03 (br. s, 1H, NH).

The present invention thus provides for the use of at least one compound selected from the group consisting of substituted 2-amidobenzimidazoles, 2-amidobenzoxazoles and 2-amidobenzothiazoles of the general formula (I), and of any desired mixtures of these substituted 2-amidobenzimidazoles, 2-amidobenzoxazoles and 2-amidobenzothiazoles of the general formula (I) according to the invention, with further agrochemically active compounds, for enhancement of the resistance of plants to abiotic stress factors, preferably drought stress, especially for invigoration of plant growth and/or for increasing plant yield.

The present invention further provides a spray solution for treatment of plants, comprising an amount, effective for enhancement of the resistance of plants to abiotic stress factors, of at least one compound selected from the group consisting of substituted 2-amidobenzimidazoles, 2-amidobenzoxazoles and 2-amidobenzothiazoles of the general formula (I). The abiotic stress conditions which can be relativized may include, for example, heat, drought, cold and aridity stress (stress caused by aridity and/or lack of water), osmotic stress, waterlogging, elevated soil salinity, elevated exposure to minerals, ozone conditions, strong light conditions, limited availability of nitrogen nutrients, limited availability of phosphorus nutrients.

In one embodiment, it is possible, for example, that the compounds envisaged in accordance with the invention, i.e. the appropriate substituted 2-amidobenzimidazoles, 2-amidobenzoxazoles and 2-amidobenzothiazoles of the general formula (I), are applied by spray application to appropriate plants or plant parts to be treated. The compounds of the general formula (I) or salts thereof are used as envisaged in accordance with the invention preferably with a dosage between 0.00005 and 3 kg/ha, more preferably between 0.0001 and 2 kg/ha, especially preferably between 0.0005 and 1 kg/ha, specifically preferably between 0.001 and 0.25 kg/ha. If, in the context of the present invention, abscisic acid is used simultaneously with substituted 2-amidobenzimidazoles, 2-amidobenzoxazoles and 2-amidobenzothiazoles of the general formula (I), for example in the context of a combined preparation or formulation, the addition of abscisic acid is preferably carried out in a dosage from 0.0001 to 3 kg/ha, particularly preferably from 0.001 to 2 kg/ha, very particularly preferably from 0.005 to 1 kg/ha, especially preferably from 0.006 to 0.25 kg/ha.

The term “resistance to abiotic stress” is understood in the context of the present invention to mean various kinds of advantages for plants. Such advantageous properties are manifested, for example, in the following improved plant characteristics: improved root growth with regard to surface area and depth, increased stolon and tiller formation, stronger and more productive stolons and tillers, improvement in shoot growth, increased lodging resistance, increased shoot base diameter, increased leaf area, higher yields of nutrients and constituents, for example carbohydrates, fats, oils, proteins, vitamins, minerals, essential oils, dyes, fibers, better fiber quality, earlier flowering, increased number of flowers, reduced content of toxic products such as mycotoxins, reduced content of residues or disadvantageous constituents of any kind, or better digestibility, improved storage stability of the harvested material, improved tolerance to disadvantageous temperatures, improved tolerance to drought and aridity, and also oxygen deficiency as a result of waterlogging, improved tolerance to elevated salt contents in soil and water, enhanced tolerance to ozone stress, improved compatibility with respect to herbicides and other plant treatment compositions, improved water absorption and photosynthesis performance, advantageous plant properties, for example acceleration of ripening, more homogeneous ripening, greater attractiveness to beneficial animals, improved pollination, or other advantages that are well known to a person skilled in the art.

More particularly, the use according to the invention of one or more compounds of the general formula (I) exhibits the advantages described in spray application to plants and plant parts. Combinations of the corresponding substituted 2-amidobenzimidazoles, 2-amidobenzoxazoles and 2-amidobenzothiazoles of the general formula (I) according to the invention with substances including insecticides, attractants, acaricides, fungicides, nematicides, herbicides, growth regulators, safeners, substances which influence plant maturity, and bactericides can likewise be employed in the control of plant disorders and/or for achieving increased yield in the context of the present invention. In addition, the combined use of substituted 2-amidobenzimidazoles, 2-amidobenzoxazoles and 2-amidobenzothiazoles of the general formula (I) according to the invention with genetically modified cultivars with a view to increased tolerance to abiotic stress is likewise possible.

As is known, the further various benefits for plants mentioned above can be combined in a known manner in component form, and generally applicable terms can be used to describe them. Such terms are, for example, the following names: phytotonic effect, resistance to stress factors, less plant stress, plant health, healthy plants, plant fitness, plant wellness, plant concept, vigor effect, stress shield, protective shield, crop health, crop health properties, crop health products, crop health management, crop health therapy, plant health, plant health properties, plant health products, plant health management, plant health therapy, greening effect or regreening effect, freshness, or other terms with which a person skilled in the art is entirely familiar.

In the context of the present invention, a good effect on resistance to abiotic stress is understood to mean, without limitation,
at least an emergence improved by generally 3%, especially more than 5%, more preferably more than 10%,
at least a yield enhanced generally 3%, especially more than 5%, more preferably more than 10%,
at least a root development improved by generally 3%, especially more than 5%, more preferably more than 10%,
at least a shoot size rising by generally 3%, especially more than 5%, more preferably more than 10%,
at least a leaf area increased by generally 3%, especially more than 5%, more preferably more than 10%,
at least a photosynthesis performance improved by generally 3%, especially more than 5%, more preferably more than 10%, and/or
at least a flower development improved by generally 3%, especially more than 5%, more preferably more than 10%,
   and the effects may occur individually or else in any combination of two or more effects.

The present invention further provides a spray solution for treatment of plants, comprising an amount, effective for enhancement of the resistance of plants to abiotic stress factors, of at least one compound from the group of the 2-amidobenzimidazoles, 2-amidobenzoxazoles and 2-amidobenzothiazoles of the general formula (I). The spray solution may comprise other customary constituents, such as solvents, formulation aids, especially water. Further constituents may include agrochemically active compounds which are described further below.

The present invention further provides for the use of corresponding spray solutions for increasing the resistance of plants to abiotic stress factors. The remarks which follow apply both to the use according to the invention of one or more compounds of the general formula (I) per se and to the corresponding spray solutions.

In accordance with the invention, it has additionally been found that the application, to plants or in their environment, of one or more compounds of the general formula (I) in combination with at least one fertilizer as defined further below is possible.

Fertilizers which can be used in accordance with the invention together with the compounds of the general formula (I) elucidated in detail above are generally organic and inorganic nitrogen-containing compounds, for example ureas, urea/formaldehyde condensation products, amino acids, ammonium salts and ammonium nitrates, potassium salts (preferably chlorides, sulfates, nitrates), salts of phosphoric acid and/or salts of phosphorous acid (preferably potassium salts and ammonium salts). Particular mention should be made in this connection of the NPK fertilizers, i.e. fertilizers which comprise nitrogen, phosphorus and potassium, calcium ammonium nitrate, i.e. fertilizers which also contain calcium, ammonium sulfate nitrate (general formula (NH₄)₂SO₄NH₄NO₃), ammonium phosphate and ammonium sulfate. These fertilizers are generally known to the person skilled in the art; see also, for example, Ullmann's Encyclopedia of Industrial Chemistry, 5th edition, Vol. A 10, pages 323 to 431, Verlagsgesellschaft, Weinheim, 1987.

The fertilizers may additionally comprise salts of micronutrients (preferably calcium, sulfur, boron, manganese, magnesium, iron, boron, copper, zinc, molybdenum and cobalt) and of phytohormones (for example vitamin B1 and indole (III)acetic acid) or mixtures of these. Fertilizers used in accordance with the invention may also contain other salts such as monoammonium phosphate (MAP), diammonium phosphate (DAP), potassium sulfate, potassium chloride, magnesium sulfate. Suitable amounts for the secondary nutrients or trace elements are amounts of 0.5 to 5% by weight, based on the overall fertilizer. Further possible ingredients are crop protection agents, insecticides or fungicides, growth regulators or mixtures thereof. Further details of these are given below.

The fertilizers can be used, for example, in the form of powders, granules, prills or compactates. However, the fertilizers can also be used in liquid form, dissolved in an aqueous medium. In this case, dilute aqueous ammonia can also be used as a nitrogen fertilizer. Further possible ingredients for fertilizers are described, for example, in Ullmann's Encyclopedia of Industrial Chemistry, 5th edition, 1987, volume A 10, pages 363 to 401, DE-A 41 28 828, DE-A 19 05 834 and DE-A 196 31 764. The general composition of the fertilizers, which, in the context of the present invention, may take the form of straight and/or compound fertilizers, for example composed of nitrogen, potassium or phosphorus, may vary within a wide range. In general, a content of from 1 to 30% by weight of nitrogen (preferably from 5 to 20% by weight), from 1 to 20% by weight of potassium (preferably from 3 to 15% by weight) and a content of from 1 to 20% by weight of phosphorus (preferably from 3 to 10% by weight) is advantageous. The microelement content is usually in the ppm range, preferably in the range from 1 to 1000 ppm.

In the context of the present invention, the fertilizer and one or more compounds of the general formula (I) may be administered simultaneously. However, it is also possible first to apply the fertilizer and then one or more compounds of the general formula (I), or first to apply one or more compounds of the general formula (I) and then the fertilizer. In the case of nonsynchronous application of one or more compounds of the general formula (I) and the fertilizer, the application in the context of the present invention is, however, effected in a functional relationship, especially within a period of generally 24 hours, preferably 18 hours, more preferably 12 hours, specifically 6 hours, more specifically 4 hours, even more specifically within 2 hours. In very particular embodiments of the present invention, one or more compounds of the formula (I) according to the invention and the fertilizer are applied within a time frame of less than 1 hour, preferably less than 30 minutes, more preferably less than 15 minutes.

Preference is given to the use of compounds of the general formula (I) on plants from the group of the useful plants, ornamentals, turfgrass types, commonly used trees which are used as ornamentals in the public and domestic sectors, and forestry trees. Forestry trees include trees for the production of timber, cellulose, paper and products made from parts of the trees. The term useful plants as used here refers to crop plants which are used as plants for obtaining foods, animal feeds, fuels or for industrial purposes.

The useful plants include, for example, the following types of plants: triticale, durum (hard wheat), turf, vines, cereals, for example wheat, barley, rye, oats, rice, corn and millet; beet, for example sugar beet and fodder beet; fruits, for example pome fruit, stone fruit and soft fruit, for example apples, pears, plums, peaches, almonds, cherries and berries, for example strawberries, raspberries, blackberries; legumes, for example beans, lentils, peas and soybeans; oil crops, for example oilseed rape, mustard, poppies, olives, sunflowers, coconuts, castor oil plants, cocoa beans and peanuts; cucurbits, for example pumpkin/squash, cucumbers and melons; fiber plants, for example cotton, flax, hemp and jute; citrus fruits, for example oranges, lemons, grapefruit and tangerines; vegetables, for example spinach, lettuce, asparagus, cabbage species, carrots, onions, tomatoes, potatoes and bell peppers; Lauraceae, for example avocado, Cinnamomum, camphor, or also plants such as tobacco, nuts, coffee, eggplant, sugar cane, tea, pepper, grapevines, hops, bananas, latex plants and ornamentals, for example flowers, shrubs, deciduous trees and coniferous trees. This enumeration does not represent any limitation.

The following plants are considered to be particularly suitable target crops for the application of the process according to the invention: oats, rye, triticale, durum, cotton, eggplant, turf, pome fruit, stone fruit, soft fruit, corn, wheat, barley, cucumber, tobacco, vines, rice, cereals, pears, pepper, beans, soybeans, oilseed rape, tomato, bell pepper, melons, cabbage, potatoes and apples.

Examples of trees which can be improved in accordance with the method according to the invention include: Abies sp., Eucalyptus sp., Picea sp., Pinus sp., Aesculus sp., Platanus sp., Tilia sp., Acer sp., Tsuga sp., Fraxinus sp., Sorbus sp., Betula sp., Crataegus sp., Ulmus sp., Quercus sp., Fagus sp., Salix sp., Populus sp.

Preferred trees which can be improved by the method according to the invention include: from the tree species Aesculus: A. hippocastanum, A. pariflora, A. carnea; from the tree species Platanus: P. aceriflora, P. occidentalis, P. racemosa; from the tree species Picea: P. abies; from the tree species Pinus: P. radiate, P. ponderosa, P. contorta, P. sylvestre, P. elliottii, P. montecola, P. albicaulis, P. resinosa, P. palustris, P. taeda, P. flexilis, P. jeffregi, P. baksiana, P. strobes; from the tree species Eucalyptus: E. grandis, E. globulus, E. camadentis, E. nitens, E. obliqua, E. regnans, E. pilularus.

Particularly preferred trees which can be improved in accordance with the method according to the invention are: from the tree species Pinus: P. radiate, P. ponderosa, P. contorta, P. sylvestre, P. strobes; from the tree species Eucalyptus: E. grandis, E. globulus and E. camadentis.

Particularly preferred trees which can be improved in accordance with the method according to the invention are: horse chestnut, Platanaceae, linden tree and maple tree.

The present invention can also be applied to any turfgrass types, including cool-season turfgrasses and warm-season turfgrasses. Examples of cool-season turfgrasses are bluegrasses (Poa spp.), such as Kentucky bluegrass (Poa pratensis L.), rough bluegrass (Poa trivialis L.), Canada bluegrass (Poa compressa L.), annual bluegrass (Poa annua L.), upland bluegrass (Poa glaucantha Gaudin), wood bluegrass (Poa nemoralis L.) and bulbous bluegrass (Poa bulbosa L.); bentgrasses (Agrostis spp.) such as creeping bentgrass (Agrostis palustris Huds.), colonial bentgrass (Agrostis tenuis Sibth.), velvet bentgrass (Agrostis canina L.), South German Mixed Bentgrass (Agrostis spp. including Agrostis tenius Sibth., Agrostis canina L., and Agrostis palustris Huds.), and redtop (Agrostis alba L.);

fescues (Festuca spp.), such as red fescue (Festuca rubra L. spp. rubra), creeping fescue (Festuca rubra L.), chewings fescue (Festuca rubra commutata Gaud.), sheep fescue (Festuca ovina L.), hard fescue (Festuca longifolia Thuill.), hair fescue (Festucu capillata Lam.), tall fescue (Festuca arundinacea Schreb.) and meadow fescue (Festuca elanor L.);

ryegrasses (Lolium spp.), such as annual ryegrass (Lolium multiflorum Lam.), perennial ryegrass (Lolium perenne L.) and Italian ryegrass (Lolium multiflorum Lam.); and wheatgrasses (Agropyron spp.), such as fairway wheatgrass (Agropyron cristatum (L.) Gaertn.), crested wheatgrass (Agropyron desertorum (Fisch.) Schult.) and western wheatgrass (Agropyron smithii Rydb.).

Examples of further cool-season turfgrasses are beachgrass (Ammophila breviligulata Fern.), smooth bromegrass (Bromus inermis Leyss.), cattails such as Timothy (Phleum pratense L.), sand cattail (Phleum subulatum L.), orchard grass (Dactylis glomerata L.), weeping alkaligrass (Puccinellia distans (L.) Parl.) and crested dog's-tail (Cynosurus cristatus L.).

Examples of warm-season turfgrasses are Bermuda grass (Cynodon spp. L. C. Rich), zoysia grass (Zoysia spp. Willd.), St. Augustine grass (Stenotaphrum secundatum Walt Kuntze), centipede grass (Eremochloa ophiuroides Munro Hack.), carpet grass (Axonopus affinis Chase), Bahia grass (Paspalum notatum Flugge), Kikuyu grass (Pennisetum clandestinum Hochst. ex Chiov.), buffalo grass (Buchloe dactyloids (Nutt.) Engelm.), Blue gramma (Bouteloua gracilis (H.B.K.) Lag. ex Griffiths), seashore paspalum (Paspalum vaginatum Swartz) and sideoats grama (Bouteloua curtipendula (Michx. Torr.)). Cool-season turfgrasses are generally preferred for the use according to the invention. Particular preference is given to bluegrass, bentgrass and redtop, fescues and ryegrasses. Bentgrass is especially preferred.

Particular preference is given to using the compounds of the general formula (I) according to the invention to treat plants of the respective commercially available or commonly used plant cultivars. Plant cultivars are understood to mean plants which have new properties (“traits”) and which have been obtained by conventional breeding, by mutagenesis or with the aid of recombinant DNA techniques. Crop plants may accordingly be plants which can be obtained by conventional breeding and optimization methods or by biotechnological and genetic engineering methods or combinations of these methods, including the transgenic plants and including the plant cultivars which are protectable or non-protectable by plant breeders' rights.

The treatment method according to the invention can thus also be used for the treatment of genetically modified organisms (GMOs), e.g. plants or seeds. Genetically modified plants (or transgenic plants) are plants in which a heterologous gene has been stably integrated into the genome. The expression “heterologous gene” essentially means a gene which is provided or assembled outside the plant and when introduced into the nuclear, chloroplastic or hypochondrial genome gives the transformed plant new or improved agronomic or other properties by expressing a protein or polypeptide of interest or by downregulating or silencing (an)other gene(s) which is/are present in the plant (using for example antisense technology, cosuppression technology or RNAi technology [RNA interference]). A heterologous gene that is located in the genome is also referred to as a transgene. A transgene that is defined by its specific presence in the plant genome is called a transformation or transgenic event.

Plants and plant varieties which are preferably treated with the compounds of the general formula (I) according to the invention include all plants which have genetic material which imparts particularly advantageous, useful traits to these plants (whether obtained by breeding and/or biotechnological means or not).

Plants and plant cultivars which can likewise be treated with the compounds of the general formula (I) according to the invention are those plants which are resistant to one or more abiotic stress factor. Abiotic stress conditions may include, for example, heat, drought, cold and aridity stress, osmotic stress, waterlogging, increased soil salinity, increased exposure to minerals, ozone conditions, strong light conditions, limited availability of nitrogen nutrients, limited availability of phosphorus nutrients or shade avoidance.

Plants and plant cultivars which can likewise be treated with the compounds of the general formula (I) according to the invention are those plants which are characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including but not limited to, early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and oil composition, nutritional value, reduction in antinutritional compounds, improved processability and better storage stability.

Plants that may also be treated with the compounds of the general formula (I) according to the invention are hybrid plants that already express the characteristics of heterosis, or hybrid effect, which results in generally higher yield, higher vigor, better health and better resistance towards biotic and abiotic stress factors. Such plants are typically produced by crossing an inbred male-sterile parent line (the female crossbreeding parent) with another inbred male-fertile parent line (the male crossbreeding parent). The hybrid seed is typically harvested from the male-sterile plants and sold to growers. Male-sterile plants can sometimes (for example in maize) be produced by detasseling (i.e. mechanical removal of the male reproductive organs or male flowers); however, it is more typical for male sterility to be the result of genetic determinants in the plant genome. In that case, and especially when seed is the desired product to be harvested from the hybrid plants, it is typically beneficial to ensure that male fertility in hybrid plants, which contain the genetic determinants responsible for male sterility, is fully restored. This can be accomplished by ensuring that the male crossbreeding parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described for Brassica species (WO 92/005251, WO 95/009910, WO 98/27806, WO 05/002324, WO 06/021972 and U.S. Pat. No. 6,229,072). However, genetic determinants for male sterility can also be located in the nuclear genome. Male-sterile plants can also be obtained by plant biotechnology methods such as genetic engineering. A particularly useful means of obtaining male-sterile plants is described in WO 89/10396 in which, for example, a ribonuclease such as barnase is selectively expressed in the tapetum cells in the stamens. Fertility can then be restored by expression in the tapetum cells of a ribonuclease inhibitor such as barstar (e.g. WO 91/002069).

Plants or plant varieties (obtained by plant biotechnology methods such as genetic engineering) which may also be treated with the compounds of the general formula (I) according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. Thus, for example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., Science (1983), 221, 370-371), the CP4 gene of the bacterium Agrobacterium sp. (Barry et al., Curr. Topics Plant Physiol. (1992), 7, 139-145), the genes encoding a petunia EPSPS (Shah et al., Science (1986), 233, 478-481), a tomato EPSPS (Gasser et al., J. Biol. Chem. (1988), 263, 4280-4289) or an Eleusine EPSPS (WO 01/66704). The EPSPS may also take the form of a mutated EPSPS as described, for example, in EP-A 0837944, WO 00/066746, WO 00/066747 or WO 02/026995. Glyphosate-tolerant plants can also be obtained by expressing a gene that encodes a glyphosate oxidoreductase enzyme as described in U.S. Pat. No. 5,776,760 and U.S. Pat. No. 5,463,175. Glyphosate-tolerant plants can also be obtained by expressing a gene that encodes a glyphosate acetyltransferase enzyme as described, for example, in WO 02/036782, WO 03/092360, WO 05/012515 and WO 07/024782. Glyphosate-tolerant plants can also be obtained by selecting plants containing naturally occurring mutations of the abovementioned genes, as described, for example, in WO 01/024615 or WO 03/013226.

Other herbicide-resistant plants are for example plants that have been made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One such effective detoxifying enzyme is, for example, an enzyme encoding a phosphinothricin acetyltransferase (such as the bar or pat protein from Streptomyces species, for example). Examples of plants which express an exogenous phosphinothricin acetyltransferase are described in U.S. Pat. No. 5,561,236; U.S. Pat. No. 5,648,477; U.S. Pat. No. 5,646,024; U.S. Pat. No. 5,273,894; U.S. Pat. No. 5,637,489; U.S. Pat. No. 5,276,268; U.S. Pat. No. 5,739,082; U.S. Pat. No. 5,908,810 and U.S. Pat. No. 7,112,665.

Further herbicide-tolerant plants are also plants that have been made tolerant to the herbicides inhibiting the enzyme hydroxyphenylpyruvate dioxygenase (HPPD). Hydroxyphenylpyruvate dioxygenases are enzymes that catalyze the reaction in which para-hydroxyphenylpyruvate (HPP) is converted to homogentisate. Plants tolerant to HPPD inhibitors can be transformed with a gene encoding a naturally occurring resistant HPPD enzyme, or a gene encoding a mutated HPPD enzyme according to WO 96/038567, WO 99/024585 and WO 99/024586. Tolerance to HPPD inhibitors can also be obtained by transforming plants with genes encoding certain enzymes enabling the formation of homogentisate despite the inhibition of the native HPPD enzyme by the HPPD inhibitor. Such plants and genes are described in WO 99/034008 and WO 2002/36787. Tolerance of plants to HPPD inhibitors can also be improved by transforming plants with a gene encoding a prephenate dehydrogenase enzyme in addition to a gene encoding an HPPD-tolerant enzyme, as described in WO 2004/024928.

Other herbicide-resistant plants are plants which have been rendered tolerant to acetolactate synthase (ALS) inhibitors. Known ALS inhibitors include, for example, sulfonylurea, imidazolinone, triazolopyrimidines, pyrimidinyloxy(thio)benzoates, and/or sulfonylaminocarbonyltriazolinone herbicides. It is known that different mutations in the ALS enzyme (also known as acetohydroxy acid synthase, AHAS) confer tolerance to a variety of herbicides and groups of herbicides, as described, for example, by Tranel and Wright, Weed Science (2002), 50, 700-712, but also in U.S. Pat. No. 5,605,011, U.S. Pat. No. 5,378,824, U.S. Pat. No. 5,141,870 and U.S. Pat. No. 5,013,659. The production of sulfonylurea-tolerant plants and imidazolinone-tolerant plants has been described in U.S. Pat. No. 5,605,011; U.S. Pat. No. 5,013,659; U.S. Pat. No. 5,141,870; U.S. Pat. No. 5,767,361; U.S. Pat. No. 5,731,180; U.S. Pat. No. 5,304,732; U.S. Pat. No. 4,761,373; U.S. Pat. No. 5,331,107; U.S. Pat. No. 5,928,937; and U.S. Pat. No. 5,378,824; and also in the international publication WO 96/033270. Further imidazolinone-tolerant plants have also been described, for example, in WO 2004/040012, WO 2004/106529, WO 2005/020673, WO 2005/093093, WO 2006/007373, WO 2006/015376, WO 2006/024351 and WO 2006/060634. Further sulfonylurea- and imidazolinone-tolerant plants have also been described, for example, in WO 2007/024782.

Further plants tolerant to ALS inhibitors, in particular to imidazolinones, sulfonylureas and/or sulfamoylcarbonyltriazolinones, can be obtained by induced mutagenesis, by selection in cell cultures in the presence of the herbicide or by mutation breeding, as described, for example, for soybeans in U.S. Pat. No. 5,084,082, for rice in WO 97/41218, for sugarbeet in U.S. Pat. No. 5,773,702 and WO 99/057965, for lettuce in U.S. Pat. No. 5,198,599 or for sunflower in WO 2001/065922.

Plants or plant cultivars (obtained by plant biotechnology methods such as genetic engineering) which may also be treated with the compounds of the general formula (I) according to the invention are insect-resistant transgenic plants, i.e. plants made resistant to attack by certain target insects. Such plants can be obtained by genetic transformation, or by selection of plants containing a mutation imparting such insect resistance.

The term “insect-resistant transgenic plant”, as used herein, includes any plant containing at least one transgene comprising a coding sequence encoding:

1) an insecticidal crystal protein from Bacillus thuringiensis or an insecticidal portion thereof, such as the insecticidal crystal proteins compiled by Crickmore et al., Microbiology and Molecular Biology Reviews (1998), 62, 807-813, updated by Crickmore et al. (2005) in the Bacillus thuringiensis toxin nomenclature (online at: http://www.lifesci.sussex.ac.uk/Home/Neil_Crickmore/Bt/), or insecticidal portions thereof, for example proteins of the Cry protein classes Cry1Ab, Cry1Ac, Cry1F, Cry2Ab, Cry3Ae or Cry3Bb or insecticidal portions thereof; or
2) a crystal protein from Bacillus thuringiensis or a portion thereof which is insecticidal in the presence of a second, other crystal protein than Bacillus thuringiensis or a portion thereof, such as the binary toxin made up of the Cy34 and Cy35 crystal proteins (Moellenbeck et al., Nat. Biotechnol. (2001), 19, 668-72; Schnepf et al., Applied Environm. Microb. (2006), 71, 1765-1774); or
3) a hybrid insecticidal protein comprising parts of two different insecticidal crystal proteins from Bacillus thuringiensis, such as a hybrid of the proteins of 1) above or a hybrid of the proteins of 2) above, for example the Cry1A.105 protein produced by maize event MON98034 (WO 2007/027777); or
4) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation, such as the Cry3Bb1 protein in maize events MON863 or MON88017, or the Cry3A protein in maize event MIR 604; or
5) an insecticidal secreted protein from Bacillus thuringiensis or Bacillus cereus, or an insecticidal portion thereof, such as the vegetative insecticidal proteins (VIPs) listed under the following link, for example proteins from the VIP3Aa protein class: http://www.lifesci.sussex.ac.uk/Home/Neil_Crickmore/Bt/vip.html; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins (WO 94/21795); or
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation (while still encoding an insecticidal protein), such as the VIP3Aa protein in cotton event COT 102.

Of course, insect-resistant transgenic plants, as used herein, also include any plant comprising a combination of genes encoding the proteins of any one of the above classes 1 to 8. In one embodiment, an insect-resistant plant contains more than one transgene encoding a protein of any one of the above classes 1 to 8, to expand the range of the target insect species affected or to delay insect resistance development to the plants, by using different proteins insecticidal to the same target insect species but having a different mode of action, such as binding to different receptor binding sites in the insect.

Plants or plant cultivars (obtained by plant biotechnology methods such as genetic engineering) which may also be treated with the compounds according to the invention of the general formula (I) are tolerant to abiotic stress factors. Such plants can be obtained by genetic transformation, or by selection of plants containing a mutation imparting such stress resistance. Particularly useful stress tolerant plants include:

a. plants which contain a transgene capable of reducing the expression and/or the activity of the poly(ADP-ribose)polymerase (PARP) gene in the plant cells or plants, as described in WO 2000/004173 or EP 04077984.5 or EP 06009836.5;
b. plants which contain a stress tolerance-enhancing transgene capable of reducing the expression and/or the activity of the PARG-encoding genes of the plants or plant cells, as described, for example, in WO 2004/090140;
c. plants which contain a stress tolerance-enhancing transgene encoding a plant-functional enzyme of the nicotinamide adenine dinucleotide salvage biosynthesis pathway, including nicotinamidase, nicotinate phosphoribosyltransferase, nicotinic acid mononucleotide adenyltransferase, nicotinamide adenine dinucleotide synthetase or nicotinamide phosphoribosyltransferase, as described, for example, in EP 04077624.7 or WO 2006/133827 or PCT/EP07/002433.

Plants or plant varieties (obtained by plant biotechnology methods such as genetic engineering) which may also be treated with the compounds of the general formula (I) according to the invention show altered quantity, quality and/or storage stability of the harvested product and/or altered properties of specific ingredients of the harvested product such as, for example:

1) transgenic plants which synthesize a modified starch which, in its physicochemical characteristics, in particular the amylose content or the amylose/amylopectin ratio, the degree of branching, the average chain length, the side chain distribution, the viscosity behavior, the gelling strength, the starch granule size and/or the starch granule morphology, is changed in comparison with the synthesized starch in wild-type plant cells or plants, so that this modified starch is better suited to specific applications. These transgenic plants which synthesize a modified starch are described, for example, in EP 0571427, WO 95/004826, EP 0719338, WO 96/15248, WO 96/19581, WO 96/27674, WO 97/11188, WO 97/26362, WO 97/32985, WO 97/42328, WO 97/44472, WO 97/45545, WO 98/27212, WO 98/40503, WO 99/58688, WO 99/58690, WO 99/58654, WO 2000/008184, WO 2000/008185, WO 2000/28052, WO 2000/77229, WO 2001/12782, WO 2001/12826, WO 2002/101059, WO 2003/071860, WO 2004/056999, WO 2005/030942, WO 2005/030941, WO 2005/095632, WO 2005/095617, WO 2005/095619, WO 2005/095618, WO 2005/123927, WO 2006/018319, WO 2006/103107, WO 2006/108702, WO 2007/009823, WO 2000/22140, WO 2006/063862, WO 2006/072603, WO 2002/034923, EP 06090134.5, EP 06090228.5, EP 06090227.7, EP 07090007.1, EP 07090009.7, WO 2001/14569, WO 2002/79410, WO 2003/33540, WO 2004/078983, WO 2001/19975, WO 95/26407, WO 96/34968, WO 98/20145, WO 99/12950, WO 99/66050, WO 99/53072, U.S. Pat. No. 6,734,341, WO 2000/11192, WO 98/22604, WO 98/32326, WO 2001/98509, WO 2001/98509, WO 2005/002359, U.S. Pat. No. 5,824,790, U.S. Pat. No. 6,013,861, WO 94/004693, WO 94/009144, WO 94/11520, WO 95/35026 or WO 97/20936.
2) transgenic plants which synthesize non-starch carbohydrate polymers or which synthesize non-starch carbohydrate polymers with altered properties in comparison to wild type plants without genetic modification. Examples are plants which produce polyfructose, especially of the inulin and levan type, as described in EP 0663956, WO 96/001904, WO 96/021023, WO 98/039460 and WO 99/024593, plants which produce alpha-1,4-glucans as described in WO 95/031553, US 2002/031826, U.S. Pat. No. 6,284,479, U.S. Pat. No. 5,712,107, WO 97/047806, WO 97/047807, WO 97/047808 and WO 2000/14249, plants which produce alpha-1,6-branched alpha-1,4-glucans as described in WO 2000/73422, and plants producing alternan, as described in WO 2000/047727, EP 06077301.7, U.S. Pat. No. 5,908,975 and EP 0728213.
3) Transgenic plants which produce hyaluronan, as described for example in WO 06/032538, WO 2007/039314, WO 2007/039315, WO 2007/039316, JP 2006/304779 and WO 2005/012529.

Plants or plant cultivars (obtained by plant biotechnology methods such as genetic engineering) which may also be treated with the compounds of the general formula (I) according to the invention are plants, such as cotton plants, with altered fiber characteristics. Such plants can be obtained by genetic transformation, or by selection of plants containing a mutation imparting such altered fibre characteristics and include:

a) plants, such as cotton plants, which contain an altered form of cellulose synthase genes, as described in WO 98/000549;
b) plants, such as cotton plants, which contain an altered form of rsw2 or rsw3 homologous nucleic acids, as described in WO 2004/053219;
c) plants, such as cotton plants, with an increased expression of sucrose phosphate synthase, as described in WO 2001/017333;
d) plants, such as cotton plants, with increased expression of sucrose synthase as described in WO 02/45485;
e) plants, such as cotton plants, wherein the timing of the plasmodesmatal gating at the basis of the fiber cell is altered, for example through downregulation of fiber-selective β-1,3-glucanase as described in WO 2005/017157;
f) plants, such as cotton plants, which have fibers with altered reactivity, for example through expression of the N-acetylglucosamine transferase gene including nodC and chitin synthase genes, as described in WO 2006/136351.

Plants or plant cultivars (obtained by plant biotechnology methods such as genetic engineering) which may also be treated with the compounds of the general formula (I) according to the invention are plants, such as oilseed rape or related Brassica plants, with altered oil profile characteristics. Such plants can be obtained by genetic transformation, or by selection of plants containing a mutation imparting such altered oil characteristics and include:

a) plants, such as oilseed rape plants, which produce oil having a high oleic acid content, as described, for example, in U.S. Pat. No. 5,969,169, U.S. Pat. No. 5,840,946 or U.S. Pat. No. 6,323,392 or U.S. Pat. No. 6,063,947;
b) plants, such as oilseed rape plants, which produce oil having a low linolenic acid content, as described in U.S. Pat. No. 6,270,828, U.S. Pat. No. 6,169,190 or U.S. Pat. No. 5,965,755;
c) plants, such as oilseed rape plants, which produce oil having a low level of saturated fatty acids, as described, for example, in U.S. Pat. No. 5,434,283.

Particularly useful transgenic plants which may be treated with the compounds of the general formula (I) according to the invention are plants containing transformation events, or a combination of transformation events, and that are listed for example in the databases of various national or regional regulatory agencies.

Particularly useful transgenic plants which may be treated with the compounds of the general formula (I) according to the invention are, for example, plants which comprise one or more genes which encode one or more toxins and are the transgenic plants available under the following trade names: YIELD GARD® (for example corn, cotton, soybeans), KnockOut® (for example corn), BiteGard® (for example corn), BT-Xtra® (for example corn), StarLink@ (for example corn), Bollgard® (cotton), Nucotn® (cotton), Nucotn 33B® (cotton), NatureGard® (for example corn), Protecta® and NewLeaf® (potato). Examples of herbicide-tolerant plants which may be mentioned are maize varieties, cotton varieties and soya bean varieties which are available under the following trade names: Roundup Ready® (tolerance to glyphosates, for example corn, cotton, soybeans), Liberty Link® (tolerance to phosphinothricin, for example oilseed rape), IMI® (tolerance to imidazolinone) and SCS® (tolerance to sulfonylurea, for example corn). Herbicide-resistant plants (plants bred in a conventional manner for herbicide tolerance) which may be mentioned include the varieties sold under the name Clearfield® (for example maize).

The compounds of the formula (I) to be used in accordance with the invention can be converted to customary formulations, such as solutions, emulsions, wettable powders, water- and oil-based suspensions, powders, dusts, pastes, soluble powders, soluble granules, granules for broadcasting, suspoemulsion concentrates, natural substances impregnated with active compound, synthetic substances impregnated with active compound, fertilizers, and also microencapsulations in polymeric substances. In the context of the present invention, it is especially preferred when the compounds of the general formula (I) are used in the form of a spray formulation.

The present invention therefore additionally also relates to a spray formulation for enhancing the resistance of plants to abiotic stress. A spray formulation is described in detail hereinafter:

The formulations for spray application are produced in a known manner, for example by mixing the compounds of the general formula (I) for use in accordance with the invention with extenders, i.e. liquid solvents and/or solid carriers, optionally with use of surfactants, i.e. emulsifiers and/or dispersants and/or foam formers. Further customary additives, for example customary extenders and solvents or diluents, dyes, wetting agents, dispersants, emulsifiers, antifoams, preservatives, secondary thickeners, stickers, gibberellins and also water, can optionally also be used. The formulations are produced either in suitable facilities or else before or during application.

The auxiliaries used may be those substances which are suitable for imparting, to the composition itself and/or to preparations derived therefrom (for example spray liquors), particular properties such as particular technical properties and/or else special biological properties. Typical auxiliaries include: extenders, solvents and carriers.

Suitable extenders are, for example, water, polar and nonpolar organic chemical liquids, for example from the classes of the aromatic and nonaromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulfones and sulfoxides (such as dimethyl sulfoxide).

If the extender used is water, it is also possible to use, for example, organic solvents as auxiliary solvents. Useful liquid solvents essentially include: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethyl sulfoxide, and also water.

It is possible to use colorants such as inorganic pigments, for example iron oxide, titanium oxide and Prussian blue, and organic colorants such as alizarin colorants, azo colorants and metal phthalocyanine colorants, and trace nutrients such as salts of iron, manganese, boron, copper, cobalt, molybdenum and zinc.

Suitable wetting agents which may be present in the formulations which can be used in accordance with the invention are all substances which promote wetting and which are conventionally used for the formulation of agrochemical active substances. Preference is given to using alkyl naphthalenesulfonates, such as diisopropyl or diisobutyl naphthalenesulfonates.

Suitable dispersants and/or emulsifiers which may be present in the formulations which can be used in accordance with the invention are all nonionic, anionic and cationic dispersants conventionally used for the formulation of agrochemically active compounds. Preference is given to using nonionic or anionic dispersants or mixtures of nonionic or anionic dispersants. Suitable nonionic dispersants which may be mentioned are, in particular, ethylene oxide/propylene oxide block polymers, alkylphenol polyglycol ethers and tristryrylphenol polyglycol ether and their phosphated or sulfated derivatives. Suitable anionic dispersants are especially lignosulfonates, polyacrylic acid salts and arylsulfonate/formaldehyde condensates.

Suitable antifoams which may be present in the formulations which can be used in accordance with the invention are all foam-inhibiting substances conventionally used for the formulation of agrochemical active substances. Silicone antifoams and magnesium stearate can be used with preference.

Preservatives which may be present in the formulations usable in accordance with the invention are all substances usable for such purposes in agrochemical compositions. Examples include dichlorophene and benzyl alcohol hemiformal.

Secondary thickeners which may be present in the formulations usable in accordance with the invention are all substances usable for such purposes in agrochemical compositions. Preferred examples include cellulose derivatives, acrylic acid derivatives, xanthan, modified clays and finely divided silica.

Stickers which may be present in the formulations usable in accordance with the invention include all customary binders usable in seed-dressing products. Preferred examples include polyvinylpyrrolidone, polyvinyl acetate, polyvinyl alcohol and tylose. Suitable gibberellins which may be present in the formulations which can be used in accordance with the invention are preferably the gibberellins A1, A3 (=gibberellic acid), A4 and A7; gibberellic acid is especially preferably used. The gibberellins are known (cf. R. Wegler “Chemie der Pflanzenschutz- and Schädlingsbekampfungsmittel”, vol. 2, Springer Verlag, 1970, pp. 401-412).

Further additives may be fragrances, mineral or vegetable, optionally modified oils, waxes and nutrients (including trace nutrients), such as salts of iron, manganese, boron, copper, cobalt, molybdenum and zinc. Additionally present may be stabilizers, such as cold stabilizers, antioxidants, light stabilizers or other agents which improve chemical and/or physical stability.

The formulations contain generally between 0.01 and 98% by weight, preferably between 0.5 and 90%, of the compound of the general formula (I).

The compounds of the general formula (I) according to the invention may be present in commercially available formulations, and also in the use forms, prepared from these formulations, as a mixture with other active compounds, such as insecticides, attractants, sterilizing agents, bactericides, acaricides, nematicides, fungicides, growth-regulating substances, herbicides, safeners, fertilizers or semiochemicals.

In addition, the described positive effect of the compounds of the formula (I) on the plants' own defences can be supported by an additional treatment with active insecticidal, fungicidal or bactericidal compounds.

Preferred times for the application of compounds of the general formula (I) to be used according to the invention or salts thereof for enhancing resistance to abiotic stress are treatments of the soil, stems and/or leaves with the approved application rates.

The active compounds of the general formula (I) to be used in accordance with the invention, or salts thereof, may generally additionally be present in their commercial formulations and in the use forms prepared from these formulations in mixtures with other active compounds, such as insecticides, attractants, sterilants, acaricides, nematicides, fungicides, bactericides, growth regulators, substances which influence plant maturity, safeners or herbicides. Particularly favorable mixing partners are, for example, the active compounds of the different classes, specified below in groups, without any preference resulting from the sequence thereof:

### Fungicides:

F1) nucleic acid synthesis inhibitors, for example benalaxyl, benalaxyl-M, bupirimate, chiralaxyl, clozylacon, dimethirimol, ethirimol, furalaxyl, hymexazole, metalaxyl, metalaxyl-M, ofurace, oxadixyl, oxolinic acid;
F2) mitosis and cell division inhibitors, for example benomyl, carbendazim, diethofencarb, fuberidazole, fluopicolid, pencycuron, thiabendazole, thiophanate-methyl, zoxamide and chloro-7-(4-methylpiperidin-1-yl)-6-(2,4,6-trifluorophenyl)[1,2,4]triazolo[1,5-a]pyrimidine;
F3) respiratory chain complex I/II inhibitors, for example diflumetorim, bixafen, boscalid, carboxin, diflumethorim, fenfuram, fluopyram, flutolanil, furametpyr, mepronil, oxycarboxin, penflufen, penthiopyrad, thifluzamid, N-[2-(1,3-dimethylbutyl)phenyl]-5-fluoro-1,3-dimethyl-1H-pyrazole-4-carboxamide, isopyrazam, sedaxan, 3-(difluoromethyl)-1-methyl-N-(3′,4′,5′-trifluorobiphenyl-2-yl)-1H-pyrazole-4-carboxamide, 3-(difluoromethyl)-1-methyl-N-[2-(1,1,2,2-tetrafluoroethoxyl)phenyl]-1H-pyrazole-4-carboxamide, 3-(difluoromethyl)-N-[4-fluoro-2-(1,1,2,3,3,3-hexafluoropropoxyl)phenyl]-1-methyl-1H-pyrazole-4-carboxamide, N-[1-(2,4-dichlorophenyl)-1-methoxypropan-2-yl]-3-(difluoromethyl)-1-methyl-1H-pyrazole-4-carboxamide and corresponding salts;
F4) respiratory chain complex III inhibitors, for example amisulbrom, azoxystrobin, cyazofamid, dimoxystrobin, enestrobin, famoxadon, fenamidon, fluoxastrobin, kresoxim-methyl, metominostrobin, orysastrobin, pyraclostrobin, pyribencarb, picoxystrobin, trifloxystrobin, (2E)-2-(2-{[6-(3-chloro-2-methylphenoxy)-5-fluoropyrimidin-4-yl]oxy}phenyl)-2-(methoxyimino)-N-methylethanamide, (2E)-2-(ethoxyimino)-N-methyl-2-(2-{[({(1E)-1-[3-(trifluoromethyl)phenyl]ethylidene}amino)oxy]methyl}phenyl)ethanamide and corresponding salts, (2E)-2-(methoxyimino)-N-methyl-2-{2-[(E)-({1-[3-(trifluoromethyl)phenyl]ethoxy}imino)methyl]phenyl}ethanamide, (2E)-2-{2-[({[(1E)-1-(3-{[(E)-1-fluoro-2-phenylethenyl]oxy}phenyhethylidene]amino}oxy)methyl]phenyl}-2-(methoxyimino)-N-methylethanamide, (2E)-2-{2-[({[(2E,3E)-4-(2,6-dichlorophenyl)but-3-en-2-ylidene]amino}oxy)methyl]phenyl}-2-(methoxyimino)-N-methylethanamide, 2-chloro-N-(1,1,3-trimethyl-2,3-dihydro-1H-inden-4-yl)pyridine-3-carboxamide, 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, 2-methyl {2-[({cyclopropyl[(4-methoxyphenyl)imino]methyl}sulfanyl)methyl]phenyl}-3-methoxyacrylate, N-(3-ethyl-3,5,5-trimethylcyclohexyl)-3-(formylamino)-2-hydroxybenzamide and corresponding salts;
F5) decouplers, for example dinocap, fluazinam;
F6) ATP production inhibitors, for example fentin acetate, fentin chloride, fentin hydroxide, silthiofam
F7) amino acid and protein biosynthesis inhibitors, for example andoprim, blasticidin-S, cyprodinil, kasugamycin, kasugamycin hydrochloride hydrate, mepanipyrim, pyrimethanil
F8) signal transduction inhibitors, for example fenpiclonil, fludioxonil, quinoxyfen
F9) lipid and membrane synthesis inhibitors, for example chlozolinate, iprodione, procymidone, vinclozolin, ampropylfos, potassium-ampropylfos, edifenphos, iprobenfos (IBP), isoprothiolane, pyrazophos, tolclofos-methyl, biphenyl, iodocarb, propamocarb, propamocarb hydrochloride
F10) ergosterol biosynthesis inhibitors, for example fenhexamid, azaconazole, bitertanol, bromuconazole, diclobutrazole, difenoconazole, diniconazole, diniconazole-M, etaconazole, fenbuconazole, fluquinconazole, flusilazole, flutriafol, furconazole, furconazole-cis, hexaconazole, imibenconazole, ipconazole, metconazole, myclobutanil, paclobutrazole, penconazole, propiconazole, prothioconazole, simeconazole, spiroxamine, tebuconazole, triadimefon, triadimenol, triticonazole, uniconazole, voriconazole, imazalil, imazalilsulfate, oxpoconazole, fenarimol, flurprimidol, nuarimol, pyrifenox, triforin, pefurazoate, prochloraz, triflumizole, viniconazole, aldimorph, dodemorph, dodemorph acetate, fenpropimorph, tridemorph, fenpropidin, naftifin, pyributicarb, terbinafin, 1-(4-chlorophenyl)-2-(1H-1,2,4-triazol-1-yl)cycloheptanol, methyl 1-(2,2-dimethyl-2,3-dihydro-1H-inden-1-yl)-1H-imidazole-5-carboxylate, N′-{5-(difluoromethyl)-2-methyl-4-[3-(trimethylsilyl)propoxy]phenyl}-N-ethyl-N-methylimidoformamide, N-ethyl-N-methyl-N′-{2-methyl-5-(trifluoromethyl)-4-[3-(trimethylsilyl)propoxy]phenyl}imidoformamide and O-{1-[(4-methoxyphenoxy)methyl]2,2-dimethylpropyl}-1H-imidazole-1-carbothioate;
F11) cell wall synthesis inhibitors, for example benthiavalicarb, bialaphos, dimethomorph, flumorph, iprovalicarb, polyoxins, polyoxorim, validamycin A
F12) melanine biosynthesis inhibitors, for example capropamide, diclocymet, fenoxanil, phthalide, pyroquilon, tricyclazole
F13) resistance induction, for example acibenzolar-S-methyl, probenazole, tiadinil
F14) multisite, for example captafol, captan, chlorothalonil, copper salts such as: copper hydroxide, copper naphthenate, copper oxychloride, copper sulfate, copper oxide, oxine-copper and Bordeaux mixture, dichlofluanid, dithianon, dodine, dodine free base, ferbam, folpet, fluorofolpet, guazatine, guazatine acetate, iminoctadine, iminoctadine albesilate, iminoctadine triacetate, mancopper, mancozeb, maneb, metiram, metiram zinc, propineb, sulfur and sulfur preparations containing calcium polysulfide, thiram, tolylfluanid, zineb, ziram
F15) unknown mechanism, for example amibromdol, benthiazole, bethoxazin, capsimycin, carvone, chinomethionat, chloropicrin, cufraneb, cyflufenamid, cymoxanil, dazomet, debacarb, diclomezine, dichlorophen, dicloran, difenzoquat, difenzoquat methyl sulfate, diphenylamine, ethaboxam, ferimzone, flumetover, flusulfamide, fluopicolid, fluoroimid, fosatyl-A1, hexachlorobenzene, 8-hydroxyquinoline sulfate, iprodione, irumamycin, isotianil, methasulfocarb, metrafenone, methyl isothiocyanate, mildiomycin, natamycin, nickel dimethyl dithiocarbamate, nitrothal-isopropyl, octhilinone, oxamocarb, oxyfenthiin, pentachlorophenol and salts, 2-phenylphenol and salts, piperalin, propanosine-sodium, proquinazid, pyrrolnitrin, quintozene, tecloftalam, tecnazene, triazoxide, trichlamide, zarilamid and 2,3,5,6-tetrachloro-4-(methylsulfonyl)pyridine, N-(4-chloro-2-nitrophenyl)-N-ethyl-4-methylbenzenesulfonamide, 2-amino-4-methyl-N-phenyl-5-thiazolecarboxamide, 2-chloro-N-(2,3-dihydro-1,1,3-trimethyl-1H-inden-4-yl)-3-pyridinecarboxamide, 3-[5-(4-chlorophenyl)-2,3-dimethylisoxazolidin-3-yl]pyridine, cis-1-(4-chlorophenyl)-2-(1H-1,2,4-triazol-1-yl)cycloheptanol, 2,4-dihydro-5-methoxy-2-methyl-4-[[[[1-[3-(trifluoromethyl)phenyl]ethylidene]amino]oxy]methyl]phenyl]-3H-1,2,3-triazol-3-one (185336-79-2), methyl 1-(2,3-dihydro-2,2-dimethyl-1H-inden-1-yl)-1H-imidazole-5-carboxylate, 3,4,5-trichloro-2,6-pyridinedicarbonitrile, methyl 2-[[[cyclopropyl[(4-methoxyphenyl)imino]methyl]thio]methyl]-.alpha.-(methoxymethylene)benzacetate, 4-chloro-alpha-propynyloxy-N-[2-[3-methoxy-4-(2-propynyloxyl)phenyl]ethyl]benzacetamide, (2S)—N-[2-[4-[[3-(4-chlorophenyl)-2-propynyl]oxy]-3-methoxyphenyl]ethyl]-3-methyl-2-[(methylsulfonyl)amino]butanamide, 5-chloro-7-(4-methylpiperidin-1-yl)-6-(2,4,6-trifluorophenyl)[1,2,4]triazolo[1,5-a]pyrimidine, 5-chloro-6-(2,4,6-trifluorophenyl)-N-[(1R)-1,2,2-trimethylpropyl][1,2,4]triazolo[1,5-a]pyrimidine-7-amine, 5-chloro-N-[(1R)-1,2-dimethylpropyl]-6-(2,4,6-trifluorophenyl)[1,2,4]triazolo[1,5-a]pyrimidine-7-amine, N-[1-(5-bromo-3-chloropyridin-2-yl)ethyl]-2,4-dichloronicotinamide, N-(5-bromo-3-chloropyridin-2-yl)methyl-2,4-dichloronicotinamide, 2-butoxy-6-iodo-3-propylbenzopyranon-4-one, N-{(Z)-[(cyclopropylmethoxy)imino][6-(difluoromethoxy)-2,3-difluorophenyl]methyl}-2-benzacetamide, N-(3-ethyl-3,5,5-trimethylcyclohexyl)-3-formylamino-2-hydroxybenzamide, 2-[[[[1-[3-(1-fluoro-2-phenylethyl)oxy]phenyl]ethylidene]amino]oxy]methyl]-alpha-(methoxyimino)-N-methyl-alphaE-benzacetamide, N-{2-[3-chloro-5-(trifluoromethyl)pyridin-2-yl]ethyl}-2-(trifluoromethyl)benzamide, N-(3′,4′-dichloro-5-fluorobiphenyl-2-yl)-3-(difluoromethyl)-1-methyl-1H-pyrazole-4-carboxamide, N-(6-methoxy-3-pyridinyl)cyclopropanecarboxamide, 1-[(4-methoxyphenoxy)methyl]-2,2-dimethylpropyl-1H-imidazole-1-carboxylic acid, O-[1-[(4-methoxyphenoxy)methyl]-2,2-dimethylpropyl]-1H-imidazole-1-carbothioic acid, 2-(2-{[6-(3-chloro-2-methylphenoxy)-5-fluoropyrimidin-4-yl]oxy}phenyl)-2-(methoxyimino)-N-methylacetamide.

### Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate and other copper preparations.

### Insecticides/Acaricides/Nematicides:

I1) Acetylcholinesterase (AChE) inhibitors, for example carbamates, e.g. alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates, e.g. acephate, azamethiphos, azinphos (-methyl, -ethyl), cadusafos, chlorethoxyfos, chlorfenvinphos, chlormephos, chlorpyrifos (-methyl), coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, isofenphos, isopropyl 0-(methoxyaminothiophosphoryl) salicylate, isoxathion, malathion, mecarbam, methamidophos, methidathion, mevinphos, monocrotophos, naled, omethoate, oxydemeton-methyl, parathion (-methyl), phenthoate, phorate, phosalone, phosmet, phosphamidon, phoxim, pirimiphos (-methyl), profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.
I2) GABA-gated chloride channel antagonists, for example organochlorines, e.g. chlordane and endosulfan (alpha-); or fiproles (phenylpyrazoles), e.g. ethiprole, fipronil, pyrafluprole and pyriprole.
I3) Sodium channel modulators/voltage-gated sodium channel blockers, for example pyrethroids, e.g. acrinathrin, allethrin (d-cis-trans, d-trans), bifenthrin, bioallethrin, bioallethrin-S-cyclopentenyl, bioresmethrin, cycloprothrin, cyfluthrin (beta-), cyhalothrin (gamma-, lambda-), cypermethrin (alpha-, beta-, theta-, zeta-), cyphenothrin [(1R)-trans-isomers], deltamethrin, dimefluthrin, empenthrin [(EZ)-(1R)-isomers], esfenvalerate, etofenprox, fenpropathrin, fenvalerate, flucythrinate, flumethrin, fluvalinate (tau-), halfenprox, imiprothrin, metofluthrin, permethrin, phenothrin [(1R)-trans-isomer], prallethrin, profluthrin, pyrethrins (pyrethrum), resmethrin, RU 15525, silafluofen, tefluthrin, tetramethrin [(1R)-isomers], tralomethrin, transfluthrin and ZXI 8901; or _DDT; or methoxychlor.
I4) Nicotinergic acetylcholine receptor agonists, for example neonicotinoids, e.g. acetamiprid, clothianidin, dinotefuran, imidacloprid, nitenpyram, thiacloprid, thiamethoxam; or nicotine.
I5) Allosteric acetylcholine receptor modulators (agonists) for example spinosyns, e.g. spinetoram and spinosad.
I6) Chloride channel activators, for example avermectins/milbemycins, e.g. abamectin, emamectin, emamectin benzoate, lepimectin and milbemectin.
I7) Juvenile hormone analogs, e.g. hydroprene, kinoprene, methoprene; or fenoxycarb; pyriproxyfen.
I8) Active compounds with unknown or non-specific mechanisms of action, for example fumigants, for example methyl bromide and other alkyl halides; or chloropicrin; sulfuryl fluoride; borax; tartar emetic.
I9) Selective antifeedants, e.g. pymetrozine; or flonicamid.
I10) Mite growth inhibitors, e.g. clofentezine, diflovidazin, hexythiazox, etoxazole.
I11) Microbial disruptors of the insect gut membrane, for example Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and BT plant proteins, for example Cry1Ab, Cry1Ac, Cry1Fa, Cry2Ab, mCry3A, Cry3Ab, Cry3Bb, Cry34/35Ab1.
I12) Oxidative phosphorylation inhibitors, ATP disruptors, for example diafenthiuron; or organotin compounds, e.g. azocyclotin, cyhexatin, fenbutatin oxide; or propargite; tetradifon.
I13) Oxidative phosphorylation decouplers through interruption of the H proton gradient, for example chlorfenapyr and DNOC.
I14) Nicotinergic acetylcholine receptor antagonists, for example bensultap, cartap (-hydrochloride), thiocyclam, and thiosultap (-sodium).
I15) Chitin biosynthesis inhibitors, type 0, for example benzoylureas, e.g. bistrifluron, chlorfluazuron, diflubenzuron, flucycloxuron, flufenoxuron, hexaflumuron, lufenuron, novaluron, noviflumuron, teflubenzuron and triflumuron.
I16) Chitin biosynthesis inhibitors, type 1, for example buprofezin.
I17) Moulting disruptors, for example cyromazine.
I18) Ecdysone agonists/disruptors, for example diacylhydrazines, for example chromafenozide, halofenozide, methoxyfenozide and tebufenozide.
I19) Octopaminergic agonists, for example amitraz.
I20) Complex III electron transport inhibitors, for example hydramethylnone; acequinocyl; fluacrypyrim.
I21) Complex I electron transport inhibitors, for example from the group of the METI acaricides, e.g. fenazaquin, fenpyroximate, pyrimidifen, pyridaben, tebufenpyrad, tolfenpyrad; or rotenone (Derris).
I22) Voltage-gated sodium channel blockers, e.g. indoxacarb; metaflumizone.
I23) Inhibitors of acetyl-CoA carboxylase, for example tetronic acid derivatives, e.g. spirodiclofen and spiromesifen; or tetramic acid derivatives, e.g. spirotetramat.
I24) Complex IV electron transport inhibitors, for example phosphines, e.g. aluminum phosphide, calcium phosphide, phosphine, zinc phosphide; or cyanide.
I25) Complex II electron transport inhibitors, for example cyenopyrafen.
I26) Ryanodine receptor effectors, for example diamides, e.g. flubendiamide, chlorantraniliprole (Rynaxypyr), cyantraniliprole (Cyazypyr) and 3-bromo-N-{2-bromo-4-chloro-6-[(1-cyclopropylethyl)carbamoyl]phenyl}-1-(3-chloropyridin-2-yl)-1H-pyrazole-5-carboxamide (known from WO2005/077934) or methyl 2-[3,5-dibromo-2-({[3-bromo-1-(3-chloropyridin-2-yl)-1H-pyrazol-5-yl]carbonyl}amino)benzoyl]-1,2-dimethylhydrazinecarboxylate (known from WO2007/043677).

Further active compounds having an unknown mechanism of action, such as, for example, azadirachtin, amidoflumet, benzoximate, bifenazate, chinomethionat, cryolite, cyflumetofen, dicofol, 5-chloro-2-[(3,4,4-trifluorobut-3-en-1-yl)sulfonyl]-1,3-thiazole, flufenerim, pyridalyl and pyrifluquinazon; furthermore preparations based on Bacillus firmus (1-1582, BioNeem, Votivo) and the following known active compounds 4-{[(6-bromopyrid-3-yl)methyl](2-fluoroethyl)amino}furan-2(5H)-one (known from WO 2007/115644), 4-{[(6-fluoropyrid-3-yl)methyl](2,2-difluoroethyl)amino}furan-2(5H)-one (known from WO 2007/115644), 4-{[(2-chloro-1,3-thiazol-5-yl)methyl](2-fluoroethyl)amino}furan-2(5H)-one (known from WO 2007/115644), 4-{[(6-chloropyrid-3-yl)methyl](2-fluoroethyl)amino}furan-2(5H)-one (known from WO 2007/115644), 4-{[(6-chloropyrid-3-yl)methyl](2,2-difluoroethyl)amino}furan-2(5H)-one (known from WO 2007/115644), 4-{[(6-chloro-5-fluoropyrid-3-yl)methyl](methyl)amino}furan-2(5H)-one (known from WO 2007/115643), 4-{[(5,6-dichloropyrid-3-yl)methyl](2-fluoroethyl)amino}furan-2(5H)-one (known from WO 2007/115646), 4-{[(6-chloro-5-fluoropyrid-3-yl)methyl](cyclopropyl)amino}furan-2(5H)-one (known from WO 2007/115643), 4-{[(6-chloropyrid-3-yl)methyl](cyclopropyl)amino}furan-2(5H)-one (known from EP0539588), 4-{[(6-chloropyrid-3-yl)methyl](methyl)amino}furan-2(5H)-one (known from EP0539588), [1-(6-chloropyridin-3-yl)ethyl](methyl)oxido-λ⁴-sulfanylidenecyanamide (known from WO 2007/149134) and its diastereomers {[(1R)-1-(6-chloropyridin-3-yl)ethyl](methyl)oxido-λ⁶-sulfanylidene}cyanamide and {[(1S)-1-(6-chloropyridin-3-yl)ethyl](methyl)oxido-λ⁶-sulfanylidene}cyanamide (likewise known from WO 2007/149134) and sulfoxaflor (likewise known from WO 2007/149134), 1-[2-fluoro-4-methyl-5-[(2,2,2-trifluoroethyl)sulfinyl]phenyl]-3-(trifluoromethyl)-1H-1,2,4-triazole-5-amine (known from WO 2006/043635), [(3S,4αR,12R,12αS,12βS)-3-[(cyclopropylcarbonyl)oxy]-6,12-dihydroxy-4,12b-dimethyl-11-oxo-9-(pyridin-3-yl)-1,3,4,4a,5,6,6α,12,12α,12β-decahydro-2H,11H-benzo[f]pyrano[4,3-b]chromen-4-yl]methylcyclopropanecarboxylate (known from WO 2006/129714), 2-cyano-3-(difluoromethoxy)-N,N-dimethylbenzenesulfonamide (known from WO2006/056433), 2-cyano-3-(difluoromethoxy)-N-methylbenzenesulfonamide (known from WO2006/100288), 2-cyano-3-(difluoromethoxy)-N-ethylbenzenesulfonamide (known from WO2005/035486), 4-(difluoromethoxy)-N-ethyl-N-methyl-1,2-benzothiazole-3-amine 1,1-dioxide (known from WO2007/057407), N-[1-(2,3-dimethylphenyl)-2-(3,5-dimethylphenyhethyl]-4,5-dihydro-1,3-thiazole-2-amine (known from WO2008/104503), {1′-[(2E)-3-(4-chlorophenyl)prop-2-en-1-yl]-5-fluorospiro[indole-3,4′-piperidin]-1 (2H)-yl}(2-chloropyridin-4-yl)methanone (known from WO2003/106457), 3-(2,5-dimethylphenyl)-4-hydroxy-8-methoxy-1,8-diazaspiro[4.5]dec-3-en-2-one (known from WO2009/049851), 3-(2,5-dimethylphenyl)-8-methoxy-2-oxo-1,8-diazaspiro[4.5]dec-3-en-4-yl ethyl carbonate (known from WO2009/049851), 4-(but-2-yn-1-yloxy)-6-(3,5-dimethylpiperidin-1-yl)-5-fluoropyrimidine (known from WO2004/099160), (2,2,3,3,4,4,5,5-octafluoropentyl)(3,3,3-trifluoropropyl)malononitrile (known from WO2005/063094), (2,2,3,3,4,4,5,5-octafluoropentyl)(3,3,4,4,4-pentafluorobutyl)malononitrile (known from WO2005/063094), 8-[2-(cyclopropylmethoxy)-4-(trifluoromethyl)phenoxy]-3-[6-(trifluoromethyl)pyridazin-3-yl]-3-azabicyclo[3.2.1]octane (known from WO2007/040280/282), 2-ethyl-7-methoxy-3-methyl-6-[(2,2,3,3-tetrafluoro-2,3-dihydro-1,4-benzodioxin-6-yl)oxy]quinolin-4-yl methyl carbonate (known from JP2008110953), 2-ethyl-7-methoxy-3-methyl-6-[(2,2,3,3-tetrafluoro-2,3-dihydro-1,4-benzodioxin-6-yl)oxy]quinolin-4-yl acetate (known from JP2008110953), PF1364 (Chemical Abstracts No 1204776-60-2, known from JP2010018586), 5-[5-(3,5-dichlorophenyl)-5-(trifluoromethyl)-4,5-dihydro-1,2-oxazol-3-yl]-2-(1H-1,2,4-triazol-1-yl)benzonitrile (known from WO2007/075459), 5-[5-(2-chloropyridin-4-yl)-5-(trifluoromethyl)-4,5-dihydro-1,2-oxazol-3-yl]-2-(1H-1,2,4-triazol-1-yl)benzonitrile (known from WO2007/075459), 4-[5-(3,5-dichlorophenyl)-5-(trifluoromethyl)-4,5-dihydro-1,2-oxazol-3-yl]-2-methyl-N-{2-oxo-2-[(2,2,2-trifluoroethyl)amino]ethyl}benzamide (known from WO2005/085216).

Safeners are preferably selected from the group consisting of:

S1) compounds of the formula (S1)

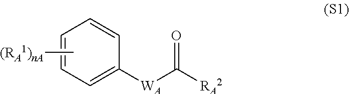

where the symbols and indices have the following meanings:
n_{A} is a natural number from 0 to 5, preferably from 0 to 3;
R_{A}¹ is halogen, (C₁-C₄)-alkyl, (C₁-C₄)-alkoxy, nitro or (C₁-C₄)-haloalkyl;

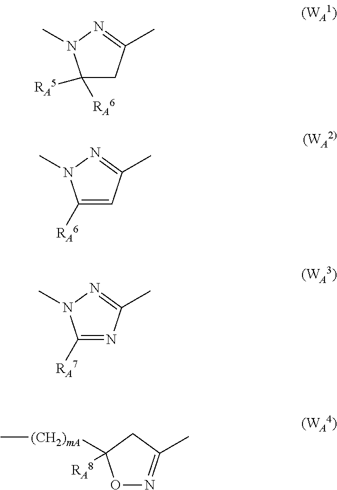

W_{A} is an unsubstituted or substituted divalent heterocyclic radical from the group of the partially unsaturated or aromatic five-membered heterocycles having 1 to 3 ring heteroatoms from the N and O group, where at least one nitrogen atom and at most one oxygen atom is present in the ring, preferably a radical from the group of (W_{A}¹) to (W_{A}⁴);
m_{A} is 0 or 1;
R_{A}² is OR_{A}³, SR_{A}³ or NR_{A}³R_{A}⁴ or a saturated or unsaturated 3- to 7-membered heterocycle having at least one nitrogen atom and up to 3 heteroatoms, preferably from the group consisting of 0 and S, which is joined to the carbonyl group in (S1) via the nitrogen atom and is unsubstituted or substituted by radicals from the group consisting of (C₁-C₄)-alkyl, (C₁-C₄)-alkoxy or optionally substituted phenyl, preferably a radical of the formula OR_{A}³, NHR_{A}⁴ or N(CH₃)₂, especially of the formula OR_{A}³;
R_{A}³ is hydrogen or an unsubstituted or substituted aliphatic hydrocarbyl radical preferably having a total of 1 to 18 carbon atoms;
R_{A}⁴ is hydrogen, (C₁-C₆)-alkyl, (C₁-C₆)-alkoxy or substituted or unsubstituted phenyl; R_{A}⁵ is H, (C₁-C₈)-alkyl, (C₁-C₈)-haloalkyl, (C₁-C₄)-alkoxy-(C₁-C₈)-alkyl, cyano or
COOR_{A}⁹ in which R_{A}⁹ is hydrogen, (C₁-C₈)-alkyl, (C₁-C₈)-haloalkyl, (C₁-C₄)-alkoxy-(C₁-C₄)-alkyl, (C₁-C₆)-hydroxyalkyl, (C₃-C₁₂)-cycloalkyl or tri-(C₁-C₄)-alkylsilyl; R_{A}⁶, R_{A}⁷, R_{A}⁸ are the same or different and are each hydrogen, (C₁-C₈)-alkyl, (C₁-C₈)-haloalkyl, (C₃-C₁₂)-cycloalkyl or substituted or unsubstituted phenyl;
preferably:
a) compounds of the dichlorophenylpyrazoline-3-carboxylic acid type (S1^{a}), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874;
b) derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described in EP-A-333 131 and EP-A-269 806;
c) derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described in EP-A-268 554, for example;
d) compounds of the triazolecarboxylic acid type (S1^{d}), preferably compounds such as fenchlorazole(-ethyl ester), i.e. ethyl 1-(2,4-dichlorophenyl)-5-trichloromethyl-(1H)-1,2,4-triazole-3-carboxylate (S1-7), and related compounds as described in EP-A-174 562 and EP-A-346 620;
e) compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazoline-3-carboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13), as described in patent application WO-A-95/07897.
S2) Quinoline derivatives of the formula (S2)

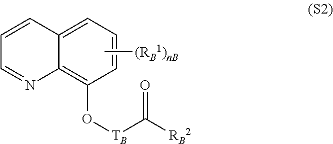

where the symbols and indices have the following meanings:
R_{B}¹ is halogen, (C₁-C₄)-alkyl, (C₁-C₄)-alkoxy, nitro or (C₁-C₄)-haloalkyl;
n_{B} is a natural number from 0 to 5, preferably from 0 to 3;
R_{B}² is OR_{B}³, SR_{B}³ or NR_{B}³R_{B}⁴ or a saturated or unsaturated 3- to 7-membered heterocycle having at least one nitrogen atom and up to 3 heteroatoms, preferably from the group of O and S, which is joined via the nitrogen atom to the carbonyl group in (S2) and is unsubstituted or substituted by radicals from the group of (C₁-C₄)-alkyl, (C₁-C₄)-alkoxy or optionally substituted phenyl, preferably a radical of the formula OR_{B}³, NHR_{B}⁴ or N(CH₃)₂, especially of the formula OR_{B}³;
R_{B}³ is hydrogen or an unsubstituted or substituted aliphatic hydrocarbyl radical preferably having a total of 1 to 18 carbon atoms;
R_{B}⁴ is hydrogen, (C₁-C₆)-alkyl, (C₁-C₆)-alkoxy or substituted or unsubstituted phenyl;
T_{B} is a (C₁ or C₂)-alkanediyl chain which is unsubstituted or substituted by one or two (C₁-C₄)-alkyl radicals or by [(C₁-C₃)-alkoxy]carbonyl;
preferably:
a) compounds of the 8-quinolinoxyacetic acid type (S2^{a}), preferably 1-methylhexyl (5-chloro-8-quinolinoxy)acetate (“cloquintocet-mexyl”) (S2-1), 1,3-dimethylbut-1-yl (5-chloro-8-quinolinoxy)acetate (S2-2), 4-allyloxybutyl (5-chloro-8-quinolinoxy)acetate (S2-3), 1-allyloxyprop-2-yl (5-chloro-8-quinolinoxy)acetate (S2-4), ethyl (5-chloro-8-quinolinoxy)acetate (S2-5), methyl (5-chloro-8-quinolinoxy)acetate (S2-6), allyl (5-chloro-8-quinolinoxy)acetate (S2-7), 2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86 750, EP-A-94 349 and EP-A-191 736 or EP-A-0 492 366, and also (5-chloro-8-quinolinoxy)acetic acid (S2-10), hydrates and salts thereof, for example the lithium, sodium, potassium, calcium, magnesium, aluminum, iron, ammonium, quaternary ammonium, sulfonium or phosphonium salts thereof, as described in WO-A-2002/34048;
b) compounds of the (5-chloro-8-quinolinoxy)malonic acid type (S2^{b}), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
S3) Compounds of the formula (S3)

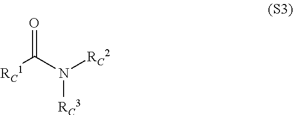

where the symbols and indices have the following meanings:
R_{C}¹ is (C₁-C₄)-alkyl, (C₁-C₄)-haloalkyl, (C₂-C₄)-alkenyl, (C₂-C₄)-haloalkenyl, (C₃-C₇)-cycloalkyl, preferably dichloromethyl; R_{C}², R_{C}³ are identical or different and are each hydrogen, (C₁-C₄)-alkyl, (C₂-C₄)-alkenyl, (C₂-C₄)-alkynyl, (C₁-C₄)-haloalkyl, (C₂-C₄)-haloalkenyl, (C₁-C₄)-alkylcarbamoyl-(C₁-C₄)-alkyl, (C₂-C₄)-alkenylcarbamoyl-(C₁-C₄)-alkyl, (C₁-C₄)-alkoxy-(C₁-C₄)-alkyl, dioxolanyl-(C₁-C₄)-alkyl, thiazolyl, furyl, furylalkyl, thienyl, piperidyl, substituted or unsubstituted phenyl, or R_{C}² and R_{C}³ together form a substituted or unsubstituted heterocyclic ring, preferably an oxazolidine, thiazolidine, piperidine, morpholine, hexahydropyrimidine or benzoxazine ring; preferably: active compounds of the dichloroacetamide type which are frequently used as pre-emergence safeners (soil-active safeners), such as, for example, “dichlormid” (N,N-diallyl-2,2-dichloroacetamide) (S3-1), “R-29148” (3-dichloroacetyl-2,2,5-trimethyl-1,3-oxazolidine) from Stauffer (S3-2), “R-28725” (3-dichloroacetyl-2,2-dimethyl-1,3-oxazolidine) from Stauffer (S3-3), “benoxacor” (4-dichloroacetyl-3,4-dihydro-3-methyl-2H-1,4-benzoxazine) (S3-4), “PPG-1292” (N-allyl-N-[(1,3-dioxolan-2-yl)methyl]dichloroacetamide) from PPG Industries (S3-5), “DKA-24” (N-allyl-N-[(allylaminocarbonyl)methyl]dichloroacetamide) from Sagro-Chem (S3-6), “AD-67” or “MON 4660” (3-dichloroacetyl-1-oxa-3-azaspiro[4,5]decane) from Nitrokemia or Monsanto (S3-7), “TI-35” (1-dichloroacetylazepane) from TRI-Chemical RT (S3-8), “diclonon” (dicyclonone) or “BAS145138” or “LAB145138” (S3-9) ((RS)-1-dichloroacetyl-3,3,8a-trimethylperhydropyrrolo[1,2-a]pyrimidin-6-one) from BASF, “furilazole” or “MON 13900” ((RS)-3-dichloroacetyl-5-(2-furyl)-2,2-dimethyloxazolidine) (S3-10); and the (R) isomer thereof (S3-11).
S4) N-acylsulfonamides of the formula (S4) and salts thereof,

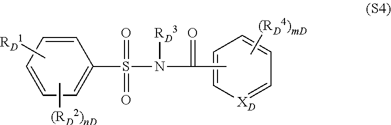

where the symbols and indices have the following meanings:

### X_{D} is CH or N;

### R_{D}¹ is CO—NR_{D}⁵R_{D}⁶ or NHCO—R_{D}⁷;

R_{D}² is halogen, (C₁-C₄)-haloalkyl, (C₁-C₄)-haloalkoxy, nitro, (C₁-C₄)-alkyl, (C₁-C₄)-alkoxy, (C₁-C₄)-alkylsulfonyl, (C₁-C₄)-alkoxycarbonyl or (C₁-C₄)-alkylcarbonyl;
R_{D}³ is hydrogen, (C₁-C₄)-alkyl, (C₂-C₄)-alkenyl or (C₂-C₄)-alkynyl;
R_{D}⁴ is halogen, nitro, (C₁-C₄)-alkyl, (C₁-C₄)-haloalkyl, (C₁-C₄)-haloalkoxy, (C₃-C₆)-cycloalkyl, phenyl, (C₁-C₄)-alkoxy, cyano, (C₁-C₄)-alkylthio, (C₁-C₄)-alkylsulfinyl, (C₁-C₄)-alkylsulfonyl, (C₁-C₄)-alkoxycarbonyl or (C₁-C₄)-alkylcarbonyl; R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl, phenyl or 3- to 6-membered heterocyclyl containing v_{D} heteroatoms from the group of nitrogen, oxygen and sulfur, where the seven latter radicals are substituted by v_{D} substituents from the group of halogen, (C₁-C₆)-alkoxy, (C₁-C₆)-haloalkoxy, (C₁-C₂)-alkylsulfinyl, (C₁-C₂)-alkylsulfonyl, (C₃-C₆)-cycloalkyl, (C₁-C₄)-alkoxycarbonyl, (C₁-C₄)-alkylcarbonyl and phenyl and, in the case of cyclic radicals, also (C₁-C₄)-alkyl and (C₁-C₄)-haloalkyl;
R_{D}⁶ is hydrogen, (C₁-C₆)-alkyl, (C₂-C₆)-alkenyl or (C₂-C₆)-alkynyl, where the three latter radicals are substituted by v_{D} radicals from the group consisting of halogen, hydroxy, (C₁-C₄)-alkyl, (C₁-C₄)-alkoxy and (C₁-C₄)-alkylthio, or
R_{D}⁵ and R_{D}⁶ together with the nitrogen atom carrying them form a pyrrolidinyl or piperidinyl radical;
R_{D}⁷ is hydrogen, (C₁-C₄)-alkylamino, di-(C₁-C₄)-alkylamino, (C₁-C₆)-alkyl, (C₃-C₆)-cycloalkyl, where the 2 latter radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;
n_{D} is 0, 1 or 2;
m_{D} is 1 or 2;
v_{D} is 0, 1, 2 or 3;
among these, preference is given to compounds of the N-acylsulfonamide type, for example of the formula (S4^{a}) below, which are known, for example, from WO-A-97/45016

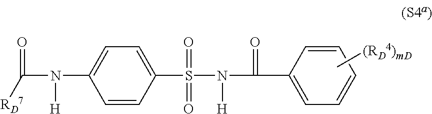

in which
R_{D}⁷ is (C₁-C₆)-alkyl, (C₃-C₆)-cycloalkyl, where the 2 latter radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;
R_{D}⁴ is halogen, (C₁-C₄)-alkyl, (C₁-C₄)-alkoxy, CF₃
m_{D} 1 or 2;
v_{D} is 0, 1, 2 or 3;
and also to acylsulfamoylbenzamides, for example of the formula (S4^{b}) below, which are known, for example, from WO-A-99/16744,

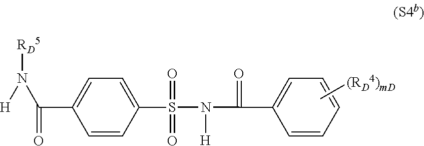

for example those in which
R_{D}⁵=cyclopropyl and (R_{D}⁴)=2-OMe (“cyprosulfamide”, S4-1),
R_{D}⁵=cyclopropyl and (R_{D}⁴)=5-Cl-2-OMe (S4-2),
R_{D}⁵=ethyl and (R_{D}⁴)=2-OMe (S4-3),
R_{D}⁵=isopropyl and (R_{D}⁴)=5-Cl-2-OMe (S4-4) and
R_{D}⁵=isopropyl and (R_{D}⁴)=2-OMe (S4-5)
and to compounds of the N-acylsulfamoylphenylurea type, of the formula (S4^{c}), which are known, for example, from EP-A-365484,

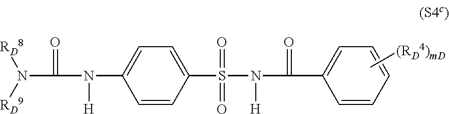

in which
R_{D}⁸ and R_{D}⁹ are each independently hydrogen, (C₁-C₈)-alkyl, (C₃-C₈)-cycloalkyl, (C₃-C₆)-alkenyl, (C₃-C₆)-alkynyl,
R_{D}⁴ is halogen, (C₁-C₄)-alkyl, (C₁-C₄)-alkoxy, CF₃
m_{D} is 1 or 2;
for example
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea.
S5) Active compounds from the class of the hydroxyaromatics and aromatic-aliphatic carboxylic acid derivatives (S5), for example ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.
S6) Active compounds from the class of the 1,2-dihydroquinoxalin-2-ones (S6), for example 1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.
S7) Compounds of the formula (S7), as described in WO-A-1998/38856,

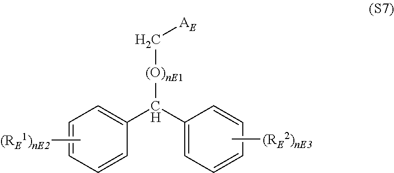

where the symbols and indices have the following meanings:
R_{E}¹, R_{E}² are each independently halogen, (C₁-C₄)-alkyl, (C₁-C₄)-alkoxy, (C₁-C₄)-haloalkyl, (C₁-C₄)-alkylamino, di-(C₁-C₄)-alkylamino, nitro;

### A_{E} is COOR_{E}³ or COSR_{E}⁴

R_{E}³, R_{E}⁴ are each independently hydrogen, (C₁-C₄)-alkyl, (C₂-C₆)-alkenyl, (C₂-C₄)-alkynyl, cyanoalkyl, (C₁-C₄)-haloalkyl, phenyl, nitrophenyl, benzyl, halobenzyl, pyridinylalkyl and alkylammonium,
n_{E}¹ is 0 or 1
n_{E}², n_{E}³ are each independently 0, 1 or 2,
preferably diphenylmethoxyacetic acid, ethyl diphenylmethoxyacetate, methyl diphenylmethoxyacetate (CAS reg. no. 41858-19-9) (S7-1).
S8) Compounds of the formula (S8), as described in WO-A-98/27049,

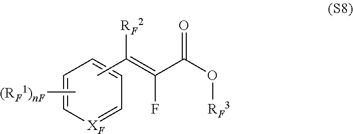

in which

### X_{E} is CH or N,

n_{F} in the case that X_{F}=N is an integer from 0 to 4 and

in the case that X_{F}=CH is an integer from 0 to 5,

R_{F}¹ is halogen, (C₁-C₄)-alkyl, (C₁-C₄)-haloalkyl, (C₁-C₄)-alkoxy, (C₁-C₄)-haloalkoxy, nitro, (C₁-C₄)-alkylthio, (C₁-C₄)-alkylsulfonyl, (C₁-C₄)-alkoxycarbonyl, optionally substituted phenyl, optionally substituted phenoxy,
R_{F}² is hydrogen or (C₁-C₄)-alkyl,
R_{F}³ is hydrogen, (C₁-C₈)-alkyl, (C₂-C₄)-alkenyl, (C₂-C₄)-alkynyl, or aryl, where each of the aforementioned carbon-containing radicals is unsubstituted or substituted by one or more, preferably up to three identical or different radicals from the group consisting of halogen and alkoxy; or salts thereof,
preferably compounds in which

### X_{F} is CH,

n_{F} is an integer from 0 to 2,
R_{F}¹ is halogen, (C₁-C₄)-alkyl, (C₁-C₄)-haloalkyl, (C₁-C₄)-alkoxy, (C₁-C₄)-haloalkoxy,
R_{F}² is hydrogen or (C₁-C₄)-alkyl,
R_{F}³ is hydrogen, (C₁-C₈)-alkyl, (C₂-C₄)-alkenyl, (C₂-C₄)-alkynyl, or aryl, where each of the aforementioned carbon-containing radicals is unsubstituted or substituted by one or more, preferably up to three identical or different radicals from the group consisting of halogen and alkoxy,
or salts thereof.
S9) Active compounds from the class of the 3-(5-tetrazolylcarbonyl)-2-quinolones (S9), for example 1,2-dihydro-4-hydroxy-1-ethyl-3-(5-tetrazolylcarbonyl)-2-quinolone (CAS reg. no. 219479-18-2), 1,2-dihydro-4-hydroxy-1-methyl-3-(5-tetrazolylcarbonyl)-2-quinolone (CAS reg. no. 95855-00-8), as described in WO-A-1999/000020.
S10) Compounds of the formula (S10^{a}) or (S10^{b})

as described in WO-A-2007/023719 and WO-A-2007/023764,

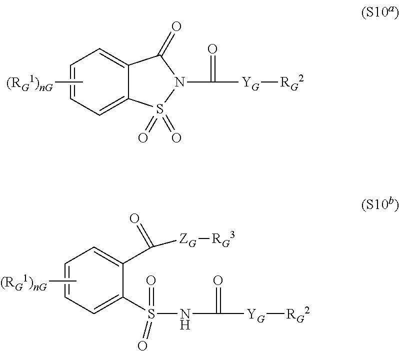

in which
R_{G}¹ is halogen, (C₁-C₄)-alkyl, methoxy, nitro, cyano, CF₃, OCF₃,
Y_{G}, Z_{G} are each independently 0 or S,
n_{G} is an integer from 0 to 4,
R_{G}² is (C₁-C₁₆)-alkyl, (C₂-C₆)-alkenyl, (C₃-C₆)-cycloalkyl, aryl; benzyl, halobenzyl,
R_{G}³ is hydrogen or (C₁-C₆)-alkyl.
S11) Active compounds of the oxyimino compounds type (S11), which are known as seed-dressing compositions, for example “oxabetrinil” ((Z)-1,3-dioxolan-2-yl-methoxyimino(phenyl)acetonitrile) (S11-1), which is known as a seed-dressing safener for millet against damage by metolachlor, “fluxofenim” (1-(4-chlorophenyl)-2,2,2-trifluoro-1-ethanone O-(1,3-dioxolan-2-ylmethyl)oxime) (S11-2), which is known as a seed-dressing safener for millet against damage by metolachlor, and “cyometrinil” or “CGA-43089” ((Z)-cyanomethoxyimino(phenyl)acetonitrile) (S11-3), which is known as a seed-dressing safener for millet against damage by metolachlor.
S12) Active compounds from the class of the isothiochromanones (S12), for example methyl[(3-oxo-1H-2-benzothiopyran-4(3H)-ylidene)methoxy]acetate (CAS reg. no. 205121-04-6) (S12-1) and related compounds from WO-A-1998/13361.
S13) One or more compounds from group (S13): “naphthalic anhydride” (1,8-naphthalenedicarboxylic anhydride) (S13-1), which is known as a seed-dressing safener for corn against damage by thiocarbamate herbicides, “fenclorim” (4,6-dichloro-2-phenylpyrimidine) (S13-2), which is known as a safener for pretilachlor in sown rice, “flurazole” (benzyl 2-chloro-4-trifluoromethyl-1,3-thiazole-5-carboxylate) (S13-3), which is known as a seed-dressing safener for millet against damage by alachlor and metolachlor, “CL 304415” (CAS reg. no. 31541-57-8) (4-carboxy-3,4-dihydro-2H-1-benzopyran-4-acetic acid) (S13-4) from American Cyanamid, which is known as a safener for corn against damage by imidazolinones, “MG 191” (CAS reg. no. 96420-72-3) (2-dichloromethyl-2-methyl-1,3-dioxolane) (S13-5) from Nitrokemia, which is known as a safener for corn, “MG-838” (CAS reg. no. 133993-74-5) (2-propenyl 1-oxa-4-azaspiro[4.5]decane-4-carbodithioate) (S13-6) from Nitrokemia, “disulfoton” (O,O-diethyl S-2-ethylthioethyl phosphorodithioate) (S13-7), “dietholate” (O,O-diethyl O-phenylphosphorothioate) (S13-8), “mephenate” (4-chlorophenyl methylcarbamate) (S13-9).
S14) Active compounds which, in addition to herbicidal action against harmful plants, also have safener action on crop plants such as rice, for example “dimepiperate” or “MY-93” (S-1-methyl-1-phenylethylpiperidine-1-carbothioate), which is known as a safener for rice against damage by the herbicide molinate, “daimuron” or “SK 23” (1-(1-methyl-1-phenylethyl)-3-p-tolylurea), which is known as a safener for rice against damage by the herbicide imazosulfuron, “cumyluron”=“JC-940” (3-(2-chlorophenylmethyl)-1-(1-methyl-1-phenylethyl)urea, see JP-A-60087254), which is known as a safener for rice against damage by some herbicides, “methoxyphenone” or “NK 049” (3,3′-dimethyl-4-methoxybenzophenone), which is known as a safener for rice against damage by some herbicides, “CSB” (1-bromo-4-(chloromethylsulfonyl)benzene) from Kumiai, (CAS reg. no. 54091-06-4), which is known as a safener against damage by some herbicides in rice.
S15) Compounds of the formula (S15) or tautomers thereof
as described in WO-A-2008/131861 and WO-A-2008/131860,

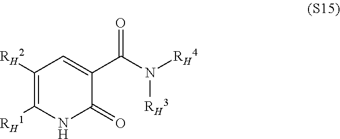

in which

R_{H}¹ is a (C₁-C₆)-haloalkyl radical and
R_{H}² is hydrogen or halogen and
R_{H}³, R_{H}⁴ are each independently of one another hydrogen, (C₁-C₁₆)-alkyl, (C₂-C₁₆)-alkenyl or (C₂-C₁₆)-alkynyl, where each of the latter 3 radicals is unsubstituted or substituted by one or more radicals from the group of halogen, hydroxy, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted, or (C₃-C₆)-cycloalkyl, (C₄-C₆)-cycloalkenyl, (C₃-C₆)-cycloalkyl which is fused on one side of the ring to a 4 to 6-membered saturated or unsaturated carbocyclic ring, or (C₄-C₆)-cycloalkenyl which is fused on one side of the ring to a 4 to 6-membered saturated or unsaturated carbocyclic ring, where each of the latter 4 radicals is unsubstituted or substituted by one or more radicals from the group of halogen, hydroxy, cyano, (C1-C4)-alkyl, (C1-C4)-haloalkyl, (C1-C4)-alkoxy, (C1-C4)-haloalkoxy, (C1-C4)-alkylthio, (C1-C4)-alkylamino, di[(C1-C4)-alkyl]amino, [(C1-C4)-alkoxy]carbonyl, [(C1-C4)-haloalkoxy]carbonyl, (C3-C6)-cycloalkyl which is unsubstituted or substituted, phenyl which is unsubstituted or substituted, and heterocyclyl which is unsubstituted or substituted,
or
R_{H}³ is (C₁-C₄)-alkoxy, (C₂-C₄)-alkenyloxy, (C₂-C₆)-alkynyloxy or (C₂-C₄)-haloalkoxy and
R_{H}⁴ is hydrogen or (C₁-C₄)-alkyl or
R_{H}³ and R_{H}⁴ together with the directly attached nitrogen atom are a four- to eight-membered heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio.
S16) Active ingredients which are used primarily as herbicides but also have safener action on crop plants, for example (2,4-dichlorophenoxy)acetic acid (2,4-D), (4-chlorophenoxy)acetic acid, (R,S)-2-(4-chloro-o-tolyloxy)propionic acid (mecoprop), 4-(2,4-dichlorophenoxy)butyric acid (2,4-DB), (4-chloro-o-tolyloxy)acetic acid (MCPA), 4-(4-chloro-o-tolyloxy)butyric acid, 4-(4-chlorophenoxy)butyric acid, 3,6-dichloro-2-methoxybenzoic acid (dicamba), 1-(ethoxycarbonyl)ethyl 3,6-dichloro-2-methoxybenzoate (lactidichlor-ethyl).
Substances which Influence Plant Maturity:

Combination partners usable for the compounds of the general formula (I) in mixture formulations or in a tankmix are, for example, known active compounds based on inhibition of, for example, 1-aminocyclopropane-1-carboxylate synthase, 1-aminocyclopropane-1-carboxylate oxidase and the ethylene receptors, for example ETR1, ETR2, ERS1, ERS2 or EIN4, as described, for example, in Biotechn. Adv. 2006, 24, 357-367; Bot. Bull. Acad. Sin. 199, 40, 1-7 or Plant Growth Reg. 1993, 13, 41-46 and literature cited therein.

Examples of known substances which influence plant maturity and can be combined with the compounds of the general formula (I) include the active compounds which follow (the compounds are designated either by the “common name” according to the International Organization for Standardization (ISO) or by the chemical name or by the code number) and always encompass all use forms, such as acids, salts, esters and isomers, such as stereoisomers and optical isomers. Here, by way of example, one and in some cases a plurality of use forms are mentioned:

rhizobitoxine, 2-aminoethoxyvinylglycine (AVG), methoxyvinylglycine (MVG), vinylglycine, aminooxyacetic acid, sinefungin, S-adenosylhomocysteine, 2-keto-4-methyl thiobutyrate, 2-(methoxy)-2-oxoethyl (isopropylidene)aminooxyacetate, 2-(hexyloxy)-2-oxoethyl (isopropylidene)aminooxyacetate, 2-(isopropyloxy)-2-oxoethyl (cyclohexylidene)aminooxyacetate, putrescine, spermidine, spermine, 1,8-diamino-4-aminoethyloctane, L-canaline, daminozide, methyl 1-aminocyclopropyl-1-carboxylate, N-methyl-1-aminocyclopropyl-1-carboxylic acid, 1-aminocyclopropyl-1-carboxamide, substituted 1-aminocyclopropyl-1-carboxylic acid derivatives as described in DE3335514, EP30287, DE2906507 or U.S. Pat. No. 5,123,951, 1-aminocyclopropyl-1-hydroxamic acid, 1-methylcyclopropene, 3-methylcyclopropene, 1-ethylcyclopropene, 1-n-propylcyclopropene, 1-cyclopropenylmethanol, carvone, eugenol, sodium cycloprop-1-en-1-ylacetate, sodium cycloprop-2-en-1-ylacetate, sodium 3-(cycloprop-2-en-1-yl)propanoate, sodium 3-(cycloprop-1-en-1-yl)propanoate, jasmonic acid, methyl jasmonate, ethyl jasmonate.

Substances which influence plant health and germination:

Examples of combination partners usable for the compounds of the general formula (I) in mixture formulations or in a tankmix include known active compounds which influence plant health (the compounds are designated by the “common name” according to the International Organization for Standardization (ISO) or by the chemical name or by the code number and always encompass all use forms, such as acids, salts, esters and isomers, such as stereoisomers and optical isomers): sarcosine, phenylalanine, tryptophan, N′-methyl-1-phenyl-1-N,N-diethylaminomethanesulfonamide, apio-galacturonans as described in WO2010017956, 4-oxo-4-[(2-phenylethyl)amino]butanoic acid, 4-{[2-(1H-indol-3-yl)ethyl]amino}-4-oxobutanoic acid, 4-[(3-methylpyridin-2-yl)amino]-4-oxobutanoic acid, allantoin, 5-aminolevulic acid, (2S,3R)-2-(3,4-dihydroxyphenyl)-3,4-dihydro-2H-chromene-3,5,7-triol and structurally related catechols as described in WO2010122956, 2-hydroxy-4-(methylsulfanyl)butanoic acid, (3E,3αR,8βS)-3-({[(2R)-4-methyl-5-oxo-2,5-dihydrofuran-2-yl]oxy}methylene)-3,3α, 4,8β-tetrahydro-2H-indeno[1,2-b]furan-2-one and analogous lactones as described in EP2248421, abscisic acid, (2Z,4E)-5-[6-ethynyl-1-hydroxy-2,6-dimethyl-4-oxocyclohex-2-en-1-yl]-3-methylpenta-2,4-dienoic acid, methyl (2Z,4E)-5-[6-ethynyl-1-hydroxy-2,6-dimethyl-4-oxocyclohex-2-en-1-yl]-3-methylpenta-2,4-dienoate, 4-phenylbutyric acid, sodium 4-phenylbutanoate, potassium 4-phenylbutanoate.

Herbicides or Plant Growth Regulators:

Combination partners usable for the compounds of the general formula (I) in mixture formulations or in a tankmix are, for example, known active compounds based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoendesaturase, photosystem I, photosystem II, protoporphyrinogen oxidase, as described, for example, in Weed Research 26 (1986) 441-445 or “The Pesticide Manual”, 14th edition, The British Crop Protection Council and the Royal Soc. of Chemistry, 2006 and literature cited therein.

Examples of known herbicides or plant growth regulators which can be combined with compounds of the general formula (I) include the active compounds which follow (the compounds are designated either by the “common name” according to the International Organization for Standardization (ISO) or by the chemical name or by the code number) and always encompass all use forms, such as acids, salts, esters and isomers, such as stereoisomers and optical isomers. Here, by way of example, one and in some cases a plurality of use forms are mentioned:

acetochlor, acibenzolar, acibenzolar-S-methyl, acifluorfen, acifluorfen-sodium, aclonifen, alachlor, allidochlor, alloxydim, alloxydim-sodium, ametryne, amicarbazone, amidochlor, amidosulfuron, aminocyclopyrachlor, aminopyralid, amitrole, ammonium sulfamate, ancymidol, anilofos, asulam, atrazine, azafenidin, azimsulfuron, aziprotryne, beflubutamid, benazolin, benazolin-ethyl, bencarbazone, benfluralin, benfuresate, bensulide, bensulfuron, bensulfuron-methyl, bentazone, benzfendizone, benzobicyclon, benzofenap, benzofluor, benzoylprop, bicyclopyrone, bifenox, bilanafos, bilanafos-sodium, bispyribac, bispyribac-sodium, bromacil, bromobutide, bromofenoxim, bromoxynil, bromuron, buminafos, busoxinone, butachlor, butafenacil, butamifos, butenachlor, butralin, butroxydim, butylate, cafenstrole, carbetamide, carfentrazone, carfentrazone-ethyl, chlomethoxyfen, chloramben, chlorazifop, chlorazifop-butyl, chlorbromuron, chlorbufam, chlorfenac, chlorfenac-sodium, chlorfenprop, chlorflurenol, chlorflurenol-methyl, chloridazon, chlorimuron, chlorimuron-ethyl, chlormequat-chloride, chlornitrofen, chlorophthalim, chlorthal-dimethyl, chlortoluron, chlorsulfuron, cinidon, cinidon-ethyl, cinmethylin, cinosulfuron, clethodim, clodinafop, clodinafop-propargyl, clofencet, clomazone, clomeprop, cloprop, clopyralid, cloransulam, cloransulam-methyl, cumyluron, cyanamide, cyanazine, cyclanilide, cycloate, cyclosulfamuron, cycloxydim, cycluron, cyhalofop, cyhalofop-butyl, cyperquat, cyprazine, cyprazole, 2,4-D, 2,4-DB, daimuron/dymron, dalapon, daminozide, dazomet, n-decanol, desmedipham, desmetryn, detosyl-pyrazolate (DTP), diallate, dicamba, dichlobenil, dichlorprop, dichlorprop-P, diclofop, diclofop-methyl, diclofop-P-methyl, diclosulam, diethatyl, diethatyl-ethyl, difenoxuron, difenzoquat, diflufenican, diflufenzopyr, diflufenzopyr-sodium, dimefuron, dikegulac-sodium, dimefuron, dimepiperate, dimethachlor, dimethametryn, dimethenamid, dimethenamid-P, dimethipin, dimetrasulfuron, dinitramine, dinoseb, dinoterb, diphenamid, dipropetryn, diquat, diquat-dibromide, dithiopyr, diuron, DNOC, eglinazine-ethyl, endothal, EPTC, esprocarb, ethalfluralin, ethametsulfuron, ethametsulfuron-methyl, ethephon, ethidimuron, ethiozin, ethofumesate, ethoxyfen, ethoxyfen-ethyl, ethoxysulfuron, etobenzanid, F-5331, i.e. N-[2-chloro-4-fluoro-5-[4-(3-fluoropropyl)-4,5-dihydro-5-oxo-1H-tetrazol-1-yl]phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoprop, fenoxaprop, fenoxaprop-P, fenoxaprop-ethyl, fenoxaprop-P-ethyl, fenoxasulfone, fentrazamide, fenuron, flamprop, flamprop-M-isopropyl, flamprop-M-methyl, flazasulfuron, florasulam, fluazifop, fluazifop-P, fluazifop-butyl, fluazifop-P-butyl, fluazolate, flucarbazone, flucarbazone-sodium, flucetosulfuron, fluchloralin, flufenacet (thiafluamide), flufenpyr, flufenpyr-ethyl, flumetralin, flumetsulam, flumiclorac, flumiclorac-pentyl, flumioxazin, flumipropyn, fluometuron, fluorodifen, fluoroglycofen, fluoroglycofen-ethyl, flupoxam, flupropacil, flupropanate, flupyrsulfuron, flupyrsulfuron-methyl-sodium, flurenol, flurenol-butyl, fluridone, flurochloridone, fluroxypyr, fluroxypyr-meptyl, flurprimidol, flurtamone, fluthiacet, fluthiacet-methyl, fluthiamide, fomesafen, foramsulfuron, forchlorfenuron, fosamine, furyloxyfen, gibberellic acid, glufosinate, glufosinate-ammonium, glufosinate-P, glufosinate-P-ammonium, glufosinate-P-sodium, glyphosate, glyphosate-isopropylammonium, H-9201, i.e. O-(2,4-dimethyl-6-nitrophenyl) O-ethyl isopropylphosphoramidothioate, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl (2,4-dichlorophenoxy)acetate, imazamethabenz, imazamethabenz-methyl, imazamox, imazamox-ammonium, imazapic, imazapyr, imazapyr-isopropylammonium, imazaquin, imazaquin-ammonium, imazethapyr, imazethapyr-ammonium, imazosulfuron, inabenfide, indanofan, indaziflam, indoleacetic acid (IAA), 4-indol-3-ylbutyric acid (IBA), iodosulfuron, iodosulfuron-methyl-sodium, ioxynil, ipfencarbazone, isocarbamid, isopropalin, isoproturon, isouron, isoxaben, isoxachlortole, isoxaflutole, isoxapyrifop, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, karbutilate, ketospiradox, lactofen, lenacil, linuron, maleic hydrazide, MCPA, MCPB, MCPB-methyl, -ethyl and -sodium, mecoprop, mecoprop-sodium, mecoprop-butotyl, mecoprop-P-butotyl, mecoprop-P-dimethylammonium, mecoprop-P-2-ethylhexyl, mecoprop-P-potassium, mefenacet, mefluidide, mepiquat-chloride, mesosulfuron, mesosulfuron-methyl, mesotrione, methabenzthiazuron, metam, metamifop, metamitron, metazachlor, metazasulfuron, methazole, methiopyrsulfuron, methiozolin, methoxyphenone, methyldymron, 1-methylcyclopropene, methyl isothiocyanate, metobenzuron, metobromuron, metolachlor, S-metolachlor, metosulam, metoxuron, metribuzin, metsulfuron, metsulfuron-methyl, molinate, monalide, monocarbamide, monocarbamide dihydrogensulfate, monolinuron, monosulfuron, monosulfuron esters, monuron, MT-128, i.e. 6-chloro-N-[(2E)-3-chloroprop-2-en-1-yl]-5-methyl-N-phenylpyridazine-3-amine, MT-5950, i.e. N-[3-chloro-4-(1-methylethyl)phenyl]-2-methylpentanamide, NGGC-011, naproanilide, napropamide, naptalam, NC-310, i.e. 4-(2,4-dichlorobenzoyl)-1-methyl-5-benzyloxypyrazole, neburon, nicosulfuron, nipyraclofen, nitralin, nitrofen, nitrophenolate-sodium (isomer mixture), nitrofluorfen, nonanoic acid, norflurazon, orbencarb, orthosulfamuron, oryzalin, oxadiargyl, oxadiazon, oxasulfuron, oxaziclomefone, oxyfluorfen, paclobutrazole, paraquat, paraquat dichloride, pelargonic acid (nonanoic acid), pendimethalin, pendralin, penoxsulam, pentanochlor, pentoxazone, perfluidone, pethoxamid, phenisopham, phenmedipham, phenmedipham-ethyl, picloram, picolinafen, pinoxaden, piperophos, pirifenop, pirifenop-butyl, pretilachlor, primisulfuron, primisulfuron-methyl, probenazole, profluazole, procyazine, prodiamine, prifluraline, profoxydim, prohexadione, prohexadione-calcium, prohydrojasmone, prometon, prometryn, propachlor, propanil, propaquizafop, propazine, propham, propisochlor, propoxycarbazone, propoxycarbazone-sodium, propyrisulfuron, propyzamide, prosulfalin, prosulfocarb, prosulfuron, prynachlor, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, secbumeton, sethoxydim, siduron, simazine, simetryn, SN-106279, i.e. methyl (2R)-2-({7-[2-chloro-4-(trifluoromethyl)phenoxy]-2-naphthyl}oxy)propanoate, sulcotrione, sulfallate (CDEC), sulfentrazone, sulfometuron, sulfometuron-methyl, sulfosate (glyphosate-trimesium), sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, tebutam, tebuthiuron, tecnazene, tefuryltrione, tembotrione, tepraloxydim, terbacil, terbucarb, terbuchlor, terbumeton, terbuthylazine, terbutryn, thenylchlor, thiafluamide, thiazafluron, thiazopyr, thidiazimin, thidiazuron, thiencarbazone, thiencarbazone-methyl, thifensulfuron, thifensulfuron-methyl, thiobencarb, thiocarbazil, topramezone, tralkoxydim, triallate, triasulfuron, triaziflam, triazofenamide, tribenuron, tribenuron-methyl, trichloroacetic acid (TCA), triclopyr, tridiphane, trietazine, trifloxysulfuron, trifloxysulfuron-sodium, trifluralin, triflusulfuron, triflusulfuron-methyl, trimeturon, trinexapac, trinexapac-ethyl, tritosulfuron, tsitodef, uniconazole, uniconazole-P, vernolate, ZJ-0862, i.e. 3,4-dichloro-N-{2-[(4,6-dimethoxypyrimidin-2-yl)oxy]benzyl}aniline, and the following compounds:

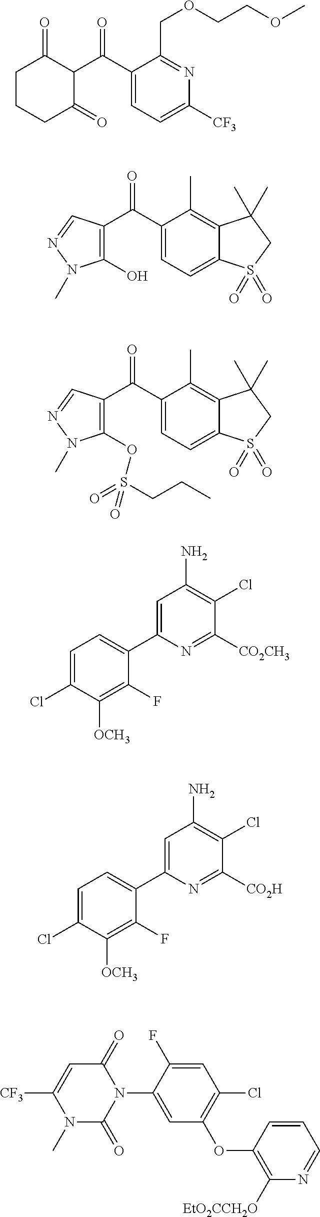

The invention is to be illustrated by the biological examples which follow, but without restricting it thereto.

### Biological Examples

Seeds of monocotyledonous and dicotyledonous crop plants were laid out in sandy loam in wood-fiber pots, covered with soil or sand and cultivated in a greenhouse under good growth conditions. The test plants were treated at the early leaf stage (BBCH10-BBCH13). To assure uniform water supply before commencement of stress, the potted plants were supplied with water by dam irrigation prior to substance application. The compounds according to the invention, formulated in the form of wettable powders (WP), were sprayed onto the green parts of the plants as an aqueous suspension at an equivalent water application rate of 600 I/ha with addition of 0.2% wetting agent (e.g. agrotin). Substance application was followed immediately by stress treatment of the plants. For this purpose, the pots were transferred into plastic inserts in order to prevent them from subsequently drying out too quickly. Drought stress was induced by gradual drying out under the following conditions:

“Day”: 14 hours with illumination at 26° C.
“Night”: 10 hours without illumination at 18° C.

The duration of the respective stress phases was guided mainly by the state of the untreated, stressed control plants and thus varied from crop to crop. It was ended (by re-irrigating and transfer to a greenhouse with good growth conditions) as soon as irreversible damage was observed on the untreated, stressed control plants. In the case of dicotyledonous crops, for example oilseed rape and soya, the duration of the drought stress phase varied between 3 and 6 days, in the case of monocotyledonous crops, for example wheat, barley or corn, between 6 and 11 days.

The end of the stress phase was followed by an approx. 5-7-day recovery phase, during which the plants were once again kept under good growth conditions in a greenhouse.

In order to rule out any influence of the effects observed by any fungicidal or insecticidal action of the test compounds, it was additionally ensured that the tests proceeded without fungal infection or insect infestation.

After the recovery phase had ended, the intensities of damage were analyzed in visual comparison to untreated, unstressed controls for the same age. The intensity of damage was first recorded as a percentage (100%=plants have died, 0%=like control plants). These values were then used to calculate the efficacy of the test compounds (=percentage reduction in the intensity of damage as a result of substance application) by the following formula:

$$EF = \frac{( {DV}_{us} - {DV}_{ts} ) \times 100}{{DV}_{us}}$$

EF: efficacy (%)
DVus: damage value of the untreated, stressed control
DVts: damage value of the plants treated with test compound

In each trial, 3 pots per crop and dosage were treated and evaluated; the resulting efficacies are thus averages. The values in tables A-1 to A-4 below are again averages from one to three independent trials.

Effects of selected compounds of the formula (I) under drought stress:

TABLE A-1

| | | | | EF |
|---|---|---|---|---|
| No. | Substance | Dosage | Unit | (HORVS) |
| 1 | A1-76 | 1000 | g/ha | >5 |
| 2 | B1-8 | 500 | g/ha | >5 |
| 3 | B1-11 | 500 | g/ha | >5 |
| 4 | B1-18 | 500 | g/ha | >5 |
| 5 | B1-50 | 500 | g/ha | >5 |

TABLE A-2

| | | | | EF |
|---|---|---|---|---|
| No. | Substance | Dosage | Unit | (BRSNS) |
| 1 | A1-10 | 50 | g/ha | >5 |
| 2 | A1-81 | 25 | g/ha | >5 |
| 3 | A1-79 | 250 | g/ha | >5 |
| 4 | B1-5 | 100 | g/ha | >5 |
| 5 | B1-8 | 50 | g/ha | >5 |
| 6 | B1-36 | 25 | g/ha | >5 |
| 7 | B1-50 | 50 | g/ha | >5 |

TABLE A-3

| | | | | EF |
|---|---|---|---|---|
| No. | Substance | Dosage | Unit | (ZEAMX) |
| 1 | A1-10 | 25 | g/ha | >5 |
| 2 | A1-79 | 25 | g/ha | >5 |
| 3 | A1-81 | 25 | g/ha | >5 |
| 4 | A1-82 | 25 | g/ha | >5 |
| 5 | B1-11 | 500 | g/ha | >5 |
| 6 | B1-18 | 250 | g/ha | >5 |
| 7 | B1-36 | 250 | g/ha | >5 |
| 8 | B1-50 | 50 | g/ha | >5 |

TABLE A-4

| | | | | EF |
|---|---|---|---|---|
| No. | Substance | Dosage | Unit | (TRZAS) |
| 1 | A1-10 | 25 | g/ha | >5 |
| 2 | A1-82 | 25 | g/ha | >5 |
| 3 | A2-81 | 250 | g/ha | >5 |
| 4 | B1-5 | 100 | g/ha | >5 |

In the above tables:

BRSNS=Brassica napus
HORVS=Hordeum vulgare
TRZAS=Triticum aestivum
ZEAMX=Zea mays

Similar results were also achieved with further compounds of the general formula (I), also in the case of application to different plant species.

1. A compound comprising a substituted 2-amidobenzimidazole, 2-amidobenzoxazole and/or 2-amidobenzothiazole of formula (I) and/or salt thereof 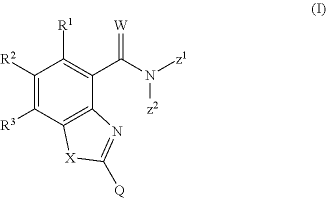
capable of being used for increasing tolerance to abiotic stress in one or more plants, where
R¹, R², R³ independently of one another represent hydrogen, halogen, alkyl, cycloalkyl, cycloalkenyl, halocycloalkyl, alkenyl, alkynyl, aryl, arylalkyl, arylalkenyl, heteroaryl, heteroarylalkyl, heterocyclyl, heterocyclylalkyl, alkoxyalkyl, alkylthio, haloalkylthio, haloalkyl, alkoxy, haloalkoxy, cycloalkoxy, cycloalkylalkoxy, aryloxy, heteroaryloxy, alkoxyalkoxy, alkynylalkoxy, alkenyloxy, bisalkylaminoalkoxy, tris[alkyl]silyl, bis[alkyl]arylsilyl, bis[alkyl]alkylsilyl, tris[alkyl]silylalkynyl, arylalkynyl, heteroarylalkynyl, alkylalkynyl, cycloalkylalkynyl, haloalkylalkynyl, heterocyclyl-N-alkoxy, nitro, cyano, amino, alkylamino, bisalkylamino, alkylcarbonylamino, cycloalkylcarbonylamino, arylcarbonylamino, alkoxycarbonylamino, heteroarylalkoxy, arylalkoxy, heterocyclylalkoxy, cycloalkylalkyl, haloalkenyl, haloalkynyl, heterocyclylalkynyl, halocycloalkoxy, haloalkynyloxy, arylthio, heteroarylthio, alkylsulfinyl, haloalkylsulfinyl, arylsulfinyl, heteroarylsulfinyl, alkylsulfonyl, haloalkylsulfonyl, arylsulfonyl, heteroarylsulfonyl, thiocyanato, isothiocyanato, cycloalkylamino, cycloalkyl(alkyl)amino, alkenylamino,
R¹ and R² with the atoms to which they are attached form a fully saturated, partially saturated or unsaturated 5 to 7-membered ring which is optionally interrupted by heteroatoms and optionally substituted further,
R² and R³ with the atoms to which they are attached form a fully saturated, partially saturated or unsaturated 5 to 7-membered ring which is optionally interrupted by heteroatoms and optionally substituted further,
W represents oxygen, sulfur,
X represents oxygen, sulfur, N—R⁴,
R⁴ represents hydrogen, alkyl, cycloalkyl, cycloalkylalkyl, cycloalkenyl, cyanoalkyl, alkenylalkyl, haloalkyl, alkynylalkyl, arylalkyl, heteroarylalkyl, heterocyclylalkyl, alkylcarbonyl, cycloalkylcarbonyl, arylcarbonyl, heteroarylcarbonyl, haloalkylcarbonyl, heterocyclylcarbonyl, arylalkylcarbonyl, alkoxycarbonyl, cycloalkylalkoxycarbonyl, cycloalkoxycarbonyl, alkoxycarbonylalkyl, alkylsulfonyl, cycloalkylsulfonyl, arylsulfonyl, heteroarylsulfonyl, alkoxycarbonylcarbonyl, arylalkoxycarbonylcarbonyl, alkylaminothiocarbonyl, alkylaminocarbonyl, cycloalkylaminocarbonyl, alkoxyalkyl, bis[alkyl]aminocarbonyl, aryl, heteroaryl, heterocyclyl, cycloalkoxycarbonylalkyl,
Q represents alkyl, alkenyl, cycloalkyl, cycloalkylalkyl, cycloalkenyl, aryl, arylalkyl, heteroarylalkyl, heterocyclylalkyl, heteroaryl, heterocyclyl, heterocyclylaryl, heterocyclylheteroaryl, heteroarylheteroaryl, heteroarylaryl, arylaryl, aryloxyaryl, arylalkenyl, heteroarylalkenyl, heterocyclylalkenyl, arylalkynyl, heteroarylalkynyl, heterocyclylalkynyl, cycloalkylalkynyl, alkylaminoalkyl, bisalkylaminoalkyl, hydroxyalkyl, alkoxyalkyl, tris[alkyl]silyloxyalkyl, bis[alkyl]arylsilyloxyalkyl, bis[alkyl]alkylsilyloxyalkyl, bisalkylaminoalkoxyalkyl, alkoxyalkoxyalkyl, aryloxyalkyl, heteroaryloxyalkyl, alkylthioalkyl, arylthioalkyl, heteroarylthioalkyl, alkoxycarbonyl-N-heterocyclyl, arylalkoxycarbonyl-N-heterocyclyl, alkyl-N-heterocyclyl, alkylsulfonyl-N-heterocyclyl, arylsulfonyl-N-heterocyclyl, heteroarylsulfonyl-N-heterocyclyl, cycloalkylsulfonyl-N-heterocyclyl, haloalkylsulfonyl-N-heterocyclyl, alkylcarbonyl-N-heterocyclyl, arylcarbonyl-N-heterocyclyl, heteroarylcarbonyl-N-heterocyclyl, cycloalkylcarbonyl-N-heterocyclyl, cycloalkyl-N-heterocyclyl, aryl-N-heterocyclyl, arylalkyl-N-heterocyclyl, bisalkylaminoalkyl-N-heterocyclyl, bisalkylaminosulfonyl-N-heterocyclyl, heteroaryloxyaryl, heteroaryloxyheteroaryl, aryloxyheteroaryl, alkylsulfinyl, alkylthio, alkylsulfonyl, cycloalkylsulfinyl, cycloalkylthio, cycloalkylsulfonyl, arylsulfinyl, arylthio, arylsulfonyl, amino, alkylamino, arylamino, arylalkylamino, cycloalkylamino, formyl, alkylcarbonyl, arylcarbonyl, Iminoalkyl, alkyliminoalkyl, aryliminoalkyl, alkoxycarbonyl, cycloalkoxycarbonyl, cycloalkylalkoxycarbonyl, arylalkoxycarbonyl, arylalkylaminocarbonyl, aminocarbonyl, alkylaminocarbonyl, cycloalkylaminocarbonyl, bisalkylaminocarbonyl heterocyclyl-N-carbonyl, imino, alkylimino, arylimino, cycloalkylimino, cycloalkylalkylimino, hydroxyimino, alkoxyimino, cycloalkoxyimino, cycloalkylalkoxyimino, heteroaryloxyimino, aryloxyimino, arylalkoxyimino, alkenyloxyimino, heteroarylalkoxyimino, heteroarylimino, heterocyclylimino, heterocyclylalkylimino, aminoimino, alkylaminoimino, arylaminoimino, heteroarylaminoimino, cycloalkylaminoimino, bisalkylaminoimino, arylalkylaminoimino, aryl(alkyl)aminoimino, cycloalkyl(alkyl)aminoimino, cycloalkylalkylaminoimino, heterocyclylaminoimino, heteroarylalkoxyalkyl, arylalkoxyalkyl, heterocyclyl-N-alkyl, aryl(alkyl)aminoalkyl, arylalkyl(alkyl)aminoalkyl, alkoxycarbonylalkylaminoalkyl, alkoxycarbonylalkyl(alkyl)aminoalkyl, heteroaryl(alkyl)aminoalkyl, heteroarylalkyl(alkyl)aminoalkyl, cycloalkyl(alkyl)aminoalkyl, cycloalkylaminoalkyl, alkoxy(alkoxy)alkyl, arylalkoxyalkylaryl, heterocyclyl-N-alkylaryl, aryl(alkyl)aminoalkylaryl, arylalkyl(alkyl)aminoalkylaryl, alkoxycarbonylalkylaminoalkylaryl, alkoxycarbonylalkyl(alkyl)aminoalkylaryl, heteroaryl(alkyl)aminoalkylaryl, heteroarylalkyl(alkyl)aminoalkylaryl, cycloalkyl(alkyl)aminoalkylaryl, cycloalkylaminoalkylaryl, alkoxy(alkoxy)alkylaryl,
Q additionally, if X represents an oxygen atom or X represents a sulfur atom, represents haloalkyl, alkoxyhaloalkyl, halocycloalkyl, haloalkoxyhaloalkyl, arylhaloalkyl, alkylthiohaloalkyl, bisalkylaminoalkoxyhaloalkyl,
Z¹ represents hydrogen, hydroxy, alkyl, cycloalkyl, halocycloalkyl, halogen, alkenylalkyl, haloalkyl, alkynyl, alkenyl, cyanoalkyl, nitroalkyl, aminoalkyl, alkylaminoalkyl, bis[alkyl]aminoalkyl, alkynylalkyl, arylalkyl, heteroarylalkyl, heterocyclylalkyl, alkylcarbonyl, alkoxycarbonyl, cycloalkoxycarbonyl, alkylsulfonyl, arylsulfonyl, heteroarylsulfonyl, cycloalkylsulfonyl, alkylsulfinyl, arylsulfinyl, cycloalkylsulfinyl, alkoxycarbonylalkyl, aryl, heteroaryl, heterocyclyl, aminocarbonylalkyl, alkylaminocarbonylalkyl, bisalkylaminocarbonylalkyl, cycloalkylaminocarbonylalkyl, hydroxycarbonylalkyl, amino, alkylamino, arylamino, alkoxy, cycloalkylalkyl
and
Z² represents hydrogen, alkyl, cycloalkyl, branched or straight-chain haloalkyl, alkynyl, alkenyl, cyanoalkyl, arylalkyl, heteroarylalkyl, alkylcarbonyl, alkoxycarbonyl
or
Z¹ and Z² together with the atoms to which they are attached form a fully saturated or partially saturated 5 to 7-membered ring which is optionally interrupted by heteroatoms and optionally substituted further
or
Z¹ and Z² together are part of an optionally substituted sulfilimine or amidine group or form an iminophosphorane.

2. The compound as claimed in claim 1, where, in formula (I),
R¹, R², R³ independently of one another represent hydrogen, halogen, (C₁-C₈)-alkyl, (C₃-C₈)-cycloalkyl, (C₄-C₈)-cycloalkenyl, (C₃-C₈)-halocycloalkyl, (C₂-C₈)-alkenyl, (C₂-C₈)-alkynyl, optionally substituted phenyl, aryl-(C₁-C₈)-alkyl, aryl-(C₂-C₈)-alkenyl, heteroaryl, heteroaryl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio, (C₁-C₈)-haloalkylthio, (C₁-C₈)-haloalkyl, (C₁-C₈)-alkoxy, (C₁-C₈)-haloalkoxy, (C₃-C₈)-cycloalkoxy, (C₃-C₈)-cycloalkyl-(C₁-C₈)-alkoxy, aryloxy, heteroaryloxy, (C₁-C₈)-alkoxy-(C₁-C₈)-alkoxy, (C₂-C₈)-alkynyl-(C₁-C₈)-alkoxy, (C₂-C₈)-alkenyloxy, bis[(C₁-C₈)-alkyl]amino-(C₁-C₈)-alkoxy, tris[(C₁-C₈)-alkyl]silyl, bis[(C₁-C₈)-alkyl]arylsilyl, bis[(C₁-C₈)-alkyl]-(C₁-C₈)-alkylsilyl, tris[(C₁-C₈)-alkyl]silyl-(C₂-C₈)-alkynyl, aryl-(C₂-C₈)-alkynyl, heteroaryl-(C₂-C₈)-alkynyl, (C₁-C₈)-alkyl-(C₂-C₈)-alkynyl, (C₃-C₈)-cycloalkyl-(C₂-C₈)-alkynyl, (C₁-C₈)-haloalkyl-(C₂-C₈)-alkynyl, heterocyclyl-N—(C₁-C₈)-alkoxy, nitro, cyano, amino, (C₁-C₈)-alkylamino, bis[(C₁-C₈)-alkyl]amino, (C₁-C₈)-alkylcarbonylamino, (C₃-C₈)-cycloalkylcarbonylamino, arylcarbonylamino, (C₁-C₈)-alkoxycarbonylamino, heterocyclyl-(C₁-C₈)-alkoxy, (C₃-C₈)-cycloalkyl-(C₁-C₈)-alkyl, (C₂-C₈)-haloalkenyl, (C₂-C₈)-haloalkynyl, heterocyclyl-(C₂-C₈)-alkynyl, (C₃-C₈)-halocycloalkoxy, (C₂-C₈)-haloalkynyloxy, arylthio, heteroarylthio, (C₁-C₈)-alkylsulfinyl, (C₁-C₈)-haloalkylsulfinyl, arylsulfinyl, heteroarylsulfinyl, (C₁-C₈)-alkylsulfonyl, (C₁-C₈)-haloalkylsulfonyl, arylsulfonyl, heteroarylsulfonyl, thiocyanato, isothiocyanato, (C₃-C₈)-cycloalkylamino, (C₃-C₈)-cycloalkyl[(C₁-C₈)-alkyl]amino, (C₂-C₈)-alkenylamino,
R¹ and R² with the atoms to which they are attached form a fully saturated, partially saturated or unsaturated 5 to 7-membered ring which is optionally interrupted by heteroatoms and optionally substituted further,
R² and R³ with the atoms to which they are attached form a fully saturated, partially saturated or unsaturated 5 to 7-membered ring which is optionally interrupted by heteroatoms and optionally substituted further,
W represents oxygen, sulfur,
X represents oxygen, sulfur, N—R⁴,
R⁴ represents hydrogen, (C₁-C₈)-alkyl, (C₃-C₈)-cycloalkyl, (C₃-C₈)-cycloalkyl-(C₁-C₈)-alkyl, (C₄-C₈)-cycloalkenyl, cyano-(C₁-C₈)-alkyl, (C₂-C₈)-alkenyl-(C₁-C₈)-alkyl, (C₁-C₈)-haloalkyl, (C₂-C₈)-alkynyl-(C₁-C₈)-alkyl, aryl-(C₁-C₈)-alkyl, heteroaryl-(C₁-C₈)-alkyl, heterocyclyl-(C₁-C₈)-alkyl, (C₁-C₈)-alkylcarbonyl, (C₃-C₈)-cycloalkylcarbonyl, arylcarbonyl, heteroarylcarbonyl, (C₁-C₈)-haloalkylcarbonyl, heterocyclylcarbonyl, aryl-(C₁-C₈)-alkylcarbonyl, (C₁-C₈)-alkoxycarbonyl, (C₃-C₈)-cycloalkyl-(C₁-C₈)-alkoxycarbonyl, (C₃-C₈)-cycloalkoxycarbonyl, (C₁-C₈)-alkoxycarbonyl-(C₁-C₈)-alkyl, (C₁-C₈)-alkylsulfonyl, (C₃-C₈)-cycloalkylsulfonyl, arylsulfonyl, heteroarylsulfonyl, (C₁-C₈)-alkoxycarbonylcarbonyl, aryl-(C₁-C₈)-alkoxycarbonylcarbonyl, (C₁-C₈)-alkylaminothiocarbonyl, (C₁-C₈)-alkylaminocarbonyl, (C₃-C₈)-cycloalkylaminocarbonyl, (C₁-C₈)-alkoxy-(C₁-C₈)-alkyl, bis[(C₁-C₈)-alkyl]aminocarbonyl, aryl, heteroaryl, heterocyclyl, (C₃-C₈)-cycloalkoxycarbonyl-(C₁-C₈)-alkyl,
Q represents (C₁-C₈)-alkyl, (C₂-C₈)-alkenyl, (C₃-C₈)-cycloalkyl, (C₃-C₈)-cycloalkyl-(C₁-C₈)-alkyl, (C₄-C₈)-cycloalkenyl, optionally substituted phenyl, aryl-(C₁-C₈)-alkyl, heteroaryl-(C₁-C₈)-alkyl, heterocyclyl-(C₁-C₈)-alkyl, heteroaryl, heterocyclyl, heterocyclylaryl, heterocyclylheteroaryl, heteroarylheteroaryl, heteroarylaryl, arylaryl, aryloxyaryl, aryl-(C₂-C₈)-alkenyl, heteroaryl-(C₂-C₈)-alkenyl, heterocyclyl-(C₂-C₈)-alkenyl, aryl-(C₂-C₈)-alkynyl, heteroaryl-(C₂-C₈)-alkynyl, heterocyclyl-(C₁-C₈)-alkynyl, (C₃-C₈)-cycloalkyl-(C₂-C₈)-alkynyl, (C₁-C₈)-alkylamino-(C₁-C₈)-alkyl, bis[(C₁-C₈)-alkyl]amino-(C₁-C₈)-alkyl, hydroxy-(C₁-C₈)-alkyl, (C₁-C₈)-alkoxy-(C₁-C₈)-alkyl, tris[(C₁-C₈)-alkyl]silyloxy-(C₁-C₈)-alkyl, bis[(C₁-C₈)-alkyl]arylsilyloxy-(C₁-C₈)-alkyl, bis[(C₁-C₈)-alkyl]-(C₁-C₈)-alkylsilyloxy-(C₁-C₈)-alkyl, bis[(C₁-C₈)-alkyl]amino-(C₁-C₈)-alkoxy-(C₁-C₈)-alkyl, (C₁-C₈)-alkoxy-(C₁-C₈)-alkoxy-(C₁-C₈)-alkyl, aryloxy-(C₁-C₈)-alkyl, heteroaryloxy-(C₁-C₈)-alkyl, (C₁-C₈)-alkylthio-(C₁-C₈)-alkyl, arylthio-(C₁-C₈)-alkyl, heteroarylthio-(C₁-C₈)-alkyl, (C₁-C₈)-alkoxycarbonyl-N-heterocyclyl, aryl-(C₁-C₈)-alkoxycarbonyl-N-heterocyclyl, (C₁-C₈)-alkyl-N-heterocyclyl, (C₁-C₈)-alkylsulfonyl-N-heterocyclyl, arylsulfonyl-N-heterocyclyl, heteroarylsulfonyl-N-heterocyclyl, (C₃-C₈)-cycloalkylsulfonyl-N-heterocyclyl, (C₁-C₈)-haloalkylsulfonyl-N-heterocyclyl, (C₁-C₈)-alkylcarbonyl-N-heterocyclyl, arylcarbonyl-N-heterocyclyl, heteroarylcarbonyl-N-heterocyclyl, (C₃-C₈)-cycloalkylcarbonyl-N-heterocyclyl, (C₁-C₈)-cycloalkyl-N-heterocyclyl, aryl-N-heterocyclyl, aryl-(C₁-C₈)-alkyl-N-heterocyclyl, bis[(C₁-C₈)-alkyl]aminoalkyl-N-heterocyclyl, bis[(C₁-C₈)-alkyl]aminosulfonyl-N-heterocyclyl, heteroaryloxyaryl, heteroaryloxyheteroaryl, aryloxyheteroaryl, (C₁-C₈)-alkylsulfinyl, (C₁-C₈)-alkylthio, (C₁-C₈)-alkylsulfonyl, (C₃-C₈)-cycloalkylsulfinyl, (C₃-C₈)-cycloalkylthio, (C₃-C₈)-cycloalkylsulfonyl, arylsulfinyl, arylthio, arylsulfonyl, amino, (C₁-C₈)-alkylamino, bis[(C₁-C₈)-alkyl]amino, arylamino, aryl-(C₁-C₈)-alkylamino, (C₃-C₈)-cycloalkylamino, formyl, (C₁-C₈)-alkylcarbonyl, arylcarbonyl, imino-(C₁-C₈)-alkyl, (C₁-C₈)-alkylimino-(C₁-C₈)-alkyl, arylimino-(C₁-C₈)-alkyl, (C₁-C₈)-alkoxycarbonyl, (C₃-C₈)-cycloalkoxycarbonyl, (C₃-C₈)-cycloalkyl-(C₁-C₈)-alkoxycarbonyl, aryl-(C₁-C₈)-alkoxycarbonyl, aryl-(C₁-C₈)-alkylaminocarbonyl, aminocarbonyl, (C₁-C₈)-alkylaminocarbonyl, (C₃-C₈)-cycloalkylaminocarbonyl, bis[(C₁-C₈)-alkyl]aminocarbonyl, heterocyclyl-N-carbonyl, imino, (C₁-C₈)-alkylimino, arylimino, (C₃-C₈)-cycloalkylimino, (C₃-C₈)-cycloalkyl-(C₁-C₈)-alkylimino, hydroxyimino, (C₁-C₈)-alkoxyimino, (C₂-C₈)-alkenyloxyimino, (C₃-C₈)-cycloalkoxyimino, (C₃-C₈)-cycloalkyl-(C₁-C₈)-alkoxyimino, aryloxyimino, aryl-(C₁-C₈)-alkoxyimino, heteroaryl-(C₁-C₈)-alkoxyimino, heteroaryloxyimino, heteroarylimino, heterocyclylimino, heterocyclyl-(C₁-C₈)-alkylimino, aminoimino, (C₁-C₈)-alkylaminoimino, arylaminoimino, heteroarylaminoimino, (C₃-C₈)-cycloalkylaminoimino, bis[(C₁-C₈)-alkyl]aminoimino, aryl-(C₁-C₈)-alkylaminoimino, aryl[(C₁-C₈)-alkyl]aminoimino, (C₃-C₈)-cycloalkyl[(C₁-C₈)-alkyl]aminoimino, (C₃-C₈)-cycloalkyl-(C₁-C₈)-alkylaminoimino, heterocyclylaminoimino, heteroaryl-(C₁-C₈)-alkoxy-(C₁-C₈)-alkyl, aryl-(C₁-C₈)-alkoxy-(C₁-C₈)-alkyl, heterocyclyl-N—(C₁-C₈)-alkyl, aryl-[(C₁-C₈)-alkyl]amino-(C₁-C₈)-alkyl, aryl-(C₁-C₈)-alkyl[(C₁-C₈)-alkyl]amino-(C₁-C₈)-alkyl, (C₁-C₈)-alkoxycarbonyl-(C₁-C₈)-alkylamino-(C₁-C₈)-alkyl, (C₁-C₈)-alkoxycarbonyl-(C₁-C₈)-alkyl[(C₁-C₈)-alkyl]amino-(C₁-C₈)-alkyl, heteroaryl-[(C₁-C₈)-alkyl]amino-(C₁-C₈)-alkyl, heteroaryl-(C₁-C₈)-alkyl-[(C₁-C₈)-alkyl]amino-(C₁-C₈)-alkyl, —(C₃-C₈)-cycloalkyl-[(C₁-C₈)-alkyl]amino-(C₁-C₈)-alkyl, (C₃-C₈)-cycloalkylamino-(C₁-C₈)-alkyl, aryl-(C₁-C₈)-alkoxy-(C₁-C₈)-alkylaryl, heterocyclyl-N—(C₁-C₈)-alkylaryl, aryl[(C₁-C₈)-alkyl]amino-(C₁-C₈)-alkylaryl, aryl-(C₁-C₈)-alkyl[(C₁-C₈)-alkyl]amino-(C₁-C₈)-alkylaryl, (C₁-C₈)-alkoxycarbonyl-(C₁-C₈)-alkylamino-(C₁-C₈)-alkylaryl, (C₁-C₈)-alkoxycarbonyl-(C₁-C₈)-alkyl[(C₁-C₈)-alkyl]amino-(C₁-C₈)-alkylaryl, heteroaryl[(C₁-C₈)-alkyl]amino-(C₁-C₈)-alkylaryl, heteroaryl-(C₁-C₈)-alkyl[(C₁-C₈)-alkyl]amino-(C₁-C₈)-alkylaryl, (C₃-C₈)-cycloalkyl[(C₁-C₈)-alkyl]amino-(C₁-C₈)-alkylaryl, (C₃-C₈)-cycloalkylamino-(C₁-C₈)-alkylaryl, (C₁-C₈)-alkoxy[(C₁-C₈)-alkoxy]-(C₁-C₈)-alkylaryl,
Q additionally, if X represents an oxygen atom or X represents a sulfur atom, represents (C₁-C₈)-haloalkyl, (C₁-C₈)-alkoxy-(C₁-C₈)-haloalkyl, (C₃-C₈)-halocycloalkyl, (C₁-C₈)-haloalkoxy-(C₁-C₈)-haloalkyl, aryl-(C₁-C₈)-haloalkyl, (C₁-C₈)-alkylthio-(C₁-C₈)-haloalkyl, bis[(C₁-C₈)-alkyl]amino-(C₁-C₈)-alkoxy-(C₁-C₈)-haloalkyl,
Z¹ represents hydrogen, hydroxy, (C₁-C₈)-alkyl, (C₃-C₈)-cycloalkyl, (C₃-C₈)-halocycloalkyl, halogen, (C₂-C₈)-alkenyl-(C₁-C₈)-alkyl, (C₁-C₈)-haloalkyl, (C₂-C₈)-alkynyl, (C₂-C₈)-alkenyl, cyano-(C₁-C₈)-alkyl, nitro-(C₁-C₈)-alkyl, amino-(C₁-C₈)-alkyl, alkyl-(C₁-C₈)-amino-(C₁-C₈)-alkyl, bis[(C₁-C₈)-alkyl]amino-(C₁-C₈)-alkyl, (C₂-C₈)-alkynyl-(C₁-C₈)-alkyl, aryl-(C₁-C₈)-alkyl, heteroaryl-(C₁-C₈)-alkyl, heterocyclyl-(C₁-C₈)-alkyl, (C₁-C₈)-alkylcarbonyl, (C₁-C₈)-alkoxycarbonyl, (C₃-C₈)-cycloalkoxycarbonyl, (C₁-C₈)-alkylsulfonyl, arylsulfonyl, heteroarylsulfonyl, (C₃-C₈)-cycloalkylsulfonyl, (C₁-C₈)-alkylsulfinyl, arylsulfinyl, (C₃-C₈)-cycloalkylsulfinyl, (C₁-C₈)-alkoxycarbonyl-(C₁-C₈)-alkyl, aryl, heteroaryl, heterocyclyl, aminocarbonyl-(C₁-C₈)-alkyl, (C₁-C₈)-alkylaminocarbonyl-(C₁-C₈)-alkyl, bis[(C₁-C₈)-alkyl]aminocarbonyl-(C₁-C₈)-alkyl, (C₃-C₈)-cycloalkylaminocarbonyl-(C₁-C₈)-alkyl, hydroxycarbonyl-(C₁-C₈)-alkyl, amino, (C₁-C₈)-alkylamino, arylamino, (C₁-C₈)-alkoxy
and
Z² represents hydrogen, (C₁-C₈)-alkyl, (C₃-C₈)-cycloalkyl, (C₁-C₈)-haloalkyl, (C₂-C₈)-alkynyl, (C₂-C₈)-alkenyl, cyano-(C₁-C₈)-alkyl, aryl-(C₁-C₈)-alkyl, heteroaryl-(C₁-C₈)-alkyl, (C₁-C₈)-alkylcarbonyl, (C₁-C₈)-alkoxycarbonyl
or
Z¹ and Z² together with the atoms to which they are attached form a fully saturated or partially saturated 5 to 7-membered ring which is optionally interrupted by heteroatoms and optionally substituted further
or
Z¹ and Z² together form an N-(bis(C₁-C₈)-alkyl)sulfanylidene, N-(aryl-(C₁-C₈)-alkyl)sulfanylidene, N-(bis(C₃-C₈)-cycloalkyl)sulfanylidene, N—((C₁-C₈)-alkyl-(C₃-C₈)-cycloalkyl)sulfanylidene group or an N,N-di-(C₁-C₈)-alkylformylidene group.

3. The compound as claimed in claim 1, where, in formula (I),
R¹, R², R³ independently of one another represent hydrogen, iodine, bromine, chlorine, fluorine, (C₁-C₇)-alkyl, (C₃-C₇)-cycloalkyl, (C₄-C₇)-cycloalkenyl, (C₃-C₇)-halocycloalkyl, (C₂-C₇)-alkenyl, (C₂-C₇)-alkynyl, optionally substituted phenyl, aryl-(C₁-C₇)-alkyl, aryl-(C₂-C₇)-alkenyl, heteroaryl, heteroaryl-(C₁-C₇)-alkyl, heterocyclyl, heterocyclyl-(C₁-C₇)-alkyl, (C₁-C₇)-alkoxy-(C₁-C₇)-alkyl, (C₁-C₇)-alkylthio, (C₁-C₇)-haloalkylthio, (C₁-C₇)-haloalkyl, (C₁-C₇)-alkoxy, (C₁-C₇)-haloalkoxy, (C₃-C₇)-cycloalkoxy, (C₃-C₇)-cycloalkyl-(C₁-C₇)-alkoxy, aryloxy, heteroaryloxy, (C₁-C₇)-alkoxy-(C₁-C₇)-alkoxy, (C₂-C₇)-alkynyl-(C₁-C₇)-alkoxy, (C₂-C₇)-alkenyloxy, bis[(C₁-C₇)-alkyl]amino-(C₁-C₇)-alkoxy, tris[(C₁-C₇)-alkyl]silyl, bis[(C₁-C₇)-alkyl]arylsilyl, bis[(C₁-C₇)-alkyl]-(C₁-C₇)-alkylsilyl, tris[(C₁-C₇)-alkyl]silyl-(C₂-C₇)-alkynyl, aryl-(C₂-C₇)-alkynyl, heteroaryl-(C₂-C₇)-alkynyl, (C₁-C₇)-alkyl-(C₂-C₇)-alkynyl, (C₃-C₇)-cycloalkyl-(C₂-C₇)-alkynyl, (C₁-C₇)-haloalkyl-(C₂-C₇)-alkynyl, heterocyclyl-N—(C₁-C₇)-alkoxy, nitro, cyano, amino, (C₁-C₇)-alkylamino, bis[(C₁-C₇)-alkyl]amino, (C₁-C₇)-alkylcarbonylamino, (C₃-C₇)-cycloalkylcarbonylamino, arylcarbonylamino, (C₁-C₇)-alkoxycarbonylamino, heterocyclyl-(C₁-C₇)-alkoxy, (C₃-C₇)-cycloalkyl-(C₁-C₇)-alkyl, (C₂-C₇)-haloalkenyl, (C₂-C₇)-haloalkynyl, heterocyclyl-(C₂-C₇)-alkynyl, (C₃-C₇)-halocycloalkoxy, (C₂-C₇)-haloalkynyloxy, arylthio, heteroarylthio, (C₁-C₇)-alkylsulfinyl, (C₁-C₇)-haloalkylsulfinyl, arylsulfinyl, heteroarylsulfinyl, (C₁-C₇)-alkylsulfonyl, (C₁-C₇)-haloalkylsulfonyl, arylsulfonyl, heteroarylsulfonyl, thiocyanato, isothiocyanato, (C₃-C₇)-cycloalkylamino, (C₃-C₇)-cycloalkyl[(C₁-C₇)-alkyl]amino, (C₂-C₇)-alkenylamino,
R¹ and R² with the atoms to which they are attached form a fully saturated, partially saturated or unsaturated 5- to 7-membered ring which is optionally interrupted by heteroatoms and optionally substituted further,
R² and R³ with the atoms to which they are attached form a fully saturated, partially saturated or unsaturated 5- to 7-membered ring which is optionally interrupted by heteroatoms and optionally substituted further,
W represents oxygen, sulfur,
X represents oxygen, sulfur, N—R⁴,
R⁴ represents hydrogen, (C₁-C₇)-alkyl, (C₃-C₇)-cycloalkyl, (C₃-C₇)-cycloalkyl-(C₁-C₇)-alkyl, (C₄-C₇)-cycloalkenyl, cyano-(C₁-C₇)-alkyl, (C₂-C₇)-alkenyl-(C₁-C₇)-alkyl, (C₁-C₇)-haloalkyl, (C₂-C₇)-alkynyl-(C₁-C₇)-alkyl, aryl-(C₁-C₇)-alkyl, heteroaryl-(C₁-C₇)-alkyl, heterocyclyl-(C₁-C₇)-alkyl, (C₁-C₇)-alkylcarbonyl, (C₃-C₇)-cycloalkylcarbonyl, arylcarbonyl, heteroarylcarbonyl, (C₁-C₇)-haloalkylcarbonyl, heterocyclylcarbonyl, aryl-(C₁-C₇)-alkylcarbonyl, (C₁-C₇)-alkoxycarbonyl, (C₃-C₇)-cycloalkyl-(C₁-C₇)-alkoxycarbonyl, (C₃-C₇)-cycloalkoxycarbonyl, (C₁-C₇)-alkoxycarbonyl-(C₁-C₇)-alkyl, (C₁-C₇)-alkylsulfonyl, (C₃-C₇)-cycloalkylsulfonyl, arylsulfonyl, heteroarylsulfonyl, (C₁-C₇)-alkoxycarbonylcarbonyl, aryl-(C₁-C₇)-alkoxycarbonylcarbonyl, (C₁-C₇)-alkylaminothiocarbonyl, (C₁-C₇)-alkylaminocarbonyl, (C₃-C₇)-cycloalkylaminocarbonyl, (C₁-C₇)-alkoxy-(C₁-C₇)-alkyl, bis[(C₁-C₇)-alkyl]aminocarbonyl, aryl, heteroaryl, heterocyclyl, (C₃-C₇)-cycloalkoxycarbonyl-(C₁-C₇)-alkyl,
Q represents (C₁-C₇)-alkyl, (C₂-C₇)-alkenyl, (C₃-C₇)-cycloalkyl, (C₃-C₇)-cycloalkyl-(C₁-C₇)-alkyl, (C₄-C₇)-cycloalkenyl, optionally substituted phenyl, aryl-(C₁-C₇)-alkyl, heteroaryl-(C₁-C₇)-alkyl, heterocyclyl-(C₁-C₇)-alkyl, heteroaryl, heterocyclyl, heterocyclylaryl, heterocyclylheteroaryl, heteroarylheteroaryl, heteroarylaryl, arylaryl, aryloxyaryl, aryl-(C₂-C₇)-alkenyl, heteroaryl-(C₂-C₇)-alkenyl, heterocyclyl-(C₂-C₇)-alkenyl, aryl-(C₂-C₇)-alkynyl, heteroaryl-(C₂-C₇)-alkynyl, heterocyclyl-(C₁-C₇)-alkynyl, (C₃-C₇)-cycloalkyl-(C₂-C₇)-alkynyl, (C₁-C₇)-alkylamino-(C₁-C₇)-alkyl, bis[(C₁-C₇)-alkyl]amino-(C₁-C₇)-alkyl, hydroxy-(C₁-C₇)-alkyl, (C₁-C₇)-alkoxy-(C₁-C₇)-alkyl, tris[(C₁-C₇)-alkyl]silyloxy-(C₁-C₇)-alkyl, bis[(C₁-C₇)-alkyl]arylsilyloxy-(C₁-C₇)-alkyl, bis[(C₁-C₇)-alkyl]-(C₁-C₇)-alkylsilyloxy-(C₁-C₇)-alkyl, bis[(C₁-C₇)-alkyl]amino-(C₁-C₇)-alkoxy-(C₁-C₇)-alkyl, (C₁-C₇)-alkoxy-(C₁-C₇)-alkoxy-(C₁-C₇)-alkyl, aryloxy-(C₁-C₇)-alkyl, heteroaryloxy-(C₁-C₇)-alkyl, (C₁-C₇)-alkylthio-(C₁-C₇)-alkyl, arylthio-(C₁-C₇)-alkyl, heteroarylthio-(C₁-C₇)-alkyl, (C₁-C₇)-alkoxycarbonyl-N-heterocyclyl, aryl-(C₁-C₇)-alkoxycarbonyl-N-heterocyclyl, (C₁-C₇)-alkyl-N-heterocyclyl, (C₁-C₇)-alkylsulfonyl-N-heterocyclyl, arylsulfonyl-N-heterocyclyl, heteroarylsulfonyl-N-heterocyclyl, (C₃-C₇)-cycloalkylsulfonyl-N-heterocyclyl, (C₁-C₇)-haloalkylsulfonyl-N-heterocyclyl, (C₁-C₇)-alkylcarbonyl-N-heterocyclyl, arylcarbonyl-N-heterocyclyl, heteroarylcarbonyl-N-heterocyclyl, (C₃-C₇)-cycloalkylcarbonyl-N-heterocyclyl, (C₁-C₇)-cycloalkyl-N-heterocyclyl, aryl-N-heterocyclyl, aryl-(C₁-C₇)-alkyl-N-heterocyclyl, bis[(C₁-C₇)-alkyl]aminoalkyl-N-heterocyclyl, bis[(C₁-C₇)-alkyl]aminosulfonyl-N-heterocyclyl, heteroaryloxyaryl, heteroaryloxyheteroaryl, aryloxyheteroaryl, (C₁-C₇)-alkylsulfinyl, (C₁-C₇)-alkylthio, (C₁-C₇)-alkylsulfonyl, (C₃-C₇)-cycloalkylsulfinyl, (C₃-C₇)-cycloalkylthio, (C₃-C₇)-cycloalkylsulfonyl, arylsulfinyl, arylthio, arylsulfonyl, amino, (C₁-C₇)-alkylamino, bis[(C₁-C₇)-alkyl]amino, arylamino, aryl-(C₁-C₇)-alkylamino, (C₃-C₇)-cycloalkylamino, formyl, (C₁-C₇)-alkylcarbonyl, arylcarbonyl, imino-(C₁-C₇)-alkyl, (C₁-C₇)-alkylimino-(C₁-C₇)-alkyl, arylimino-(C₁-C₇)-alkyl, (C₁-C₇)-alkoxycarbonyl, (C₃-C₇)-cycloalkoxycarbonyl, (C₃-C₇)-cycloalkyl-(C₁-C₇)-alkoxycarbonyl, aryl-(C₁-C₇)-alkoxycarbonyl, aryl-(C₁-C₇)-alkylaminocarbonyl, aminocarbonyl, (C₁-C₇)-alkylaminocarbonyl, (C₃-C₇)-cycloalkylaminocarbonyl, bis[(C₁-C₇)-alkyl]aminocarbonyl, heterocyclyl-N-carbonyl, imino, (C₁-C₇)-alkylimino, arylimino, (C₃-C₇)-cycloalkylimino, (C₃-C₇)-cycloalkyl-(C₁-C₇)-alkylimino, hydroxyimino, (C₁-C₇)-alkoxyimino, (C₂-C₇)-alkenyloxyimino, (C₃-C₇)-cycloalkoxyimino, (C₃-C₇)-cyloalkyl-(C₁-C₇)-alkoxyimino, aryloxyimino, aryl-(C₁-C₇)-alkoxyimino, heteroaryl-(C₁-C₇)-alkoxyimino, heteroaryloxyimino, heteroarylimino, heterocyclylimino, heterocyclyl-(C₁-C₇)-alkylimino, aminoimino, (C₁-C₇)-alkylaminoimino, arylaminoimino, heteroarylaminoimino, (C₃-C₇)-cycloalkylaminoimino, bis[(C₁-C₇)-alkyl]aminoimino, aryl-(C₁-C₇)-alkylaminoimino, aryl[(C₁-C₇)-alkyl]aminoimino, (C₃-C₇)-cycloalkyl[(C₁-C₇)-alkyl]aminoimino, (C₃-C₇)-cycloalkyl-(C₁-C₇)-alkylaminoimino, heterocyclylaminoimino, heteroaryl-(C₁-C₇)-alkoxy-(C₁-C₇)-alkyl, aryl-(C₁-C₇)-alkoxy-(C₁-C₇)-alkyl, heterocyclyl-N—(C₁-C₇)-alkyl, aryl-[(C₁-C₇)-alkyl]amino-(C₁-C₇)-alkyl, aryl-(C₁-C₇)-alkyl[(C₁-C₇)-alkyl]amino-(C₁-C₇)-alkyl, (C₁-C₇)-alkoxycarbonyl-(C₁-C₇)-alkylamino-(C₁-C₇)-alkyl, (C₁-C₇)-alkoxycarbonyl-(C₁-C₇)-alkyl[(C₁-C₇)-alkyl]amino-(C₁-C₇)-alkyl, heteroaryl-[(C₁-C₇)-alkyl]amino-(C₁-C₇)-alkyl, heteroaryl-(C₁-C₇)-alkyl-[(C₁-C₇)-alkyl]amino-(C₁-C₇)-alkyl, —(C₃-C₇)-cycloalkyl-[(C₁-C₇)-alkyl]amino-(C₁-C₇)-alkyl, (C₃-C₇)-cycloalkylamino-(C₁-C₇)-alkyl, aryl-(C₁-C₇)-alkoxy-(C₁-C₇)-alkylaryl, heterocyclyl-N—(C₁-C₇)-alkylaryl, aryl[(C₁-C₇)-alkyl]amino-(C₁-C₇)-alkylaryl, aryl-(C₁-C₇)-alkyl[(C₁-C₇)-alkyl]amino-(C₁-C₇)-alkylaryl, (C₁-C₇)-alkoxycarbonyl-(C₁-C₇)-alkylamino-(C₁-C₇)-alkylaryl, (C₁-C₇)-alkoxycarbonyl-(C₁-C₇)-alkyl[(C₁-C₇)-alkyl]amino-(C₁-C₇)-alkylaryl, heteroaryl[(C₁-C₇)-alkyl]amino-(C₁-C₇)-alkylaryl, heteroaryl-(C₁-C₇)-alkyl[(C₁-C₇)-alkyl]amino-(C₁-C₇)-alkylaryl, (C₃-C₇)-cycloalkyl[(C₁-C₇)-alkyl]amino-(C₁-C₇)-alkylaryl, (C₃-C₇)-cycloalkylamino-(C₁-C₇)-alkylaryl, (C₁-C₇)-alkoxy[(C₁-C₇)-alkoxy]-(C₁-C₇)-alkylaryl,
Q additionally, if X represents an oxygen atom or X represents a sulfur atom, represents (C₁-C₇)-haloalkyl, (C₁-C₇)-alkoxy-(C₁-C₇)-haloalkyl, (C₃-C₇)-halocycloalkyl, (C₁-C₇)-haloalkoxy-(C₁-C₇)-haloalkyl, aryl-(C₁-C₇)-haloalkyl, (C₁-C₇)-alkylthio-(C₁-C₇)-haloalkyl, bis[(C₁-C₇)-alkyl]amino-(C₁-C₇)-alkoxy-(C₁-C₇)-haloalkyl,
Z¹ represents hydrogen, hydroxy, (C₁-C₇)-alkyl, (C₃-C₇)-cycloalkyl, (C₃-C₇)-halocycloalkyl, bromine, chlorine, (C₂-C₇)-alkenyl-(C₁-C₇)-alkyl, (C₁-C₇)-haloalkyl, (C₂-C₇)-alkynyl, (C₂-C₇)-alkenyl, cyano-(C₁-C₇)-alkyl, nitro-(C₁-C₇)-alkyl, amino-(C₁-C₇)-alkyl, alkyl-(C₁-C₇)-amino-(C₁-C₇)-alkyl, bis[(C₁-C₇)-alkyl]amino-(C₁-C₇)-alkyl, (C₂-C₇)-alkynyl-(C₁-C₇)-alkyl, aryl-(C₁-C₇)-alkyl, heteroaryl-(C₁-C₇)-alkyl, heterocyclyl-(C₁-C₇)-alkyl, (C₁-C₇)-alkylcarbonyl, (C₁-C₇)-alkoxycarbonyl, (C₃-C₇)-cycloalkoxycarbonyl, (C₁-C₇)-alkylsulfonyl, arylsulfonyl, heteroarylsulfonyl, (C₃-C₇)-cycloalkylsulfonyl, (C₁-C₇)-alkylsulfinyl, arylsulfinyl, (C₃-C₇)-cycloalkylsulfinyl, (C₁-C₇)-alkoxycarbonyl-(C₁-C₇)-alkyl, aryl, heteroaryl, heterocyclyl, aminocarbonyl-(C₁-C₇)-alkyl, (C₁-C₇)-alkylaminocarbonyl-(C₁-C₇)-alkyl, bis[(C₁-C₇)-alkyl]aminocarbonyl-(C₁-C₇)-alkyl, (C₃-C₇)-cycloalkylaminocarbonyl-(C₁-C₇)-alkyl, hydroxycarbonyl-(C₁-C₇)-alkyl, amino, (C₁-C₇)-alkylamino, arylamino, (C₁-C₇)-alkoxy
and
Z² represents hydrogen, (C₁-C₇)-alkyl, (C₃-C₇)-cycloalkyl, (C₁-C₇)-haloalkyl, (C₂-C₇)-alkynyl, (C₂-C₇)-alkenyl, cyano-(C₁-C₇)-alkyl, heteroaryl-(C₁-C₇)-alkyl, (C₁-C₇)-alkylcarbonyl, (C₁-C₇)-alkoxycarbonyl
or
Z¹ and Z² together with the atoms to which they are attached form a fully saturated or partially saturated 5 to 7-membered ring which is optionally interrupted by heteroatoms and optionally substituted further
or
Z¹ and Z² together form an N-(bis(C₁-C₇)-alkyl)sulfanylidene, N-(aryl-(C₁-C₇)-alkyl)sulfanylidene, N-(bis(C₃-C₇)-cycloalkyl)sulfanylidene, N—((C₁-C₇)-alkyl-(C₃-C₇)-cycloalkyl)sulfanylidene group or an N,N-di-(C₁-C₇)-alkylformylidene group.

4. A method of treatment for plants, comprising applying a nontoxic amount, effective for increasing resistance of one or more plants to abiotic stress factors, of one or more of the compound as claimed in claim 1.

5. The method of treatment as claimed in claim 4, wherein the abiotic stress conditions are one or more conditions selected from the group consisting of aridity, cold stress, heat stress, drought stress, osmotic stress, waterlogging, elevated soil salinity, elevated exposure to minerals, ozone conditions, strong light conditions, limited availability of nitrogen nutrients and limited availability of phosphorus nutrients.

6. The compound as claimed in claim 1 capable of being used in spray application to one or more of plants and/or parts of plants in combination with one or more active compounds selected from the group consisting of the insecticides, attractants, acaricides, fungicides, nematicides, herbicides, growth regulators, safeners, substances which affect plant maturity and bactericides.

7. The compound as claimed in claim 1 capable of being used in spray application to one or more plants and/or parts of plants in combination with fertilizer.

8. The compound as claimed in claim 1 capable of being used for application to one or more genetically modified cultivars, seed thereof, and/or to a cultivated area on which cultivars grow.

9. A spray solution comprising one or more of the compound as claimed in claim 1 capable of being used for enhancing resistance of one or more plants to one or more abiotic stress factors.

10. A method for increasing stress tolerance in plants selected from the group of useful plants, ornamental plants, turfgrasses and trees, which comprises applying a sufficient nontoxic amount of one or more of the compounds as claimed in claim 1 to an area where a corresponding effect is desired, to the plants, the seed thereof and/or to an area on which plants grow.

11. The method as claimed in claim 10, where the resistance of thus-treated plants to abiotic stress is increased by at least 3% in comparison with untreated plants under otherwise identical physiological conditions.

12. A haloalkyl-substituted 2-amidobenzoxazole or 2-amidobenzothiazole of formula (I) and/or a salt thereof 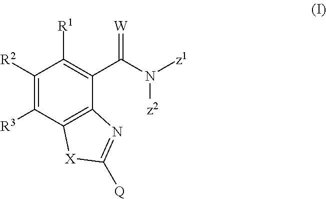
in which
R¹, R², R³ independently of one another represent hydrogen, halogen, (C₁-C₈)-alkyl, (C₃-C₈)-cycloalkyl, (C₄-C₈)-cycloalkenyl, (C₃-C₈)-halocycloalkyl, (C₂-C₈)-alkenyl, (C₂-C₈)-alkynyl, optionally substituted phenyl, aryl-(C₁-C₈)-alkyl, aryl-(C₂-C₈)-alkenyl, heteroaryl, heteroaryl-(C₁-C₈)-alkyl, heterocyclyl, heterocyclyl-(C₁-C₈)-alkyl, (C₁-C₈)-alkoxy-(C₁-C₈)-alkyl, (C₁-C₈)-alkylthio, (C₁-C₈)-haloalkylthio, (C₁-C₈)-haloalkyl, (C₁-C₈)-alkoxy, (C₁-C₈)-haloalkoxy, (C₃-C₈)-cycloalkoxy, (C₃-C₈)-cycloalkyl-(C₁-C₈)-alkoxy, aryloxy, heteroaryloxy, (C₁-C₈)-alkoxy-(C₁-C₈)-alkoxy, (C₂-C₈)-alkynyl-(C₁-C₈)-alkoxy, (C₂-C₈)-alkenyloxy, bis[(C₁-C₈)-alkyl]amino-(C₁-C₈)-alkoxy, tris[(C₁-C₈)-alkyl]silyl, bis[(C₁-C₈)-alkyl]arylsilyl, bis[(C₁-C₈)-alkyl]-(C₁-C₈)-alkylsilyl, tris[(C₁-C₈)-alkyl]silyl-(C₂-C₈)-alkynyl, aryl-(C₂-C₈)-alkynyl, heteroaryl-(C₂-C₈)-alkynyl, (C₁-C₈)-alkyl-(C₂-C₈)-alkynyl, (C₃-C₈)-cycloalkyl-(C₂-C₈)-alkynyl, (C₁-C₈)-haloalkyl-(C₂-C₈)-alkynyl, heterocyclyl-N—(C₁-C₈)-alkoxy, nitro, cyano, amino, (C₁-C₈)-alkylamino, bis[(C₁-C₈)-alkyl]amino, (C₁-C₈)-alkylcarbonylamino, (C₃-C₈)-cycloalkylcarbonylamino, arylcarbonylamino, (C₁-C₈)-alkoxycarbonylamino, heterocyclyl-(C₁-C₈)-alkoxy, (C₃-C₈)-cycloalkyl-(C₁-C₈)-alkyl, (C₂-C₈)-haloalkenyl, (C₂-C₈)-haloalkynyl, heterocyclyl-(C₂-C₈)-alkynyl, (C₃-C₈)-halocycloalkoxy, (C₂-C₈)-haloalkynyloxy, arylthio, heteroarylthio, (C₁-C₈)-alkylsulfinyl, (C₁-C₈)-haloalkylsulfinyl, arylsulfinyl, heteroarylsulfinyl, (C₁-C₈)-alkylsulfonyl, (C₁-C₈)-haloalkylsulfonyl, arylsulfonyl, heteroarylsulfonyl, thiocyanato, isothiocyanato, (C₃-C₈)-cycloalkylamino, (C₃-C₈)-cycloalkyl[(C₁-C₈)-alkyl]amino, (C₂-C₈)-alkenylamino,
R¹ and R² with the atoms to which they are attached form a fully saturated, partially saturated or unsaturated 5- to 7-membered ring which is optionally interrupted by heteroatoms and optionally substituted further,
R² and R³ with the atoms to which they are attached form a fully saturated, partially saturated or unsaturated 5- to 7-membered ring which is optionally interrupted by heteroatoms and optionally substituted further,
W represents oxygen, sulfur,
X represents oxygen, sulfur,
Q represents (C₁-C₈)-haloalkyl, (C₁-C₈)-alkoxy-(C₁-C₈)-haloalkyl, (C₃-C₈)-halocycloalkyl, (C₁-C₈)-haloalkoxy-(C₁-C₈)-haloalkyl, aryl-(C₁-C₈)-haloalkyl, (C₁-C₈)-alkylthio-(C₁-C₈)-haloalkyl, bis[(C₁-C₈)-alkyl]amino-(C₁-C₈)-alkoxy-(C₁-C₈)-haloalkyl,
Z¹ represents hydrogen, hydroxy, (C₁-C₈)-alkyl, (C₃-C₈)-cycloalkyl, (C₃-C₈)-halocycloalkyl, halogen, (C₂-C₈)-alkenyl-(C₁-C₈)-alkyl, (C₁-C₈)-haloalkyl, (C₂-C₈)-alkynyl, (C₂-C₈)-alkenyl, cyano-(C₁-C₈)-alkyl, nitro-(C₁-C₈)-alkyl, amino-(C₁-C₈)-alkyl, alkyl-(C₁-C₈)-amino-(C₁-C₈)-alkyl, bis[(C₁-C₈)-alkyl]amino-(C₁-C₈)-alkyl, (C₂-C₈)-alkynyl-(C₁-C₈)-alkyl, aryl-(C₁-C₈)-alkyl, heteroaryl-(C₁-C₈)-alkyl, heterocyclyl-(C₁-C₈)-alkyl, (C₁-C₈)-alkylcarbonyl, (C₁-C₈)-alkoxycarbonyl, (C₃-C₈)-cycloalkoxycarbonyl, (C₁-C₈)-alkylsulfonyl, arylsulfonyl, heteroarylsulfonyl, (C₃-C₈)-cycloalkylsulfonyl, (C₁-C₈)-alkylsulfinyl, arylsulfinyl, (C₃-C₈)-cycloalkylsulfinyl, (C₁-C₈)-alkoxycarbonyl-(C₁-C₈)-alkyl, aryl, heteroaryl, heterocyclyl, aminocarbonyl-(C₁-C₈)-alkyl, (C₁-C₈)-alkylaminocarbonyl-(C₁-C₈)-alkyl, bis[(C₁-C₈)-alkyl]aminocarbonyl-(C₁-C₈)-alkyl, (C₃-C₈)-cycloalkylaminocarbonyl-(C₁-C₈)-alkyl, hydroxycarbonyl-(C₁-C₈)-alkyl, amino, (C₁-C₈)-alkylamino, aryl amino, (C₁-C₈)-alkoxy
and
Z² represents hydrogen, (C₁-C₈)-alkyl, (C₃-C₈)-cycloalkyl, (C₁-C₈)-haloalkyl, (C₂-C₈)-alkynyl, (C₂-C₈)-alkenyl, cyano-(C₁-C₈)-alkyl, aryl-(C₁-C₈)-alkyl, heteroaryl-(C₁-C₈)-alkyl, (C₁-C₈)-alkylcarbonyl, (C₁-C₈)-alkoxycarbonyl
or
Z¹ and Z² together with the atoms to which they are attached form a fully saturated or partially saturated 5 to 7-membered ring which is optionally interrupted by heteroatoms and optionally substituted further
or
Z¹ and Z² together form an N-(bis(C₁-C₆)-alkyl)sulfanylidene, N-(aryl-(C₁-C₆)-alkyl)sulfanylidene, N-(bis(C₃-C₇)-cycloalkyl)sulfanylidene, N—((C₁-C₆)-alkyl-(C₃-C₇)-cycloalkyl)sulfanylidene group or an N,N-di-(C₁-C₆)-alkylformylidene group.

13. The haloalkyl-substituted 2-amidobenzoxazole or 2-amidobenzothiazole and/or salt as claimed in claim 12
in which
R¹, R², R³ independently of one another represent hydrogen, halogen, (C₁-C₄)-alkyl, (C₃-C₈)-cycloalkyl, (C₄-C₈)-cycloalkenyl, (C₃-C₈)-halocycloalkyl, (C₂-C₄)-alkenyl, (C₂-C₄)-alkynyl, optionally substituted phenyl, aryl-(C₁-C₄)-alkyl, aryl-(C₂-C₄)-alkenyl, heteroaryl, heteroaryl-(C₁-C₄)-alkyl, heterocyclyl, heterocyclyl-(C₁-C₄)-alkyl, (C₁-C₄)-alkoxy-(C₁-C₄)-alkyl, (C₁-C₄)-alkylthio, (C₁-C₄)-haloalkylthio, (C₁-C₄)-haloalkyl, (C₁-C₄)-alkoxy, (C₁-C₄)-halo alkoxy, (C₃-C₈)-cycloalkoxy, (C₃-C₈)-cycloalkyl-(C₁-C₄)-alkoxy, aryloxy, heteroaryloxy, (C₁-C₄)-alkoxy-(C₁-C₄)-alkoxy, (C₂-C₄)-alkynyl-(C₁-C₄)-alkoxy, (C₂-C₄)-alkenyloxy, bis[(C₁-C₄)-alkyl]amino-(C₁-C₄)-alkoxy, tris[(C₁-C₄)-alkyl]silyl, bis[(C₁-C₄)-alkyl]arylsilyl, bis[(C₁-C₄)-alkyl]-(C₁-C₄)-alkylsilyl, tris[(C₁-C₄)-alkyl]silyl-(C₂-C₄)-alkynyl, aryl-(C₂-C₄)-alkynyl, heteroaryl-(C₂-C₄)-alkynyl, (C₁-C₄)-alkyl-(C₂-C₄)-alkynyl, (C₃-C₈)-cycloalkyl-(C₂-C₄)-alkynyl, (C₁-C₄)-haloalkyl-(C₂-C₄)-alkynyl, heterocyclyl-N—(C₁-C₄)-alkoxy, nitro, cyano, amino, (C₁-C₄)-alkylamino, bis[(C₁-C₄)-alkyl]amino, (C₁-C₄)-alkylcarbonylamino, (C₃-C₈)-cycloalkylcarbonylamino, arylcarbonylamino, (C₁-C₄)-alkoxycarbonyl amino, heterocyclyl-(C₁-C₄)-alkoxy, (C₃-C₈)-cycloalkyl-(C₁-C₄)-alkyl, (C₂-C₄)-haloalkenyl, (C₂-C₄)-haloalkynyl, heterocyclyl-(C₂-C₄)-alkynyl, (C₃-C₈)-halocycloalkoxy, (C₂-C₄)-haloalkynyloxy, arylthio, heteroarylthio, (C₁-C₄)-alkylsulfinyl, (C₁-C₄)-haloalkylsulfinyl, arylsulfinyl, heteroarylsulfinyl, (C₁-C₄)-alkylsulfonyl, (C₁-C₄)-haloalkylsulfonyl, arylsulfonyl, heteroarylsulfonyl, thiocyanato, isothiocyanato, (C₃-C₈)-cycloalkylamino, (C₃-C₈)-cycloalkyl[(C₁-C₄)-alkyl]amino, (C₂-C₄)-alkenylamino,
R¹ and R² with the atoms to which they are attached form a fully saturated, partially saturated or unsaturated 5- to 7-membered ring which is optionally interrupted by heteroatoms and optionally substituted further,
R² and R³ with the atoms to which they are attached form a fully saturated, partially saturated or unsaturated 5- to 7-membered ring which is optionally interrupted by heteroatoms and optionally substituted further,
W represents oxygen, sulfur,
X represents oxygen, sulfur,
Q represents (C₁-C₇)-haloalkyl, (C₁-C₄)-alkoxy-(C₁-C₄)-haloalkyl, (C₃-C₈)-halocycloalkyl, (C₁-C₄)-haloalkoxy-(C₁-C₄)-haloalkyl, aryl-(C₁-C₄)-haloalkyl, (C₁-C₄)-alkylthio-(C₁-C₄)-haloalkyl, bis[(C₁-C₄)-alkyl]amino-(C₁-C₄)-alkoxy-(C₁-C₄)-haloalkyl,
Z¹ represents hydrogen, hydroxy, (C₁-C₄)-alkyl, (C₃-C₈)-cycloalkyl, (C₃-C₈)-halocycloalkyl, halogen, (C₂-C₄)-alkenyl-(C₁-C₄)-alkyl, (C₁-C₄)-haloalkyl, (C₂-C₄)-alkynyl, (C₂-C₄)-alkenyl, cyano-(C₁-C₄)-alkyl, nitro-(C₁-C₄)-alkyl, amino-(C₁-C₄)-alkyl, alkyl-(C₁-C₄)-amino-(C₁-C₄)-alkyl, bis[(C₁-C₄)-alkyl]amino-(C₁-C₄)-alkyl, (C₂-C₄)-alkynyl-(C₁-C₄)-alkyl, aryl-(C₁-C₄)-alkyl, heteroaryl-(C₁-C₄)-alkyl, heterocyclyl-(C₁-C₄)-alkyl, (C₁-C₄)-alkylcarbonyl, (C₁-C₄)-alkoxycarbonyl, (C₃-C₈)-cycloalkoxycarbonyl, (C₁-C₄)-alkylsulfonyl, arylsulfonyl, heteroarylsulfonyl, (C₃-C₆)-cycloalkylsulfonyl, (C₁-C₄)-alkylsulfinyl, arylsulfinyl, (C₃-C₈)-cycloalkylsulfinyl, (C₁-C₄)-alkoxycarbonyl-(C₁-C₄)-alkyl, aryl, heteroaryl, heterocyclyl, aminocarbonyl-(C₁-C₄)-alkyl, (C₁-C₄)-alkylaminocarbonyl-(C₁-C₄)-alkyl, bis[(C₁-C₄)-alkyl]aminocarbonyl-(C₁-C₄)-alkyl, (C₃-C₈)-cycloalkylaminocarbonyl-(C₁-C₄)-alkyl, hydroxycarbonyl-(C₁-C₄)-alkyl, amino, (C₁-C₄)-alkylamino, arylamino, (C₁-C₄)-alkoxy
and
Z² represents hydrogen, (C₁-C₄)-alkyl, (C₃-C₈)-cycloalkyl, (C₁-C₄)-haloalkyl, (C₂-C₄)-alkynyl, (C₂-C₄)-alkenyl, cyano-(C₁-C₄)-alkyl, aryl-(C₁-C₄)-alkyl, heteroaryl-(C₁-C₄)-alkyl, (C₁-C₄)-alkylcarbonyl, (C₁-C₄)-alkoxycarbonyl
or
Z¹ and Z² together with the atoms to which they are attached form a fully saturated or partially saturated 5 to 7-membered ring which is optionally interrupted by heteroatoms and optionally substituted further
or
Z¹ and Z² together form an N-(bis(C₁-C₆)-alkyl)sulfanylidene, N-(aryl-(C₁-C₆)-alkyl)sulfanylidene, N-(bis(C₃-C₇)-cycloalkyl)sulfanylidene, N—((C₁-C₆)-alkyl-(C₃-C₇)-cycloalkyl)sulfanylidene group or an N,N-di-(C₁-C₆)-alkylformylidene group.

14. A spray solution for treatment of plants, comprising an amount, effective for enhancing the resistance of plants to abiotic stress factors, of at least one of haloalkyl-substituted 2-amidobenzoxazole or 2-amidobenzothiazole and/or salt thereof as claimed in claim 12.
